(19) United States
(12) Patent Application Publication
(10) Pub. No.: US 2025/0320493 A1
(43) Pub. Date: Oct. 16, 2025

(54) METHODS FOR REDUCING TAU EXPRESSION

(71) Applicant: Biogen MA Inc., Cambridge, MA (US)

(72) Inventors: Daniel A. Norris, Carlsbad, CA (US); Holly Kordasiewicz, Carlsbad, CA (US); Roger Lane, Carlsbad, CA (US); Lin Lin, Cambridge, MA (US)

(21) Appl. No.: 18/291,214

(22) PCT Filed: Jul. 21, 2022
(86) PCT No.: PCT/US2022/074014

§ 371 Date: Jan. 22, 2024

Related U.S. Application Data
(60) Provisional application No. 63/225,404, filed on Jul. 23, 2021.
(60) Provisional application No. 63/246,706, filed on Sep. 21, 2021.
(60) Provisional application No. 63/331,650, filed on Apr. 15, 2022.
(60) Provisional application No. 63/345,511, filed on May 25, 2022.

(51) Int. Cl. C12N 15/113; A61P 25/28

(52) U.S. Cl. CPC C12N 15/113; A61P 25/28; C12N 2310/11; C12N 2310/315; C12N 2310/3341; C12N 2320/32; C12N 2320/35

(57) ABSTRACT

Provided herein are methods of administering ISIS 814907 for ameliorating Alzheimer's disease, reducing Tau RNA, or reducing Tau protein in a human subject in need thereof. In certain embodiments, the Alzheimer's disease is mild Alzheimer's disease, Mild Cognitive Impairment (MCI) Due to Alzheimer's Disease, and/or Alzheimer's Disease Dementia (e.g., Mild Alzheimer's Disease Dementia). In certain instances, methods are useful for ameliorating at least one symptom or hallmark of a disease or disorder associated with Tau protein. In certain instances, the disease or disorder associated with Tau protein is a neurodegenerative disease or disorder. In certain instances, the disease or disorder associated with Tau protein is Alzheimer's disease or Fronto-temporal Dementia (FTD). In certain embodiments, the Alzheimer's disease is mild Alzheimer's disease, Mild Cognitive Impairment (MCI) Due to Alzheimer's Disease, and/or Alzheimer's Disease Dementia (e.g., Mild Alzheimer's Disease Dementia). In certain instances, the disease or disorder associated with Tau protein is a tauopathy. In certain instances, the disease or disorder associated with Tau protein is Frontotemporal Dementia with Parkinsonism-17 (FTDP-17), Progressive Supranuclear Palsy (PSP), Chronic Traumatic Encephalopathy (CTE), Corticobasal Ganglionic Degeneration (CBD), Pick Disease, Argyrophilic Grain Disease (AGD), Globular Glial Tauopathies, Epilepsy, and/or Dravet's Syndrome. Such symptoms or hallmarks include loss of memory, cognitive decline, loss of ability to understand or express speech, abnormal behavior, loss of and impaired motor function, or increase in the number and/or volume of neurofibrillary inclusions.

### CROSS-REFERENCE TO RELATED APPLICATIONS

This application is a U.S. national stage application under 35 U.S.C. § 371 of International PCT Application No. PCT/US2022/074014, filed Jul. 21, 2022, which claims the benefit of priority to U.S. Provisional Appl. Nos. 63/225,404; 63/246,706; 63/331,650; and 63/345,511, filed on Jul. 23, 2021; Sep. 21, 2021; Apr. 15, 2022; and May 25, 2022, respectively, each of which are incorporated by reference in their entireties herein.

### SEQUENCE LISTING

This application contains a Sequence Listing that has been submitted electronically as an XML file named 13751-0362WO1.xml. The XML file, created on Jul. 11, 2022, is 149,519 bytes in size. The material in the XML file is hereby incorporated by reference in its entirety.

### FIELD

Provided herein are methods of administering ISIS 814907 for ameliorating Alzheimer's disease, reducing Tau RNA, or reducing Tau protein in a human subject in need thereof. In certain instances, methods are useful for ameliorating at least one symptom or hallmark of a disease or disorder associated with Tau protein. In certain instances, the disease or disorder associated with Tau protein is a neurodegenerative disease or disorder. In certain instances, the disease or disorder associated with Tau protein is Alzheimer's disease or Fronto-temporal Dementia (FTD). In some embodiments, the Alzheimer's disease is mild Alzheimer's disease, Mild Cognitive Impairment (MCI) Due to Alzheimer's disease, and/or Alzheimer's Disease Dementia (e.g., Mild Alzheimer's Disease Dementia). In certain instances, the disease or disorder associated with Tau protein is a tauopathy. In certain instances, the disease or disorder associated with Tau protein is Frontotemporal Dementia with Parkinsonism-17 (FTDP-17), Progressive Supranuclear Palsy (PSP), Chronic Traumatic Encephalopathy (CTE), Corticobasal Ganglionic Degeneration (CBD), Pick Disease, Argyrophilic Grain Disease (AGD), Globular Glial Tauopathies, Epilepsy, and/or Dravet's Syndrome. Symptoms or hallmarks of a disease or disorder associated with Tau protein include loss of memory, cognitive decline, loss of ability to understand or express speech, abnormal behavior, impaired motor function, or increase in the number and/or volume of neurofibrillary inclusions.

### BACKGROUND

Alzheimer's disease (AD) is a progressive neurodegenerative disorder characterized by cognitive and functional decline resulting in significant disability (Lane, et al., 2018, Eur. J. Neurol. 25: 59-70). Symptom onset typically occurs in patients aged 65 and older, while symptom onset before age 65 comprises <5% of all patients with AD (Alzheimer's Association, 2021, 2021 Alzheimer's disease facts and figures, Alzheimers Dement. 17: 327-406). Current management for AD is limited to multidisciplinary management of symptoms including pharmacological therapies. Accumulating evidence suggests that aggregated, hyperphosphorylated Tau may be a key driver of neurodegeneration in AD. Tau protein is encoded by the MAPT gene and is a microtubule-associated protein primarily expressed in neurons (Dixit, et al., 2008, Science 319: 1086-1089). Under pathogenic conditions, hyperphosphorylated Tau accumulates intracellularly and extracellularly, and aggregates into oligomers and fibrils resulting in intraneuronal neurofibrillary tangles (NFTs), which spread through specific neural networks via a trans-synaptic route (Braak and Del Tredici, 2016, Cold Spring Harb. Perspect. Biol. 8: a023630; Ossenkoppele, et al., 2019, Neuroimage Clin. 23: 101848) and cause neurodegeneration, synaptic dysfunction and synaptic loss (DeVos, et al., 2018, Front. Neurosci. 12: 267; Guo, et al., 2018, Acta Neuropathologica 133: 665-704; Wilcock, et al., 1982, J Neurol. Sci. 56: 343-56; Hanseeuw, et al. 2019, JAMA Neurol. 76: 915-924; Gordon, et al., 2019, Brain 142: 1063-1076). Neuronal Tau inclusions are a pathological characteristic of other neurodegenerative diseases or disorders, including tauopathies, Fronto-temporal Dementia (FTD), Progressive Supranuclear Palsy (PSP), FTDP-17, Chronic Traumatic Encephalopathy (CTE), Corticobasal Degeneration (CBD), Epilepsy, and Dravet's Syndrome (G. G. Kovacs. Chapter 25—Tauopathies. In Handbook of Clinical Neurology, Vol. 145 (3rd series). 2018).

### BRIEF DESCRIPTION OF THE DRAWINGS

FIG. 1A, FIG. 1B, FIG. 1C, FIG. 1D, and FIG. 1E show the concentrations of total Tau protein in CSF over time for individual patients in each dose group; absolute values, measured in picograms per milliliter (pg/mL).

FIG. 2A, FIG. 2B, FIG. 2C, FIG. 2D, and FIG. 2E show the percentage changes from baseline of total Tau protein. Arrowheads indicate the days on which ISIS 814907 or placebo was administered.

FIG. 3 shows the percentage change in the concentration of total Tau protein in CSF from baseline to the last available time point 56 days after the last dose (Day 141). Circles indicate individual patients, and horizontal lines indicate group means.

FIG. 4A shows the mean concentration of total Tau protein in CSF, FIG. 4B shows the mean percentage change from baseline of total Tau protein in CSF, and FIG. 4C shows the mean percentage change from baseline of phospho-Tau protein over time according to dose group. Error bars indicate the standard error of the mean.

### SUMMARY

Provided herein are methods for ameliorating diseases or disorders associated with Tau protein, and methods of reducing Tau RNA and/or Tau protein in a human subject in need thereof. Also provided herein are methods of treating or preventing a disease or disorder associated with Tau protein. In certain embodiments, the disease or disorder associated with Tau protein is a neurodegenerative disease or disorder. In certain embodiments, the disease or disorder associated with Tau protein is a tauopathy. In certain embodiments, the disease or disorder associated with Tau protein is Alzheimer's disease or Fronto-temporal Dementia (FTD). In some embodiments, the Alzheimer's disease is mild Alzheimer's disease, Mild Cognitive Impairment (MCI) Due to Alzheimer's Disease, and/or Alzheimer's Disease Dementia (e.g., Mild Alzheimer's Disease Dementia). In certain instances, the disease or disorder associated with Tau protein is Frontotemporal Dementia with Parkinsonism-17 (FTDP-17), Progressive Supranuclear Palsy (PSP), Chronic Traumatic Encephalopathy (CTE), Corticobasal Ganglionic Degeneration (CBD), Pick Disease, Argyrophilic Grain Disease (AGD), Globular Glial Tauopathies, Epilepsy, and/or Dravet's Syndrome. In certain embodiments, the disease or disorder associated with Tau protein is Alzheimer's disease. In some embodiments, the Alzheimer's disease is mild Alzheimer's disease, Mild Cognitive Impairment (MCI) Due to Alzheimer's Disease, and/or Alzheimer's Disease Dementia (e.g., Mild Alzheimer's Disease Dementia). In certain embodiments, the disease or disorder associated with Tau protein is FTD. In certain embodiments, methods comprise administering a therapeutically effective amount of a modified oligonucleotide. In certain embodiments, the modified oligonucleotide is ISIS 814907. In certain embodiments, the therapeutically effective amount is within the range of about 10 mg to about 115 mg. In certain embodiments, the therapeutically effective amount is within the range of about 60 mg to about 115 mg. In certain embodiments, the therapeutically effective amount is about 10 mg, about 30 mg, about 60 mg, about 90 mg, or about 115 mg. In certain embodiments, the therapeutically effective amount is administered once about every 4 weeks. In certain embodiments, the therapeutically effective amount is administered once monthly. In certain embodiments, the therapeutically effective amount is administered once every other month. In certain embodiments, the therapeutically effective amount is administered once every quarter. In certain embodiments, the therapeutically effective amount is administered once about every 8 weeks. In certain embodiments, the therapeutically effective amount is administered once about every 12 weeks. In certain embodiments, the therapeutically effective amount is administered once about every 16 weeks. In certain embodiments, the therapeutically effective amount is administered once about every 24 weeks. In certain embodiments, the therapeutically effective amount is administered once about every 6 months. In certain embodiments, the therapeutically effective amount is administered monthly. In certain embodiments, the therapeutically effective amount is administered once every two months. In certain embodiments, the therapeutically effective amount is administered once every three months. In certain embodiments, the therapeutically effective amount is administered quarterly. In certain embodiments, the therapeutically effective amount is administered twice a year (semiannually). In certain embodiments, the therapeutically effective amount is administered annually. In certain embodiments, the therapeutically effective amount is administered once every two years.

In some embodiments, the disclosure features methods of treating Alzheimer's disease in a human subject in need thereof, the method comprising administering to the human subject a dose of ISIS 814907. In some embodiments, the Alzheimer's disease is mild Alzheimer's disease, Mild Cognitive Impairment (MCI) Due to Alzheimer's Disease, and/or Alzheimer's Disease Dementia (e.g., Mild Alzheimer's Disease Dementia). In some embodiments, the dose is 10 mg of ISIS 814907 monthly. In some embodiments, the dose is 30 mg of ISIS 814907 monthly. In some embodiments, the dose is 60 mg of ISIS 814907 monthly. In some embodiments, the dose is 115 mg of ISIS 814907 once every 3 months, once every quarter, or four times yearly. The administration is performed intrathecally (e.g., bolus IT administrations). In some embodiments, the human subject is administered 1, 2, 3, 4, 5, 6, 7, 8, 9, 10, 11, 12, 13, 14, 15, 16, 17, 18, 19, 20, 21, 22, 23, 24, or more than 24 monthly doses. In some embodiments, the human subject is administered 1, 2, 3, 4, 5, 6, 7, 8, 9, 10, 11, 12, 13, 14, 15, 16, 17, 18, 19, 20, 21, 22, 23, 24, or more than 24 quarterly doses. In some embodiments, the human subject is over 90 years of age. In some embodiments, the human subject is between 40 and 90 years of age. In some embodiments, the human subject is between 50 and 80 years of age. In some embodiments, the human subject is 50 to 74 years of age. In some embodiments, the human subject has one or more (1, 2, 3, 4, 5, 6, 7, 8, 9, or 10) of the following criteria: 1) has mild Alzheimer's disease; 2) Repeatable Battery for the Assessment of Neuropsychological Status (RBANS) Delayed Memory Index score of ≤85, indicative of objective evidence of memory impairment; 3) CDR global score of 0.5 for MCI due to AD or 0.5 or 1 for mild AD dementia; 4) CDR Overall Global Score of 1 or Global Score of 0.5 with a Memory Score of 1; 5) MMSE score of 22 to 30 (inclusive); 6) MMSE score of 20-27 inclusive; 7) CDR Memory Box score of ≥0.5; 8) Evidence of amyloid pathology as measured by positive emission tomography (PET) or cerebrospinal fluid (CSF) sampling; 9) cerebrospinal fluid (CSF) pattern of low Aβ42 and elevated t-tau and phosphorylated tau (p-tau); and 10) diagnosis of probable Alzheimer's Disease based on National Institute of Aging-Alzheimer Association (NIA-AA) criteria.

In some embodiments, the disclosure features other methods of treating Alzheimer's disease in a human subject in need thereof, the method comprising administering to the human subject a dose of ISIS 814907. In some embodiments, the Alzheimer's disease is mild Alzheimer's disease, Mild Cognitive Impairment (MCI) Due to Alzheimer's Disease, and/or Alzheimer's Disease Dementia (e.g., Mild Alzheimer's Disease Dementia). In some embodiments, the dose is 60 mg of ISIS 814907. In some embodiments, the dose is 115 mg of ISIS 814907. In some embodiments, the dose is administered once ever 24 weeks (Q24W). In some embodiments, the dose is administered once ever 12 weeks (Q12W). In some embodiments, the dose of 60 mg ia administered Q12W. In some embodiments, the dose of 60 mg is administered Q24W. In some embodiments, the dose of 115 mg is administered Q24W. In some embodiments, the dose of 115 mg is administered Q12W. The administration is performed intrathecally (e.g., bolus IT administrations). In some embodiments, the human subject is administered 1, 2, 3, 4, 5, 6, 7, 8, 9, 10, 11, 12, 13, 14, 15, 16, 17, 18, 19, 20, 21, 22, 23, 24, or more than 24 doses Q12W. In some embodiments, the human subject is administered 1, 2, 3, 4, 5, 6, 7, 8, 9, 10, 11, 12, 13, 14, 15, 16, 17, 18, 19, 20, 21, 22, 23, 24, or more than 24 doses Q24W. In some embodiments, the human subject is over 90 years of age. In some embodiments, the doses are administered for up to 72 weeks. In some embodiments, the human subject is between 40 and 90 years of age. In some embodiments, the human subject is between 50 and 80 years of age. In some embodiments, the human subject is 50 to 72 years of age. In some embodiments, the human subject has one or more (1, 2, 3, 4, 5, 6, 7, 8, 9, or 10) of the following criteria: 1) has mild Alzheimer's disease; 2) Repeatable Battery for the Assessment of Neuropsychological Status (RBANS) Delayed Memory Index score of ≤85, indicative of objective evidence of memory impairment; 3) CDR global score of 0.5 for MCI due to AD or 0.5 or 1 for mild AD dementia; 4) CDR Overall Global Score of 1 or Global Score of 0.5 with a Memory Score of 1; 5) MMSE score of 22 to 30 (inclusive); 6) MMSE score of 20-27 inclusive; 7) CDR Memory Box score of ≥0.5; and 8) Evidence of amyloid pathology as measured by positive emission tomography (PET) or cerebrospinal fluid (CSF) sampling; 9) cerebrospinal fluid (CSF) pattern of low A042 and elevated t-tau and phosphorylated tau (p-tau); and 10) diagnosis of probable Alzheimer's Disease based on National Institute of Aging-Alzheimer Association (NIA-AA) criteria.

In certain embodiments, methods comprise administering a loading dose of about 10 mg, about 30 mg, about 60 mg, about 90 mg, or about 115 mg of ISIS 814907 once about every 4 weeks, and subsequently administering a maintenance dose of about 10 mg, about 30 mg, about 60 mg, about 90 mg, or about 115 mg of ISIS 814907 once about every 8 weeks, once about every 16 weeks, once about every 24 weeks, or once about every 6 months. In certain embodiments, methods comprise administering a loading dose of about 10 mg, about 30 mg, about 60 mg, about 90 mg, or about 115 mg of ISIS 814907 once about every 8 weeks, and subsequently administering a maintenance dose of about 10 mg, about 30 mg, about 60 mg, about 90 mg, or about 115 mg of ISIS 814907 once about every 16 weeks, once about every 24 weeks, or once about every 6 months. In certain embodiments, methods comprise administering a loading dose of about 10 mg, about 30 mg, about 60 mg, about 90 mg, or about 115 mg of ISIS 814907 once about every 12 weeks, and subsequently administering a maintenance dose of about 10 mg, about 30 mg, about 60 mg, about 90 mg, or about 115 mg of ISIS 814907 once about every 16 weeks, once about every 24 weeks, or once about every 6 months. In some embodiments, methods comprise administering at least 2 loading doses, at least 3 loading doses, at least 4 loading doses, at least 5 loading doses, or at least 6 loading doses.

In certain embodiments, methods comprise administering a loading dose of about 60 mg to about 115 mg once about every 4 weeks, and subsequently administering a maintenance dose of about 60 mg to about 115 mg of ISIS 814907 once about every 8 weeks, once about every 16 weeks, once about every 24 weeks, or once about every 6 months. In certain embodiments, methods comprise administering a loading dose of about 60 mg to about 115 mg of ISIS 814907 once about every 8 weeks, and subsequently administering a maintenance dose of about 10 mg, about 30 mg, about 60 mg, about 90 mg, or about 115 mg of ISIS 814907 once about every 16 weeks, once about every 24 weeks, or once about every 6 months. In certain embodiments, methods comprise administering a loading dose of about 60 mg to about 115 mg of ISIS 814907 once about every 12 weeks, and subsequently administering a maintenance dose of about 60 mg to about 115 mg of ISIS 814907 once about every 16 weeks, once about every 24 weeks, or once about every 6 months.

In certain embodiments, methods comprise administering a loading dose of about 60 mg of ISIS 814907 once about every 12 weeks, and subsequently administering a maintenance dose of about 60 mg of ISIS 814907 once about every 6 months. In certain embodiments, methods comprise administering a loading dose of about 115 mg of ISIS 814907 once about every 12 weeks, and subsequently administering a maintenance dose of about 115 mg of ISIS 814907 once about every 6 months. In certain embodiments, methods comprise administering a loading dose of about 60 mg to about 115 mg of ISIS 814907 once about every 12 weeks, and subsequently administering a maintenance dose of about 60 mg to about 115 mg of ISIS 814907 once about every 6 months.

### DETAILED DESCRIPTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, “2′-deoxyribonucleoside” means a nucleoside comprising a 2′-H(H) deoxyribosyl sugar moiety. In certain embodiments, a 2′-deoxyribonucleoside is a 2′-β-D deoxyribonucleoside and comprises a 2′-β-D-deoxyribosyl sugar moiety, which has the β-D configuration as found in naturally occurring deoxyribonucleic acids (DNA). In certain embodiments, a 2′-deoxyribonucleoside may comprise a modified nucleobase or may comprise an RNA nucleobase (uracil).

As used herein, “2′-MOE” means a 2′-OCH₂CH₂OCH₃ group in place of the 2′-OH group of a ribosyl sugar moiety. A “2′-MOE sugar moiety” is a sugar moiety with a 2′-OCH₂CH₂OCH₃ group in place of the 2′-OH group of a ribosyl sugar moiety. Unless otherwise indicated, a 2′-MOE sugar moiety is in the β-D configuration. “MOE” means O-methoxyethyl.

As used herein, “2′-MOE nucleoside” means a nucleoside comprising a 2′-MOE sugar moiety.

As used herein, “5-methyl cytosine” means a cytosine modified with a methyl group attached to the 5 position. A 5-methyl cytosine is a modified nucleobase.

As used herein, “about” means plus or minus 7% of the provided value.

As used herein, “administering” means providing a pharmaceutical agent to a human subject.

As used herein, “ameliorate” in reference to a treatment means improvement in at least one symptom or hallmark relative to the same symptom or hallmark in the absence of the treatment. In certain embodiments, amelioration is the reduction in the severity or frequency of a symptom or hallmark, or the delayed onset or slowing of progression in the severity or frequency of a symptom or hallmark.

As used herein, “CAG repeat” means one of multiple contiguous trinucleotide units, wherein each trinucleotide unit consists of three contiguous nucleosides having a nucleobase sequence from 5′ to 3′ of cytosine (C), adenine (A), and guanine (G).

As used herein, “dose” means a quantity of a pharmaceutical agent administered.

As used herein, the term “internucleoside linkage” means the covalent linkage between contiguous nucleosides in an oligonucleotide. As used herein “modified internucleoside linkage” means any internucleoside linkage other than a phosphodiester internucleoside linkage. “Phosphorothioate internucleoside linkage” is a modified internucleoside linkage in which one of the non-bridging oxygen atoms of a phosphodiester internucleoside linkage is replaced with a sulfur atom.

As used herein, “loading dose” means a therapeutically effective amount of a pharmaceutical agent administered during an initial dosing phase during which steady state concentration of the pharmaceutical agent is achieved. “Initial loading dose” means the first loading dose administered. “Last loading dose” means the loading dose administered most recently prior to administering a first maintenance dose.

As used herein, “maintenance dose” means a therapeutically effective amount of a pharmaceutical agent administered during a dosing phase after steady state concentration of the pharmaceutical agent has been achieved.

As used herein, the terms “MAPT_{Rx}” and “ISIS 814907” are interchangeable.

As used herein, “nucleobase” means an unmodified nucleobase or modified nucleobase. An “unmodified nucleobase” is adenine (A), thymine (T), cytosine (C), uracil (U), or guanine (G). A “modified nucleobase” is group of atoms other than unmodified A, T, C, U, or G capable of pairing with at least one unmodified nucleobase. A “5-methyl cytosine” is a modified nucleobase. As used herein, “nucleobase sequence” means the order of contiguous nucleobases in a target nucleic acid or oligonucleotide independent of any sugar or internucleoside linkage modification.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. “Linked nucleosides” are nucleosides that are connected in a contiguous sequence (i.e., no additional nucleosides are presented between those that are linked). As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified.

As used herein, “pharmaceutically acceptable carrier or diluent” means any substance suitable for use in administering to a human subject. Certain such carriers enable pharmaceutical compositions to be formulated as, for example, tablets, pills, dragees, capsules, liquids, gels, syrups, slurries, suspension, and lozenges for the oral ingestion by a human subject. In certain embodiments, a pharmaceutically acceptable carrier or diluent is sterile water, sterile saline, sterile buffer solution, or sterile artificial cerebrospinal fluid (aCSF).

As used herein, “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein, “potassium salt” means a salt of a modified oligonucleotide, wherein the cation of the salt is potassium.

As used herein, “RNA” means an RNA transcript and includes pre-mRNA and mature mRNA unless otherwise specified.

As used herein, “sodium salt” means a salt of a modified oligonucleotide, wherein the cation of the salt is sodium.

As used herein, “subject” means a human or non-human animal. In certain embodiments, the subject is a human subject. A “subject in need thereof,” is a subject who would benefit from administration of a modified oligonucleotide disclosed herein. In certain embodiments, the subject in need thereof has AD.

As used herein, “sugar moiety” means an unmodified sugar moiety or a modified sugar moiety. “Unmodified sugar moiety” means a 2′-OH(H) β-D ribosyl moiety, as found in RNA (an “unmodified RNA sugar moiety”), or a 2′-H(H) β-D deoxyribosyl moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. “Modified sugar moiety” or “modified sugar” means a modified furanosyl sugar moiety or a sugar surrogate.

As used herein, “symptom or hallmark” means any physical feature or test result that indicates the existence or extent of a disease or disorder. In certain embodiments, a symptom is apparent to a subject or to a medical professional examining or testing said subject. In certain embodiments, a hallmark is apparent upon invasive diagnostic testing, including, but not limited to, post-mortem tests. In certain embodiments, a hallmark is apparent on a brain MRI scan.

As used herein, “Tau RNA” is equivalent to “MAPT RNA” and is the RNA expression product of the human gene, MAPT. As used herein, “MAPT gene” refers to a genomic sequence encoding a Tau RNA.

As used herein, “Tau protein” is the protein expression product of Tau RNA.

As used herein, “therapeutically effective amount” means an amount of a pharmaceutical agent that provides a therapeutic benefit to a human subject. For example, a therapeutically effective amount improves a symptom or hallmark of a disease or disorder.

As used herein, “trough concentration” means the concentration of an analyte (e.g., Tau protein in a biological sample taken from a dosed human subject immediately prior to the human subject receiving a subsequent dose or the concentration of an analyte on the last study day.

As used herein, “week” means 7 days.

### Certain Embodiments

Embodiment 1. A method of ameliorating a disease or disorder associated with Tau protein in a human subject in need thereof, the method comprising administering to the human subject a therapeutically effective amount of a modified oligonucleotide according to the following chemical structure:

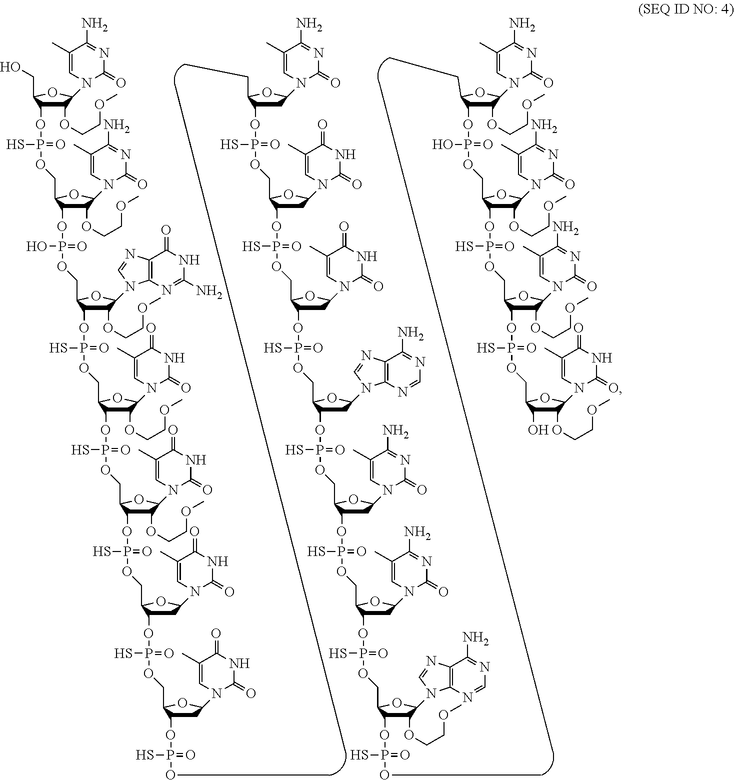

or a salt thereof.

Embodiment 2. The method of embodiment 1, wherein the modified oligonucleotide is the sodium salt or the potassium salt.

Embodiment 3. A method of ameliorating a disease or disorder associated with Tau protein in a human subject in need thereof, the method comprising administering to the human subject a therapeutically effective amount of a modified oligonucleotide according to the following chemical structure:

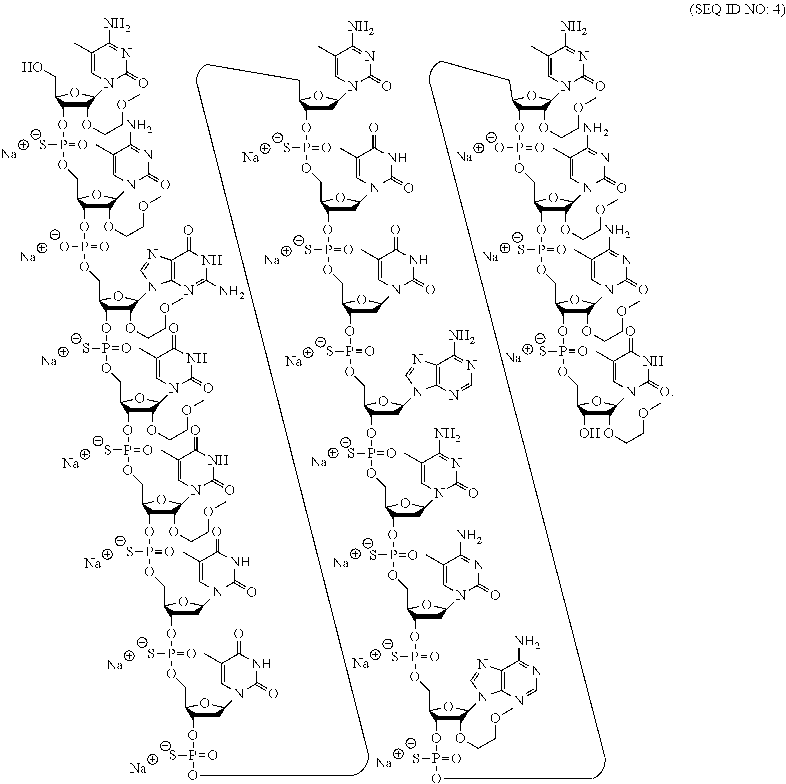

Embodiment 4. A method of ameliorating a disease or disorder associated with Tau protein in a human subject in need thereof, the method comprising administering to the human subject a therapeutically effective amount of a modified oligonucleotide, wherein the modified oligonucleotide has the following chemical notation (5′ to 3′): mCes mCeo Ges Tes Tes Tds Tds mCds Tds Tds Ads mCds mCds Aes mCeo mCes mCes Te (SEQ TD NO: 4); wherein,
A=an adenine nucleobase,
mC=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

Embodiment 5. The method of any of embodiments 1-4, wherein the disease or disorder associated with Tau protein is a neurodegenerative disease or disorder.

Embodiment 6. The method of any of embodiments 1-5, wherein the disease or disorder associated with Tau protein is a tauopathy.

Embodiment 7. The method of any one of embodiments 1-6, wherein the disease or disorder associated with Tau protein is any of Alzheimer's disease or Fronto-temporal Dementia (FTD), Frontotemporal Dementia with Parkinsonism-17 (FTDP-17), Progressive Supranuclear Palsy (PSP), Chronic Traumatic Encephalopathy (CTE), Corticobasal Ganglionic Degeneration (CBD), Pick Disease, Argyrophilic Grain Disease (AGD), Globular Glial Tauopathies, Epilepsy, and/or Dravet's Syndrome. In some embodiments, the Alzheimer's disease is Mild Cognitive Impairment (MCI) Due to Alzheimer's Disease and/or Alzheimer's Disease Dementia (e.g., Mild Alzheimer's Disease Dementia).

Embodiment 8. The method of any of embodiments 1-6, wherein the disease or disorder associated with Tau protein is any of Down syndrome, Prion diseases (sCJD, vCJD, gCJD, GSS, FFI), Diffuse neurofibrillary tangles with calcification, Familial British and Danish dementia, Postencephalitic parkinsonism, Subacute sclerosing panencephalitis, Myotonic dystrophy (DM1) and PROMM (DM2), Aging-related tau astrogliopathy, Traumatic brain injury, Chronic traumatic encephalopathy, IgLON5-related tauopathy, Guadeloupean parkinsonism, Parkinson—dementia complex of Guam, Non-Guamanian motor neuron disease with NFTs, Amyotrophic lateral sclerosis of Guam, X-linked parkinsonism with spasticity, Cerebrotendinous xanthomatosis, Niemann-Pick disease type C, PANK2-associated Neurodegeneration with Brain Iron Accumulation (NBIA), PLA2G6-assiciated NBIA, SLC9A6 mental retardation, or diseases or disorders associated with gene mutations in any of LRRK2, PRKN, SNCA, TARDBP, or C9orf72.

Embodiment 9. The method of any of embodiments 1-8, wherein the disease is Alzheimer's disease. In some embodiments, the Alzheimer's disease is mild Alzheimer's disease, Mild Cognitive Impairment (MCI) Due to Alzheimer's Disease, and/or Alzheimer's Disease Dementia (e.g., Mild Alzheimer's Disease Dementia).

Embodiment 10. The method of any of embodiments 1-8, wherein the disease is FTD.

Embodiment 11. The method of any of embodiments 1-10, wherein at least one symptom or hallmark of the disease or disorder associated with Tau protein is ameliorated.

Embodiment 12. The method of embodiment 11, wherein the at least one symptom or hallmark comprises loss of memory, cognitive decline, loss of ability to understand or express speech, abnormal behavior, impaired motor function, loss of cognitive function, neuropsychiatric behavior dysfunction, impaired global function, loss of motor function, impaired cognitive function, impaired neuropsychiatric function, impaired daily function, impaired attention, impaired visuoperceptual processing, impaired memory, impaired degree of independence, increased apathy, impaired learning ability, impaired mental concentration, impaired understanding and expression of speech, impaired behavior, depression, irritability, anger, impaired mobility, impaired self-care, pain, discomfort, anxiety, seizures, suicidal ideation, suicidal behavior, or increase in the number and/or volume of neurofibrillary inclusions.

Embodiment 13. A method of reducing Tau RNA in a human subject in need thereof, the method comprising administering to the human subject a therapeutically effective amount of a modified oligonucleotide according to the following chemical structure:

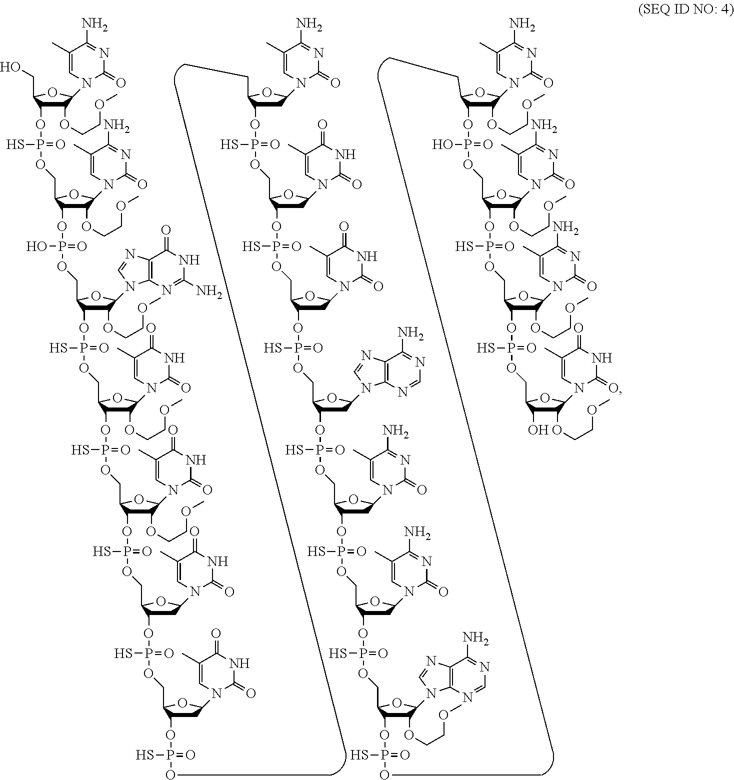

or a salt thereof.

Embodiment 14. The method of embodiment 13, wherein the modified oligonucleotide is the sodium salt or the potassium salt.

Embodiment 15. A method of reducing Tau RNA in a human subject in need thereof, the method comprising administering to the human subject a therapeutically effective amount of a modified oligonucleotide according to the following chemical structure:

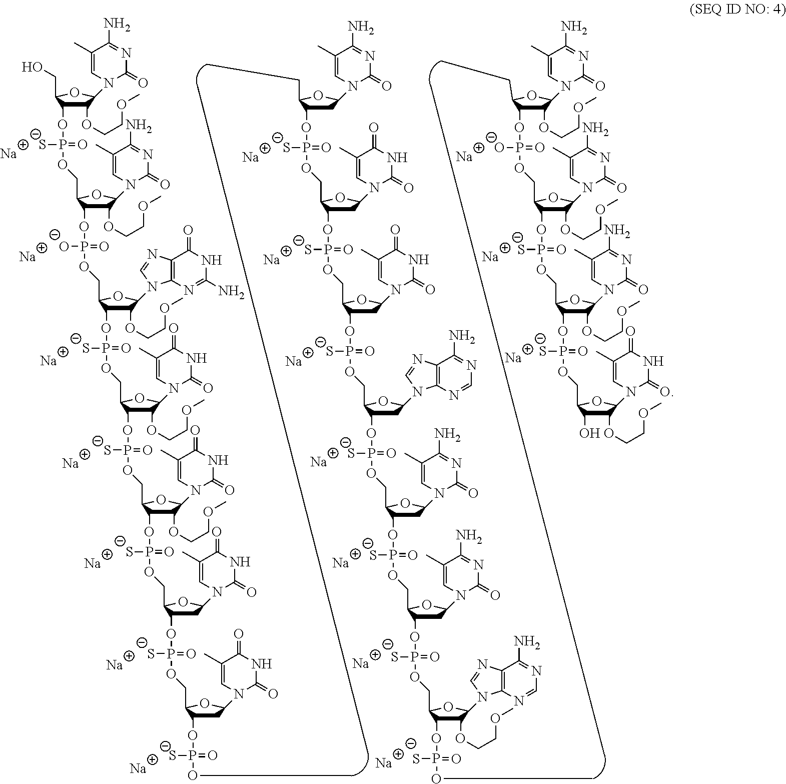

Embodiment 16. A method of reducing Tau RNA in a human subject in need thereof, the method comprising administering to the human subject a therapeutically effective amount of a modified oligonucleotide, wherein the modified oligonucleotide has the following chemical notation (5′ to 3′): mCes mCeo Ges Tes Tes Tds Tds mCds Tds Tds Ads mCds mCds Aes mCeo mCes mCes Te (SEQ TD NO: 4); wherein,
A=an adenine nucleobase,
mC=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

Embodiment 17. A method of reducing Tau protein in a human subject in need thereof, the method comprising administering to the human subject a therapeutically effective amount of a modified oligonucleotide according to the following chemical structure:

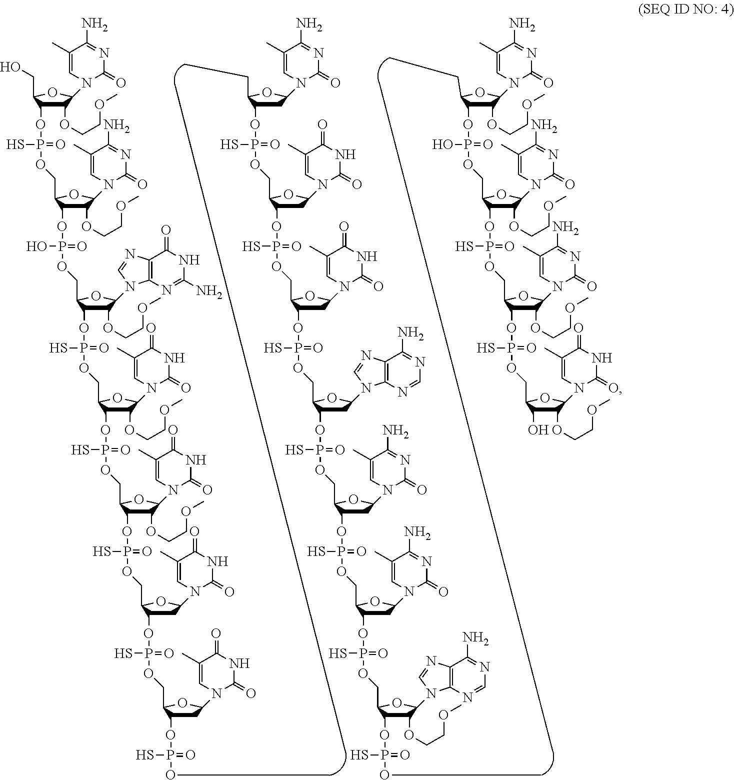

or a salt thereof.

Embodiment 18. A method of embodiment 17, wherein the modified oligonucleotide is the sodium salt or the potassium salt.

Embodiment 19. A method of reducing Tau protein in a human subject in need thereof, the method comprising administering to the human subject a therapeutically effective amount of a modified oligonucleotide according to the following chemical structure:

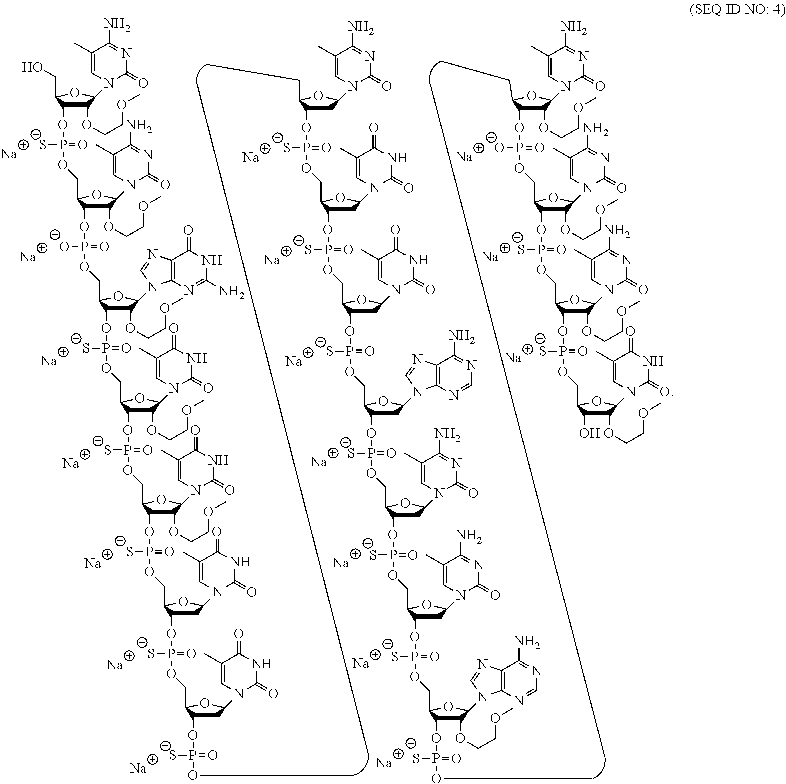

Embodiment 20. A method of reducing Tau protein in a human subject in need thereof, the method comprising administering to the human subject a therapeutically effective amount of a modified oligonucleotide, wherein the modified oligonucleotide has the following chemical notation (5′ to 3′): mCes mCeo Ges Tes Tes Tds Tds mCds Tds Tds Ads mCds mCds Aes mCeo mCes mCes Te (SEQ ID NO: 4); wherein,
A=an adenine nucleobase,
mC=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

Embodiment 21. The method of any of embodiments 1-20, wherein the therapeutically effective amount is 10 mg.

Embodiment 22. The method of any of embodiments 1-20, wherein the therapeutically effective amount is 30 mg.

Embodiment 23. The method of any of embodiments 1-20, wherein the therapeutically effective amount is 60 mg.

Embodiment 24. The method of any of embodiments 1-20, wherein the therapeutically effective amount is 90 mg.

Embodiment 25. The method of any of embodiments 1-20, wherein the therapeutically effective amount is 115 mg.

Embodiment 26. The method of any of embodiments 1-20, wherein the therapeutically effective amount is about 10 mg.

Embodiment 27. The method of any of embodiments 1-20, wherein the therapeutically effective amount is about 30 mg.

Embodiment 28. The method of any of embodiments 1-20, wherein the therapeutically effective amount is about 60 mg.

Embodiment 29. The method of any of embodiments 1-20, wherein the therapeutically effective amount is about 90 mg.

Embodiment 30. The method of any of embodiments 1-20, wherein the therapeutically effective amount is about 115 mg.

Embodiment 31. The method of any of embodiments 1-20, wherein the therapeutically effective amount is any of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, and 350 mg.

Embodiment 32. The method of any of embodiments 1-20 wherein the therapeutically effective amount is any of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, and about 350 mg.

Embodiment 33. The method of any of embodiments 1-20, wherein the therapeutically effective amount is any of 50 mg, 50.1 mg, 50.2 mg, 50.3 mg, 50.4 mg, 50.5 mg, 50.6 mg, 50.7 mg, 50.8 mg, 50.9 mg, 51 mg, 51.1 mg, 51.2 mg, 51.3 mg, 51.4 mg, 51.5 mg, 51.6 mg, 51.7 mg, 51.8 mg, 51.9 mg, 52 mg, 52.1 mg, 52.2 mg, 52.3 mg, 52.4 mg, 52.5 mg, 52.6 mg, 52.7 mg, 52.8 mg, 52.9 mg, 53 mg, 53.1 mg, 53.2 mg, 53.3 mg, 53.4 mg, 53.5 mg, 53.6 mg, 53.7 mg, 53.8 mg, 53.9 mg, 54 mg, 54.1 mg, 54.2 mg, 54.3 mg, 54.4 mg, 54.5 mg, 54.6 mg, 54.7 mg, 54.8 mg, 54.9 mg, 55 mg, 55.1 mg, 55.2 mg, 55.3 mg, 55.4 mg, 55.5 mg, 55.6 mg, 55.7 mg, 55.8 mg, 55.9 mg, 56 mg, 56.1 mg, 56.2 mg, 56.3 mg, 56.4 mg, 56.5 mg, 56.6 mg, 56.7 mg, 56.8 mg, 56.9 mg, 57 mg, 57.1 mg, 57.2 mg, 57.3 mg, 57.4 mg, 57.5 mg, 57.6 mg, 57.7 mg, 57.8 mg, 57.9 mg, 58 mg, 58.1 mg, 58.2 mg, 58.3 mg, 58.4 mg, 58.5 mg, 58.6 mg, 58.7 mg, 58.8 mg, 58.9 mg, 59 mg, 59.1 mg, 59.2 mg, 59.3 mg, 59.4 mg, 59.5 mg, 59.6 mg, 59.7 mg, 59.8 mg, 59.9 mg, 60 mg, 60.1 mg, 60.2 mg, 60.3 mg, 60.4 mg, 60.5 mg, 60.6 mg, 60.7 mg, 60.8 mg, 60.9 mg, 61 mg, 61.1 mg, 61.2 mg, 61.3 mg, 61.4 mg, 61.5 mg, 61.6 mg, 61.7 mg, 61.8 mg, 61.9 mg, 62 mg, 62.1 mg, 62.2 mg, 62.3 mg, 62.4 mg, 62.5 mg, 62.6 mg, 62.7 mg, 62.8 mg, 62.9 mg, 63 mg, 63.1 mg, 63.2 mg, 63.3 mg, 63.4 mg, 63.5 mg, 63.6 mg, 63.7 mg, 63.8 mg, 63.9 mg, 64 mg, 64.1 mg, 64.2 mg, 64.3 mg, 64.4 mg, 64.5 mg, 64.6 mg, 64.7 mg, 64.8 mg, 64.9 mg, 65 mg, 65.1 mg, 65.2 mg, 65.3 mg, 65.4 mg, 65.5 mg, 65.6 mg, 65.7 mg, 65.8 mg, 65.9 mg, 66 mg, 66.1 mg, 66.2 mg, 66.3 mg, 66.4 mg, 66.5 mg, 66.6 mg, 66.7 mg, 66.8 mg, 66.9 mg, 67 mg, 67.1 mg, 67.2 mg, 67.3 mg, 67.4 mg, 67.5 mg, 67.6 mg, 67.7 mg, 67.8 mg, 67.9 mg, 68 mg, 68.1 mg, 68.2 mg, 68.3 mg, 68.4 mg, 68.5 mg, 68.6 mg, 68.7 mg, 68.8 mg, 68.9 mg, 69 mg, 69.1 mg, 69.2 mg, 69.3 mg, 69.4 mg, 69.5 mg, 69.6 mg, 69.7 mg, 69.8 mg, 69.9 mg, and 70 mg.

Embodiment 34. The method of any of embodiments 1-20, wherein the therapeutically effective amount is any of about 50 mg, about 50.1 mg, about 50.2 mg, about 50.3 mg, about 50.4 mg, about 50.5 mg, about 50.6 mg, about 50.7 mg, about 50.8 mg, about 50.9 mg, about 51 mg, about 51.1 mg, about 51.2 mg, about 51.3 mg, about 51.4 mg, about 51.5 mg, about 51.6 mg, about 51.7 mg, about 51.8 mg, about 51.9 mg, about 52 mg, about 52.1 mg, about 52.2 mg, about 52.3 mg, about 52.4 mg, about 52.5 mg, about 52.6 mg, about 52.7 mg, about 52.8 mg, about 52.9 mg, about 53 mg, about 53.1 mg, about 53.2 mg, about 53.3 mg, about 53.4 mg, about 53.5 mg, about 53.6 mg, about 53.7 mg, about 53.8 mg, about 53.9 mg, about 54 mg, about 54.1 mg, about 54.2 mg, about 54.3 mg, about 54.4 mg, about 54.5 mg, about 54.6 mg, about 54.7 mg, about 54.8 mg, about 54.9 mg, about 55 mg, about 55.1 mg, about 55.2 mg, about 55.3 mg, about 55.4 mg, about 55.5 mg, about 55.6 mg, about 55.7 mg, about 55.8 mg, about 55.9 mg, about 56 mg, about 56.1 mg, about 56.2 mg, about 56.3 mg, about 56.4 mg, about 56.5 mg, about 56.6 mg, about 56.7 mg, about 56.8 mg, about 56.9 mg, about 57 mg, about 57.1 mg, about 57.2 mg, about 57.3 mg, about 57.4 mg, about 57.5 mg, about 57.6 mg, about 57.7 mg, about 57.8 mg, about 57.9 mg, about 58 mg, about 58.1 mg, about 58.2 mg, about 58.3 mg, about 58.4 mg, about 58.5 mg, about 58.6 mg, about 58.7 mg, about 58.8 mg, about 58.9 mg, about 59 mg, about 59.1 mg, about 59.2 mg, about 59.3 mg, about 59.4 mg, about 59.5 mg, about 59.6 mg, about 59.7 mg, about 59.8 mg, about 59.9 mg, about 60 mg, about 60.1 mg, about 60.2 mg, about 60.3 mg, about 60.4 mg, about 60.5 mg, about 60.6 mg, about 60.7 mg, about 60.8 mg, about 60.9 mg, about 61 mg, about 61.1 mg, about 61.2 mg, about 61.3 mg, about 61.4 mg, about 61.5 mg, about 61.6 mg, about 61.7 mg, about 61.8 mg, about 61.9 mg, about 62 mg, about 62.1 mg, about 62.2 mg, about 62.3 mg, about 62.4 mg, about 62.5 mg, about 62.6 mg, about 62.7 mg, about 62.8 mg, about 62.9 mg, about 63 mg, about 63.1 mg, about 63.2 mg, about 63.3 mg, about 63.4 mg, about 63.5 mg, about 63.6 mg, about 63.7 mg, about 63.8 mg, about 63.9 mg, about 64 mg, about 64.1 mg, about 64.2 mg, about 64.3 mg, about 64.4 mg, about 64.5 mg, about 64.6 mg, about 64.7 mg, about 64.8 mg, about 64.9 mg, about 65 mg, about 65.1 mg, about 65.2 mg, about 65.3 mg, about 65.4 mg, about 65.5 mg, about 65.6 mg, about 65.7 mg, about 65.8 mg, about 65.9 mg, about 66 mg, about 66.1 mg, about 66.2 mg, about 66.3 mg, about 66.4 mg, about 66.5 mg, about 66.6 mg, about 66.7 mg, about 66.8 mg, about 66.9 mg, about 67 mg, about 67.1 mg, about 67.2 mg, about 67.3 mg, about 67.4 mg, about 67.5 mg, about 67.6 mg, about 67.7 mg, about 67.8 mg, about 67.9 mg, about 68 mg, about 68.1 mg, about 68.2 mg, about 68.3 mg, about 68.4 mg, about 68.5 mg, about 68.6 mg, about 68.7 mg, about 68.8 mg, about 68.9 mg, about 69 mg, about 69.1 mg, about 69.2 mg, about 69.3 mg, about 69.4 mg, about 69.5 mg, about 69.6 mg, about 69.7 mg, about 69.8 mg, about 69.9 mg, and about 70 mg.

Embodiment 35. The method of any of embodiments 1-20, wherein the therapeutically effective amount is any of 80 mg, 80.1 mg, 80.2 mg, 80.3 mg, 80.4 mg, 80.5 mg, 80.6 mg, 80.7 mg, 80.8 mg, 80.9 mg, 81 mg, 81.1 mg, 81.2 mg, 81.3 mg, 81.4 mg, 81.5 mg, 81.6 mg, 81.7 mg, 81.8 mg, 81.9 mg, 82 mg, 82.1 mg, 82.2 mg, 82.3 mg, 82.4 mg, 82.5 mg, 82.6 mg, 82.7 mg, 82.8 mg, 82.9 mg, 83 mg, 83.1 mg, 83.2 mg, 83.3 mg, 83.4 mg, 83.5 mg, 83.6 mg, 83.7 mg, 83.8 mg, 83.9 mg, 84 mg, 84.1 mg, 84.2 mg, 84.3 mg, 84.4 mg, 84.5 mg, 84.6 mg, 84.7 mg, 84.8 mg, 84.9 mg, 85 mg, 85.1 mg, 85.2 mg, 85.3 mg, 85.4 mg, 85.5 mg, 85.6 mg, 85.7 mg, 85.8 mg, 85.9 mg, 86 mg, 86.1 mg, 86.2 mg, 86.3 mg, 86.4 mg, 86.5 mg, 86.6 mg, 86.7 mg, 86.8 mg, 86.9 mg, 87 mg, 87.1 mg, 87.2 mg, 87.3 mg, 87.4 mg, 87.5 mg, 87.6 mg, 87.7 mg, 87.8 mg, 87.9 mg, 88 mg, 88.1 mg, 88.2 mg, 88.3 mg, 88.4 mg, 88.5 mg, 88.6 mg, 88.7 mg, 88.8 mg, 88.9 mg, 89 mg, 89.1 mg, 89.2 mg, 89.3 mg, 89.4 mg, 89.5 mg, 89.6 mg, 89.7 mg, 89.8 mg, 89.9 mg, 90 mg, 90.1 mg, 90.2 mg, 90.3 mg, 90.4 mg, 90.5 mg, 90.6 mg, 90.7 mg, 90.8 mg, 90.9 mg, 91 mg, 91.1 mg, 91.2 mg, 91.3 mg, 91.4 mg, 91.5 mg, 91.6 mg, 91.7 mg, 91.8 mg, 91.9 mg, 92 mg, 92.1 mg, 92.2 mg, 92.3 mg, 92.4 mg, 92.5 mg, 92.6 mg, 92.7 mg, 92.8 mg, 92.9 mg, 93 mg, 93.1 mg, 93.2 mg, 93.3 mg, 93.4 mg, 93.5 mg, 93.6 mg, 93.7 mg, 93.8 mg, 93.9 mg, 94 mg, 94.1 mg, 94.2 mg, 94.3 mg, 94.4 mg, 94.5 mg, 94.6 mg, 94.7 mg, 94.8 mg, 94.9 mg, 95 mg, 95.1 mg, 95.2 mg, 95.3 mg, 95.4 mg, 95.5 mg, 95.6 mg, 95.7 mg, 95.8 mg, 95.9 mg, 96 mg, 96.1 mg, 96.2 mg, 96.3 mg, 96.4 mg, 96.5 mg, 96.6 mg, 96.7 mg, 96.8 mg, 96.9 mg, 97 mg, 97.1 mg, 97.2 mg, 97.3 mg, 97.4 mg, 97.5 mg, 97.6 mg, 97.7 mg, 97.8 mg, 97.9 mg, 98 mg, 98.1 mg, 98.2 mg, 98.3 mg, 98.4 mg, 98.5 mg, 98.6 mg, 98.7 mg, 98.8 mg, 98.9 mg, 99 mg, 99.1 mg, 99.2 mg, 99.3 mg, 99.4 mg, 99.5 mg, 99.6 mg, 99.7 mg, 99.8 mg, 99.9 mg, and 100 mg.

Embodiment 36. The method of any of embodiments 1-20, wherein the therapeutically effective amount is any of about 80 mg, about 80.1 mg, about 80.2 mg, about 80.3 mg, about 80.4 mg, about 80.5 mg, about 80.6 mg, about 80.7 mg, about 80.8 mg, about 80.9 mg, about 81 mg, about 81.1 mg, about 81.2 mg, about 81.3 mg, about 81.4 mg, about 81.5 mg, about 81.6 mg, about 81.7 mg, about 81.8 mg, about 81.9 mg, about 82 mg, about 82.1 mg, about 82.2 mg, about 82.3 mg, about 82.4 mg, about 82.5 mg, about 82.6 mg, about 82.7 mg, about 82.8 mg, about 82.9 mg, about 83 mg, about 83.1 mg, about 83.2 mg, about 83.3 mg, about 83.4 mg, about 83.5 mg, about 83.6 mg, about 83.7 mg, about 83.8 mg, about 83.9 mg, about 84 mg, about 84.1 mg, about 84.2 mg, about 84.3 mg, about 84.4 mg, about 84.5 mg, about 84.6 mg, about 84.7 mg, about 84.8 mg, about 84.9 mg, about 85 mg, about 85.1 mg, about 85.2 mg, about 85.3 mg, about 85.4 mg, about 85.5 mg, about 85.6 mg, about 85.7 mg, about 85.8 mg, about 85.9 mg, about 86 mg, about 86.1 mg, about 86.2 mg, about 86.3 mg, about 86.4 mg, about 86.5 mg, about 86.6 mg, about 86.7 mg, about 86.8 mg, about 86.9 mg, about 87 mg, about 87.1 mg, about 87.2 mg, about 87.3 mg, about 87.4 mg, about 87.5 mg, about 87.6 mg, about 87.7 mg, about 87.8 mg, about 87.9 mg, about 88 mg, about 88.1 mg, about 88.2 mg, about 88.3 mg, about 88.4 mg, about 88.5 mg, about 88.6 mg, about 88.7 mg, about 88.8 mg, about 88.9 mg, about 89 mg, about 89.1 mg, about 89.2 mg, about 89.3 mg, about 89.4 mg, about 89.5 mg, about 89.6 mg, about 89.7 mg, about 89.8 mg, about 89.9 mg, about 90 mg, about 90.1 mg, about 90.2 mg, about 90.3 mg, about 90.4 mg, about 90.5 mg, about 90.6 mg, about 90.7 mg, about 90.8 mg, about 90.9 mg, about 91 mg, about 91.1 mg, about 91.2 mg, about 91.3 mg, about 91.4 mg, about 91.5 mg, about 91.6 mg, about 91.7 mg, about 91.8 mg, about 91.9 mg, about 92 mg, about 92.1 mg, about 92.2 mg, about 92.3 mg, about 92.4 mg, about 92.5 mg, about 92.6 mg, about 92.7 mg, about 92.8 mg, about 92.9 mg, about 93 mg, about 93.1 mg, about 93.2 mg, about 93.3 mg, about 93.4 mg, about 93.5 mg, about 93.6 mg, about 93.7 mg, about 93.8 mg, about 93.9 mg, about 94 mg, about 94.1 mg, about 94.2 mg, about 94.3 mg, about 94.4 mg, about 94.5 mg, about 94.6 mg, about 94.7 mg, about 94.8 mg, about 94.9 mg, about 95 mg, about 95.1 mg, about 95.2 mg, about 95.3 mg, about 95.4 mg, about 95.5 mg, about 95.6 mg, and about 95.7 mg,

Embodiment 37. The method of any of embodiments 1-20, wherein the therapeutically effective amount is any of 105 mg, 105.1 mg, 105.2 mg, 105.3 mg, 105.4 mg, 105.5 mg, 105.6 mg, 105.7 mg, 105.8 mg, 105.9 mg, 106 mg, 106.1 mg, 106.2 mg, 106.3 mg, 106.4 mg, 106.5 mg, 106.6 mg, 106.7 mg, 106.8 mg, 106.9 mg, 107 mg, 107.1 mg, 107.2 mg, 107.3 mg, 107.4 mg, 107.5 mg, 107.6 mg, 107.7 mg, 107.8 mg, 107.9 mg, 108 mg, 108.1 mg, 108.2 mg, 108.3 mg, 108.4 mg, 108.5 mg, 108.6 mg, 108.7 mg, 108.8 mg, 108.9 mg, 109 mg, 109.1 mg, 109.2 mg, 109.3 mg, 109.4 mg, 109.5 mg, 109.6 mg, 109.7 mg, 109.8 mg, 109.9 mg, 110 mg, 110.1 mg, 110.2 mg, 110.3 mg, 110.4 mg, 110.5 mg, 110.6 mg, 110.7 mg, 110.8 mg, 110.9 mg, 111 mg, 111.1 mg, 111.2 mg, 111.3 mg, 111.4 mg, 111.5 mg, 111.6 mg, 111.7 mg, 111.8 mg, 111.9 mg, 112 mg, 112.1 mg, 112.2 mg, 112.3 mg, 112.4 mg, 112.5 mg, 112.6 mg, 112.7 mg, 112.8 mg, 112.9 mg, 113 mg, 113.1 mg, 113.2 mg, 113.3 mg, 113.4 mg, 113.5 mg, 113.6 mg, 113.7 mg, 113.8 mg, 113.9 mg, 114 mg, 114.1 mg, 114.2 mg, 114.3 mg, 114.4 mg, 114.5 mg, 114.6 mg, 114.7 mg, 114.8 mg, 114.9 mg, 115 mg, 115.1 mg, 115.2 mg, 115.3 mg, 115.4 mg, 115.5 mg, 115.6 mg, 115.7 mg, 115.8 mg, 115.9 mg, 116 mg, 116.1 mg, 116.2 mg, 116.3 mg, 116.4 mg, 116.5 mg, 116.6 mg, 116.7 mg, 116.8 mg, 116.9 mg, 117 mg, 117.1 mg, 117.2 mg, 117.3 mg, 117.4 mg, 117.5 mg, 117.6 mg, 117.7 mg, 117.8 mg, 117.9 mg, 118 mg, 118.1 mg, 118.2 mg, 118.3 mg, 118.4 mg, 118.5 mg, 118.6 mg, 118.7 mg, 118.8 mg, 118.9 mg, 119 mg, 119.1 mg, 119.2 mg, 119.3 mg, 119.4 mg, 119.5 mg, 119.6 mg, 119.7 mg, 119.8 mg, 119.9 mg, 120 mg, 120.1 mg, 120.2 mg, 120.3 mg, 120.4 mg, 120.5 mg, 120.6 mg, 120.7 mg, 120.8 mg, 120.9 mg, 121 mg, 121.1 mg, 121.2 mg, 121.3 mg, 121.4 mg, 121.5 mg, 121.6 mg, 121.7 mg, 121.8 mg, 121.9 mg, 122 mg, 122.1 mg, 122.2 mg, 122.3 mg, 122.4 mg, 122.5 mg, 122.6 mg, 122.7 mg, 122.8 mg, 122.9 mg, 123 mg, 123.1 mg, 123.2 mg, 123.3 mg, 123.4 mg, 123.5 mg, 123.6 mg, 123.7 mg, 123.8 mg, 123.9 mg, 124 mg, 124.1 mg, 124.2 mg, 124.3 mg, 124.4 mg, 124.5 mg, 124.6 mg, 124.7 mg, 124.8 mg, 124.9 mg, and 125 mg.

Embodiment 38. The method of any of embodiments 1-20, wherein the therapeutically effective amount is any of about 105 mg, about 105.1 mg, about 105.2 mg, about 105.3 mg, about 105.4 mg, about 105.5 mg, about 105.6 mg, about 105.7 mg, about 105.8 mg, about 105.9 mg, about 106 mg, about 106.1 mg, about 106.2 mg, about 106.3 mg, about 106.4 mg, about 106.5 mg, about 106.6 mg, about 106.7 mg, about 106.8 mg, about 106.9 mg, about 107 mg, about 107.1 mg, about 107.2 mg, about 107.3 mg, about 107.4 mg, about 107.5 mg, about 107.6 mg, about 107.7 mg, about 107.8 mg, about 107.9 mg, about 108 mg, about 108.1 mg, about 108.2 mg, about 108.3 mg, about 108.4 mg, about 108.5 mg, about 108.6 mg, about 108.7 mg, about 108.8 mg, about 108.9 mg, about 109 mg, about 109.1 mg, about 109.2 mg, about 109.3 mg, about 109.4 mg, about 109.5 mg, about 109.6 mg, about 109.7 mg, about 109.8 mg, about 109.9 mg, about 110 mg, about 110.1 mg, about 110.2 mg, about 110.3 mg, about 110.4 mg, about 110.5 mg, about 110.6 mg, about 110.7 mg, about 110.8 mg, about 110.9 mg, about 111 mg, about 111.1 mg, about 111.2 mg, about 111.3 mg, about 111.4 mg, about 111.5 mg, about 111.6 mg, about 111.7 mg, about 111.8 mg, about 111.9 mg, about 112 mg, about 112.1 mg, about 112.2 mg, about 112.3 mg, about 112.4 mg, about 112.5 mg, about 112.6 mg, about 112.7 mg, about 112.8 mg, about 112.9 mg, about 113 mg, about 113.1 mg, about 113.2 mg, about 113.3 mg, about 113.4 mg, about 113.5 mg, about 113.6 mg, about 113.7 mg, about 113.8 mg, about 113.9 mg, about 114 mg, about 114.1 mg, about 114.2 mg, about 114.3 mg, about 114.4 mg, about 114.5 mg, about 114.6 mg, about 114.7 mg, about 114.8 mg, about 114.9 mg, about 115 mg, about 115.1 mg, about 115.2 mg, about 115.3 mg, about 115.4 mg, about 115.5 mg, about 115.6 mg, about 115.7 mg, about 115.8 mg, about 115.9 mg, about 116 mg, about 116.1 mg, about 116.2 mg, about 116.3 mg, about 116.4 mg, about 116.5 mg, about 116.6 mg, about 116.7 mg, about 116.8 mg, about 116.9 mg, about 117 mg, about 117.1 mg, about 117.2 mg, about 117.3 mg, about 117.4 mg, about 117.5 mg, about 117.6 mg, about 117.7 mg, about 117.8 mg, about 117.9 mg, about 118 mg, about 118.1 mg, about 118.2 mg, about 118.3 mg, about 118.4 mg, about 118.5 mg, about 118.6 mg, about 118.7 mg, about 118.8 mg, about 118.9 mg, about 119 mg, about 119.1 mg, about 119.2 mg, about 119.3 mg, about 119.4 mg, about 119.5 mg, about 119.6 mg, about 119.7 mg, about 119.8 mg, about 119.9 mg, about 120 mg, about 120.1 mg, about 120.2 mg, about 120.3 mg, about 120.4 mg, about 120.5 mg, about 120.6 mg, about 120.7 mg, about 120.8 mg, about 120.9 mg, about 121 mg, about 121.1 mg, about 121.2 mg, about 121.3 mg, about 121.4 mg, about 121.5 mg, about 121.6 mg, about 121.7 mg, about 121.8 mg, about 121.9 mg, about 122 mg, about 122.1 mg, about 122.2 mg, about 122.3 mg, about 122.4 mg, about 122.5 mg, about 122.6 mg, about 122.7 mg, about 122.8 mg, about 122.9 mg, about 123 mg, about 123.1 mg, about 123.2 mg, about 123.3 mg, about 123.4 mg, about 123.5 mg, about 123.6 mg, about 123.7 mg, about 123.8 mg, about 123.9 mg, about 124 mg, about 124.1 mg, about 124.2 mg, about 124.3 mg, about 124.4 mg, about 124.5 mg, about 124.6 mg, about 124.7 mg, about 124.8 mg, about 124.9 mg, and about 125 mg.

Embodiment 39. The method of any of embodiments 1-20, wherein the therapeutically effective amount is within the range of any of 40 mg to 200 mg, 40 mg to 190 mg, 40 mg to 180 mg, 40 mg to 170 mg, from 40 mg to 160 mg, 40 mg to 150 mg, 40 mg to 140 mg, 40 mg to 120 mg, 40 mg to 110 mg, 40 mg to 100 mg, 40 mg to 80 mg, 40 mg to 70 mg, 40 mg to 60 mg, 40 mg to 50 mg, 50 mg to 200 mg, 50 mg to 190 mg, 50 mg to 180 mg, 50 mg to 170 mg, 50 mg to 160 mg, 50 mg to 150 mg, 50 mg to 140 mg, 50 mg to 120 mg, 50 mg to 110 mg, 50 mg to 100 mg, 50 mg to 80 mg, 50 mg to 70 mg, 50 mg to 60 mg, 60 mg to 200 mg, 60 mg to 190 mg, 60 mg to 180 mg, 60 mg to 170 mg, 60 mg to 160 mg, 60 mg to 150 mg, 60 mg to 140 mg, 60 mg to 120 mg, 60 mg to 110 mg, 60 mg to 100 mg, 60 mg to 80 mg, 60 mg to 70 mg, 70 mg to 200 mg, 70 mg to 190 mg, 70 mg to 180 mg, 70 mg to 170 mg, 70 mg to 160 mg, 70 mg to 150 mg, 70 mg to 140 mg, 70 mg to 120 mg, 70 mg to 110 mg, 70 mg to 100 mg, 70 mg to 80 mg, 80 mg to 200 mg, 80 mg to 190 mg, 80 mg to 180 mg, 80 mg to 170 mg, 80 mg to 160 mg, 80 mg to 150 mg, 80 mg to 140 mg, 80 mg to 120 mg, 80 mg to 110 mg, 80 mg to 100 mg, 80 mg to 90 mg, 90 mg to 200 mg, 90 mg to 190 mg, 90 mg to 180 mg, 90 mg to 170 mg, 90 mg to 160 mg, 90 mg to 150 mg, 90 mg to 140 mg, 90 mg to 120 mg, 90 mg to 110 mg, 90 mg to 100 mg, 100 mg to 200 mg, 100 mg to 190 mg, 100 mg to 180 mg, 100 mg to 170 mg, 100 mg to 160 mg, 100 mg to 150 mg, 100 mg to 140 mg, 100 mg to 120 mg, 100 mg to 110 mg, 110 mg to 200 mg, 110 mg to 190 mg, 110 mg to 180 mg, 110 mg to 170 mg, 110 mg to 160 mg, 110 mg to 150 mg, 110 mg to 140 mg, 110 mg to 130 mg, 110 mg to 120 mg, 120 mg to 200 mg, 120 mg to 190 mg, 120 mg to 180 mg, 120 mg to 170 mg, 120 mg to 160 mg, 120 mg to 150 mg, 120 mg to 140 mg, 120 mg to 130 mg, 130 mg to 200 mg, 130 mg to 190 mg, 130 mg to 180 mg, 130 mg to 170 mg, 130 mg to 160 mg, 130 mg to 150 mg, 130 mg to 140 mg, 140 mg to 200 mg, 140 mg to 190 mg, 140 mg to 180 mg, 140 mg to 170 mg, 140 mg to 160 mg, 140 mg to 150 mg, 150 mg to 200 mg, 150 mg to 190 mg, 150 mg to 180 mg, 150 mg to 170 mg, 150 mg to 160 mg, 160 mg to 200 mg, 160 mg to 190 mg, 160 mg to 180 mg, 160 mg to 170 mg, 180 mg to 200 mg, 180 mg to 190 mg, 190 mg to 200 mg, 105 mg to 135 mg, 105 mg to 130 mg, 105 mg to 125 mg 105 mg to 120 mg, 110 mg to 135 mg, 110 mg to 130 mg, 110 mg to 125 mg, 110 mg to 120 mg, 115 mg to 135 mg, 115 mg to 130 mg, 115 mg to 125 mg, 115 mg to 120 mg, 115 mg to 125 mg, 115 mg to 120 mg, 120 mg to 135 mg, 120 mg to 125 mg, 125 mg to 140 mg, 125 mg to 130 mg, 130 mg to 135 mg, 135 mg to 140 mg, 120 mg to 129 mg, 120 mg to 128 mg, 120 mg to 127 mg, 120 mg to 86 mg, 120 mg to 124 mg, 120 mg to 123 mg, 120 mg to 122 mg, 120 mg to 121 mg, 121 mg to 130 mg, 122 mg to 129 mg, 122 mg to 128 mg, 122 mg to 127 mg, 122 mg to 126 mg, 122 mg to 125 mg, 122 mg to 124 mg, 122 mg to 123 mg, 123 mg to 130 mg, 123 mg to 129 mg, 123 mg to 128 mg, 123 mg to 127 mg, 123 mg to 126 mg, 123 mg to 125 mg, 123 mg to 124 mg, 124 mg to 130 mg, 124 mg to 129 mg, 124 mg to 128 mg, 124 mg to 127 mg, 124 mg to 126 mg, 124 mg to 125 mg, 125 mg to 129 mg, 125 mg to 128 mg, 125 mg to 127 mg, 125 mg to 126 mg, 126 mg to 130 mg, 126 mg to 129 mg, 126 mg to 128 mg, 126 mg to 127 mg, 127 mg to 130 mg, 127 mg to 129 mg, 127 mg to 128 mg, 128 mg to 130 mg, 128 mg to 129 mg, and 129 mg to 130 mg.

Embodiment 40. The method of any of embodiments 1-20, wherein the therapeutically effective amount is any of less than 350 mg, less than 345 mg, less than 340 mg, less than 335 mg, less than 330 mg, less than 325 mg, less than 320 mg, less than 315 mg, less than 310 mg, less than 305 mg, less than 300 mg, less than 295 mg, less than 290 mg, less than 285 mg, less than 280 mg, less than 275 mg, less than 270 mg, less than 265 mg, less than 260 mg, less than 255 mg, less than 250 mg, less than 245 mg, less than 240 mg, less than 235 mg, less than 230 mg, less than 225 mg, less than 220 mg, less than 215 mg, less than 210 mg, less than 205 mg, less than 200 mg, less than 195 mg, less than 190 mg, less than 185 mg, less than 180 mg, less than 175 mg, less than 170 mg, less than 165 mg, less than 160 mg, less than 150 mg, less than 145 mg, less than 140 mg, less than 135 mg, less than 130 mg, less than 125 mg, less than 120 mg, less than 115 mg, less than 110 mg, less than 105 mg, less than 100 mg, less than 95 mg, less than 90 mg, less than 85 mg, less than 80 mg, less than 75 mg, less than 70 mg, less than 65 mg, less than 60 mg, less than 55 mg, less than 50 mg, less than 45 mg, less than 40 mg, less than 35 mg, less than 30 mg, less than 25 mg, less than 20 mg, less than 15 mg, less than 10 mg, and less than 5 mg.

Embodiment 41. The method of any of embodiments 1-20, wherein the therapeutically effective amount is any of less than about less than about 350 mg, less than about 345 mg, less than about 340 mg, less than about 335 mg, less than about 330 mg, less than about 325 mg, less than about 320 mg, less than about 315 mg, less than about 310 mg, less than about 305 mg, less than about 300 mg, less than about 295 mg, less than about 290 mg, less than about 285 mg, less than about 280 mg, less than about 275 mg, less than about 270 mg, less than about 265 mg, less than about 260 mg, less than about 255 mg, less than about 250 mg, less than about 245 mg, less than about 240 mg, less than about 235 mg, less than about 230 mg, less than about 225 mg, less than about 220 mg, less than about 215 mg, less than about 210 mg, less than about 205 mg, less than about 200 mg, less than about 195 mg, less than about 190 mg, less than about 185 mg, less than about 180 mg, less than about 175 mg, less than about 170 mg, less than about 165 mg, less than about 160 mg, less than about 150 mg, less than about 145 mg, less than about 140 mg, less than about 135 mg, less than about 130 mg, less than about 125 mg, less than about 120 mg, less than about 115 mg, less than about 110 mg, less than about 105 mg, less than about 100 mg, less than about 95 mg, less than about 90 mg, less than about 85 mg, less than about 80 mg, less than about 75 mg, less than about 70 mg, less than about 65 mg, less than about 60 mg, less than about 55 mg, less than about 50 mg, less than about 45 mg, less than about 40 mg, less than about 35 mg, less than about 30 mg, less than about 25 mg, less than about 20 mg, less than about 15 mg, less than about 10 mg, and less than about 5 mg.

Embodiment 42. The method of any of embodiments 1-20, wherein the therapeutically effective amount is any of at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least about 100 mg, at least 105 mg, at least 115 mg, at least 120 mg, at least 125 mg, at least 130 mg, at least 135 mg, at least 140 mg, at least 145 mg, at least 150 mg, at least 155 mg, at least 160 mg, at least 165 mg, at least 170 mg, at least 175 mg, at least 180 mg, at least 185, at least 190 mg, at least 195 mg, and at least 200 mg.

Embodiment 43. The method of any of embodiments 1-20, wherein the therapeutically effective amount is any of at least about 5 mg, at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 105 mg, at least about 115 mg, at least about 120 mg, at least about 125 mg, at least about 130 mg, at least about 135 mg, at least about 140 mg, at least about 145 mg, or at least about 150 mg, at least about 155 mg, at least about 160 mg, at least about 165 mg, at least about 170 mg, at least about 175 mg, at least about 180 mg, at least about 185, at least about 190 mg, at least about 195 mg, and at least about 200 mg.

Embodiment 44. The method of any of embodiments 1-43, comprising administering the modified oligonucleotide once every 4 weeks.

Embodiment 45. The method of any of embodiments 1-43, comprising administering the modified oligonucleotide once every 8 weeks.

Embodiment 46. The method of any of embodiments 1-43, comprising administering the modified oligonucleotide once every 12 weeks.

Embodiment 47. The method of any of embodiments 1-43, comprising administering the modified oligonucleotide once every 16 weeks.

Embodiment 48. The method of any of embodiments 1-43, comprising administering the modified oligonucleotide once every 20 weeks.

Embodiment 49. The method of any of embodiments 1-43, comprising administering the modified oligonucleotide once every 24 weeks.

Embodiment 50. The method of any of embodiments 1-43, comprising administering the modified oligonucleotide once every 6 months.

Embodiment 51. The method of any of embodiments 1-43, comprising administering the modified oligonucleotide about once every 4 weeks.

Embodiment 52. The method of any of embodiments 1-43, comprising administering the modified oligonucleotide about once every 8 weeks.

Embodiment 53. The method of any of embodiments 1-43, comprising administering the modified oligonucleotide about once every 12 weeks.

Embodiment 54. The method of any of embodiments 1-43, comprising administering the modified oligonucleotide about once every 16 weeks.

Embodiment 55. The method of any of embodiments 1-43, comprising administering the modified oligonucleotide about once every 20 weeks.

Embodiment 56. The method of any of embodiments 1-43, comprising administering the modified oligonucleotide about once every 24 weeks.

Embodiment 57. The method of any of embodiments 1-43, comprising administering the modified oligonucleotide once about every 6 months.

Embodiment 58. The method of any of embodiments 1-43, comprising administering the modified oligonucleotide monthly.

Embodiment 59. The method of any of embodiments 1-43, comprising administering the modified oligonucleotide once every two months.

Embodiment 60. The method of any of embodiments 1-43, comprising administering the modified oligonucleotide once every three months.

Embodiment 61. The method of any of embodiments 1-43, comprising administering the modified oligonucleotide quarterly.

Embodiment 62. The method of any of embodiments 1-43, comprising administering the modified oligonucleotide semiannually.

Embodiment 63. The method of any of embodiments 1-43, comprising administering the modified oligonucleotide annually.

Embodiment 64. The method of any of embodiments 1-43, comprising administering the modified oligonucleotide once every two years.

Embodiment 65. The method of any of embodiments 1-43, comprising administering the modified oligonucleotide any of once every 1 week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks, once every 17 weeks, once every 18 weeks, once every 19 weeks, once every 20 weeks, once every 21 weeks, once every 22 weeks, once every 23 weeks, once every 24 weeks, once every month, once every 2 months, once every 3 months, once every 4 months, once every 5 months, once every 6 months, once every 7 months, once every 8 months, once every 9 months, once every 10 months, once every 11 months, or once every year.

Embodiment 66. The method of any of embodiments 1-43, comprising administering the modified oligonucleotide any of once about every 1 week, once about every 2 weeks, once about every 3 weeks, once about every 4 weeks, once about every 5 weeks, once about every 6 weeks, once about every 7 weeks, once about every 8 weeks, once about every 9 weeks, once about every 10 weeks, once about every 11 weeks, once about every 12 weeks, once about every 13 weeks, once about every 14 weeks, once about every 15 weeks, once about every 16 weeks, once about every 17 weeks, once about every 18 weeks, once about every 19 weeks, once about every 21 weeks, once about every 22 weeks, once about every 23 weeks, once about every 24 weeks, once about every month, once about every 2 months, once about every 3 months, once about every 4 months, once about every 5 months, and once about every 6 months, once about every 7 months, once about every 8 months, once about every 9 months, once about every 10 months, once about every 11 months, or once about every year.

Embodiment 67. The method of any of embodiments 1-43 comprising administering to the human subject a dose of 10 mg of the modified oligonucleotide once every four weeks.

Embodiment 68. The method of any of embodiments 1-43 comprising administering to the human subject a dose of 30 mg of the modified oligonucleotide once every four weeks.

Embodiment 69. The method of any of embodiments 1-43 comprising administering to the human subject a dose of 60 mg of the modified oligonucleotide once every four weeks.

Embodiment 70. The method of any of embodiments 1-43 comprising administering to the human subject a dose of 115 mg of the modified oligonucleotide once every 12 weeks.

Embodiment 71. The method of any of embodiments 67-70 wherein four doses of the modified oligonucleotide are administered.

Embodiment 72. The method of embodiment 71, wherein two doses of the modified oligonucleotide are administered.

Embodiment 73. The method of any of embodiments 1-43 comprising administering to the human subject a dose of 10 mg of the modified oligonucleotide once every four weeks for total of four doses, then administering a dose of 60 mg of the modified oligonucleotide once every 12 weeks thereafter.

Embodiment 74. The method of any of embodiments 1-43 comprising administering to the human subject a dose of 30 mg of the modified oligonucleotide once every four weeks for total of four doses, then administering a dose of 60 mg of the modified oligonucleotide once every 12 weeks thereafter.

Embodiment 75. The method of any of embodiments 1-43 comprising administering to the human subject a dose of 60 mg of the modified oligonucleotide once every four weeks for total of four doses, then administering a dose of 60 mg of the modified oligonucleotide once every 12 weeks thereafter.

Embodiment 76. The method of any of embodiments 1-43 comprising administering to the human subject a dose of 115 mg of the modified oligonucleotide once every 12 weeks.

Embodiment 77. The method of any of embodiments 1-43, comprising administering to the human subject a dose of 60 mg of the modified oligonucleotide once every three months, once every 6 months, or twice a year, once every quarter, or four times yearly.

Embodiment 78. The method of any of embodiments 1-43, comprising administering to the human subject a dose of 115 mg of the modified oligonucleotide once every three months, once every quarter, or four times yearly.

Embodiment 79. The method of any of embodiments 1-43, comprising administering to the human subject a dose of 115 mg of the modified oligonucleotide once every 6 months, or twice a year.

Embodiment 80. The method of any of embodiments 1-43, comprising administering to the human subject a dose of 115 mg of the modified oligonucleotide once every 12 weeks, once every three months, or once a quarter.

Embodiment 81. The method of any of embodiments 1-43, wherein the human subject has Progressive Supranuclear Palsy (PSP), comprising administering to the subject a first dosing regimen comprising administering a dose of 115 mg of the modified oligonucleotide once every 12 weeks, once every 3 months, once every quarter, or four times yearly.

Embodiment 82. The method of embodiment 81, further comprising monitoring safety of the dosing regimen, ceasing the administration of the first dosing regimen, then administering to the subject a second dosing regimen comprising administering
a) a dose of 60 mg of the modified oligonucleotide every 8 weeks or every two months,
b) a dose of 90 mg of the modified oligonucleotide every 12 weeks, every three months, or quarterly, or
c) a dose of 60 mg of the modified oligonucleotide every 4 weeks or every month.

Embodiment 83. The method of any of embodiments 67-82 wherein 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 doses are administered.

Embodiment 84. The method of any of embodiments 1-43 comprising administering to the human subject an initial loading dose of 10 mg of the modified oligonucleotide.

Embodiment 85. The method of embodiment 84, comprising administering to the human subject a second loading dose of 10 mg of the modified oligonucleotide 4 weeks after the initial loading dose.

Embodiment 86. The method of embodiment 85, comprising administering to the human subject a maintenance dose of 10 mg of the modified oligonucleotide 4 weeks after the second loading dose.

Embodiment 87. The method of embodiment 85, comprising administering to the human subject a maintenance dose of 10 mg of the modified oligonucleotide 8 weeks after the second loading dose.

Embodiment 88. The method of embodiment 85, comprising administering to the human subject a maintenance dose of 10 mg of the modified oligonucleotide 12 weeks after the second loading dose.

Embodiment 89. The method of embodiment 85, comprising administering to the human subject a maintenance dose of 10 mg of the modified oligonucleotide 16 weeks after the second loading dose.

Embodiment 90. The method of embodiment 85, comprising administering to the human subject a maintenance dose of 10 mg of the modified oligonucleotide 24 weeks after the second loading dose.

Embodiment 91. The method of embodiment 85, comprising administering to the human subject a maintenance dose of 10 mg of the modified oligonucleotide 6 months after the second loading dose.

Embodiment 92. The method of any of embodiments 1-43 comprising administering to the human subject an initial loading dose of 30 mg of the modified oligonucleotide.

Embodiment 93. The method of embodiment 92, comprising administering to the human subject a second loading dose of 30 mg of the modified oligonucleotide 4 weeks after the initial loading dose.

Embodiment 94. The method of embodiment 93, comprising administering to the human subject a maintenance dose of 30 mg of the modified oligonucleotide 4 weeks after the second loading dose.

Embodiment 95. The method of embodiment 93, comprising administering to the human subject a maintenance dose of 30 mg of the modified oligonucleotide 8 weeks after the second loading dose.

Embodiment 96. The method of embodiment 93, comprising administering to the human subject a maintenance dose of 30 mg of the modified oligonucleotide 12 weeks after the second loading dose.

Embodiment 97. The method of embodiment 93, comprising administering to the human subject a maintenance dose of 30 mg of the modified oligonucleotide 16 weeks after the second loading dose.

Embodiment 98. The method of embodiment 93, comprising administering to the human subject a maintenance dose of 30 mg of the modified oligonucleotide 24 weeks after the second loading dose.

Embodiment 99. The method of embodiment 93, comprising administering to the human subject a maintenance dose of 30 mg of the modified oligonucleotide 6 months after the second loading dose.

Embodiment 100. The method of any of embodiments 1-43 comprising administering to the human subject an initial loading dose of 60 mg of the modified oligonucleotide.

Embodiment 101. The method of embodiment 100, comprising administering to the human subject a second loading dose of 60 mg of the modified oligonucleotide 4 weeks after the initial loading dose.

Embodiment 102. The method of embodiment 101, comprising administering to the human subject a maintenance dose of 60 mg of the modified oligonucleotide 4 weeks after the second loading dose.

Embodiment 103. The method of embodiment 101, comprising administering to the human subject a maintenance dose of 60 mg of the modified oligonucleotide 8 weeks after the second loading dose.

Embodiment 104. The method of embodiment 101, comprising administering to the human subject a maintenance dose of 60 mg of the modified oligonucleotide 12 weeks after the second loading dose.

Embodiment 105. The method of embodiment 101, comprising administering to the human subject a maintenance dose of 60 mg of the modified oligonucleotide 16 weeks after the second loading dose.

Embodiment 106. The method of embodiment 101, comprising administering to the human subject a maintenance dose of 60 mg of the modified oligonucleotide 24 weeks after the second loading dose.

Embodiment 107. The method of embodiment 101, comprising administering to the human subject a maintenance dose of 60 mg of the modified oligonucleotide 6 months after the second loading dose.

Embodiment 108. The method of any of embodiments 1-43 comprising administering to the human subject an initial loading dose of 90 mg of the modified oligonucleotide.

Embodiment 109. The method of embodiment 108, comprising administering to the human subject a second loading dose of 90 mg of the modified oligonucleotide 4 weeks after the initial loading dose.

Embodiment 110. The method of embodiment 109, comprising administering to the human subject a maintenance dose of 90 mg of the modified oligonucleotide 4 weeks after the second loading dose.

Embodiment 111. The method of embodiment 109, comprising administering to the human subject a maintenance dose of 90 mg of the modified oligonucleotide 8 weeks after the second loading dose.

Embodiment 112. The method of embodiment 109, comprising administering to the human subject a maintenance dose of 90 mg of the modified oligonucleotide 12 weeks after the second loading dose.

Embodiment 113. The method of embodiment 109, comprising administering to the human subject a maintenance dose of 90 mg of the modified oligonucleotide 16 weeks after the second loading dose.

Embodiment 114. The method of embodiment 109, comprising administering to the human subject a maintenance dose of 90 mg of the modified oligonucleotide 24 weeks after the second loading dose.

Embodiment 115. The method of embodiment 109, comprising administering to the human subject a maintenance dose of 90 mg of the modified oligonucleotide 6 months after the second loading dose.

Embodiment 116. The method of any of embodiments 1-43 comprising administering to the human subject an initial loading dose of 115 mg of the modified oligonucleotide.

Embodiment 117. The method of embodiment 116, comprising administering to the human subject a second loading dose of 115 mg of the modified oligonucleotide 12 weeks after the initial loading dose.

Embodiment 118. The method of embodiment 117, comprising administering to the human subject a maintenance dose of 115 mg of the modified oligonucleotide 12 weeks after the second loading dose.

Embodiment 119. The method of embodiment 117, comprising administering to the human subject a maintenance dose of 115 mg of the modified oligonucleotide 16 weeks after the second loading dose.

Embodiment 120. The method of embodiment 117, comprising administering to the human subject a maintenance dose of 115 mg of the modified oligonucleotide 24 weeks after the second loading dose.

Embodiment 121. The method of embodiment 117, comprising administering to the human subject a maintenance dose of 115 mg of the modified oligonucleotide 6 months after the second loading dose.

Embodiment 122. The method of any of embodiments 85, 93, 101, 109, or 117, comprising administering to the human subject a maintenance dose of 10 mg, 30 mg, 60 mg, 90 mg, or 115 mg of the modified oligonucleotide 4 weeks after the second loading dose and every 4 weeks thereafter.

Embodiment 123. The method of any of embodiments 85, 93, 101, 109, or 117, comprising administering to the human subject a maintenance dose of 10 mg, 30 mg, 60 mg, 90 mg, or 115 mg of the modified oligonucleotide 8 weeks after the second loading dose and every 4 weeks thereafter.

Embodiment 124. The method of any of embodiments 85, 93, 101, 109, or 117, comprising administering to the human subject a maintenance dose of 10 mg, 30 mg, 60 mg, 90 mg, or 115 mg of the modified oligonucleotide 12 weeks after the second loading dose and every 4 weeks thereafter.

Embodiment 125. The method of any of embodiments 85, 93, 101, 109, or 117, comprising administering to the human subject a maintenance dose of 10 mg, 30 mg, 60 mg, 90 mg, or 115 mg of the modified oligonucleotide 16 weeks after the second loading dose and every 4 weeks thereafter.

Embodiment 126. The method of any of embodiments 85, 93, 101, 109, or 117, comprising administering to the human subject a maintenance dose of 10 mg, 30 mg, 60 mg, 90 mg, or 115 mg of the modified oligonucleotide 24 weeks after the second loading dose and every 4 weeks thereafter.

Embodiment 127. The method of any of embodiments 85, 93, 101, 109, or 117, comprising administering to the human subject a maintenance dose of 10 mg, 30 mg, 60 mg, 90 mg, or 115 mg of the modified oligonucleotide 6 months after the second loading dose and every 4 weeks thereafter.

Embodiment 128. The method of any of embodiments 122-127, wherein at least 2, at least 3, at least 4, at least 5, or at least 6 maintenance doses are administered to the human subject.

Embodiment 129. A method of ameliorating Alzheimer's disease (e.g., mild Alzheimer's disease, mild cognitive impairment due to Alzheimer's disease, and/or mild Alzheimer's disease dementia), reducing Tau RNA, or reducing Tau protein, in a human subject in need thereof, the method comprising intrathecally administering to the human subject a therapeutically effective amount of 10 mg, 30 mg, 60 mg, 90 mg, 115 mg, or about 10 mg, about 30 mg, about 60 mg, about 90 mg, or about 115 mg, or about 60 mg to about 115 mg of a modified oligonucleotide according to the following chemical structure:

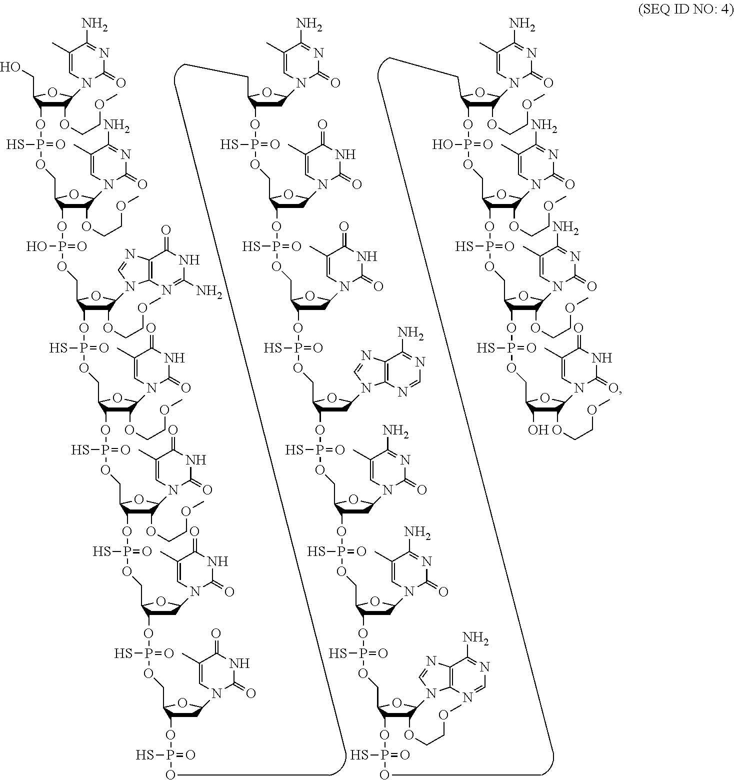

Embodiment 130. The method of embodiment 129, wherein the modified oligonucleotide is the sodium salt or the potassium salt.

Embodiment 131. A method of ameliorating Alzheimer's disease (e.g., mild Alzheimer's disease, mild cognitive impairment due to Alzheimer's disease, and/or mild Alzheimer's disease dementia), reducing Tau RNA, or reducing Tau protein, in a human subject in need thereof, the method comprising intrathecally administering to the human subject a therapeutically effective amount of 10 mg, 30 mg, 60 mg, 90 mg, 115 mg, or about 10 mg, about 30 mg, about 60 mg, about 90 mg, or about 115 mg, or about 60 mg to about 115 mg of a modified oligonucleotide according to the following chemical structure:

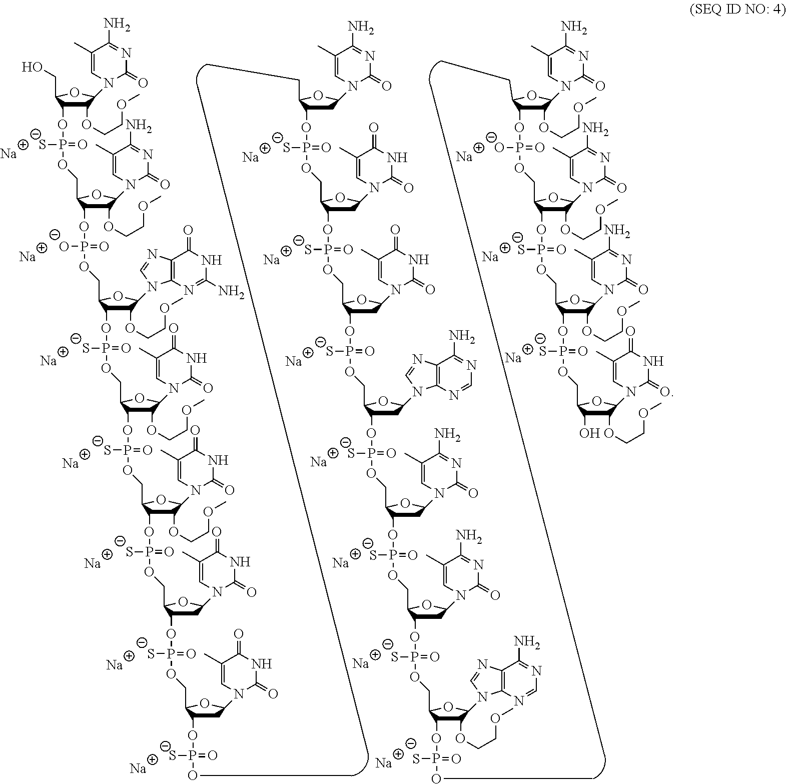

Embodiment 132. A method of ameliorating Alzheimer's disease (e.g., mild Alzheimer's disease, mild cognitive impairment due to Alzheimer's disease, and/or mild Alzheimer's disease dementia), reducing Tau RNA, or reducing Tau protein, in a human subject in need thereof, the method comprising intrathecally administering to the human subject a therapeutically effective amount of 10 mg, 30 mg, 60 mg, 90 mg, 115 mg, or about 10 mg, about 30 mg, about 60 mg, about 90 mg, or about 115 mg, or about 60 mg to about 115 mg of a modified oligonucleotide, wherein the modified oligonucleotide has the following chemical notation (5′ to 3′): mCes mCeo Ges Tes Tes Tds Tds mCds Tds Tds Ads mCds mCds Aes mCeo mCes mCes Te (SEQ TD NO: 4); wherein,
A=an adenine nucleobase,
mC=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

Embodiment 133. The method of any of embodiments 129-133, comprising administering the modified oligonucleotide about once every 4 weeks.

Embodiment 134. The method of any of embodiments 129-133, comprising administering the modified oligonucleotide about once every 8 weeks.

Embodiment 135. The method of any of embodiments 129-133, comprising administering the modified oligonucleotide about once every 12 weeks.

Embodiment 136. The method of any of embodiments 129-133, comprising administering the modified oligonucleotide about once every 16 weeks.

Embodiment 137. The method of any of embodiments 129-133, comprising administering the modified oligonucleotide about once every 24 weeks.

Embodiment 138. The method of any of embodiments 129-133, comprising administering the modified oligonucleotide about once every 6 months.

Embodiment 139. The method of any of embodiments 129-133, comprising administering the modified oligonucleotide once about every 6 months.

Embodiment 140. The method of any of embodiments 129-133, comprising administering the modified oligonucleotide monthly.

Embodiment 141. The method of any of embodiments 129-133, comprising administering the modified oligonucleotide once every two months.

Embodiment 142. The method of any of embodiments 129-133, comprising administering the modified oligonucleotide once every three months.

Embodiment 143. The method of any of embodiments 129-133, comprising administering the modified oligonucleotide quarterly.

Embodiment 144. The method of any of embodiments 129-133, comprising administering the modified oligonucleotide semiannually.

Embodiment 145. The method of any of embodiments 129-133, comprising administering the modified oligonucleotide annually.

Embodiment 146. The method of any of embodiments 129-133, comprising administering the modified oligonucleotide once every two years.

Embodiment 147. The method of any of embodiments 129-133 comprising administering to the human subject a dose of 10 mg of the modified oligonucleotide once every four weeks.

Embodiment 148. The method of any of embodiments 129-133 comprising administering to the human subject a dose of 30 mg of the modified oligonucleotide once every four weeks.

Embodiment 149. The method of any of embodiments 129-133 comprising administering to the human subject a dose of 60 mg of the modified oligonucleotide once every four weeks.

Embodiment 150. The method of any of embodiments 129-133 comprising administering to the human subject a dose of 115 mg of the modified oligonucleotide once every 12 weeks.

Embodiment 151. The method of any of embodiments 147-150 wherein four doses of the modified oligonucleotide are administered.

Embodiment 152. The method of embodiment 151, wherein two doses of the modified oligonucleotide are administered.

Embodiment 153. The method of any of embodiments 129-133 comprising administering to the human subject a dose of 10 mg of the modified oligonucleotide once every four weeks for total of four doses, then administering a dose of 60 mg of the modified oligonucleotide once every 12 weeks thereafter.

Embodiment 154. The method of any of embodiments 129-133 comprising administering to the human subject a dose of 30 mg of the modified oligonucleotide once every four weeks for total of four doses, then administering a dose of 60 mg of the modified oligonucleotide once every 12 weeks thereafter.

Embodiment 155. The method of any of embodiments 129-133 comprising administering to the human subject a dose of 60 mg of the modified oligonucleotide once every four weeks for total of four doses, then administering a dose of 60 mg of the modified oligonucleotide once every 12 weeks thereafter.

Embodiment 156. The method of any of embodiments 129-133 comprising administering to the human subject a dose of 115 mg of the modified oligonucleotide once every 12 weeks.

Embodiment 157. The method of any of embodiments 129-133, comprising administering to the human subject a dose of 60 mg of the modified oligonucleotide once every three months, once every quarter, or four times yearly.

Embodiment 158. The method of any of embodiments 129-133, comprising administering to the human subject a dose of 115 mg of the modified oligonucleotide once every three months, once every quarter, or four times yearly.

Embodiment 159. The method of any of embodiments 129-133, comprising administering to the human subject a dose of 115 mg of the modified oligonucleotide once every 6 months, or twice a year.

Embodiment 160. The method of any of embodiments 129-133, comprising administering to the human subject a dose of 115 mg of the modified oligonucleotide once every 12 weeks, once every three months, or once a quarter.

Embodiment 161. The method of any of embodiments 129-133, wherein the human subject has Progressive Supranuclear Palsy (PSP), comprising administering to the subject a first dosing regimen comprising administering a dose of 115 mg of the modified oligonucleotide once every 12 weeks, once every 3 months, once every quarter, or four times yearly.

Embodiment 162. The method of embodiment 161, further comprising monitoring safety of the dosing regimen, ceasing the administration of the first dosing regimen, then administering to the subject a second dosing regimen comprising administering
a) a dose of 60 mg of the modified oligonucleotide every 8 weeks or every two months,
b) a dose of 90 mg of the modified oligonucleotide every 12 weeks, every three months, or quarterly, or
c) a dose of 60 mg of the modified oligonucleotide every 4 weeks or every month.

Embodiment 163. The method of any of embodiments 153-162 wherein 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 doses are administered.

Embodiment 164. The method of any of embodiments 129-133, comprising administering to the human subject:
a) an initial loading dose of about 10 mg, about 30 mg, about 60 mg, about 90 mg, or about 115 mg of the modified oligonucleotide,
b) a second loading dose of about 10 mg, about 30 mg, about 60 mg, about 90 mg, or about 115 mg of the modified oligonucleotide about 4 weeks, about 8 weeks, or about 12 weeks after administering the initial loading dose;
c) a first maintenance dose of about 10 mg, about 30 mg, about 60 mg, about 90 mg, or about 115 mg of the modified oligonucleotide about 8 or about 12 weeks after administering the second loading dose;
d) a second maintenance dose of about 10 mg, about 30 mg, about 60 mg, about 90 mg, or about 115 mg of the modified oligonucleotide about 8 weeks or about 12 weeks after administering the first maintenance dose.

Embodiment 165. The method of any of embodiments 129-133, comprising administering to the human subject:
a) an initial loading dose of about 10 mg, about 30 mg, about 60 mg, about 90 mg, or about 115 mg of the modified oligonucleotide,
b) a second loading dose of about 10 mg, about 30 mg, about 60 mg, about 90 mg, or about 115 mg of the modified oligonucleotide about 4 weeks, about 8 weeks, or about 12 weeks after administering the initial loading dose;
c) a first maintenance dose of about 10 mg, about 30 mg, about 60 mg, about 90 mg, or about 115 mg of the modified oligonucleotide about 12 weeks after administering the second loading dose;
d) a second maintenance dose of about 10 mg, about 30 mg, about 60 mg, about 90 mg, or about 115 mg of the modified oligonucleotide about 12 weeks after administering the first maintenance dose.

Embodiment 166. The method of any of embodiments 129-165, wherein the subject has AD (e.g., mild Alzheimer's disease, mild cognitive impairment due to Alzheimer's disease, and/or mild Alzheimer's disease dementia), FTD, or PSP, and at least one symptom or hallmark of AD, FTD, or PSP is ameliorated.

Embodiment 167. The method of embodiment 166, wherein the at least one symptom or hallmark comprises loss of memory, cognitive decline, loss of ability to understand or express speech, abnormal behavior, impaired motor function, loss of cognitive function, neuropsychiatric behavior dysfunction, impaired global function, loss of motor function, impaired cognitive function, impaired neuropsychiatric function, impaired daily function, impaired attention, impaired visuoperceptual processing, impaired memory, impaired degree of independence, increased apathy, impaired learning ability, impaired mental concentration, impaired understanding and expression of speech, impaired behavior, depression, irritability, anger, impaired mobility, impaired self-care, pain, discomfort, anxiety, seizures, suicidal ideation, suicidal behavior, or increase in the number and/or volume of neurofibrillary inclusions.

Embodiment 168. The method of any of embodiments 1-167, wherein the human subject has a mutation in at least one gene selected from MAPT, APOE, APP, PSEN1, PSEN2, LRRK2, STX6, EIF2AK3, and MOBP.

Embodiment 169. The method of any of embodiments 1-167, further comprising identifying a mutation in at least one gene selected from MAPT, APOE, APP, PSEN1, and PSEN2 of the human subject.

Embodiment 170. The method of any of embodiments 1-169, wherein the modified oligonucleotide is administered to the CNS of the human subject.

Embodiment 171. The method of any of embodiments 1-170, wherein the modified oligonucleotide is administered by intrathecal administration.

Embodiment 172. The method of any of embodiments 1-170, wherein the modified oligonucleotide is administered by bolus intrathecal administration.

Embodiment 173. The method of any of embodiments 1-172, wherein Tau RNA is reduced.

Embodiment 174. The method of any of embodiments 1-173, wherein Tau protein is reduced.

Embodiment 175. The method of any of embodiments 1-174, comprising detecting an amount of Tau RNA in a biological sample from the human subject.

Embodiment 176. The method of any of embodiments 1-175, comprising detecting an amount of Tau protein in a biological sample from the human subject.

Embodiment 177. The method of embodiment 175 or embodiment 176, wherein the biological sample comprises cerebrospinal fluid.

Embodiment 178. The method of any of embodiments 175-177, wherein the detecting occurs before the administering.

Embodiment 179. The method of any of embodiments 175-177, wherein the detecting occurs after the administering.

Embodiment 180. The method of any of embodiments 175-177, wherein the detecting occurs before and after the administering.

Embodiment 181. The method of any of embodiments 175-180, comprising adjusting the initial loading dose, the loading dose, maintenance dose, or therapeutically effective amount administered after detecting the amount of Tau RNA, Tau protein, or combination thereof.

Embodiment 182. The method of any of embodiments 1-181, comprising analyzing brain activity, brain size, size of neurofibrillary inclusions, volume of neurofibrillary inclusions, amount or concentration of total tau protein, phosphorylated tau protein, or amyloid beta protein in the cerebral spinal fluid, or a combination thereof by performing a magnetic resonance imaging (MRI), Positron Emission Tomography (PET), Electroencephalogram (EEG), or CSF analysis of the subject.

Embodiment 183. The method of embodiment 182, wherein performing the MRI, PET, EEG, or CSF analysis occurs before administering, after administering, or a combination thereof.

Embodiment 184. The method of embodiment 183, comprising determining or adjusting the therapeutically effective amount after performing the MRI, PET, EEG, or CSF analysis.

Embodiment 185. The method of embodiment 184, comprising performing the MRI, PET, EEG, or CSF analysis after administering, and adjusting the frequency of administering after performing the MRI, PET, EEG, or CSF analysis.

Embodiment 186. The method of any of embodiments 182-185, wherein the MRI, PET, EEG, or CSF analysis is performed within 1, 2, 4, 6, 8, 12 or 24 hours of administering.

Embodiment 187. The method of embodiment 186, comprising administering a loading dose once about every 4 weeks and administering a maintenance dose once about every 8 or 16 weeks before performing the first MRI, PET, EEG, or CSF analysis, and administering the maintenance dose less than about every 8 weeks or less than about every 16 weeks.

### I. MAPT

In certain embodiments, described herein are methods of reducing Tau RNA and/or Tau protein in a cell or a biological fluid of a subject. Tau RNA is encoded by the human MAPT gene, located on human 17 (17q21.31). Tau protein is the protein expression product of Tau RNA. Tau protein is highly expressed in neurons relative to other cell types. A representative nucleobase sequence for a human MAPT gene is provided at GENBANK Accession No. NT_010783.14 truncated from nucleotides 2624000 to 2761000 incorporated herein as SEQ ID NO: 2 A representative nucleobase sequence for a human Tau RNA is provided at GENBANK Accession No. NM_001377265., incorporated herein as SEQ ID NO: 1. A representative protein sequence for a human Tau protein is provided at GENBANK Accession No. NP_001364194.1, incorporated here in as SEQ ID NO: 3.

### II. ISIS 814907

In certain embodiments, described herein are methods of administering modified oligonucleotide ISIS 814907 (MAPT_{Rx}), to a subject in need thereof. In certain embodiments, ISIS 814907 is characterized as a 5-8-5 MOE gapmer, having a sequence of (from 5′ to 3′) CCGTTTTCTTACCACCCT (incorporated herein as SEQ ID NO: 4), wherein each of nucleosides 1-5 and 14-18 are 2′-MOE nucleosides and each of nucleosides 6-13 are 2′-deoxynucleosides, wherein the internucleoside linkages between nucleoside 2 and nucleoside 3 and nucleoside 15 to nucleoside 16 are phosphodiester internucleoside linkages and the remainder of the internucleoside linkages are phosphorothioate internucleoside linkages, and wherein each cytosine is a 5-methylcytosine.

In certain embodiments, ISIS 814907 is characterized by the following chemical notation: mCes mCeo Ges Tes Tes Tds Tds mCds Tds Tds Ads mCds mCds Aes mCeo mCes mCes Te (SEQ ID NO: 4); wherein,
A=an adenine,
mC=a 5-methylcytosine,
G=a guanine,
T=a thymine,
e=a 2′-MOE nucleoside,
d=a 2′-deoxynucleoside,
s=a phosphorothioate internucleoside linkage, and
o=a phosphodiester internucleoside linkage.

In certain embodiments, ISIS 814907 is characterized by the following chemical structure:

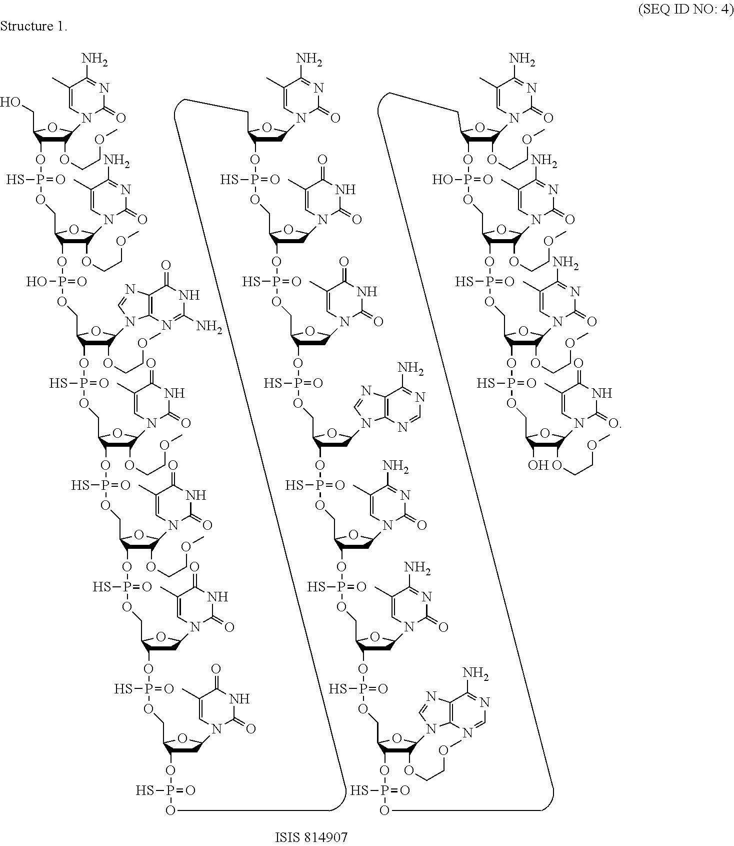

In certain embodiments, the sodium salt of ISIS 814907 is represented by the following chemical structure:

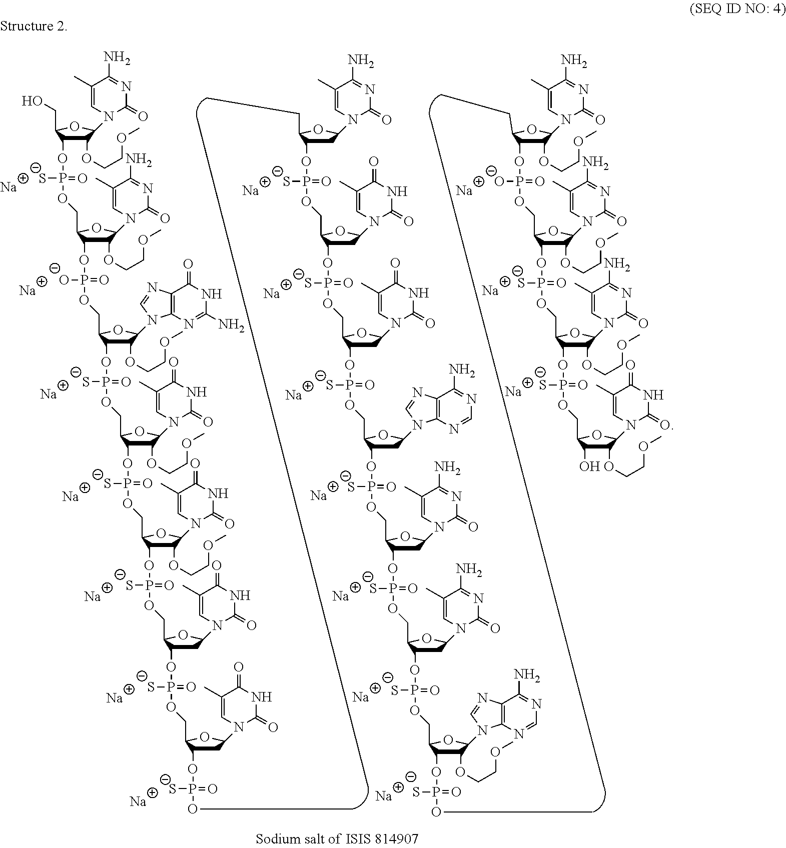

### III. Certain Pharmaceutical Compositions

In certain embodiments, described herein are methods of administering to a subject a pharmaceutical composition comprising the modified oligonucleotide ISIS 814907. In certain embodiments, the pharmaceutical composition comprises a pharmaceutically acceptable diluent or carrier. In certain embodiments, the pharmaceutical composition comprises or consists essentially of a sterile saline solution and the modified oligonucleotide ISIS 814907. In certain embodiments, the sterile saline is pharmaceutical grade saline. In certain embodiments, the pharmaceutical composition comprises or consists essentially of sterile water and the modified oligonucleotide ISIS 814907. In certain embodiments, the sterile water is pharmaceutical grade water. In certain embodiments, the pharmaceutical composition comprises or consists essentially of artificial cerebrospinal fluid (aCSF) and the modified oligonucleotide ISIS 814907. In certain embodiments, the artificial cerebrospinal fluid is pharmaceutical grade. In certain embodiments, the pharmaceutical composition comprises a certain concentration of modified oligonucleotide ISIS 814907 in aCSF, and is diluted in aCSF diluent to achieve the intended clinical dose. In certain embodiments, ISIS 814907 is formulated in aCSF at 30 mg/mL and is diluted in aCSF diluent to achieve the intended clinical dose. In certain embodiments, ISIS 814907 is formulated in aCSF at 20 mg/mL and is diluted in aCSF diluent to achieve the intended clinical dose.

In certain embodiments, pharmaceutical compositions comprise one or more excipients and the modified oligonucleotide ISIS 814907. In certain embodiments, excipients are selected from water, salt solutions, alcohol, polyethylene glycols, gelatin, lactose, amylase, magnesium stearate, talc, silicic acid, viscous paraffin, hydroxymethylcellulose, and polyvinylpyrrolidone.

In certain embodiments, pharmaceutical compositions comprising the modified oligonucleotide ISIS 814907 encompass any pharmaceutically acceptable salt of the modified oligonucleotide ISIS 814907, esters of the modified oligonucleotide ISIS 814907, or salts of such esters. In certain embodiments, pharmaceutical compositions comprising the modified oligonucleotide ISIS 814907 are capable of providing (directly or indirectly) the biologically active metabolite or residue thereof upon administration to a human subject. Accordingly, for example, the disclosure is also drawn to pharmaceutically acceptable salts of the modified oligonucleotide ISIS 814907, prodrugs of the modified oligonucleotide ISIS 814907, pharmaceutically acceptable salts of such prodrugs, and other bioequivalents. Suitable pharmaceutically acceptable salts include, but are not limited to, sodium and potassium salts.

In certain embodiments, pharmaceutical compositions comprise one or more lipid moieties and the modified oligonucleotide ISIS 814907. In certain embodiments, lipid moieties are used to increase distribution of ISIS 814907 to a particular cell or tissue. In certain such methods, the modified oligonucleotide ISIS 814907 is introduced into preformed liposomes or lipoplexes made of mixtures of cationic lipids and neutral lipids. In certain methods, DNA complexes with mono- or poly-cationic lipids are formed without the presence of a neutral lipid.

In certain embodiments, pharmaceutical compositions disclosed herein comprise a delivery system. Examples of delivery systems include, but are not limited to, liposomes and emulsions. Certain delivery systems are useful for preparing pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver modified oligonucleotides described herein to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV)). In certain of such embodiments, a pharmaceutical composition comprises a carrier and is formulated in aqueous solution, such as aCSF, water, or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, the modified oligonucleotide ISIS 814907 acts as an acid. Although ISIS 814907 may be drawn or described in protonated (free acid) form, or ionized and in association with a cation (salt) form, aqueous solutions of ISIS 814907 exist in equilibrium among such forms. For example, a phosphate linkage of ISIS 814907 in aqueous solution exists in equilibrium among free acid, anion, and salt forms. Unless otherwise indicated, the term, “ISIS 814907,” is intended to include all such forms. Moreover, ISIS 814907 has several such linkages, each of which is in equilibrium. Thus, ISIS 814907 exists in solution in an ensemble of forms at multiple positions all at equilibrium. The term “ISIS 814907” is intended to include all such forms. Drawn structures necessarily depict a single form. Nevertheless, unless otherwise indicated, such drawings are likewise intended to include corresponding forms. Herein, a structure depicting the free acid of ISIS 814907 followed by the term “or a salt thereof” expressly includes all such forms that may be fully or partially protonated/de-protonated/in association with a cation. In certain instances, one or more specific cation is identified.

In certain embodiments, ISIS 814907 is in aqueous solution with sodium. In certain embodiments, ISIS 814907 is in aqueous solution with potassium. In certain embodiments, ISIS 814907 is in PBS. In certain embodiments, ISIS 814907 is in water. In certain such embodiments, the pH of the solution is adjusted with NaOH and/or HCl to achieve a desired pH.

Herein, certain specific doses are described. For clarity, a dose of ISIS 814907 in milligrams indicates the mass of the free acid form of ISIS 814907. As described above, in aqueous solution, the free acid is in equilibrium with anionic and salt forms. However, for the purpose of calculating dose, it is assumed that ISIS 814907 exists as a solvent-free, sodium-acetate free, anhydrous, free acid. For example, where ISIS 814907 is in solution comprising sodium (e.g., saline), ISIS 814907 may be partially or fully de-protonated and in association with Na+ ions. However, the mass of the protons is nevertheless counted toward the weight of the dose, and the mass of the Na+ ions are not counted toward the weight of the dose. Thus, for example, a dose of 60 mg of ISIS 814907 equals the number of fully protonated molecules that weighs 60 mg. This would be equivalent to 63.48 mg of solvent-free, sodium-acetate free, anhydrous sodiated ISIS 814907. Similarly, a dose of 115 mg of ISIS 814907 equals the number of fully protonated molecules that weighs 115 mg. This would be equivalent to 121.67 mg of solvent-free, sodium-acetate free, anhydrous sodiated ISIS 814907.

### IV. Certain Dosage Amounts

In certain embodiments, described herein are methods of administering to a subject a therapeutically effective amount of the modified oligonucleotide ISIS 814907. In certain embodiments, the therapeutically effective amount is 10 mg. In certain embodiments, the therapeutically effective amount is 30 mg. In certain embodiments, the therapeutically effective amount is 60 mg. In certain embodiments, the therapeutically effective amount is 90 mg. In certain embodiments, the therapeutically effective amount is 115 mg.

In certain embodiments, the therapeutically effective amount is any of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, or 350 mg.

In certain embodiments, the therapeutically effective amount is any of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, and about 350 mg.

In certain embodiments, the therapeutically effective amount is any of 50 mg, 50.1 mg, 50.2 mg, 50.3 mg, 50.4 mg, 50.5 mg, 50.6 mg, 50.7 mg, 50.8 mg, 50.9 mg, 51 mg, 51.1 mg, 51.2 mg, 51.3 mg, 51.4 mg, 51.5 mg, 51.6 mg, 51.7 mg, 51.8 mg, 51.9 mg, 52 mg, 52.1 mg, 52.2 mg, 52.3 mg, 52.4 mg, 52.5 mg, 52.6 mg, 52.7 mg, 52.8 mg, 52.9 mg, 53 mg, 53.1 mg, 53.2 mg, 53.3 mg, 53.4 mg, 53.5 mg, 53.6 mg, 53.7 mg, 53.8 mg, 53.9 mg, 54 mg, 54.1 mg, 54.2 mg, 54.3 mg, 54.4 mg, 54.5 mg, 54.6 mg, 54.7 mg, 54.8 mg, 54.9 mg, 55 mg, 55.1 mg, 55.2 mg, 55.3 mg, 55.4 mg, 55.5 mg, 55.6 mg, 55.7 mg, 55.8 mg, 55.9 mg, 56 mg, 56.1 mg, 56.2 mg, 56.3 mg, 56.4 mg, 56.5 mg, 56.6 mg, 56.7 mg, 56.8 mg, 56.9 mg, 57 mg, 57.1 mg, 57.2 mg, 57.3 mg, 57.4 mg, 57.5 mg, 57.6 mg, 57.7 mg, 57.8 mg, 57.9 mg, 58 mg, 58.1 mg, 58.2 mg, 58.3 mg, 58.4 mg, 58.5 mg, 58.6 mg, 58.7 mg, 58.8 mg, 58.9 mg, 59 mg, 59.1 mg, 59.2 mg, 59.3 mg, 59.4 mg, 59.5 mg, 59.6 mg, 59.7 mg, 59.8 mg, 59.9 mg, 60 mg, 60.1 mg, 60.2 mg, 60.3 mg, 60.4 mg, 60.5 mg, 60.6 mg, 60.7 mg, 60.8 mg, 60.9 mg, 61 mg, 61.1 mg, 61.2 mg, 61.3 mg, 61.4 mg, 61.5 mg, 61.6 mg, 61.7 mg, 61.8 mg, 61.9 mg, 62 mg, 62.1 mg, 62.2 mg, 62.3 mg, 62.4 mg, 62.5 mg, 62.6 mg, 62.7 mg, 62.8 mg, 62.9 mg, 63 mg, 63.1 mg, 63.2 mg, 63.3 mg, 63.4 mg, 63.5 mg, 63.6 mg, 63.7 mg, 63.8 mg, 63.9 mg, 64 mg, 64.1 mg, 64.2 mg, 64.3 mg, 64.4 mg, 64.5 mg, 64.6 mg, 64.7 mg, 64.8 mg, 64.9 mg, 65 mg, 65.1 mg, 65.2 mg, 65.3 mg, 65.4 mg, 65.5 mg, 65.6 mg, 65.7 mg, 65.8 mg, 65.9 mg, 66 mg, 66.1 mg, 66.2 mg, 66.3 mg, 66.4 mg, 66.5 mg, 66.6 mg, 66.7 mg, 66.8 mg, 66.9 mg, 67 mg, 67.1 mg, 67.2 mg, 67.3 mg, 67.4 mg, 67.5 mg, 67.6 mg, 67.7 mg, 67.8 mg, 67.9 mg, 68 mg, 68.1 mg, 68.2 mg, 68.3 mg, 68.4 mg, 68.5 mg, 68.6 mg, 68.7 mg, 68.8 mg, 68.9 mg, 69 mg, 69.1 mg, 69.2 mg, 69.3 mg, 69.4 mg, 69.5 mg, 69.6 mg, 69.7 mg, 69.8 mg, 69.9 mg, and 70 mg.

In certain embodiments, the therapeutically effective amount is any of about 50 mg, about 50.1 mg, about 50.2 mg, about 50.3 mg, about 50.4 mg, about 50.5 mg, about 50.6 mg, about 50.7 mg, about 50.8 mg, about 50.9 mg, about 51 mg, about 51.1 mg, about 51.2 mg, about 51.3 mg, about 51.4 mg, about 51.5 mg, about 51.6 mg, about 51.7 mg, about 51.8 mg, about 51.9 mg, about 52 mg, about 52.1 mg, about 52.2 mg, about 52.3 mg, about 52.4 mg, about 52.5 mg, about 52.6 mg, about 52.7 mg, about 52.8 mg, about 52.9 mg, about 53 mg, about 53.1 mg, about 53.2 mg, about 53.3 mg, about 53.4 mg, about 53.5 mg, about 53.6 mg, about 53.7 mg, about 53.8 mg, about 53.9 mg, about 54 mg, about 54.1 mg, about 54.2 mg, about 54.3 mg, about 54.4 mg, about 54.5 mg, about 54.6 mg, about 54.7 mg, about 54.8 mg, about 54.9 mg, about 55 mg, about 55.1 mg, about 55.2 mg, about 55.3 mg, about 55.4 mg, about 55.5 mg, about 55.6 mg, about 55.7 mg, about 55.8 mg, about 55.9 mg, about 56 mg, about 56.1 mg, about 56.2 mg, about 56.3 mg, about 56.4 mg, about 56.5 mg, about 56.6 mg, about 56.7 mg, about 56.8 mg, about 56.9 mg, about 57 mg, about 57.1 mg, about 57.2 mg, about 57.3 mg, about 57.4 mg, about 57.5 mg, about 57.6 mg, about 57.7 mg, about 57.8 mg, about 57.9 mg, about 58 mg, about 58.1 mg, about 58.2 mg, about 58.3 mg, about 58.4 mg, about 58.5 mg, about 58.6 mg, about 58.7 mg, about 58.8 mg, about 58.9 mg, about 59 mg, about 59.1 mg, about 59.2 mg, about 59.3 mg, about 59.4 mg, about 59.5 mg, about 59.6 mg, about 59.7 mg, about 59.8 mg, about 59.9 mg, about 60 mg, about 60.1 mg, about 60.2 mg, about 60.3 mg, about 60.4 mg, about 60.5 mg, about 60.6 mg, about 60.7 mg, about 60.8 mg, about 60.9 mg, about 61 mg, about 61.1 mg, about 61.2 mg, about 61.3 mg, about 61.4 mg, about 61.5 mg, about 61.6 mg, about 61.7 mg, about 61.8 mg, about 61.9 mg, about 62 mg, about 62.1 mg, about 62.2 mg, about 62.3 mg, about 62.4 mg, about 62.5 mg, about 62.6 mg, about 62.7 mg, about 62.8 mg, about 62.9 mg, about 63 mg, about 63.1 mg, about 63.2 mg, about 63.3 mg, about 63.4 mg, about 63.5 mg, about 63.6 mg, about 63.7 mg, about 63.8 mg, about 63.9 mg, about 64 mg, about 64.1 mg, about 64.2 mg, about 64.3 mg, about 64.4 mg, about 64.5 mg, about 64.6 mg, about 64.7 mg, about 64.8 mg, about 64.9 mg, about 65 mg, about 65.1 mg, about 65.2 mg, about 65.3 mg, about 65.4 mg, about 65.5 mg, about 65.6 mg, about 65.7 mg, about 65.8 mg, about 65.9 mg, about 66 mg, about 66.1 mg, about 66.2 mg, about 66.3 mg, about 66.4 mg, about 66.5 mg, about 66.6 mg, about 66.7 mg, about 66.8 mg, about 66.9 mg, about 67 mg, about 67.1 mg, about 67.2 mg, about 67.3 mg, about 67.4 mg, about 67.5 mg, about 67.6 mg, about 67.7 mg, about 67.8 mg, about 67.9 mg, about 68 mg, about 68.1 mg, about 68.2 mg, about 68.3 mg, about 68.4 mg, about 68.5 mg, about 68.6 mg, about 68.7 mg, about 68.8 mg, about 68.9 mg, about 69 mg, about 69.1 mg, about 69.2 mg, about 69.3 mg, about 69.4 mg, about 69.5 mg, about 69.6 mg, about 69.7 mg, about 69.8 mg, about 69.9 mg, and about 70 mg.

In certain embodiments, the therapeutically effective amount is any of 80 mg, 80.1 mg, 80.2 mg, 80.3 mg, 80.4 mg, 80.5 mg, 80.6 mg, 80.7 mg, 80.8 mg, 80.9 mg, 81 mg, 81.1 mg, 81.2 mg, 81.3 mg, 81.4 mg, 81.5 mg, 81.6 mg, 81.7 mg, 81.8 mg, 81.9 mg, 82 mg, 82.1 mg, 82.2 mg, 82.3 mg, 82.4 mg, 82.5 mg, 82.6 mg, 82.7 mg, 82.8 mg, 82.9 mg, 83 mg, 83.1 mg, 83.2 mg, 83.3 mg, 83.4 mg, 83.5 mg, 83.6 mg, 83.7 mg, 83.8 mg, 83.9 mg, 84 mg, 84.1 mg, 84.2 mg, 84.3 mg, 84.4 mg, 84.5 mg, 84.6 mg, 84.7 mg, 84.8 mg, 84.9 mg, 85 mg, 85.1 mg, 85.2 mg, 85.3 mg, 85.4 mg, 85.5 mg, 85.6 mg, 85.7 mg, 85.8 mg, 85.9 mg, 86 mg, 86.1 mg, 86.2 mg, 86.3 mg, 86.4 mg, 86.5 mg, 86.6 mg, 86.7 mg, 86.8 mg, 86.9 mg, 87 mg, 87.1 mg, 87.2 mg, 87.3 mg, 87.4 mg, 87.5 mg, 87.6 mg, 87.7 mg, 87.8 mg, 87.9 mg, 88 mg, 88.1 mg, 88.2 mg, 88.3 mg, 88.4 mg, 88.5 mg, 88.6 mg, 88.7 mg, 88.8 mg, 88.9 mg, 89 mg, 89.1 mg, 89.2 mg, 89.3 mg, 89.4 mg, 89.5 mg, 89.6 mg, 89.7 mg, 89.8 mg, 89.9 mg, 90 mg, 90.1 mg, 90.2 mg, 90.3 mg, 90.4 mg, 90.5 mg, 90.6 mg, 90.7 mg, 90.8 mg, 90.9 mg, 91 mg, 91.1 mg, 91.2 mg, 91.3 mg, 91.4 mg, 91.5 mg, 91.6 mg, 91.7 mg, 91.8 mg, 91.9 mg, 92 mg, 92.1 mg, 92.2 mg, 92.3 mg, 92.4 mg, 92.5 mg, 92.6 mg, 92.7 mg, 92.8 mg, 92.9 mg, 93 mg, 93.1 mg, 93.2 mg, 93.3 mg, 93.4 mg, 93.5 mg, 93.6 mg, 93.7 mg, 93.8 mg, 93.9 mg, 94 mg, 94.1 mg, 94.2 mg, 94.3 mg, 94.4 mg, 94.5 mg, 94.6 mg, 94.7 mg, 94.8 mg, 94.9 mg, 95 mg, 95.1 mg, 95.2 mg, 95.3 mg, 95.4 mg, 95.5 mg, 95.6 mg, 95.7 mg, 95.8 mg, 95.9 mg, 96 mg, 96.1 mg, 96.2 mg, 96.3 mg, 96.4 mg, 96.5 mg, 96.6 mg, 96.7 mg, 96.8 mg, 96.9 mg, 97 mg, 97.1 mg, 97.2 mg, 97.3 mg, 97.4 mg, 97.5 mg, 97.6 mg, 97.7 mg, 97.8 mg, 97.9 mg, 98 mg, 98.1 mg, 98.2 mg, 98.3 mg, 98.4 mg, 98.5 mg, 98.6 mg, 98.7 mg, 98.8 mg, 98.9 mg, 99 mg, 99.1 mg, 99.2 mg, 99.3 mg, 99.4 mg, 99.5 mg, 99.6 mg, 99.7 mg, 99.8 mg, 99.9 mg, and 100 mg.

In certain embodiments, the therapeutically effective amount is any of about 80 mg, about 80.1 mg, about 80.2 mg, about 80.3 mg, about 80.4 mg, about 80.5 mg, about 80.6 mg, about 80.7 mg, about 80.8 mg, about 80.9 mg, about 81 mg, about 81.1 mg, about 81.2 mg, about 81.3 mg, about 81.4 mg, about 81.5 mg, about 81.6 mg, about 81.7 mg, about 81.8 mg, about 81.9 mg, about 82 mg, about 82.1 mg, about 82.2 mg, about 82.3 mg, about 82.4 mg, about 82.5 mg, about 82.6 mg, about 82.7 mg, about 82.8 mg, about 82.9 mg, about 83 mg, about 83.1 mg, about 83.2 mg, about 83.3 mg, about 83.4 mg, about 83.5 mg, about 83.6 mg, about 83.7 mg, about 83.8 mg, about 83.9 mg, about 84 mg, about 84.1 mg, about 84.2 mg, about 84.3 mg, about 84.4 mg, about 84.5 mg, about 84.6 mg, about 84.7 mg, about 84.8 mg, about 84.9 mg, about 85 mg, about 85.1 mg, about 85.2 mg, about 85.3 mg, about 85.4 mg, about 85.5 mg, about 85.6 mg, about 85.7 mg, about 85.8 mg, about 85.9 mg, about 86 mg, about 86.1 mg, about 86.2 mg, about 86.3 mg, about 86.4 mg, about 86.5 mg, about 86.6 mg, about 86.7 mg, about 86.8 mg, about 86.9 mg, about 87 mg, about 87.1 mg, about 87.2 mg, about 87.3 mg, about 87.4 mg, about 87.5 mg, about 87.6 mg, about 87.7 mg, about 87.8 mg, about 87.9 mg, about 88 mg, about 88.1 mg, about 88.2 mg, about 88.3 mg, about 88.4 mg, about 88.5 mg, about 88.6 mg, about 88.7 mg, about 88.8 mg, about 88.9 mg, about 89 mg, about 89.1 mg, about 89.2 mg, about 89.3 mg, about 89.4 mg, about 89.5 mg, about 89.6 mg, about 89.7 mg, about 89.8 mg, about 89.9 mg, about 90 mg, about 90.1 mg, about 90.2 mg, about 90.3 mg, about 90.4 mg, about 90.5 mg, about 90.6 mg, about 90.7 mg, about 90.8 mg, about 90.9 mg, about 91 mg, about 91.1 mg, about 91.2 mg, about 91.3 mg, about 91.4 mg, about 91.5 mg, about 91.6 mg, about 91.7 mg, about 91.8 mg, about 91.9 mg, about 92 mg, about 92.1 mg, about 92.2 mg, about 92.3 mg, about 92.4 mg, about 92.5 mg, about 92.6 mg, about 92.7 mg, about 92.8 mg, about 92.9 mg, about 93 mg, about 93.1 mg, about 93.2 mg, about 93.3 mg, about 93.4 mg, about 93.5 mg, about 93.6 mg, about 93.7 mg, about 93.8 mg, about 93.9 mg, about 94 mg, about 94.1 mg, about 94.2 mg, about 94.3 mg, about 94.4 mg, about 94.5 mg, about 94.6 mg, about 94.7 mg, about 94.8 mg, about 94.9 mg, about 95 mg, about 95.1 mg, about 95.2 mg, about 95.3 mg, about 95.4 mg, about 95.5 mg, about 95.6 mg, about 95.7 mg, about 95.8 mg, about 95.9 mg, about 96 mg, about 96.1 mg, about 96.2 mg, about 96.3 mg, about 96.4 mg, about 96.5 mg, about 96.6 mg, about 96.7 mg, about 96.8 mg, about 96.9 mg, about 97 mg, about 97.1 mg, about 97.2 mg, about 97.3 mg, about 97.4 mg, about 97.5 mg, about 97.6 mg, about 97.7 mg, about 97.8 mg, about 97.9 mg, about 98 mg, about 98.1 mg, about 98.2 mg, about 98.3 mg, about 98.4 mg, about 98.5 mg, about 98.6 mg, about 98.7 mg, about 98.8 mg, about 98.9 mg, about 99 mg, about 99.1 mg, about 99.2 mg, about 99.3 mg, about 99.4 mg, about 99.5 mg, about 99.6 mg, about 99.7 mg, about 99.8 mg, about 99.9 mg, and about 100 mg.

In certain embodiments, the therapeutically effective amount is any of 105 mg, 105.1 mg, 105.2 mg, 105.3 mg, 105.4 mg, 105.5 mg, 105.6 mg, 105.7 mg, 105.8 mg, 105.9 mg, 106 mg, 106.1 mg, 106.2 mg, 106.3 mg, 106.4 mg, 106.5 mg, 106.6 mg, 106.7 mg, 106.8 mg, 106.9 mg, 107 mg, 107.1 mg, 107.2 mg, 107.3 mg, 107.4 mg, 107.5 mg, 107.6 mg, 107.7 mg, 107.8 mg, 107.9 mg, 108 mg, 108.1 mg, 108.2 mg, 108.3 mg, 108.4 mg, 108.5 mg, 108.6 mg, 108.7 mg, 108.8 mg, 108.9 mg, 109 mg, 109.1 mg, 109.2 mg, 109.3 mg, 109.4 mg, 109.5 mg, 109.6 mg, 109.7 mg, 109.8 mg, 109.9 mg, 110 mg, 110.1 mg, 110.2 mg, 110.3 mg, 110.4 mg, 110.5 mg, 110.6 mg, 110.7 mg, 110.8 mg, 110.9 mg, 111 mg, 111.1 mg, 111.2 mg, 111.3 mg, 111.4 mg, 111.5 mg, 111.6 mg, 111.7 mg, 111.8 mg, 111.9 mg, 112 mg, 112.1 mg, 112.2 mg, 112.3 mg, 112.4 mg, 112.5 mg, 112.6 mg, 112.7 mg, 112.8 mg, 112.9 mg, 113 mg, 113.1 mg, 113.2 mg, 113.3 mg, 113.4 mg, 113.5 mg, 113.6 mg, 113.7 mg, 113.8 mg, 113.9 mg, 114 mg, 114.1 mg, 114.2 mg, 114.3 mg, 114.4 mg, 114.5 mg, 114.6 mg, 114.7 mg, 114.8 mg, 114.9 mg, 115 mg, 115.1 mg, 115.2 mg, 115.3 mg, 115.4 mg, 115.5 mg, 115.6 mg, 115.7 mg, 115.8 mg, 115.9 mg, 116 mg, 116.1 mg, 116.2 mg, 116.3 mg, 116.4 mg, 116.5 mg, 116.6 mg, 116.7 mg, 116.8 mg, 116.9 mg, 117 mg, 117.1 mg, 117.2 mg, 117.3 mg, 117.4 mg, 117.5 mg, 117.6 mg, 117.7 mg, 117.8 mg, 117.9 mg, 118 mg, 118.1 mg, 118.2 mg, 118.3 mg, 118.4 mg, 118.5 mg, 118.6 mg, 118.7 mg, 118.8 mg, 118.9 mg, 119 mg, 119.1 mg, 119.2 mg, 119.3 mg, 119.4 mg, 119.5 mg, 119.6 mg, 119.7 mg, 119.8 mg, 119.9 mg, 120 mg, 120.1 mg, 120.2 mg, 120.3 mg, 120.4 mg, 120.5 mg, 120.6 mg, 120.7 mg, 120.8 mg, 120.9 mg, 121 mg, 121.1 mg, 121.2 mg, 121.3 mg, 121.4 mg, 121.5 mg, 121.6 mg, 121.7 mg, 121.8 mg, 121.9 mg, 122 mg, 122.1 mg, 122.2 mg, 122.3 mg, 122.4 mg, 122.5 mg, 122.6 mg, 122.7 mg, 122.8 mg, 122.9 mg, 123 mg, 123.1 mg, 123.2 mg, 123.3 mg, 123.4 mg, 123.5 mg, 123.6 mg, 123.7 mg, 123.8 mg, 123.9 mg, 124 mg, 124.1 mg, 124.2 mg, 124.3 mg, 124.4 mg, 124.5 mg, 124.6 mg, 124.7 mg, 124.8 mg, 124.9 mg, and 125 mg.

In certain embodiments, the therapeutically effective amount is any of about 105 mg, about 105.1 mg, about 105.2 mg, about 105.3 mg, about 105.4 mg, about 105.5 mg, about 105.6 mg, about 105.7 mg, about 105.8 mg, about 105.9 mg, about 106 mg, about 106.1 mg, about 106.2 mg, about 106.3 mg, about 106.4 mg, about 106.5 mg, about 106.6 mg, about 106.7 mg, about 106.8 mg, about 106.9 mg, about 107 mg, about 107.1 mg, about 107.2 mg, about 107.3 mg, about 107.4 mg, about 107.5 mg, about 107.6 mg, about 107.7 mg, about 107.8 mg, about 107.9 mg, about 108 mg, about 108.1 mg, about 108.2 mg, about 108.3 mg, about 108.4 mg, about 108.5 mg, about 108.6 mg, about 108.7 mg, about 108.8 mg, about 108.9 mg, about 109 mg, about 109.1 mg, about 109.2 mg, about 109.3 mg, about 109.4 mg, about 109.5 mg, about 109.6 mg, about 109.7 mg, about 109.8 mg, about 109.9 mg, about 110 mg, about 110.1 mg, about 110.2 mg, about 110.3 mg, about 110.4 mg, about 110.5 mg, about 110.6 mg, about 110.7 mg, about 110.8 mg, about 110.9 mg, about 111 mg, about 111.1 mg, about 111.2 mg, about 111.3 mg, about 111.4 mg, about 111.5 mg, about 111.6 mg, about 111.7 mg, about 111.8 mg, about 111.9 mg, about 112 mg, about 112.1 mg, about 112.2 mg, about 112.3 mg, about 112.4 mg, about 112.5 mg, about 112.6 mg, about 112.7 mg, about 112.8 mg, about 112.9 mg, about 113 mg, about 113.1 mg, about 113.2 mg, about 113.3 mg, about 113.4 mg, about 113.5 mg, about 113.6 mg, about 113.7 mg, about 113.8 mg, about 113.9 mg, about 114 mg, about 114.1 mg, about 114.2 mg, about 114.3 mg, about 114.4 mg, about 114.5 mg, about 114.6 mg, about 114.7 mg, about 114.8 mg, about 114.9 mg, about 115 mg, about 115.1 mg, about 115.2 mg, about 115.3 mg, about 115.4 mg, about 115.5 mg, about 115.6 mg, about 115.7 mg, about 115.8 mg, about 115.9 mg, about 116 mg, about 116.1 mg, about 116.2 mg, about 116.3 mg, about 116.4 mg, about 116.5 mg, about 116.6 mg, about 116.7 mg, about 116.8 mg, about 116.9 mg, about 117 mg, about 117.1 mg, about 117.2 mg, about 117.3 mg, about 117.4 mg, about 117.5 mg, about 117.6 mg, about 117.7 mg, about 117.8 mg, about 117.9 mg, about 118 mg, about 118.1 mg, about 118.2 mg, about 118.3 mg, about 118.4 mg, about 118.5 mg, about 118.6 mg, about 118.7 mg, about 118.8 mg, about 118.9 mg, about 119 mg, about 119.1 mg, about 119.2 mg, about 119.3 mg, about 119.4 mg, about 119.5 mg, about 119.6 mg, about 119.7 mg, about 119.8 mg, about 119.9 mg, about 120 mg, about 120.1 mg, about 120.2 mg, about 120.3 mg, about 120.4 mg, about 120.5 mg, about 120.6 mg, about 120.7 mg, about 120.8 mg, about 120.9 mg, about 121 mg, about 121.1 mg, about 121.2 mg, about 121.3 mg, about 121.4 mg, about 121.5 mg, about 121.6 mg, about 121.7 mg, about 121.8 mg, about 121.9 mg, about 122 mg, about 122.1 mg, about 122.2 mg, about 122.3 mg, about 122.4 mg, about 122.5 mg, about 122.6 mg, about 122.7 mg, about 122.8 mg, about 122.9 mg, about 123 mg, about 123.1 mg, about 123.2 mg, about 123.3 mg, about 123.4 mg, about 123.5 mg, about 123.6 mg, about 123.7 mg, about 123.8 mg, about 123.9 mg, about 124 mg, about 124.1 mg, about 124.2 mg, about 124.3 mg, about 124.4 mg, about 124.5 mg, about 124.6 mg, about 124.7 mg, about 124.8 mg, about 124.9 mg, and about 125 mg.

In certain embodiments, the therapeutically effective amount is any of 40 mg to 200 mg, 40 mg to 190 mg, 40 mg to 180 mg, 40 mg to 170 mg, from 40 mg to 160 mg, 40 mg to 150 mg, 40 mg to 140 mg, 40 mg to 120 mg, 40 mg to 110 mg, 40 mg to 100 mg, 40 mg to 80 mg, 40 mg to 70 mg, 40 mg to 60 mg, 40 mg to 50 mg, 50 mg to 200 mg, 50 mg to 190 mg, 50 mg to 180 mg, 50 mg to 170 mg, 50 mg to 160 mg, 50 mg to 150 mg, 50 mg to 140 mg, 50 mg to 120 mg, 50 mg to 110 mg, 50 mg to 100 mg, 50 mg to 80 mg, 50 mg to 70 mg, 50 mg to 60 mg, 60 mg to 200 mg, 60 mg to 190 mg, 60 mg to 180 mg, 60 mg to 170 mg, 60 mg to 160 mg, 60 mg to 150 mg, 60 mg to 140 mg, 60 mg to 120 mg, 60 mg to 115 mg, 60 mg to 110 mg, 60 mg to 100 mg, 60 mg to 80 mg, 60 mg to 70 mg, 70 mg to 200 mg, 70 mg to 190 mg, 70 mg to 180 mg, 70 mg to 170 mg, 70 mg to 160 mg, 70 mg to 150 mg, 70 mg to 140 mg, 70 mg to 120 mg, 70 mg to 110 mg, 70 mg to 100 mg, 70 mg to 80 mg, 80 mg to 200 mg, 80 mg to 190 mg, 80 mg to 180 mg, 80 mg to 170 mg, 80 mg to 160 mg, 80 mg to 150 mg, 80 mg to 140 mg, 80 mg to 120 mg, 80 mg to 110 mg, 80 mg to 100 mg, 80 mg to 90 mg, 90 mg to 200 mg, 90 mg to 190 mg, 90 mg to 180 mg, 90 mg to 170 mg, 90 mg to 160 mg, 90 mg to 150 mg, 90 mg to 140 mg, 90 mg to 120 mg, 90 mg to 110 mg, 90 mg to 100 mg, 100 mg to 200 mg, 100 mg to 190 mg, 100 mg to 180 mg, 100 mg to 170 mg, 100 mg to 160 mg, 100 mg to 150 mg, 100 mg to 140 mg, 100 mg to 120 mg, 100 mg to 110 mg, 110 mg to 200 mg, 110 mg to 190 mg, 110 mg to 180 mg, 110 mg to 170 mg, 110 mg to 160 mg, 110 mg to 150 mg, 110 mg to 140 mg, 110 mg to 130 mg, 110 mg to 120 mg, 120 mg to 200 mg, 120 mg to 190 mg, 120 mg to 180 mg, 120 mg to 170 mg, 120 mg to 160 mg, 120 mg to 150 mg, 120 mg to 140 mg, 120 mg to 130 mg, 130 mg to 200 mg, 130 mg to 190 mg, 130 mg to 180 mg, 130 mg to 170 mg, 130 mg to 160 mg, 130 mg to 150 mg, 130 mg to 140 mg, 140 mg to 200 mg, 140 mg to 190 mg, 140 mg to 180 mg, 140 mg to 170 mg, 140 mg to 160 mg, 140 mg to 150 mg, 150 mg to 200 mg, 150 mg to 190 mg, 150 mg to 180 mg, 150 mg to 170 mg, 150 mg to 160 mg, 160 mg to 200 mg, 160 mg to 190 mg, 160 mg to 180 mg, 160 mg to 170 mg, 180 mg to 200 mg, 180 mg to 190 mg, 190 mg to 200 mg, 105 mg to 135 mg, 105 mg to 130 mg, 105 mg to 125 mg 105 mg to 120 mg, 110 mg to 135 mg, 110 mg to 130 mg, 110 mg to 125 mg, 110 mg to 120 mg, 115 mg to 135 mg, 115 mg to 130 mg, 115 mg to 125 mg, 115 mg to 120 mg, 115 mg to 125 mg, 115 mg to 120 mg, 120 mg to 135 mg, 120 mg to 125 mg, 125 mg to 140 mg, 125 mg to 130 mg, 130 mg to 135 mg, 135 mg to 140 mg, 120 mg to 129 mg, 120 mg to 128 mg, 120 mg to 127 mg, 120 mg to 86 mg, 120 mg to 124 mg, 120 mg to 123 mg, 120 mg to 122 mg, 120 mg to 121 mg, 121 mg to 130 mg, 122 mg to 129 mg, 122 mg to 128 mg, 122 mg to 127 mg, 122 mg to 126 mg, 122 mg to 125 mg, 122 mg to 124 mg, 122 mg to 123 mg, 123 mg to 130 mg, 123 mg to 129 mg, 123 mg to 128 mg, 123 mg to 127 mg, 123 mg to 126 mg, 123 mg to 125 mg, 123 mg to 124 mg, 124 mg to 130 mg, 124 mg to 129 mg, 124 mg to 128 mg, 124 mg to 127 mg, 124 mg to 126 mg, 124 mg to 125 mg, 125 mg to 129 mg, 125 mg to 128 mg, 125 mg to 127 mg, 125 mg to 126 mg, 126 mg to 130 mg, 126 mg to 129 mg, 126 mg to 128 mg, 126 mg to 127 mg, 127 mg to 130 mg, 127 mg to 129 mg, 127 mg to 128 mg, 128 mg to 130 mg, 128 mg to 129 mg, and 129 mg to 130 mg.

In certain embodiments, the therapeutically effective amount is any of less than 350 mg, less than 345 mg, less than 340 mg, less than 335 mg, less than 330 mg, less than 325 mg, less than 320 mg, less than 315 mg, less than 310 mg, less than 305 mg, less than 300 mg, less than 295 mg, less than 290 mg, less than 285 mg, less than 280 mg, less than 275 mg, less than 270 mg, less than 265 mg, less than 260 mg, less than 255 mg, less than 250 mg, less than 245 mg, less than 240 mg, less than 235 mg, less than 230 mg, less than 225 mg, less than 220 mg, less than 215 mg, less than 210 mg, less than 205 mg, less than 200 mg, less than 195 mg, less than 190 mg, less than 185 mg, less than 180 mg, less than 175 mg, less than 170 mg, less than 165 mg, less than 160 mg, less than 150 mg, less than 145 mg, less than 140 mg, less than 135 mg, less than 130 mg, less than 125 mg, less than 120 mg, less than 115 mg, less than 110 mg, less than 105 mg, less than 100 mg, less than 95 mg, less than 90 mg, less than 85 mg, less than 80 mg, less than 75 mg, less than 70 mg, less than 65 mg, less than 60 mg, less than 55 mg, less than 50 mg, less than 45 mg, less than 40 mg, less than 35 mg, less than 30 mg, less than 25 mg, less than 20 mg, less than 15 mg, less than 10 mg, and less than 5 mg.

In certain embodiments, the therapeutically effective amount is any of less than about 350 mg, less than about 345 mg, less than about 340 mg, less than about 335 mg, less than about 330 mg, less than about 325 mg, less than about 320 mg, less than about 315 mg, less than about 310 mg, less than about 305 mg, less than about 300 mg, less than about 295 mg, less than about 290 mg, less than about 285 mg, less than about 280 mg, less than about 275 mg, less than about 270 mg, less than about 265 mg, less than about 260 mg, less than about 255 mg, less than about 250 mg, less than about 245 mg, less than about 240 mg, less than about 235 mg, less than about 230 mg, less than about 225 mg, less than about 220 mg, less than about 215 mg, less than about 210 mg, less than about 205 mg, less than about 200 mg, less than about 195 mg, less than about 190 mg, less than about 185 mg, less than about 180 mg, less than about 175 mg, less than about 170 mg, less than about 165 mg, less than about 160 mg, less than about 150 mg, less than about 145 mg, less than about 140 mg, less than about 135 mg, less than about 130 mg, less than about 125 mg, less than about 120 mg, less than about 115 mg, less than about 110 mg, less than about 105 mg, less than about 100 mg, less than about 95 mg, less than about 90 mg, less than about 85 mg, less than about 80 mg, less than about 75 mg, less than about 70 mg, less than about 65 mg, less than about 60 mg, less than about 55 mg, less than about 50 mg, less than about 45 mg, less than about 40 mg, less than about 35 mg, less than about 30 mg, less than about 25 mg, less than about 20 mg, less than about 15 mg, less than about 10 mg, and less than about 5 mg.

In certain embodiments, the therapeutically effective amount is any of at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 105 mg, at least 115 mg, at least 120 mg, at least 125 mg, at least 130 mg, at least 135 mg, at least 140 mg, at least 145 mg, at least 150 mg, at least 155 mg, at least 160 mg, at least 165 mg, at least 170 mg, at least 175 mg, at least 180 mg, at least 185, at least 190 mg, at least 195 mg, and at least 200 mg.

In certain embodiments, the therapeutically effective amount is any of at least about 5 mg, at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 105 mg, at least about 115 mg, at least about 120 mg, at least about 125 mg, at least about 130 mg, at least about 135 mg, at least about 140 mg, at least about 145 mg, or at least about 150 mg, at least about 155 mg, at least about 160 mg, at least about 165 mg, at least about 170 mg, at least about 175 mg, at least about 180 mg, at least about 185, at least about 190 mg, at least about 195 mg, and at least about 200 mg.

### V. Certain Dosing Regimens

In certain embodiments, described herein are methods of administering to a subject a therapeutically effective amount of the modified oligonucleotide ISIS 814907 one or more times. In certain embodiments, methods comprise administering the therapeutically effective amount at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 times. In certain embodiments, methods comprise administering the therapeutically effective amount once every 4 weeks. In certain embodiments, methods comprise administering the therapeutically effective amount once every 8 weeks. In certain embodiments, methods comprise administering the therapeutically effective amount once every 12 weeks. In certain embodiments, methods comprise administering the therapeutically effective amount once every 16 weeks. In certain embodiments, methods comprise administering the therapeutically effective amount once every 20 weeks. In certain embodiments, methods comprise administering the therapeutically effective amount once every 24 weeks. In certain embodiments, methods comprise administering the therapeutically effective amount once every 6 months. In certain embodiments, methods comprise administering the therapeutically effective amount monthly. In certain embodiments, methods comprise administering the therapeutically effective amount once every two months. In certain embodiments, methods comprise administering the therapeutically effective amount once every three months. In certain embodiments, methods comprise administering the therapeutically effective amount quarterly. In certain embodiments, methods comprise administering the therapeutically effective amount semiannually. In certain embodiments, methods comprise administering the therapeutically effective amount annually. In certain embodiments, methods comprise administering the therapeutically effective amount once every two years.

In certain embodiments, methods comprise administering the therapeutically effective amount about every 1 week, about every 2 weeks, about every 3 weeks, about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, about every 16 weeks, about every 17 weeks, about every 18 weeks, about every 19 weeks, about every 20 weeks, about every 21 weeks, about every 22 weeks, about every 23 weeks, about every 24 weeks, or about every 6 months. In certain embodiments, methods comprise administering the therapeutically effective amount about monthly. In certain embodiments, methods comprise administering the therapeutically effective amount once about every two months. In certain embodiments, methods comprise administering the therapeutically effective amount once about every three months. In certain embodiments, methods comprise administering the therapeutically effective amount about quarterly. In certain embodiments, methods comprise administering the therapeutically effective amount about semiannually. In certain embodiments, methods comprise administering the therapeutically effective amount about annually. In certain embodiments, methods comprise administering the therapeutically effective amount once about every two years.

In certain embodiments, methods comprise administering the therapeutically effective amount for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months.

In certain embodiments, methods comprise administering ISIS 814907 at a dose of 10 mg once monthly. In certain embodiments, methods comprise administering ISIS 814907 at a dose of 30 mg once monthly. In certain embodiments, methods comprise administering ISIS 814907 at a dose of 60 mg once monthly. In certain embodiments, methods comprise administering ISIS 814907 at a dose of 115 mg once every quarter. In some embodiments, the human subject has mild Alzheimer's disease. In some embodiments, the human subject has MCI due to Alzheimer's disease. In some embodiments, the human subject has mild Alzheimer's disease dementia.

In certain embodiments, methods comprise administering ISIS 814907 at a dose of 10 mg once every four weeks. In certain embodiments, methods comprise administering ISIS 814907 at a dose of 30 mg once every four weeks. In certain embodiments, methods comprise administering ISIS 814907 at a dose of 60 mg once every four weeks. In certain embodiments, methods comprise administering ISIS 814907 at a dose of 115 mg once every three months. In certain embodiments, methods comprise administering ISIS 814907 at a dose of 115 mg four times per year. In some embodiments, the human subject has mild Alzheimer's disease. In some embodiments, the human subject has MCI due to Alzheimer's disease. In some embodiments, the human subject has mild Alzheimer's disease dementia. In certain embodiments, methods comprise administering ISIS 814907 at a dose of 60 mg once every 24 weeks. In certain embodiments, methods comprise administering ISIS 814907 at a dose of 115 mg once every 24 weeks. In certain embodiments, methods comprise administering ISIS 814907 at a dose of 115 mg once every 12 weeks. In some embodiments, the human subject has mild Alzheimer's disease. In some embodiments, the human subject has MCI due to Alzheimer's disease. In some embodiments, the human subject has mild Alzheimer's disease dementia.

In certain embodiments, methods comprise administering ISIS 814907 at a dose of 60 mg twice per year (i.e., biannually). In certain embodiments, methods comprise administering ISIS 814907 at a dose of 115 mg twice per year (i.e., biannually). In certain embodiments, methods comprise administering ISIS 814907 at a dose of 115 mg once every three months (i.e., four times per year). In some embodiments, the human subject has mild Alzheimer's disease. In some embodiments, the human subject has MCI due to Alzheimer's disease. In some embodiments, the human subject has mild Alzheimer's disease dementia.

### Loading and Maintenance Doses

In certain embodiment, the therapeutically effective amount is administered as a loading dose and/or a maintenance dose. In certain embodiments, methods comprise administering a loading dose or doses and subsequently administering a maintenance dose or doses. In certain embodiments, methods comprise administering a loading dose once about every 4 weeks, and subsequently administering a maintenance dose once about every 4 weeks, about every 8 weeks, about every 12 weeks, about every 16 weeks, about 20 weeks, about 24 weeks, or about 6 months. In certain embodiments, methods comprise administering a loading dose once about every 4 weeks, and subsequently administering a maintenance dose once about every 6 months. In certain embodiments, methods comprise administering a loading dose once about every 12 weeks, and subsequently administering a maintenance dose once about every 6 months.

In certain embodiments, methods comprise administering at least 2 loading doses, at least 3 loading doses, at least 4 loading doses, at least 5 loading doses, or at least 6 load ing doses. In certain embodiments, methods comprise administering 2, 3, 4, 5, 6, 7, 8, 9, 10, 11, 12, 13, 14, 15, or 16 loading doses. In certain embodiments, methods comprise administering a loading dose or doses about every 1 week, about every 2 weeks, about every 3 weeks, about every 4 weeks, about every 5 weeks, about every 6 weeks, about every 7 weeks, about every 8 weeks, about every 9 weeks, about every 10 weeks, about every 11 weeks, or about every 12 weeks. In certain embodiments, methods comprise administering an initial loading dose and administering a second loading dose about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks after administering the initial loading dose.

In certain embodiments, methods comprise administering at least 2 maintenance doses, at least 3 maintenance doses, at least 4 maintenance doses, at least 5 maintenance doses, or at least 6 maintenance doses. In certain embodiments, methods comprise administering 2, 3, 4, 5, 6, 7, 8, 9, 10, 11, 12, 13, 14, 15, or 16 maintenance doses. In some instances, methods comprise administering a maintenance dose or doses about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, about every 16 weeks, about every 17 weeks, about every 18 weeks, about every 19 weeks, about every 20 weeks, about every 21 weeks, about every 22 weeks, about every 23 weeks, about every 24 weeks, or about every 6 months. In certain embodiments, methods comprise administering a first maintenance dose and administering a second maintenance dose about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, or about 6 months after administering the first maintenance dose.

In certain embodiments, methods comprise administering a first maintenance dose or doses about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, or about 24 weeks after administering the last loading dose.

### VI. Potency and Efficacy

In certain embodiments, described herein are methods of reducing Tau RNA and/or Tau protein in a cell or biological fluid of a human subject, wherein the methods comprise administering a therapeutically effective amount of ISIS 814907 to the subject. In certain embodiments, methods reduce Tau RNA and/or Tau protein in the cerebrospinal fluid of the human subject. One may determine whether or not methods reduce Tau RNA and/or Tau protein, e.g., by detecting/quantifying a first amount of Tau RNA or Tau protein in a first biological sample obtained before administering and detecting/quantifying a second amount of Tau RNA or Tau protein in a second biological sample obtained after administering, and detecting or quantifying a reduction in Tau RNA or Tau protein by comparing the first amount to the second amount.

In certain embodiments, methods comprise reducing Tau RNA and/or Tau protein by 1-100%, or a range defined by any two of these values. In certain embodiments, methods comprise reducing Tau RNA and/or Tau protein by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

In certain embodiments, methods comprise reducing Tau RNA or Tau protein by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%.

In certain embodiments, methods comprise reducing Tau RNA or Tau protein by about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% to 100%.

In certain embodiments, methods comprise administering ISIS 814907 to a subject and detecting or quantifying an amount of Tau RNA or Tau protein in a cell or a biological fluid of the subject. In certain embodiments, methods comprise detecting/quantifying a first amount of Tau RNA or Tau protein in a first biological sample obtained before administering and detecting/quantifying a second amount of Tau RNA or Tau protein in a second biological sample obtained after administering, and detecting or quantifying a reduction in Tau RNA or Tau protein by comparing the first amount to the second amount. In certain embodiments, the second biological sample is obtained less than about 24 hours after administering. In certain embodiments, the second biological sample is obtained less than about 1 week after administering. In certain embodiments, the second biological sample is obtained about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, or about 18 weeks after administering. In certain embodiments, methods comprise increasing or decreasing the dose after comparing the first amount to the second amount. In certain embodiments, methods comprise administering more frequently or less frequently after comparing the first amount to the second amount.

### Assessing Efficacy of ISIS 814907

In certain embodiments, methods described herein are sufficiently effective to ameliorate at least one symptom or hallmark of a disease or disorder associated with Tau protein in a human subject. In certain embodiments, methods described herein are sufficiently effective to prevent or decrease the rate of progression, or delay the onset of at least one symptom or hallmark associated with Tau protein in a human subject. In certain embodiments, the at least one symptom or hallmark is loss of memory, cognitive decline, loss of ability to understand or express speech, abnormal behavior, impaired motor function, falls, impaired balance, impaired swallowing, impaired eating, feeding tube placement, hospitalization, death, increase in the number and/or volume of neurofibrillary inclusions, loss of memory, loss of cognitive function, neuropsychiatric behavior dysfunction, or loss of motor function. In certain embodiments, the at least one symptom or hallmark is loss of cognitive function, neuropsychiatric behavior dysfunction, impaired global function, impaired loss of motor function, impaired cognitive function, impaired neuropsychiatric function, impaired daily function, impaired attention, impaired visuoperceptual processing, impaired working memory, impaired psychomotor speed, impaired verbal motor output, impaired degree of independence, increased apathy, impaired learning ability, impaired mental concentration, impaired understanding and expression of speech, impaired behavior, depression, irritability, anger, impaired mobility, impaired self-care, pain, discomfort, anxiety, seizures, suicidal ideation, or suicidal behavior. In certain embodiments, the disease or disorder associated with Tau protein is a neurodegenerative disease or disorder. In certain embodiments, the disease or disorder associated with Tau protein is a tauopathy. In certain embodiments, the disease or disorder associated with Tau protein is any of Alzheimer's disease (e.g., mild AD, MCI due to AD, or mild AD dementia) or Fronto-temporal Dementia (FTD). The clinical criteria for MCI due to AD or mild AD dementia are according to the National Institute on Aging at National Institutes of Health and the Alzheimer's Association (NIA-AA). In certain instances, the disease or disorder associated with Tau protein is Frontotemporal Dementia with Parkinsonism-17 (FTDP-17), non-fluent primary progressive aphasia, Progressive Supranuclear Palsy (PSP), Chronic Traumatic Encephalopathy (CTE), Corticobasal Ganglionic Degeneration (CBD), Pick Disease, Argyrophilic Grain Disease (AGD), Globular Glial Tauopathies, Epilepsy, and/or Dravet's Syndrome. In certain embodiments, the disease or disorder associated with Tau protein is any of Down syndrome, Prion diseases (sCJD, vCJD, gCJD, GSS, FFI), Diffuse neurofibrillary tangles with calcification, Familial British and Danish dementia, Postencephalitic parkinsonism, Subacute sclerosing panencephalitis, Myotonic dystrophy (DM1) and PROMM (DM2), Aging-related tau astrogliopathy, Traumatic brain injury, Chronic traumatic encephalopathy, IgLON5-related tauopathy, Guadeloupean parkinsonism, Parkinson-dementia complex of Guam, Non-Guamanian motor neuron disease with NFTs, Amyotrophic lateral sclerosis of Guam, X-linked parkinsonism with spasticity, Cerebrotendinous xanthomatosis, Niemann-Pick disease type C, PANK2-associated Neurodegeneration with Brain Iron Accumulation (NBIA), PLA2G6-assiciated NBIA, SLC9A6 mental retardation, or diseases or disorders associated with gene mutations in any of LRRK2, PRKN, SNCA, TARDBP, or C9orf72. In certain embodiments, the disease or disorder associated with Tau protein is Alzheimer's disease or FTD.

In certain embodiments, methods described herein are sufficiently effective to maintain memory, improve memory, maintain cognitive function, improve cognitive function, maintain neuropsychiatric behaviors, improve neuropsychiatric behaviors, maintain motor function, improve motor function, reduce falls, improve balance, improve swallowing, improve eating, reduce feeding tube placement, reduce hospitalization, prolong life, maintain the number and/or volume of neurofibrillary inclusions, or reduce the number and/or volume of neurofibrillary inclusions in a human subject having a disease or disorder associated with Tau protein. In certain embodiments, the disease or disorder associated with Tau protein is a tauopathy. In certain embodiments, the disease or disorder associated with Tau protein is any of Alzheimer's disease (e.g., mild AD, MCI due to AD, mild AD dementia) or Fronto-temporal Dementia (FTD). In certain instances, the disease or disorder associated with Tau protein is Frontotemporal Dementia with Parkinsonism-17 (FTDP-17), Progressive Supranuclear Palsy (PSP), Chronic Traumatic Encephalopathy (CTE), Corticobasal Ganglionic Degeneration (CBD), Pick Disease, Argyrophilic Grain Disease (AGD), Globular Glial Tauopathies, Epilepsy, and/or Dravet's Syndrome. In certain embodiments, the disease or disorder associated with Tau protein is Alzheimer's disease or FTD. In certain embodiments, the disease or disorder associated with Tau protein is Alzheimer's disease.

In certain embodiments, methods described herein are sufficiently effective to ameliorate at least one symptom or hallmark of a disease or disorder associated with Tau protein in a human subject having the disease or disorder associated with Tau protein relative to a healthy control subject. In certain embodiments, methods described herein are sufficiently effective to prevent or decrease the rate of progression, or delay the onset of at least one symptom or hallmark of a disease or disorder associated with Tau protein in a human subject having a disease or disorder associated with Tau protein relative to a healthy control subject. In certain embodiments, the disease or disorder associated with Tau protein is a tauopathy. In certain embodiments, the disease or disorder associated with Tau protein is any of Alzheimer's disease (e.g., mild AD, MCI due to AD, or mild AD dementia) or Fronto-temporal Dementia (FTD). In certain instances, the disease or disorder associated with Tau protein is Frontotemporal Dementia with Parkinsonism-17 (FTDP-17), Progressive Supranuclear Palsy (PSP), Chronic Traumatic Encephalopathy (CTE), Corticobasal Ganglionic Degeneration (CBD), Pick Disease, Argyrophilic Grain Disease (AGD), Globular Glial Tauopathies, Epilepsy, and/or Dravet's Syndrome. In certain embodiments, the disease or disorder associated with Tau protein is any of Down syndrome, Prion diseases (sCJD, vCJD, gCJD, GSS, FFI), Diffuse neurofibrillary tangles with calcification, Familial British and Danish dementia, Postencephalitic parkinsonism, Subacute sclerosing panencephalitis, Myotonic dystrophy (DM1) and PROMM (DM2), Aging-related tau astrogliopathy, Traumatic brain injury, Chronic traumatic encephalopathy, IgLON5-related tauopathy, Guadeloupean parkinsonism, Parkinson-dementia complex of Guam, Non-Guamanian motor neuron disease with NFTs, Amyotrophic lateral sclerosis of Guam, X-linked parkinsonism with spasticity, Cerebrotendinous xanthomatosis, Niemann-Pick disease type C, PANK2-associated Neurodegeneration with Brain Iron Accumulation (NBIA), PLA2G6-assiciated NBIA, SLC9A6 mental retardation, or diseases or disorders associated with gene mutations in any of LRRK2, PRKN, SNCA, TARDBP, or C9orf72. In certain embodiments, the disease or disorder associated with Tau protein is Alzheimer's disease. In certain embodiments, the healthy control subject is a subject that does not have a disease or disorder associated with Tau protein.

In certain embodiments, methods are sufficiently effective to ameliorate the increase in the amount or concentration of total tau protein, phosphorylated tau protein, neurofilament light (NfL), or amyloid beta protein in the cerebral spinal fluid of a human subject having a disease or disorder associated with Tau protein, as determined by CSF analysis. In certain embodiments, methods are sufficiently effective to maintain the amount or concentration of total tau protein, phosphorylated tau protein, or amyloid beta protein in the cerebral spinal fluid of a human subject having a disease or disorder associated with Tau protein, as determined by CSF analysis. In certain embodiments, methods are sufficiently effective to delay the increase of the amount or concentration of total tau protein, phosphorylated tau protein, or amyloid beta protein in the cerebral spinal fluid of a human subject having a disease or disorder associated with Tau protein, as determined by CSF analysis. In certain embodiments, methods are sufficiently effective to reduce the amount or concentration of total tau protein, phosphorylated tau protein, or amyloid beta protein in the cerebral spinal fluid of a human subject having a disease or disorder associated with Tau protein, as determined by CSF analysis. Methods for CSF analysis are known to those of skill in the art and examples of such methods are provided in Example 1.

In certain embodiments, methods are sufficiently effective to ameliorate the increase in the number and/or volume of neurofibrillary inclusions in a human subject having a disease or disorder associated with Tau protein as determined by performing magnetic resonance imaging (MRI) and/or Positron Emission Tomography (PET) on the human subject. In certain embodiments, methods are sufficiently effective to maintain the number and/or volume of neurofibrillary inclusions, delay the onset of an increase in the number and/or volume of neurofibrillary inclusions, slow the increase in the number and/or volume of neurofibrillary inclusions, or reduce the number an/or volume of neurofibrillary inclusions in a human subject having a disease or disorder associated with Tau protein as determined by performing magnetic resonance imaging (MRI) and/or Positron Emission Tomography (PET) on the human subject. In certain embodiments, the disease or disorder associated with Tau protein is a tauopathy. In certain embodiments, the disease or disorder associated with Tau protein is any of Alzheimer's disease (e.g., mild AD, MCI due to AD, or mild AD dementia) or Fronto-temporal Dementia (FTD). In certain instances, the disease or disorder associated with Tau protein is Frontotemporal Dementia with Parkinsonism-17 (FTDP-17), Progressive Supranuclear Palsy (PSP), Chronic Traumatic Encephalopathy (CTE), Corticobasal Ganglionic Degeneration (CBD), Pick Disease, Argyrophilic Grain Disease (AGD), Globular Glial Tauopathies, Epilepsy, and/or Dravet's Syndrome. In certain embodiments, the disease or disorder associated with Tau protein is any of Down syndrome, Prion diseases (sCJD, vCJD, gCJD, GSS, FFI), Diffuse neurofibrillary tangles with calcification, Familial British and Danish dementia, Postencephalitic parkinsonism, Subacute sclerosing panencephalitis, Myotonic dystrophy (DM1) and PROMM (DM2), Aging-related tau astrogliopathy, Traumatic brain injury, Chronic traumatic encephalopathy, IgLON5-related tauopathy, Guadeloupean parkinsonism, Parkinson-dementia complex of Guam, Non-Guamanian motor neuron disease with NFTs, Amyotrophic lateral sclerosis of Guam, X-linked parkinsonism with spasticity, Cerebrotendinous xanthomatosis, Niemann-Pick disease type C, PANK2-associated Neurodegeneration with Brain Iron Accumulation (NBIA), PLA2G6-assiciated NBIA, SLC9A6 mental retardation, or diseases or disorders associated with gene mutations in any of LRRK2, PRKN, SNCA, TARDBP, or C9orf72. In certain embodiments, the disease or disorder associated with Tau protein is Alzheimer's disease. In certain embodiments, methods reduce the number of neurofibrillary inclusions or reduce the volume of neurofibrillary inclusions by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

In certain embodiments, methods described herein are sufficiently effective to ameliorate at least one symptom or hallmark of a disease or disorder associated with Tau protein in a human subject as assessed by a clinically relevant test, score or scale. In certain embodiments, the disease or disorder associated with Tau protein is a tauopathy. In certain embodiments, the disease or disorder associated with Tau protein is any of Alzheimer's disease (e.g., mild AD, MCI due to AD, or mild AD dementia) or Fronto-temporal Dementia (FTD). In certain instances, the disease or disorder associated with Tau protein is Frontotemporal Dementia with Parkinsonism-17 (FTDP-17), Progressive Supranuclear Palsy (PSP), Chronic Traumatic Encephalopathy (CTE), Corticobasal Ganglionic Degeneration (CBD), Pick Disease, Argyrophilic Grain Disease (AGD), Globular Glial Tauopathies, Epilepsy, and/or Dravet's Syndrome. In certain embodiments, the disease or disorder associated with Tau protein is any of Down syndrome, Prion diseases (sCJD, vCJD, gCJD, GSS, FFI), Diffuse neurofibrillary tangles with calcification, Familial British and Danish dementia, Postencephalitic parkinsonism, Subacute sclerosing panencephalitis, Myotonic dystrophy (DM1) and PROMM (DM2), Aging-related tau astrogliopathy, Traumatic brain injury, Chronic traumatic encephalopathy, IgLON5-related tauopathy, Guadeloupean parkinsonism, Parkinson-dementia complex of Guam, Non-Guamanian motor neuron disease with NFTs, Amyotrophic lateral sclerosis of Guam, X-linked parkinsonism with spasticity, Cerebrotendinous xanthomatosis, Niemann-Pick disease type C, PANK2-associated Neurodegeneration with Brain Iron Accumulation (NBIA), PLA2G6-assiciated NBIA, SLC9A6 mental retardation, or diseases or disorders associated with gene mutations in any of LRRK2, PRKN, SNCA, TARDBP, or C9orf72. In certain embodiments, the disease or disorder associated with Tau protein is Alzheimer's disease. In certain embodiments, the at least one symptom or hallmark is loss of memory, cognitive decline, loss of ability to understand or express speech, abnormal behavior, impaired motor function, or increase in the number and/or volume of neurofibrillary inclusions. In certain embodiments, at least one symptom or hallmark is loss of cognitive function, neuropsychiatric behavior dysfunction, impaired global function, loss of motor function, impaired cognitive function, impaired neuropsychiatric function, impaired daily function, impaired attention, impaired visuoperceptual processing, impaired memory, impaired degree of independence, increased apathy, impaired learning ability, impaired mental concentration, impaired understanding and expression of speech, impaired behavior, depression, irritability, anger, impaired mobility, impaired self-care, pain, discomfort, anxiety, seizures, suicidal ideation, or suicidal behavior. Non-limiting examples of such clinically relevant tests, scores and scales include the following.

### Symbol Digit Modalities Test

In certain embodiments, methods described herein are sufficiently effective to ameliorate impaired attention, impaired visuoperceptual processing, impaired working memory, impaired psychomotor speed, or a combination thereof, in a subject having a disease or disorder associated with Tau protein, as assessed by the Symbol Digit Modalities Test (SDMT). In SMDT, the subject pairs abstract symbols with specific numbers according to a translation key. The test measures the number of items correctly paired (maximum of 110 correct pairs) in 90 seconds. SDMT has been shown to have strong reliability and validity. SDMT is described in greater detail by Smith, A. Symbol Digit Modalities Test (SDMT). Manual (rev.) Los Angeles: Western Psychological Services, 1982. In certain embodiments, methods improve the number of items correctly paired by at least 1, 2, 3, 4, 5, 6, 7, 8, 9, or 10 items.

### Stroop Word Reading Test

In certain embodiments, methods described herein are sufficiently effective to ameliorate impaired attention, impaired processing, impaired psychomotor speed, impaired verbal motor output, or a combination thereof, in a subject having a disease or disorder associated with Tau protein, as assessed by the Stroop Word Reading Test (SWR) Test. During a SWR Test, the subject is presented with a page of color names (i.e., “BLUE,” “RED,” or “GREEN”) printed in black ink and is asked to read aloud as many words as possible within a given amount of time (in 45 seconds). The number of words read correctly is counted, with a higher score indicating better cognitive performance. See Stroop, J. R., J. Exp. Psychol. 1935, 18, 643-662 for additional description of the SWR Test. In certain embodiments, methods improve the number of words the subject can read aloud in the given amount of time by at least 1, 2, 3, 4, 5, 6, 7, 8, 9, or 10 words.

### Global Impression, Severity and Change Scales

In certain embodiments, methods described herein are sufficiently effective to improve a subject's score on a Global Impression, Severity and Change Scale. This assessment can be conducted by a clinician (CGI-S), a companion (CrGI-S), or the subject (PGI-S). The subject is assessed using an 11-point numeric rating scale (NRS), where higher scores indicate greater severity. The CGI-S is described in greater detail by Guy W: ECDEU Assessment Manual for Psychopharmacology Rockville, MD: U. S. Department of Health, Education, and Welfare; 1976. In certain embodiments, methods reduce the subject's NRS score by 1, 2, 3, 4, 5, 6, 7, 8, 9 or 10 points.

### Montreal Cognitive Assessment

In certain embodiments, methods described herein are sufficiently effective to ameliorate impaired cognitive function in a subject having a disease or disorder associated with Tau protein, as assessed by the Montreal Cognitive Assessment (MoCA). The MoCA is a subject-completed assessment used to detect cognitive impairment. It contains a series of basic assessments, including attention and visuospatial tasks. The total score ranges from 0 to 30, where lower scores indicate greater impairment. The MoCA is described in greater detail by Nasreddine et al., 2015, J. Am. Geriatr. Soc. 53:695-9. In certain embodiments, methods increase the subject's MoCA score by at least 1, 2, 3, 4, 5, 6, 7, 8, 9 or 10 points.

### Work Productivity and Activity Impairment Test

In certain embodiments, methods described herein are sufficiently effective to ameliorate impaired global function in a subject having a disease or disorder associated with Tau protein, as assessed by the Work Productivity and Activity Impairment Test (WPAI). The WPAI contains 6 items assessing the impact of disease on employment status (yes/no), hours missed due to disease, hours missed due to other reasons, hours worked, and impact on productivity and on daily activities (both using an 11-point NRS, where higher scores indicate greater impact). The WPAI is described in greater detail by Reilly et al., 1993, Pharmacoeconomics 4:353-65. In certain embodiments, methods reduce the subject's WPAI score by at least 1, 2, 3, 4, 5, 6, 7, 8, 9 or 10 points.

### Apathy Evaluation Scale

In certain embodiments, methods described herein are sufficiently effective to ameliorate increased apathy in a subject having a disease or disorder associated with Tau protein, as assessed by the Apathy Evaluation Scale (AES). The AES is an 18-item assessment of apathy, including overt behavior, cognitive aspects of motivation, and emotional responsivity. Each item is scored on a 4-point Likert scale, from 1 (“Not at all”) to 4 (“A lot”). A total score is created by summing the 18 items (scores range from 18 to 72; 3 items are reverse scored), with higher scores indicating greater apathy. The AES is described in greater detail by Marin et al., 1991, Psychiatry Res. 38:143-62. In certain embodiments, methods decrease the subject's AES score by at least 1, 2, 3, 4, 5, 6, 7, 8, 9 or 10 points.

### Neuro-Qol Cognition Function Short Form, Version 2

In certain embodiments, methods described herein are sufficiently effective to ameliorate impaired mental concentration and/or impaired learning ability in a subject having a disease or disorder associated with Tau protein, as assessed by the Neuro-Qol Cognition Function Short Form. The Neuro-Qol Cognition Function Short Form contains 8 items (including “trouble concentrating” and difficulty “learning new tasks or instructions”), each assessed using a 5-point Likert scale, where lower scores indicate greater difficulty (4 items) or greater frequency (4 items). The raw sum score is converted to a T-score distribution (mean of 50, standard deviation of 10). See National Institute of Neurological Disorders and Stroke (NINDS). User Manual for the Quality of Life in Neurological Disorders (Neuro-QoL) Measures, Version 2.0, March 2015.

### Symptoms of Major Depressive Disorder Scale

In certain embodiments, methods described herein are sufficiently effective to ameliorate depression in a subject having a disease or disorder associated with Tau protein, as assessed by the Symptoms of Major Depressive Disorder Scale (SMDDS). SMDDS is a self-report assessment of depression (McCarrier et al., 2016, Patient 9:117-134). It contains 16 items, measuring concepts such as sadness, irritability, worry, and sleep disturbance. Each item is assessed on a 5-point Likert scale, from “Not at all” to “Extremely” (9 items) and from “Never” to “Always” (7 items). Item scores from 15 of the items (the least severe of the two eating items is not included) are summed to create a 0 to 60 score, where higher scores indicate more severe depressive symptomatology. The SMDDS is described in greater detail by Bushnell et al., 2019, Value in Health 22:906-915. In certain embodiments, methods reduce the subject's score by at least 1, 2, 3, 4, 5, 6, 7, 8, 9 or 10 points.

### EuroQol 5-Dimension, 5-Level Questionnaire

In certain embodiments, methods described herein are sufficiently effective to ameliorate impaired mobility, impaired self-care, impaired global function, pain/discomfort, anxiety, depression, or a combination thereof, in a subject having a disease or disorder associated with Tau protein, as assessed by the EuroQol 5-Dimension, 5-Level Questionnaire (EQ-5D-5L). EQ-5D-5L is a validated self-report health status questionnaire used to calculate a health status utility score for use in health economic analyses (Brooks, 1996, Health Policy 37:53-72 and Herdman et al., 2011, Qual Life Res. 20:1727-36). There are two components to the EQ-5D-5L: a 5-item health state profile that assesses mobility, self-care, usual activities, pain/discomfort, and anxiety/depression, as well as a visual analog scale (VAS) that measures health state. Published weighting systems allow for creation of a single composite score of the subject's health status (Index score) from the 5-item scores (i.e., does not include the VAS). In certain embodiments, methods improve the subject's EQ-5D-5L score.

### Health Utilities Index

In certain embodiments, methods described herein are sufficiently effective to improve the health status of a subject having a disease or disorder associated with Tau protein, as assessed by the Health Utilities Index (HUI). The HUI is a multi-attribute system of health status (Feeny et al., 1995, Pharmacoeconomics 7:490-502). The HUI2 and HUI3 questionnaire (commonly referred to as HUI2/3) contains 15 items with Likert-type response options. From these items, two scores can be produced: HUI2 (7 items) and HUI3 (8 items). Both scores are health utility indexes, where 0=death, and 1=perfect health. In certain embodiments, methods improve the subject's HUI score.

### Columbia-Suicide Severity Rating Scale

In certain embodiments, methods described herein are sufficiently effective to ameliorate suicidal ideation or suicidal behavior of a subject having a disease or disorder associated with Tau protein, as assessed by the Columbia-Suicide Severity Rating Scale (C-SSRS). The C-SSRS is a structured tool to assess suicidal ideation and behavior. Four constructs are measured: severity of ideation, intensity of ideation, behavior, and lethality of actual suicide attempts. Binary (yes/no) data are collected for 10 categories, and composite endpoints based on the categories are followed over time to monitor subject safety (Posner et al., 2011, Am. J. Psychiatry 168:1266-77). It maps to the Columbia-Classification Algorithm for Suicide Assessment and meets the criteria listed in the U.S. FDA draft guidance for assessment of suicidality in clinical trials (FDA 2012). In certain embodiments, methods improve the subject's C-SSRS score.

### Repeatable Battery for the Assessment of Neuropsychological Status (RBANS)

In certain embodiments, methods described herein are sufficiently effective to ameliorate loss of memory, cognitive decline, impaired cognitive function or loss of cognitive function of a subject having a disease or disorder associated with Tau protein, as assessed by the Repeatable Battery for the Assessment of Neuropsychological Status (RBANS) assessment. The RBANS is a neurological assessment designed to identify abnormal cognitive decline in older adults and to differentiate between different dementia etiologies (Randolph et al., J. Clin. Exp. Neuropsychol. 1998 20: 310-319). The RBANS has been shown to correlate with functional limitations in dementia populations (Hobson et al., 2010, Int. J. Geriatr. Psychiatry 25:525-530) and to adequately detect cognitive impairment associated with AD (Duff et al., Arch. Clin. Neuropsychol. 2008 23:603-612).

The assessment yields 5 index scores, 1 for each of the domains tested: attention, visuospatial/constructional abilities, language, immediate memory and delayed memory. The index scores for the domains can be used to determine a total scale score.

### Mini-Mental State Examination (MMSE)

In certain embodiments, methods described herein are sufficiently effective to ameliorate loss of memory, cognitive decline, impaired cognitive function or loss of cognitive function of a subject having a disease or disorder associated with Tau protein, as assessed by the Mini-Mental State Examination (MMSE) (Tombaugh et al., 2996, Psychological Assessment 8:48-59). The MMSE is used to quantify cognitive function and to screen for cognitive loss. It is a severity scale, not a diagnostic scale, and can be confounded by level of education such that dementia may be present and diagnosed despite relatively high MMSE score. The test administrator presents the patient with a series of questions and tests related to orientation, attention, calculation, recall, language and motor skills with a maximum possible score of 30 points.

### Neuropsychiatric Inventory—Questionnaire (NPI-Q)

In certain embodiments, methods described herein are sufficiently effective to ameliorate abnormal behavior, neuropsychiatric behavior dysfunction, or impaired neuropsychiatric function, of a subject having a disease or disorder associated with Tau protein, as assessed by the Neuropsychiatric Inventory (NPI). The NPI assesses behavioral disturbances occurring in dementia patients to evaluate a wide range of psychopathology and distinguish among different etiologies of dementia (Cummings et al., 1997, Neurology 71:337-343). The NPI-Questionnaire (NPI-Q) is a brief questionnaire form of the NPI intended to identify clinically-significant neuropsychiatric disturbances and their associated impact on caregivers (Kaufer et al., 2000, J. Neuropsychiatry Clin. Neuro. 12:233-239). It is completed by the test administrator after discussion with the trial partner about the presence/absence in the patient of 12 behaviors (e.g., anxiety, disinhibition, agitation/aggression) and, for each behavior that is present, its severity (scale of 1-3, with 3 being the most severe) and the associated caregiver distress (scale of 0-5, representing no distress through extreme distress).

### Functional Activities Questionnaire (FAQ)

In certain embodiments, methods described herein are sufficiently effective to ameliorate impaired degree of independence, impaired mobility, or impaired self-care of a subject having a disease or disorder associated with Tau protein, as assessed by the Functional Activities Questionnaire (FAQ). The FAQ is a widely-used scale to assess activities of daily living in patients with mild AD (Brown et al., 2011, Arch. Gen. Psychiatry 68:617-626; Marshall et al., 2011, Alzheimers Dement. 7:300-308). It is a brief questionnaire in which the trial partner rates the patient's abilities in ten areas, such as keeping track of current events and preparing a balanced meal, on a scale of 0 (normal) to 3 (dependent). A score of 30 represents maximal dependence, and a score of 0 represents complete independence (Pfeffer et al., 1982, J. Gerontol. 37:323-329).

### Clinical Dementia Rating (CDR) Scale

In certain embodiments, methods described herein are sufficiently effective to ameliorate loss of memory, cognitive decline, impaired cognitive function or loss of cognitive function of a subject having a disease or disorder associated with Tau protein, as assessed by the Clinical Dementia Rating (CDR). The CDR is a global scale used to categorize the severity of Alzheimer's type dementia (Morris, 1993, Neurol. 43:2412-2414; Morris, 1997, Int. Psychogeriatr. 9 Suppl 1:173-173). It utilizes a semi-structured test administrator interview with the patient and the trial partner to obtain the information necessary to rate the patient's cognitive performance in 6 domains: memory, orientation, judgment and problem solving, community affairs, home and hobbies, and personal care. Categorical scores for each domain are 0 (none), 0.5 (questionable), 1 (mild), 2 (moderate) and 3 (severe). A summed total score (sum of boxes) is produced, and a global score (using the same 5 grades of dementia) is derived from the category scores according to the practice described by Morris (Morris 1993).

In certain embodiments, methods described herein are sufficiently effective to ameliorate a symptom or hallmark of a disease or disorder associated with Tau protein, as assessed by one or more of the following assessments known to those of skill in the art: Alzheimer's Disease Cooperative Study Activities of Daily Living for Mild Cognitive Impairment ADCS-(ADL MCI); Alzheimer's Disease Assessment Scale Cognitive Subscale (ADAS Cog13); Alzheimer's Disease Composite Score (ADCOMS); Integrated Alzheimer's Disease Rating Scale (iADRS); Zarit Burden Interview (ZBI); Resource Utilization in Dementia (RUD-Lite); PSP Rating Scale (PSPRS); Movement Disorder Society (MDS)-sponsored revision of the Unified Parkinson's Disease Rating Scale (MDS-UPDRS) Part II; Clinical Global Impression (CGI) of Severity and Change; Progressive Supranuclear Palsy Quality of Life scale (PSP-QoL); Schwab and England Activities of Daily Living (SEADL) scale; Phonemic Fluency; Letter-Number Sequencing; Color Trails Test; The Montreal Cognitive Assessment (MoCA); European Quality of Life (EuroQol); and Short Form Survey (SF-36).

### VII. Certain Combination Therapies

In certain embodiments, methods comprise co-administering ISIS 814907 with at least one other pharmaceutical agent. In certain embodiments, the at least one other pharmaceutical agent ameliorates a disease or disorder associated with Tau protein or a symptom or hallmark thereof. In certain embodiments, ISIS 814907 is co-administered with the at least one other pharmaceutical agent to produce a combinational effect. In certain embodiments, ISIS 814907 is co-administered with the at least one other pharmaceutical agent to produce a synergistic effect.

In certain embodiments, ISIS 814907 and the at least one other pharmaceutical agent are administered at the same time. In certain embodiments, ISIS 814907 and the at least one other pharmaceutical agent are administered at different times. In certain embodiments, ISIS 814907 and the at least one other pharmaceutical agent are prepared together in a single formulation. In certain embodiments, ISIS 814907 and the at least one other pharmaceutical agent are administered are prepared separately.

In certain embodiments, pharmaceutical agents that may be co-administered with ISIS 814907 include antipsychotic agents, such as, e.g., haloperidol, chlorpromazine, clozapine, quetapine, and olanzapine; antidepressant agents, such as, e.g., fluoxetine, sertraline hydrochloride, venlafaxine and nortriptyline; tranquilizing agents such as, e.g., benzodiazepines, clonazepam, paroxetine, venlafaxin, and beta-blockers; mood-stabilizing agents such as, e.g., lithium, valproate, lamotrigine, and carbamazepine; paralytic agents such as, e.g., Botulinum toxin; and/or other experimental agents including, but not limited to, tetrabenazine (Xenazine), creatine, conezyme Q10, trehalose, docosahexanoic acids, ACR16, ethyl-EPA, atomoxetine, citalopram, dimebon, memantine, sodium phenylbutyrate, ramelteon, ursodiol, zyprexa, xenasine, tiapride, riluzole, amantadine, [123I]MNI-420, atomoxetine, tetrabenazine, digoxin, detromethorphan, warfarin, alprozam, ketoconazole, omeprazole, and minocycline.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: Phase 1b Human Clinical Trial with ISIS 814907

A randomized, double-blind, placebo-controlled multiple-ascending-dose, Phase 1b trial involving adult human subjects with AD was conducted.

Human subjects were randomly assigned in a 3:1 ratio to receive ISIS 814907 in artificial CSF or placebo (artificial CSF) as a bolus intrathecal administration every 4 weeks for a total of four doses (Days 1, 29, 57 and 85) (Cohorts A, B, and C) or every 12 weeks for a total of two doses (Days 1 and 85) (Cohort D) during the 13 week Treatment Evaluation (TE) period. There was a 23-week Post-Treatment (PT) period during which no study drug was administered. The primary endpoint was safety. The secondary endpoint was pharmacokinetics of ISIS 814907 in CSF. Prespecified exploratory endpoints included the concentrations of total Tau protein (t-tau) protein in CSF.

Forty-six human subjects enrolled in the trial. Twelve subjects received placebo and thirty-four received ISIS 814907 at ascending dose levels of 10 mg (Cohort A), 30 mg (Cohort B), or 60 mg (Cohort C) or 115 mg (Cohort D). Each human subject received all protocol-specified doses and completed the 13-week TE period. Three patients discontinued the study during the 23-week PT period, one each from the placebo, 60-mg monthly (Cohort C), and 115 mg quarterly (Cohort D) groups. All adverse events in human subjects receiving ISIS 814907 were mild (88%) or moderate (12%) in severity (e.g., procedural pain and post-lumbar puncture headache). No serious adverse events were observed in human subjects receiving ISIS 814907. There were no clinically relevant adverse changes in laboratory variables.

Male or female patients aged 50-74 years with mild AD defined by a Clinical Dementia Rating (CDR) Overall Global Score of 1 or Global Score of 0.5 with a Memory Score of 1, MMSE score of 20-27 inclusive, and confirmed AD biomarker positivity (amyloid, tau& phospho-tau via CSF) at Screening were considered eligible.

The characteristics of patients at baseline were representative of relatively younger, patients with mild AD patients and were generally similar across trial groups (Table 1). The mean CDR Sum of Boxes score at baseline was numerically lower for ISIS 814907 60 mg and 115-mg groups due to an amendment during the study, which allowed inclusion of patients with a CDR Global Score of 0.5 and Memory Score of 1 in addition to patients with a CDR Global Score of 1.0. Table 1 shows the characteristics of human subjects at baseline.

TABLE 1
Characteristics of Human Subjects at Baseline*

| | | ISIS 814907 | | | | |
|---|---|---|---|---|---|---|
| | | ISIS | | | 60 mg | |
| | | 814907 | 10 mg | 30 mg | Monthly | 115 mg |
| | Placebo | Groups | Monthly | Monthly | Q4W | Quarterly |
| | (N = 12) | (N = 34) | (N = 6) | (N = 6) | (N = 9) | (N = 13) |
| Characteristic | | | | | | |
| Age - year | 66 ± 4.6 | 66 ± 6.1 | 64 ± 5.2 | 65 ± 6.1 | 66 ± 6.8 | 67 ± 6.3 |
| Female, no. (%) | 6 (50) | 17 (50) | 2 (33) | 4 (67) | 5 (56) | 6 (46) |
| Race - White, | 12 (100) | 34 (100) | 6 (100) | 6 (100) | 9 (100) | 13 (100) |
| no. (%) | | | | | | |
| MMSE Total Score | 24.2 ± 1.7 | 23.5 ± 2.4 | 21.5 ± 1.6 | 24.5 ± 1.4 | 24.6 ± 2.5 | 23.2 ± 2.5 |
| RBANS Total Score | 64.9 ± 10.2 | 68.2 ± 12.1 | 58.8 ± 11.2 | 69.2 ± 12.1 | 69.9 ± 9.1 | 70.9 ± 13.4 |
| CDR Global Score, | | | | | | |
| no. (%) | | | | | | |
| 0.5 | 7 (58) | 23 (68) | 0 (0) | 3 (50) | 9 (100) | 11 (85) |
| 1 | 5 (42) | 11 (32) | 6 (100) | 3 (50) | 0 (0) | 2 (15) |
| CDR Sum of Boxes | 4.1 ± 1.3 | 3.7 ± 1.1 | 4.8 ± 0.5 | 4.7 ± 1.0 | 2.9 ± 0.6 | 3.3 ± 1.1 |
| Concomitant | | | | | | |
| Medications no. (%) | | | | | | |
| Anticholinesterases | 7 (58) | 21 (62) | 4 (67) | 5 (83) | 4 (44) | 8 (62) |
| Memantine | 1 (8) | 7 (21) | 2 (33) | 0 (0) | 3 (33) | 2 (15) |
| Estrogen Replacement | 0 (0) | 3 (9) | 1 (17) | 0 (0) | 1 (11) | 1 (8) |
| APOE4 Carrier (%) | 8 (67) | 25 (74) | 5 (83) | 3 (50) | 6 (67) | 11 (85) |
| t-tau Concentration | 387.3 ± 120.9 | 405.6 ± 132.7 | 364.6 ± 98.1 | 386.4 ± 152.3 | 391.0 ± 111.8 | 443.4 ± 153.8 |
| in CSF - pg/mL | | | | | | |
| p-tau concentration | 38.7 ± 13.0 | 40.7 ± 14.2 | 39.1 ± 13.0 | 38.6 ± 16.6 | 39.5 ± 12.6 | 43.2 ± 15.915 |
| in CSF - pg/mL | | | | | | |
| t-tau/Ab42 | 0.6 ± 0.2 | 0.6 ± 0.2 | 0.6 ± 0.2 | 0.6 ± 0.1 | 0.5 ± 0.1 | 0.6 ± 0.2 |

*Plus-minus values are means ± SD. Patients were assigned to receive either placebo or ascending doses of the antisense oligonucleotide drug ISIS 814907. Percentages may not total 100 because of rounding.
CSF (cerebrospinal fluid); MMSE (Mini Mental State Examination); RBNAS (Repeatable Battery for the Assessment of Neuropsychological Status); CDR (Clinical Demential Rating); APOE4 (Apolipoprotein E4); t-tau (total Tau); p-tau (phosphorylated Tau); Ab42 (amyloid b 42).

CSF (20 mL) was obtained from patients using a standard lumbar puncture collection kit immediately prior to dosing on days 1, 29, 57, and 85 (Cohorts A, B, and C) or immediately prior to dosing on days 1 and 85 (Cohort D) to obtain concentrations of total Tau (t-tau) protein in CSF. CSF was similarly collected during the post treatment period on study days 113, 141, and 253 (Cohorts A and B) or on study days 141, 197, and 253 (Cohorts C and D). The CSF samples obtained during screening and on day 1 were analyzed and results averaged to serve as the baseline sample, and the CSF samples on days 29, 57, and 85 served as 28-day post-dose trough samples.

CSF was obtained via lumbar puncture from patients receiving ISIS 814907 or placebo and tau protein concentration was measured with the Elecsys Total-Tau CSF. The Elecsys Total-Tau CSF assay is an in vitro diagnostic immunoassay intended for the quantitative determination of the total Tau protein in human CSF. Intended uses include the following:
1. The Elecsys Total-Tau CSF assay is intended to be used alone or in combination with Elecsys β-Amyloid (1-42) CSF assay as a ratio in adult subjects with mild cognitive impairment (MCI) as an aid to identify subjects who are at lower vs. higher risk of cognitive decline as defined by change in a clinical score within a 2 year period.
2. The Elecsys Total-Tau CSF assay is intended to be used in combination with Elecsys β-Amyloid (1-42) CSF assay as a ratio in adult subjects with cognitive impairment being evaluated for AD and other causes of cognitive impairment wherein a positive and negative CSF result is concordant with positive and negative amyloid Positron Emission Tomography (PET) scan result, respectively.

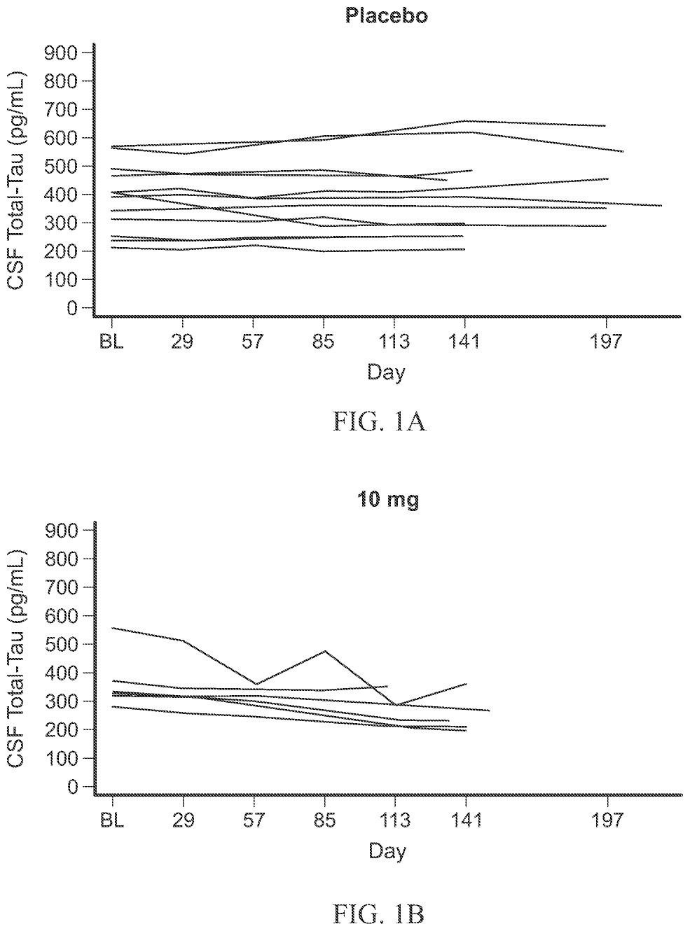

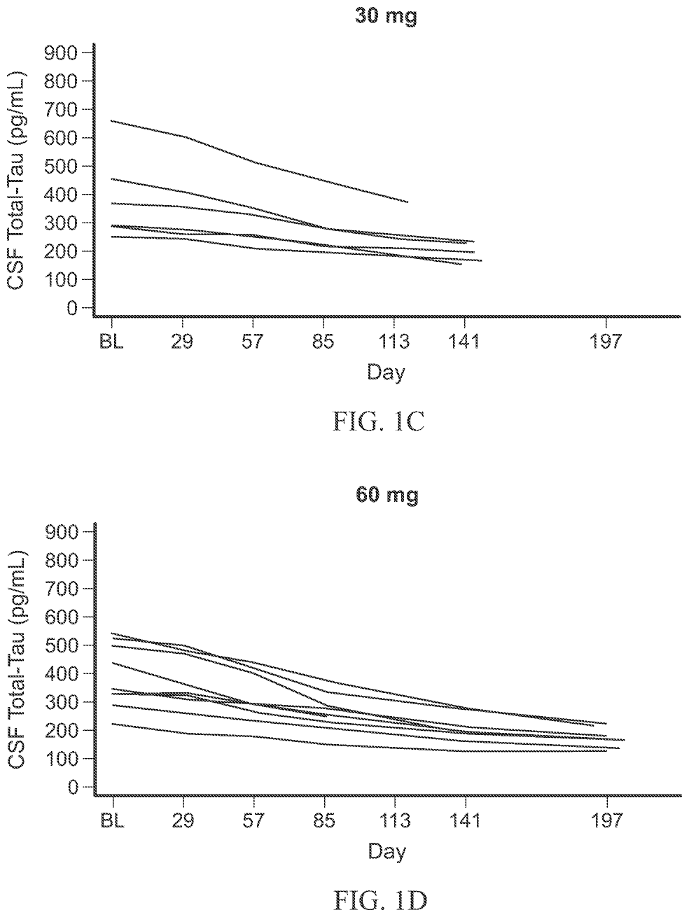

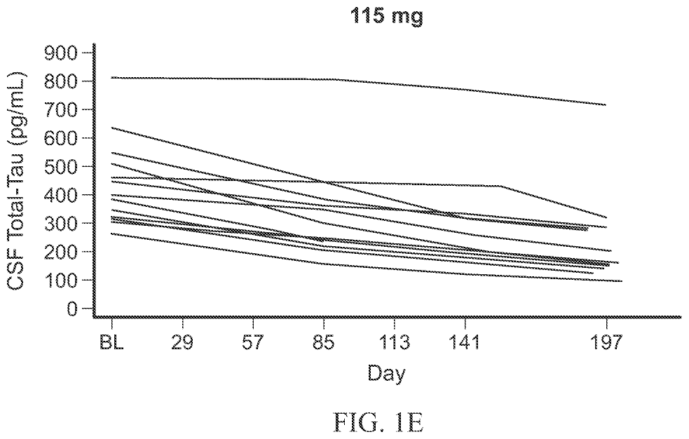

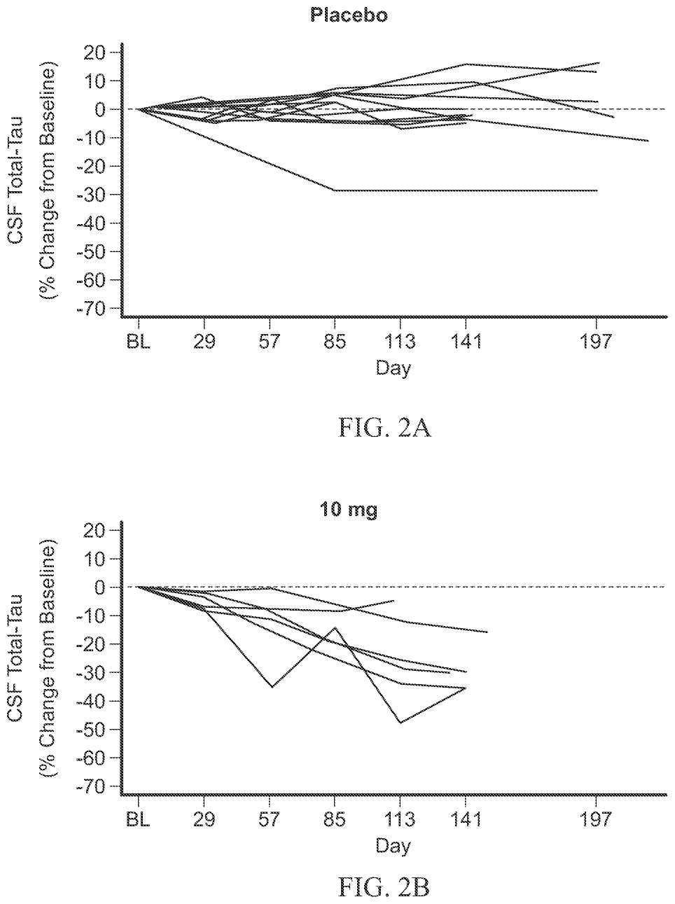

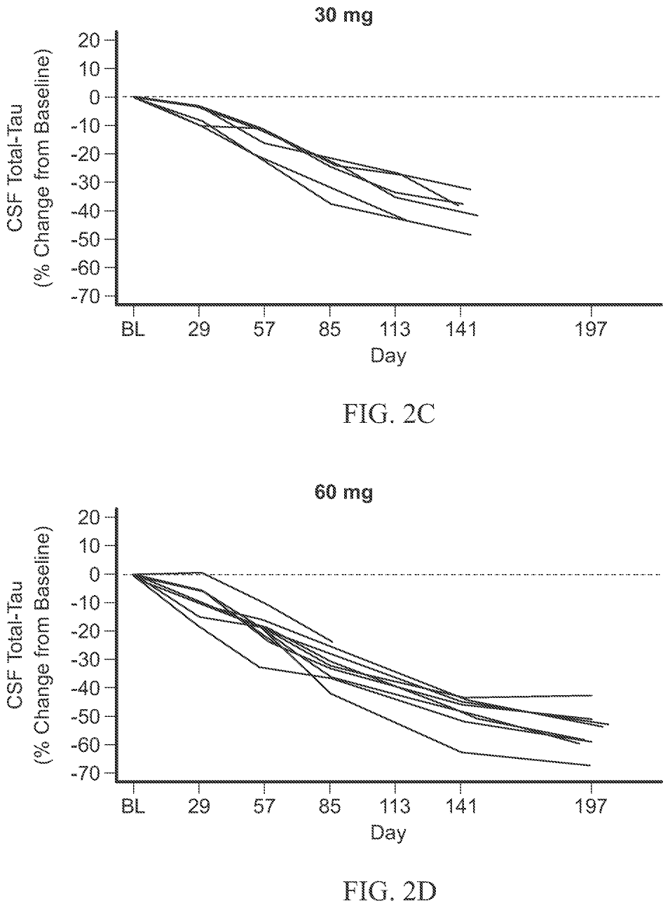

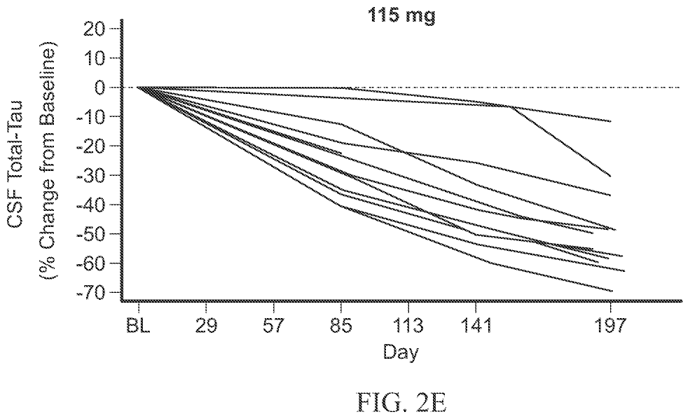

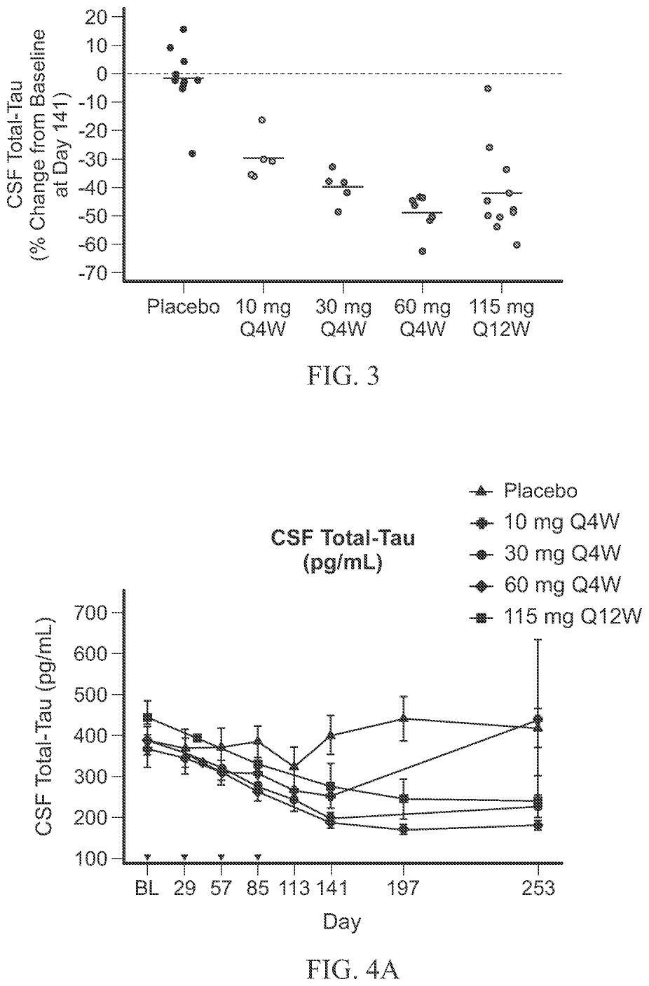

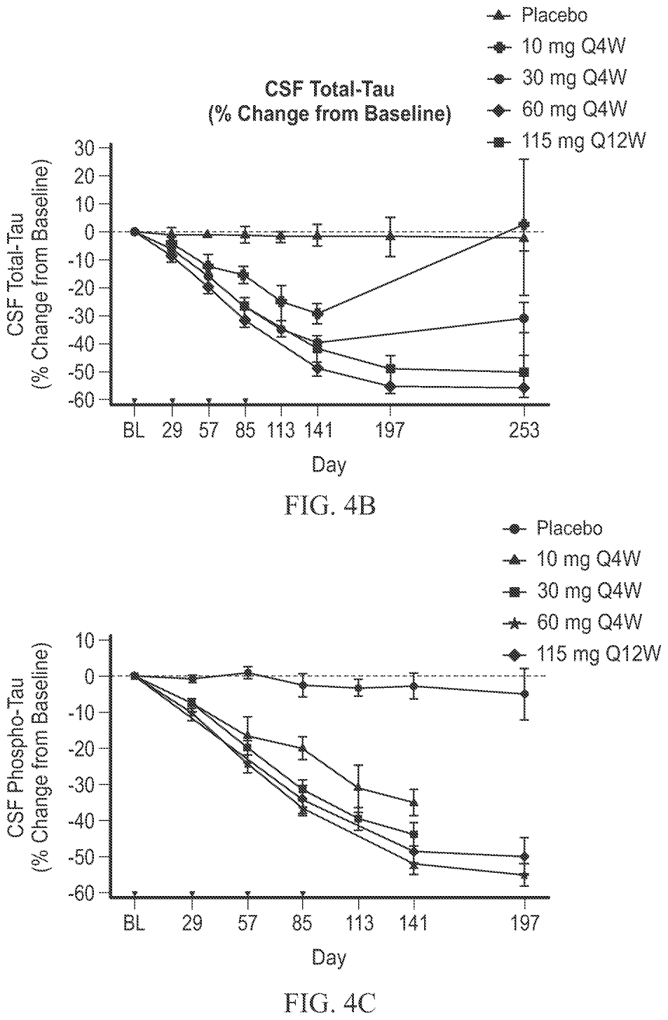

In patients receiving ISIS 814907, there were dose-dependent decreases in the concentration of t-tau in CSF 8-weeks post-last dose, with a mean percentage change from baseline of −30%, −40%, −49% and −42% in ISIS 814907 Cohorts A, B, C, and D, respectively. CSF t-tau continued to decline 16-weeks post-last dose in patients treated with ISIS 814907 in Cohort C (60 mg) and Cohort D (115 mg) (−55% and −49%, respectively; CSF was not collected 16-weeks post-last dose in Cohort A (10 mg) and Cohort B (30-mg) groups, with a maximum reduction of 69% in any individual patient Cohort D (115 mg); FIGS. 1A and 1B). In patients receiving placebo, the mean percentage change from baseline ranged from −1% to −2.4% among all post-baseline visits represented in FIG. 1A. Steady-state maximal reduction of the concentration of CSF t-tau did not appear to have been reached during the 3-month treatment emergent (TE) period. Although stable reductions of total Tau in CSF were observed for the 60-mg monthly dose group during the 6-month post-treatment (PT) period (beginning Day 113), a continued reduction in t-tau CSF concentration was observed in the 115-mg quarterly dose group.

TABLE 2
Summary of percent change from baseline of
CSF Total Tau (t-tau) protein by dose group

CSF Total-

| Tau (% | | ISIS 814907 | | | |
|---|---|---|---|---|---|
| change from | Placebo | 10 mg | 30 mg | 60 mg | 115 mg |
| baseline) | (N = 12) | (N = 6) | (N = 6) | (N = 9) | (N = 13) |
| Mean pg/mL | −1.3 ± | −29.5 ± | −39.7 ± | −48.8 ± | −41.9 ± |
| (SD) | 11.4 | 8.0 | 5.8 | 6.9 | 15.4 |
| Median | −2.1 | −30.5 | −38.1 | −45.9 | −47.6 |
| Min, Max | −27.9, | −35.7, | −48.4, | −62.5, | −60.0, |
| | 15.9 | −16.0 | −32.6 | −43.3 | −5.0 |

* For CSF Total-Tau, endpoint is defined as Study Day 141

Additional exploratory endpoints included assessment of CSF concentrations of p-tau, Aβ42, neurofilament light (NfL), neurofilament heavy (NfH), neurogranin (NRGN), and YKL-40; results are presented in Table 3. CSF from participants was analyzed with the following assays: Elecsys β-Amyloid (1-42) CSF, Elecsys Total-Tau CSF, and Elecsys Phospho-Tau (181P) CSF performed at Roche Diagnostics, Indianapolis, IN); NfL (Uman), NfH (Protein Simple, ELLA), YKL40 (Protein Simple, ELLA), and Neurogranin (Euroimmune) at Immunologix (Tampa, FL). Exploratory CSF parameters including neurofilament light (NfL) and heavy (NfH), neurogranin (NRGN) and YKL-40 showed no dose-responsive effects following treatment with MAPTRx (data not shown).

Reductions similar to those observed for t-tau were observed for p-tau181 concentration and the ratio of t-tau to A42 in CSF. In participants receiving ISIS 814907, there were dose-dependent decreases in the concentration of p-tau181 in CSF 8-weeks post-last dose with mean percentage change from baseline of −35%, −44%, −52% and −49% in ISIS 814907 10-mg, 30-mg, and 60-mg monthly and 115-mg quarterly groups, respectively. CSF p-tau181 continued to decline in participants treated with ISIS 814907 in 60-mg monthly and 115-mg quarterly groups 24-weeks (Day 1 LTE Part 2) post-last dose with mean percentage change from baseline of −56% and −46%, respectively. Performance on functional, cognitive, psychiatric, and neurologic clinical outcomes was as expected for participants with mild AD over the duration of the Treatment and Post-Treatment Periods. No clinically meaningful differences in these parameters were observed between participants who received placebo and those who received ISIS 814907, regardless of the dose level. The mean change from baseline in ventricular volume (VV) as a percentage of total intracranial volume 6-months post-baseline was greater in the 10-mg, 30-mg, 60-mg, and 115-mg ISIS 814907 dose groups (0.5%, 0.7%, 0.7% and 0.6%, respectively) than that observed in the placebo group (0.2%). Ventricular enlargement (VE) was not observed in qualitative neuroradiological review of safety MRIs from participants in ISIS 814907 or placebo groups. Clinical findings potentially associated with VE were not observed during the Treatment or Post-Treatment Periods. Whole brain volume declined slightly from baseline in all groups and did not differ between participants who received placebo and those who received ISIS 814907.

CSF was obtained via lumbar puncture from patients receiving ISIS 814907 or placebo and phosphorylated tau protein concentration was measured with the Elecsys Phospho-Tau (181P) CSF assay. The Elecsys Phospho-Tau (181P) CSF assay is an in vitro diagnostic immunoassay intended for the quantitative determination of the phosphorylated Tau protein in human CSF. Intended uses include the following:

1. The Elecsys Phospho-Tau (181P) CSF assay is intended to be used alone or in combination with Elecsys β-Amyloid (1-42) CSF assay as a ratio in adult subjects with mild cognitive impairment (MCI) as an aid to identify subjects who are at lower vs. higher risk of cognitive decline as defined by change in a clinical score within a 2 year period.

2. The Elecsys Phospho-Tau (181P) CSF assay is intended to be used in combination with Elecsys β-Amyloid (1-42) CSF assay as a ratio in adult subjects with cognitive impairment being evaluated for AD and other causes of cognitive impairment wherein a positive and negative CSF result is concordant with positive and negative amyloid Positron Emission Tomography (PET) scan result, respectively.

CSF was obtained via lumbar puncture from patients receiving ISIS 814907 or placebo and b-amyloid (1-42) protein concentration was measured with the Elecsys β-Amyloid (1-42) CSF assay. Elecsys β-Amyloid (1-42) CSF is an in vitro diagnostic immunoassay intended for the quantitative determination of the β-amyloid (1-42) protein concentration in human cerebrospinal fluid (CSF). Intended uses include the following:

1. The Elecsys β-Amyloid (1-42) CSF assay is intended to be used in adult subjects with cognitive impairment being evaluated for Alzheimer disease (AD) and other causes of cognitive impairment. Result above the cutoff is consistent with a negative amyloid positron emission tomography (PET) scan. Negative β-amyloid PET scans indicate sparse to no neuritic plaques and are inconsistent with a neuropathological diagnosis of AD at the time of image acquisition; a negative scan result reduces the likelihood that a patient's cognitive impairment is due to AD.

2. The Elecsys β-Amyloid (1-42) CSF assay is intended to be used in combination with Elecsys Phospho-Tau (181P) CSF or Elecsys Total-Tau CSF assay as a ratio in adult subjects with cognitive impairment being evaluated for AD and other causes of cognitive impairment wherein a positive and negative CSF result are concordant with positive and negative amyloid Positron Emission Tomography (PET) scan result, respectively.

3. Elecsys β-Amyloid (1-42) CSF assay is intended to be used alone or in combination with Elecsys Phospho-Tau (181P) CSF or Elecsys Total-Tau CSF assay as a ratio in adult subjects with mild cognitive impairment (MCI) as an aid to identify subjects who are at lower vs. higher risk of cognitive decline as defined by change in a clinical score within a 2 year period.

TABLE 3
Summary of percent change from baseline of CSF concentrations
of p-tau, Aβ42, and neurofilament light (NfL) by dose group

| | Change from Baseline to Endpoint* | | | | |
|---|---|---|---|---|---|
| | | ISIS | ISIS | ISIS | ISIS |
| | | 814907 | 814907 | 814907 | 814907 |
| | Placebo | 10 mg | 30 mg | 60 mg | 115 mg |
| | (N = 12) | (N = 6) | (N = 6) | (N = 9) | (N = 13) |
| CSF | | | | | |
| ABeta1-42 | | | | | |
| Mean pg/mL | −1.5 ± 9.2 | −14.4 ± 5.8 | −23.3 ± 4.1 | −22.5 ± 8.8 | −10.4 ± 17.5 |
| (SD) | | | | | |
| Median | −2.6 | −13.5 | −21.7 | −19.6 | −12.7 |
| Min, Max | −17.0, 12.7 | −23.2, −7.1 | −30.5, −20.7 | −35.9, −12.2 | −35.8, 20.0 |
| CSF | | | | | |
| Phospho- | | | | | |
| Tau | | | | | |
| Mean pg/mL | −2.7 ± 11.5 | −35.0 ± 8.1 | −43.8 ± 7.2 | −52.0 ± 7.6 | −48.5 ± 15.2 |
| (SD) | | | | | |
| Median | −1.8 | −34.8 | −46.2 | −51.3 | −55.2 |
| Min, Max | −32.2, 7.8 | −44.1, −22.5 | −51.2, −32.4 | −66.8, −44.3 | −67.2, −14.2 |
| CSF Tau to | | | | | |
| ABeta-42 | | | | | |
| Ratio | | | | | |
| Mean (SD) | 0.6 ± 11.9 | −17.1 ± 14.9 | −21.3 ± 9.7 | −33.8 ± 9.8 | −35.5 ± 10.8 |
| Median | 2.2 | −24.2 | −21.9 | −36.7 | −37.2 |
| Min, Max | −24.4, 20.3 | −25.7, 9.3 | −34.7, −10.6 | −44.5, −13.9 | −52.8, −17.8 |
| CSF NfL | | | | | |
| Mean pg/mL | 2.8 ± 26.2 | 8.4 ± 30.6 | 17.7 ± 14.5 | 15.5 ± 29.6 | 10.9 ± 16.6 |
| (SD) | | | | | |
| Median | 5.6 | −3.6 | 15.3 | 9.2 | 8.7 |
| Min, Max | −33.7, 45.1 | −23.0, 57.0 | 0.9, 40.5 | −13.1, 59.3 | −9.6, 46.0 |

*For CSF parameters (ABeta1-42, Phospho-Tau, ABeta-42 Ratio), endpoint is defined as Study Day 141. For CSF NfL, endpoint is defined as Study Day 169.

In conclusion, the ASO ISIS 814907 engaged its target, as evidenced by the marked dose-dependent and sustained reductions in the concentration of CSF t-tau, and had an acceptable safety profile in participants with AD. More generally, these data demonstrate antisense-mediated suppression of protein synthesis in the CNS of participants with a neurodegenerative disease.

### Example 2: Phase 2 Trial Human Clinical Trial with ISIS 814907 for Alzheimer's Disease (AD)

A randomized, double-blind, placebo-controlled Ph2 clinical trial in adult human subjects with Mild Cognitive Impairment (MCI) due to AD and mild AD dementia is to be conducted. All doses of ISIS 814907 are delivered via intrathecal administration.

### Example 3: Phase 3 Human Clinical Trial with ISIS 814907 for Alzheimer's Disease

A randomized, double-blind, placebo-controlled Phase 3 clinical trial is conducted to evaluate the efficacy and safety of intrathecally administered ISIS 814907 in adult human subjects with MCI due to AD or mild AD dementia.

### Example 4: Phase 2 Trial Human Clinical Trial with ISIS 814907 for Progressive Supranuclear Palsy (PSP)

A randomized, double-blind, placebo-controlled Ph2 clinical trial in adult human subjects with PSP is to be conducted. All doses of ISIS 814907 are delivered via intrathecal administration.

### Example 5: Phase 3 Human Clinical Trial with ISIS 814907 for PSP

A randomized, double-blind, placebo-controlled Phase 3 clinical trial is conducted to evaluate the efficacy and safety of intrathecally administered ISIS 814907 in adult human subjects with PSP.

### Example 6: Phase 2 Trial Human Clinical Trial with ISIS 814907 for Fronto-Temporal Demential (FTD)

A randomized, double-blind, placebo-controlled Ph2 clinical trial in adult human subjects with FTD is to be conducted. All doses of ISIS 814907 are delivered via intrathecal administration.

### Example 7: Phase 3 Human Clinical Trial with ISIS 814907 for FTD

A randomized, double-blind, placebo-controlled Phase 3 clinical trial is conducted to evaluate the efficacy and safety of intrathecally administered ISIS 814907 in adult human subjects with FTD.

### Example 8: Phase 2 Study of ISIS 814907

### Rationale for Study Population

The study population will include participants with MCI due to AD (Stage 3) and with mild AD dementia (Stage 4) between the ages of 50 to 80 years. Review of published reports on tau PET (flortaucipir, ¹⁸F-AV-1451) have identified several factors associated with increased brain tau levels (e.g., amyloidosis, disease severity), a negative correlation with age in amyloid-positive populations, as well as the relationship between baseline tau level, tau progression, and cognitive decline. Due to these complex interacting factors, considerable heterogeneity and a high degree of overlap between MCI and mild AD dementia on tau PET and CDR-SB was evident from a study targeting a similar study population. ISIS 814907 is expected to provide benefit to patients in these early symptomatic stages of AD due to the reduction of the levels of pathogenic tau expression at a stage when significant neurodegeneration has not yet been established. Hence, presently maintaining approximately an even split of participants with MCI/mild AD dementia for the study balances the goal of targeting early disease with the need to measure change over time both on clinical measures and tau PET.

### Rationale for Phase 2 Dose Selection

The 3 doses of ISIS 814907 to be assessed in this Phase 2 study are 60 mg Q24W, 115 mg Q24W and 115 mg Q12W via intrathecal (IT) administration. The doses were chosen based on PK-PD modeling with exposure and biomarker data from earlier studies and are supported by nonclinical toxicology and clinical safety data.

In a double-blind multiple ascending dose (MAD) Study, 34 participants received treatment with ISIS 814907 at 10 mg, 30 mg, or 60 mg IT dose Q4W or 115 mg Q12W for 12 weeks (from the first to the last dose) with data collected up to 24 weeks after the last dose. Robust, dose-dependent decreases in CSF total and phosphorylated tau were observed. At Day 141 (8 weeks after the last dose), the mean reduction of CSF total tau from baseline was 29.5% in the 10 mg Q4W dose cohort, 39.7% in the 30 mg Q4W dose cohort, 48.8% in the 60 mg Q4W dose cohort, and 41.9% in the 115 mg Q12W dose cohort. In the 2 highest dose cohorts where CSF samples were collected for 24 weeks after the last dose in the MAD part and participants seamlessly transitioned to the long term extension (LTE), CSF total tau continued to decrease after the last dose and the reduction was sustained for 24 weeks after the last dose (mean reduction: 56.2% with 60 mg Q4W dose, 50.6% with 115 mg Q12W dose). In most of the participants (7 out of 8) receiving lower doses of 10 mg and 30 mg in the MAD part who eventually entered the LTE, CSF tau reduction was still evident for periods that exceeded 12 months after their last dose in the MAD study. The mean percentage changes of total tau and phosphorylated tau from baseline were comparable.

With data from the above Study, a population PK-PD model was developed to understand the relationship between different dose regimens and the resulting CSF tau reduction and therefore help select the dose regimens for the Phase 2 AD study. The model was built using the available ISIS 814907 concentration and total tau data in CSF. The model was then used to simulate CSF tau reduction with 72-week dosing and predicted approximately 53%, 64%, and 76% mean reduction of CSF tau from baseline at 76 weeks (end of study scheduled) with 60 mg Q24W, 115 mg Q24W and 115 mg Q12W, respectively. In addition to tau reduction, the following considerations were taken into account for dose selection:
The 115 mg Q12W dose was chosen as the highest dose, as 115 mg was the highest dose level tested in earlier studies, and Q12W is the most frequent IT dosing considered for the AD patient population. The 115 mg Q12W has safety margins of 3 to 5.2-fold based on the no-observed-adverse-effect-level (NOAEL) (35 mg Q4W, human equivalent dose (HED)=350 mg) and maximal tolerated dose (MTD) (60 mg single dose, HED=600 mg), respectively, in NHPs.
The 115 mg Q24W dose allows study of biannual dosing. It has safety margins that are the same as for the 115 mg Q12W dose.
The 60 mg Q24W dose provides additional data to potentially inform the minimum efficacious dose. It has safety margins of 5.8- to 10-fold based on the NOAEL and MTD of ISIS 814907.
   The safety margins based on the HED of the NOAEL in NHPs do not take dose frequency into account, and all the proposed dose regimens are less frequent than the regimens in the repeated dose studies in NHPs. The proposed doses are expected to adequately characterize the relationship among dose, exposure, tau, and clinical outcomes.

### Eligibility Criteria

### Key Inclusion Criteria:

Must meet all of the clinical criteria for MCI due to AD or mild AD dementia according to the National Institute on Aging at National Institutes of Health and the Alzheimer's Association (NIA-AA) and must have the following at Screening Visit 1:
   1) Repeatable Battery for the Assessment of Neuropsychological Status (RBANS) Delayed Memory Index score of ≤85, indicative of objective evidence of memory impairment
   2) CDR global score of 0.5 for MCI due to AD or 0.5 or 1 for mild AD dementia
   3) MMSE score of 22 to 30 (inclusive)
   4) CDR Memory Box score of ≥0.5
      Evidence of amyloid pathology as measured by positive emission tomography (PET) or cerebrospinal fluid (CSF) sampling.

### Study Overview

Approximately 735 participants will be randomized. Randomized participants will receive placebo Q12W, ISIS 814907 60 mg Q24W, ISIS 814907 115 mg Q24W, or ISIS 814907 115 mg Q12W.

### Treatment Arms:

Placebo Q12W
ISIS 814907 60 mg Q24W
ISIS 814907 115 mg Q24W
ISIS 814907 115 mg Q12W

Participants will receive study treatment via IT injection every 12 weeks, starting on Study Day 1. Participants in the ISIS 814907 60 mg Q24W and ISIS 814907 115 mg Q24W arms will receive ISIS 814907 at Weeks 1, 24, 48, and 72 and placebo at Weeks 12, 36, and 60. Participants in the Placebo arm will receive placebo at each dosing visit. Participants in the ISIS 814907 115 mg Q12W arm will receive ISIS 814907 115 mg at each dosing visit. CSF and blood will be collected from all participants for analysis of ISIS 814907 PK and relevant biomarkers. Tau levels in certain areas of the brain as measured by tau PET imaging will be assessed in participants in the tau PET substudy.

| Arms | Assigned Interventions |
|---|---|
| Placebo Comparator: Placebo Q12W | Drug: ISIS 814907- |
| Participants will receive ISIS 814907 - | matching placebo |
| matching placebo, intrathecal (IT) | Administered as specified |
| injection, once on Day 1 and then once | in the treatment arm. |
| every 12 weeks (Q12W) for up to 72 | |
| weeks. | |
| Experimental: ISIS 814907 60 mg | Drug: ISIS 814907 |
| Q24W | Administered as specified |
| Participants will receive ISIS 814907 | in the treatment arm. |
| 60 milligrams (mg), IT injection, once | Drug: ISIS 814907- |
| every 24 weeks (Q24W) from Week 1 | matching placebo |
| up to 72 weeks and ISIS 814907- | Administered as specified |
| matching placebo, IT injection, once | in the treatment arm. |
| at Weeks 12, 36 and 60. | |
| Experimental: ISIS 814907 115 mg | Drug: ISIS 814907 |
| Q24W | Administered as specified |
| Participants will receive ISIS 814907 | in the treatment arm. |
| 115 mg, IT injection, Q24W from | Drug: ISIS 814907- |
| Week 1 up to 72 weeks and ISIS | matching placebo |
| 814907-matching placebo, IT injection, | Administered as specified |
| once at Weeks 12, 36 and 60. | in the treatment arm. |
| Experimental: ISIS 814907 115 mg | Drug: ISIS 814907 |
| Q12W | Administered as specified |
| Participants will receive ISIS 814907 | in the treatment arm. |
| 115 mg, IT injection, once on Day 1 | |
| and then Q12W for up to 72 weeks. | |

### Primary Outcome Measures:

Dose-response in Change From Baseline to Week 76 on the CDR-SB [Time Frame: Baseline to Week 76]

The Clinical Dementia Rating (CDR) scale is a clinician-rated dementia staging system that tracks the progression of cognitive impairment in 6 categories (memory, orientation, judgment and problem solving, community affairs, home and hobbies, and personal care). Each category is scored on a 5-point scale in which None=0, Questionable=0.5, Mild=1, Moderate=2, and Severe=3. The global CDR score is established by clinical scoring rules and has values of 0 (no dementia), 0.5, (questionable dementia), 1 (mild dementia), 2 (moderate dementia), and 3 (severe dementia). The CDR-SB is obtained by adding the ratings in each of the 6 categories and ranges from 0 to 18 with higher scores indicative of greater impairment.

### Secondary Outcome Measures:

Change From Baseline to Week 76 on the CDR-SB [Time Frame: Baseline to Week 76]
   The CDR scale is a clinician-rated dementia staging system that tracks the progression of cognitive impairment in 6 categories (memory, orientation, judgment and problem solving, community affairs, home and hobbies, and personal care). Each category is scored on a 5-point scale in which None=0, Questionable=0.5, Mild=1, Moderate=2, and Severe=3. The global CDR score is established by clinical scoring rules and has values of 0 (no dementia), 0.5, (questionable dementia), 1 (mild dementia), 2 (moderate dementia), and 3 (severe dementia). The CDR-SB is obtained by adding the ratings in each of the 6 categories and ranges from 0 to 18 with higher scores indicative of greater impairment. Positive change from baseline indicates clinical decline.
Change From Baseline to Week 76 on the Alzheimer's Disease Cooperative Study Activities of Daily Living for Mild Cognitive Impairment (ADCS-ADL-MCI) [Time Frame: Baseline to Week 76]
   The ADCS-ADL-MCI consists of 17 instrumental items (e.g., shopping, preparing meals, using household appliances) and 1 basic item (getting dressed). Ratings reflect caregiver observations about the participant's actual functioning and provide an assessment of change in the functional state of the participant over time. The total score ranges from 0 to 53, with lower values over time reflecting functional deterioration. Positive change from baseline indicates clinical improvement.
Change From Baseline to Week 76 on the Alzheimer's Disease Assessment Scale Cognitive Subscale (ADAS-Cog 13) [Time Frame: Baseline to Week 76]
   ADAS-Cog13 comprises both cognitive tasks and clinical ratings of cognitive performance. The scale items capture word recall, ability to follow commands, the ability to correctly copy or draw an image, naming, the ability to interact with everyday objects, orientation, word recognition, memory, comprehension of spoken language, word-finding, and language ability, with a measure for delayed word recall and concentration/distractibility. The total score ranges from 0 to 85. An increase in score over time indicates increasing cognitive impairment. Positive change from baseline indicates clinical decline.
Change From Baseline to Week 76 on the Mini Mental State Examination (MMSE) [Time Frame: Baseline to Week 76]
   The MMSE is a widely used performance-based test of global cognitive status. It consists of 11 tasks that assess orientation, word recall, attention and calculation, language abilities, and visuospatial functions. The scores from the 11 tests are combined to obtain the total score, which ranges from 0 to 30, with lower scores over time indicating increasing cognitive impairment. Positive change from baseline indicates clinical improvement.
Change From Baseline to Week 76 on the Modified Integrated Alzheimer's Disease Rating Scale (iADRS) [Time Frame: Baseline to Week 76]
   iADRS is a composite and is calculated as a linear combination of total scores of ADAS-Cog13 and Alzheimer's Disease Cooperative Study Instrumental Activities of Daily Living Inventory (ADCS-iADL) that measures cognition and daily function. ADCS-iADL is calculated from a subset of questions from ADCS-ADL. Range for ADCS-iADL is 0-59 and higher scores reflect better performance. ADAS-Cog13 comprises cognitive tasks and clinical ratings of cognitive performance. Scale items capture word recall, ability to follow commands, ability to correctly copy/draw, naming, ability to interact with everyday objects, orientation, word recognition, memory, spoken language comprehension, word-finding, and language ability, with a measure for delayed word recall and concentration/distractibility. Score ranges from 0 to 85 with higher scores reflecting cognitive impairment. iADRS score range is 0-144 and higher scores indicate greater impairment. Positive change from baseline indicates clinical decline.
Change From Baseline to Week 76 on the Alzheimer's Disease Composite Score (ADCOMS) [Time Frame: Baseline to Week 76]
   ADCOMS is a composite score comprised of ADAS-cog (4 items), MMSE (2 items) and CDR-SB (6 items). The total scores on the scale range from 0 to 1.97 with higher scores indicating greater impairment. Positive change from baseline indicates clinical decline.

1.-200. (canceled)

201. A method of reducing Tau RNA, reducing Tau protein, or ameliorating a disease or disorder associated with Tau protein in a human subject in need thereof, the method comprising administering to the human subject a dose of a composition comprising a modified oligonucleotide according to the following chemical structure: 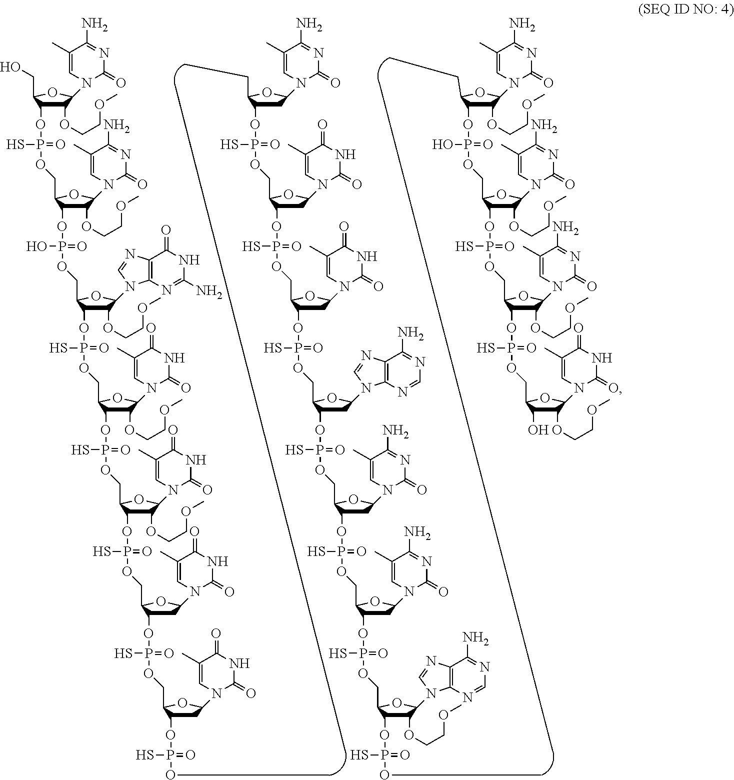
or a salt thereof,
wherein the composition comprises about 10 mg, about 30 mg, about 60 mg or about 115 mg of the modified oligonucleotide.

202. The method of claim 201, wherein the salt is a sodium or potassium salt.

203. The method of claim 201, wherein a dose of the composition is administered once every 4 weeks, 8 weeks, 12 weeks, 16 weeks, 20 weeks, or 24 weeks.

204. The method of claim 201, wherein at least 4 doses of the composition are administered.

205. The method of claim 201, wherein the composition is administered to the CNS of the human subject.

206. The method of claim 201, wherein the composition is administered by intrathecal administration.

207. The method of claim 201, wherein the composition is administered by bolus intrathecal administration.

208. The method of claim 201, wherein the disease or disorder associated with Tau protein is a neurodegenerative disease or disorder, or a tauopathy.

209. The method of claim 201, wherein the disease or disorder associated with Tau protein is Alzheimer's disease (AD), mild AD, MCI due to AD, mild AD dementia, Fronto-temporal Dementia (FTD), Frontotemporal Dementia with Parkinsonism-17 (FTDP-17), Progressive Supranuclear Palsy (PSP), Chronic Traumatic Encephalopathy (CTE), Corticobasal Ganglionic Degeneration (CBD), Pick Disease, Argyrophilic Grain Disease (AGD), Globular Glial Tauopathies, Epilepsy, Dravet's Syndrome, Down syndrome, Prion diseases (sCJD, vCJD, gCJD, GSS, FFI), Diffuse neurofibrillary tangles with calcification, Familial British and Danish dementia, Postencephalitic parkinsonism, Subacute sclerosing panencephalitis, Myotonic dystrophy (DM1), PROMM (DM2), Aging-related tau astrogliopathy, Traumatic brain injury, Chronic traumatic encephalopathy, IgLON5-related tauopathy, Guadeloupean parkinsonism, Parkinson—dementia complex of Guam, Non-Guamanian motor neuron disease with NFTs, Amyotrophic lateral sclerosis of Guam, X-linked parkinsonism with spasticity, Cerebrotendinous xanthomatosis, Niemann—Pick disease type C, PANK2-associated Neurodegeneration with Brain Iron Accumulation (NBIA), PLA2G6-assiciated NBIA, SLC9A6 mental retardation, or diseases or disorders associated with mutations in LRRK2, PRKN, SNCA, TARDBP, or C9orf72 genes.

210. The method of claim 201, wherein at least one symptom or hallmark of the disease or disorder associated with Tau protein is ameliorated.

211. The method of claim 210, wherein the at least one symptom or hallmark comprises loss of memory, cognitive decline, loss of ability to understand or express speech, abnormal behavior, impaired motor function, loss of cognitive function, neuropsychiatric behavior dysfunction, impaired global function, loss of motor function, impaired cognitive function, impaired neuropsychiatric function, impaired daily function, impaired attention, impaired visuoperceptual processing, impaired memory, impaired degree of independence, increased apathy, impaired learning ability, impaired mental concentration, impaired understanding and expression of speech, impaired behavior, depression, irritability, anger, impaired mobility, impaired self-care, pain, discomfort, anxiety, seizures, suicidal ideation, suicidal behavior, increase in the number and/or volume of neurofibrillary inclusions.

212. The method of claim 201, wherein the human subject has a mutation in at least one gene selected from MAPT, APOE, APP, PSEN1, PSEN2, LRRK2, STX6, EIF2AK3, and MOBP.

213. The method of claim 201, comprising detecting an amount of Tau RNA or Tau protein in a biological sample from the human subject.

214. The method of claim 213, wherein the biological sample comprises cerebrospinal fluid.

215. The method of claim 213, wherein the detecting occurs before the administering and/or after the administering.

216. The method of claim 201, comprising analyzing brain activity, brain size, size of neurofibrillary inclusions, volume of neurofibrillary inclusions, amount or concentration of total tau protein, phosphorylated tau protein, or amyloid beta protein in the cerebral spinal fluid, or a combination thereof by performing magnetic resonance imaging (MRI), Positron Emission Tomography (PET), Electroencephalogram (EEG), or CSF analysis of the human subject.

217. A method of reducing Tau RNA, reducing Tau protein, or ameliorating a disease or disorder associated with Tau protein in a human subject in need thereof, the method comprising intrathecally administering to the human subject a therapeutically effective amount of about 10 mg, about 30 mg, about 60 mg, about 90 mg, or about 115 mg of a modified oligonucleotide, wherein the modified oligonucleotide has the following chemical notation (5′ to 3′): mCes mCeo Ges Tes Tes Tds Tds mCds Tds Tds Ads mCds mCds Aes mCeo mCes mCes Te (SEQ ID NO: 4); wherein,
A=an adenine nucleobase,
mC=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

218. A method of ameliorating Alzheimer's disease in a human subject in need thereof, the method comprising intrathecally administering to the human subject a therapeutically effective amount of about 10 mg, about 30 mg, about 60 mg, about 90 mg, or about 115 mg of a modified oligonucleotide according to the following chemical structure: 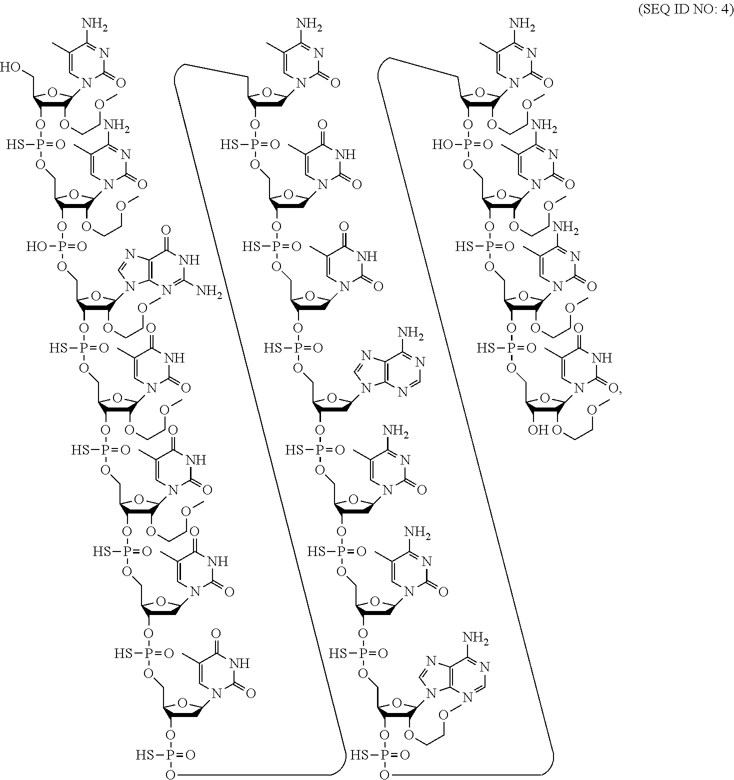
or a salt thereof,
wherein the salt is a sodium or potassium salt.

219. A method of ameliorating Alzheimer's disease in a human subject in need thereof, the method comprising intrathecally administering to the human subject a therapeutically effective amount of about 10 mg, about 30 mg, about 60 mg, about 90 mg, or about 115 mg of a modified oligonucleotide, wherein the modified oligonucleotide has the following chemical notation (5′ to 3′): mCes mCeo Ges Tes Tes Tds Tds mCds Tds Tds Ads mCds mCds Aes mCeo mCes mCes Te (SEQ ID NO: 4); wherein,
A=an adenine nucleobase,
mC=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.


SEQUENCE LISTING (4 sequences)

SEQ ID NO: 1 - DNA, 6815 bp, Homo sapiens
  source 1..6815: mol_type = unassigned DNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagaggagttgagagttccgggccggcagaggaaggcgcctgaaaggcccctggccaatgagattagcgcccacgtccagcctggaccctgcggagaggcctctggggtctctgggccgtgcctcggggagaaagagccagaagctcccgtcccgctgaccgcgagccttcctcagcaccgtcccgtttgcccagcgcctcctccaacaggaggccctcaggagccctccctggagtggggacaaaaaggcggggactgggccgagaagggtccggcctttccgaagcccgccaccactgcgtatctccacacagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 2 - DNA, 137001 bp, Homo sapiens
  source 1..137001: mol_type = unassigned DNA
caaaaattagccgggagtggtggcatatgcctgtaatcccagtagctgggaggctgagacaggaaaatcgcttgaacccgggaaacaggttgcagtgagccgagatcgtgccactgcactccagcctgggcaacagagcgagactccatctcaaaaaaacaaaacaaacacacacaaaaaaccaaaaataaataaataaaatgatcacttctgaatactgatctaactaggggttgcagggtgggctgatatagggagaaactggagagcaaggagatcactaaggtccctacatgtccagaaccaagatagaggtcttgaactaggatggtggcagttagaacaacaacaacaaaaagtcaattccaggctgagtgcagtggctcatgcttgtaatcccaacgctttgggaggctgaggtgggagttagaaagcagcctgggcaacactgcaagacctcctctctaaaaaaaaaaaaaaaaaaaagttagccaggtgtggtggtgcccacctgtagtcccagcaactcagaaggctgaggtgggaagattgcttgagccccaggagttcaagcttgccgtgagctacgattgtgccactgcactccagcctgagcaagaccttgtctccaaaaaaaggtcaattccactgacttttctaaggtgtacaccatcaaggggcagctccatctccaggccattggctcatgagacattctgtagtcagaaggctagggcagattgctttgagcaagcccccatggtggttctcactcctacttctttgggtatatgcccctctgtttaaaaataaagttaatatgcatttaaaaaaaaaaaggagaaaaaggtcagttccagaaactgtgtgaataaagcattttacttgctttttctattaatctataacatatgttgattttttaaaaagaatataagagctatgcaaattggagcttcaagacaacttcccatctccctaggaggagatggctgccctaaacccccctacatagaaatcatcccactgcttgggcttaaacttgatgttggggaaatgaaaaatccaagctaaggccgaagcctggggcctgggcgaccagcagaatgaggaccactggtcagtttcaggctgaggtgcgtcttccaggggacaatctctagctggcccttaaacattcagacttcaagctctatttacagcataaaggtgtttcaaaagacgtgatacaaataactgcaaatgctctgcgatgtgttaagcactgtttgaaattcgtctaatttaagatttttttttctgacgtaacggttagattcacgtttctttttttttaagtacagttctactgtattgtaactgagttagcttgctttaagccgatttgttaaggaaaggattcaccttggtcagtaacaaaaaaggtgggaaaaaagcaaggagaaaggaagcagcctgggggaaagagaccttagccaggggggcggtttcgggactacgaagggtcggggcggacggactcgagggccggccacgtggaaggccgctcaggacttctgtaggagaggacaccgccccaggctgactgaaagtaaagggcagcggacccagcggcggagccactggccttgccccgaccccgcatggcccgaaggaggacacccacccccacaacgacacaaagactccaactacaggaggtggagaaagcgcgtgcgccacggaacgcgcgtgcgcgctgcggtcagcgccgcggcctgaggcgtagcgggagggggaccgcgaaagggcagcgccgagaggaacgagccgggagacgccggacggccgagcggcagggcgctcgcgcgcgcccactagtggccggaggagaaggctcccgcggaggccgcgctgcccgccccctcccctggggaggctcgcgttcccgctgctcgcgcctgcgccgcccgccggcctcaggaacgcgccctcttcgccggcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtaagcgccgcggctccgaaatctgcctcgccgtccgcctctgtgcacccctgcgccgccgcccctcgccctccctctccgcagactggggcttcgtgcgccgggcatcggtcggggccaccgcagggcccctccctgcctcccctgctcgggggctggggccagggcggcctggaaagggacctgagcaagggatgcacgcacgcgtgagtgcgcgcgtgtgtgtgtgctggagggtcttcaccaccagattcgcgcagaccccaggtggaggctgtgccggcagggtggggcgcggcggcggtgacttgggggagggggctgcccttcactctcgactgcagccttttgccgcaatgggcgtgtgtgtgtgtgtgtgtgtgtgtgtgtgtgtgtgtgtgtgtgtggaggggtccgataacgacccccgaaaccgaatctgaaatccgctgtccctgccgctgttcgccatcagctctaagaaagacgtggatcgggttctagaaaagatgactccctgcacgcccctccctgcacctcccgagcagtgattccgacagggccttcactgcccctgattttaggcgggggccggccccctccccttttcctccttcagaaacccgtaggggacatttgggggctgggagaaatcgaggagatggggaggggtccacgcgctgtcactttagttgcccttccccctgcgcacgcctggcacagagacgcgagcagcgccgtgcctgagaacagtgcgcggatcccactgtgcacgctcgcaaaggcagggttcacctggcctggcgatgtggacggactcggcggccgctggtccccgttcgcgggcacgcacagccgcagccacgcacggatgggcgcggggctgcaggtgcatctcggggcggatttctttctcagcgctcggagcgcagggcgcccggcgtgtgcgctccctgccggaggcgcggggctggcgcgcagggctcgcccctcactgcggcagtgggtgtggaccctggtgggcgaggaagggggaggataggctgtgcctcctcccactcccgcccccagcccccctttttttccccctcggaacgcgaggtgccatcttttttcggcgtgtcacgtctttacggtgccatgccaaaccgggtggccgggcttcataggacagggcggggcctggcattaaagggagggggacaatcagcgctgaaatcttggcgttttgctgctgcgggcgtgagcactgggggcgttcgcccagcaccttcttcgggggctctttgctttgtctgtagaggttacgtgatctgcgctcccagccctggtttctggcttttattctgagggtgttcagtcaacctcccccctacgcccatgcgcctctctttcctttttcgctcctcatttccgagcccattgttggatctcgaggcttgctgggttcgatgaactcgagtcaaccccccgacccccggcacgcatggaacgggcgtgaccgcgcgcagcctcgtctcggagtctgccggcgccgggaagcttctgaagggatgggattcgagtctccgtgcgcgctgcgggcggcggcagagggatctcgcccctccctacaccccaagtgtcctgagggccacgccacaccaggttgcccagcgagggacgctggctacccatccggggatgggtggggagccctggcggggcctctccggctttacgccctgttgcttcgcctggccggagaatgtgaggaaggggcataaggttactggtgcttcggccacacccatctttctgagcccactggactgggcgcagaggggggattgccatggaaaccacaggtgtccggagaggggatcttggggctggcctcaccccttccctgcggagattggggaccctggggtagggggagccgcgcccagtcggcctcctggaggacacgggaggaagccccgaacccccgcgcctgaggctgtttctgattggcccctggaggccgcagacacgcagataggcggccctgggtgtatttttattaatattatgtccgtactgattaatattatttatcttaaataaatttcacccgtgtccaagttcaccgcgcccccaaaaccgagtctggggcggcagggggaactcctggccaacgaatccatgcctcgccctcctgtgatgaacctggtacgcacggttttctggttaattctatcgctgaaaactggtgcggggggcgcacttctgagacggaagagcatctaggagctgaatcctccacgcgggtcgcccaggttgatctgaatttctggggaatggcttggctgcccgcccgggaccaggccgaccctccttgacggtggcgtagagggctggagcctgggtactgcgaggctcctcgcatggctgggcccgccgcgaggggttgcagagcggctcagggatcgattcaagcatcgtctctcctccctcgcccccagacagagctgggcgcggggttccccttccagatggagcgagggtctcggggtggccccggaaaaggggagcccgcggccacggctacgtattgccatctcgcgagcagagatgtcacctcctgcctttggaggaaagggagcccggtggggatgagcgcatttagcccaatgctgggaacaaagcgcactccgcgcttctgcgatttcgctccattttgaaatgtgttggcgctttggtggggccgctgcggtgggcaaggccgggggcgctgttaatggaggaacctcagggggacggtccttcgtaggaaactctatcctggctctgcgcgcgctttaaggaaatggcttccctccaggacctcgagggatgcagcttttgcgcggatgacggtggggtgctgaaccagccggtgcgcctctggaaatgtctgggcacggatcctggggccatcgacgactcctccccattcccagcaggcgggagctcttacattccgagcgagtgacccctctcaccctctggcgctcacacacctgtaactccaaacctccgtctcagaatggtccaggctggaagggatgatgggggctccgacagcgactgcctagctcacccctctgcgtgctcaggctccaggctcagcaggaccaatttgagttctatctgatccccctcggccccttaactgacccatcctacaggagacagggaaatgtctttcctaccgcggttgattctggggtgtcattttgtgttttgtgatggctgcttatatttactgtataagcattgtatttactgtataagcattgtattataattactgtataagctgcttatatttactgtataagcatctccaaatcctccctctacgtaaacaaattaatggataaacagataagtgtatcccctgcccccacccctgctacgcaggtccggagtgactcttgaagctcatacattccttggccaagtttgcttctctaacagatgtttatatagcaataacctggcttggctcttgggttcacctttggacgatttggggaaggggcttgttggctttgctgggttttggatgagtgacagtccatgactgttcctgctggaagggcgtgacttttaagtggtttctaatatcaggcattgctcctccgacaggaacaaaagaaatggatactgcccataaattgttagaaaacttagaatcgctttgattgaggaaaggttagatttattccggttggaaaaagtggcctttctattaaacgtgccctttgaccctcatgcccttggaggtcggtgccagcctggagatgggataagattgtggttttccttctgcctttttaacatctgttgttacagtccatttgttgaaaatttaaagaaactgttttattccactttccctcagcatttatgtgtgtggtttcagtagctctgtggctatatgtacgaacacgtgttatttttccaattggacatgtgataattttccaactggaccttgccttctattgatgtatttatttagcatcttccttactccctccttgaaaaagaatcactcaaaaacaaataaaaacagccgtaggggcctaatacagtgctagacatacaagaggtattcggtccataccaaatggattttatccatgaaggataaatggggaaatacagtgggaagcaggtgggaaactgcgtttgactctgctctttcctccaccaccactttcctcatcaccgtgttcagagacccccaaagccccctcacactcccagaaacacccccctggccactcctaacttgccatgcccaggagttaggtgcttccactagtgacatggagctggcgtttggggggcacctcagcaggtgacgggaagagaagaccccagcctcaccagctgggctgcagcagggagaggagtcctcatgttccagcagggactctcagctgttttcctgtaaaaccatggttctcaactgggggccactgagatgtctagagagatgtttttgttttcacaactcggggagggtgctactgacatcttgtgggtagaggccaggaatgctgttaaacatcctacaaggaaggcacaggacagtctcctacatcaaaatatgacccagtcccaatgtcaccactgctggggttgacactggcactgctatcttaattacattcattgagtgtcttttaggaggccctattctaagtgcttgctaagattatctcatttaatcctcacaacacttccgctatgtagcaggtgctgttattatctccgtgatggggaaactgaagcacagagagggttagtaacttgctaaaggtcacagagccagtgggtggtggagctggttgcctgacactagttccctcccctctcagccacatgtgggtttacttggccattgtggactagtctgggaacccagatatgatctataacattgacccagtagaatattgattccaaaaccactgtctcacaaatgaatttttacaagagtctgtaatcggagcatgacccagaataaggttagggagatgtggagttaaagctctcaatttcttatctggccccgacacagagagcaaggcatttcactctacattggtgctctgtttataaaacaaagagcaaatatctcttcctaaggtccttaaacctcttcccccaatccagggtttctggactgctctgccatatgacggggcagctggtttgattgacccagggaaggctggaaatcaagactgggggatcaagacgtagattcagtgtggccaaggtcaagtctctgaggtttagggacatcagatccccagcttaggttctgtacctcggcaaggtgaaagcgttggcgcccactgatgaggcctgctctgagattgtgggtgtgggttgagttgggtgggcataggcaagtcctcttgtaagaatcttttggcaaagatgggcctgggaggcttttctcacttcctggggcccaggctttgcaataagtattccattatactgtggtaccttggggctacctgagaatcctctgtctcgcccctgttgccttgccaaagagtttgctgtccaagaattcctttcctgtctccaggtgccatgctcctgccacctctgccaggttccctgcctgcccagatggctcccaactgagtgtgaggaggaatttgagacaggttttgagctttctgggttctccagttaggaaactttctgtaagcatgcagatagaatgggcttcagcaaaatacaaactcgaacaacttccatgtatagtcccttaattttctttgcttttttcatatttcatcaggctccatgctgagcccaatcagggacccgatagaaatccaaacaccatgtcagcgagtccccaagaaatgcattttgtgccaaggctattcaaggaaggtttgggagcagctcaagggcagacactgttaccctcccccaggtccccagtgcagggcagtgttctgcatgtggaggcagtttggcctaatggttaaggaggtaggctctgatcgggcctcctgggcacaaatcccagctccctgctcactgtgagacctaagccatattgtttagctgcttggagagttttttgtcatccacaacttggagtatgatggtacctgtctcacgggttgccatggggttcacacaagctaacccggtactcactagggccaagcacatagtaactgctcagtaaatggcatcatcggcggtgtcctgtggatgagtgcttgtgattggctgaatgaccagaggggtctaaagatcctggtgatggaatcagttgtacagataaattgttacactgagtagggatcaagataggaaaagtcggcaactacccagctcccctgcaccaaactgggcagaagtggatcctctgaaaattgcacacacccatgtttaaatgtacacacagaactcttgccacaggcaagcggagatttgtcatctgctgtccctgcctcatcttcttcctgaaatccactccatgccaggaataaactgcatgctctccaccagcccaaactgacctgccttcccgccagccatcccgggcagggtgacctggcttagtacatcgggttcagagatctttccagtttactcgttgaataaaaagtgagggctgatcgagaaagtaatggcagtcagggaaggcgaaggaggtaaagaagagattttacaaatgaagtaattcaacagagtgctgacattggtaaactggcaaacagatttcagggtggttggttgagagtagagtagaaaaggattaaataaagcaaacttgtggtgtactgaatcttaggaattccatgtatccaataagtatagtcatttatgaattaataaattcggcctaagaagccttcttatcgcttaaatcaagactaagtaacaatatatcagttttaaaaagtcattatatcagaaaatcatttaaatgatacacatagatttccaagattttactttaaccgaaactatataaatgtgaatttgttcacccatcttttgacacagggctcaggtcttctcttggtgtctggatcagccagttgaaatttcttgtctgttttgcctatgccacattaataatgcactgtctgggtcctccgatttcagtttggattttgggtttacattgtggagtcatctgaatgcagaatccttcagggattttacttttttttttttttttcatggtctttaccatcccatttgatagtaaatattactcacctttatgaagtctttccaaaacattcaactaaattttcttaaaatcattgaatgatttgaagagcttattcctcagcacttttactccatcagcttgcaccttattttttaatctttttttgagacggagtctcgctctatcgcccaggcttaagtgcaatggcgcgatcttggctcactgcgacctccacctcctgggttcaagcaattccgcctcagcctccgccgtagccgggactacaggtacacaccataatgctcggctgatttttgtatttttgtagggatggggtatcgccatgttggccaggctggtcccgaacttctgacccaagtgatccacccacctcggcctcccaaagtgctgggattacaggtgtgagccaccgcgcccggccagcttgcaccttatttaggatatgtgattattatagcaagtctggtgtacatacaagattttgaatgggcacagatgacctttagtaagtgcttggctgtgataagaggcagtcctgactgcagatcaggctgtgtggaccccagccttgcatgtttacagaccttcatgtcttattcttacagggtatcagaagaacacctactggggaaacttataaattagtaaaaggtgggcattctccccgcccatcttctgtctgtctgccaggactagcacagcactttgaagtcattcacatagaatcccaacttaagagggtaaaatcctcctcaacagactgaaaataagtttaaattccctttgctatattaactcccctgaggaaagagtcttagatcaatgtccaacactaaaaacagttttaaatcagcaagtgagaattaaatctgaagcaattgataataatgtttcattcattcctctcctttggccccgtccaccctactgctaaatccaggcatcaaagagaagagggacataattatctctagtcccagctgctggttttccttccagcctatggcccagttttctgttttactgagaaggctggtgatgttatcttgggatctaagtctgcagtttcaccacaaaaagtccagggatgcactttcatgcttgtgtcctcctccctgggatagcaaggatattagaagacccctggctctgtaattgcttgtcatgtgctctacagacgccacagaatgccaagaacgaagtgctgggaaggacaaattcatggaaccgtgggacggtgctcctcccccagcgtaaaggacagctcctcctcctgaattggagccagcgttctaaatcatgtgtcaacagagttgtcctggatcggatccagttctgccattgatttgcaggtcatttcagtggtacctgtttccagttgttcttaattgaacagtggcaccaaactattgtcttgcctcatccccctcccatggcctgtcccccaaaaagagacttcttgggtaattaatcagggcaacatcaggcagtctgggcgcggtggctcacgcctgtaatcccagcactttgggaggccgaggcgggcagatcatgaggttaggagattgagaccatcctggctttgtgaaaccccgtctctactaaaaatacaaaaaattagccgggcgtggtggcgggcgcctgtagtcccagctactcgagaggctgaggcaggggaatggcgtgaacccgggaggtggaggttgcagtgagccgagatcgcaccactgcactctagcctgggcgacagagctagacttcttctcaaaaaaaaaaaaaaaaaggaatctctttggttttatatatttttttttatatatataatatatattaaaatataatatatatatttatataatataatatataaatatattatatattatatatttttatatattatatattatatatattatatattatatatttatatatttatatattatatatatttatatattatatatttatatatattatatatttatatataatatatattatatattatatattatatattatatattatatatttatatatattatatattatatatattatatattatatatttatatattatatatttatatatattatatattatatattatatatttatatattatatatttatatattatatatatttatatatattatatattatatattatatatgtatatattatatatgttatatattatatatatttatatatataatatattgtatatattatatatctaatatattatatatattatatatattatatattataatatatattatatattatatatatttttatatatataatatgtataatatataatatatataaaaacatatataatatatattatatattatatatatattatatatattatatatattaaatatattttatatatattatatatattatatatattaaatatattttatatatattatatatatatacacatatatatatataaatgaggccaggctcggtggctcacacttgtaatcccagcactgtgggaggatcacttgaagccaggagtctgagactagcctgggcaacaaaacaagatcctgtctctacaaaaggaaactgtaaaaattagctgggcatgatggcatgtgtctgtagccctagctacttgggaggccgaagcaggaggatcgcttgagcccaggagttcaaggctacagtgagctatgattgtcccatagcactccagcctgggtaacacagcaaggccctgtctctaaactttttttttttaattctatttatatttacatgtatttaaatgtgaatattcactacctatttgttgcatgcctgcattttttatactgggcttgccaaaaacccgaacagctttctactttgacaatgtatcagaatttaaatcagcaatatgttaataagccaagcaaaggttatatatgcaaataaaactgttgtctataacctcctgttacactggggcacagcaaaagtcatggtgtagtcgcatgtgaacctgtccctttcatagctgctcattgccaggaaacatcaggaatagccatttggaagagtcatcagccctcccaccatccgttttctgtcttgtcttttccctatgagcaggggaaattccacgctggccccaatccccagtgcagcggctcagcctctgcctctgctgctggtccccatgaggccagcttagaaacggaggattttgcagaacatccctaaatccgcttgaataatgaagtgatcattcataaactcacctgaaccttattaaaacctatttaatatttttcctggataatcctatagggataacttgcctcctgggcttctctccaccgggttcagttcttcctttagtggtgaagttcctcccttcttagcatctcaactgtgcctgagaaaaggccagtggcggctgcactctgttccctgtggagtgttaataaagactgaataaattgaaataaatccctttcaatgtcattaagtgctataaataatcatgaaccaatgttcgatggctgatgagaaatgcaagaaaaaatttttaatcagtaggattcataagttgacaatctgggccaagttaaaaaaaataaaaataaaaagacttttaaaaagatcttatcgtttgttaccagtaagactgaattccagaagcaagctactccctcatttgtgggcccctgttatcactggctgcttagggttgccaagccctgaattcatttgtcaactaagagatttttggccaagattaagatttcccatgcctccatatttccatctgagaaatggagattatactgtcttccccctcagaatggatgataatgtggtctctcttctgttcgcatagtcatagaactgaaataaaacaacttaagagaattcctttgagcttctcagaagtgctgcagggctgggggatgcctcccaggagccgcagtcaggtgctgatctgaagtctttggtgggctgactttagcctgacctgaaatagtatagctgctgccacctggctcccttagcgtcagtcagacggtgcagctggttcctaggggtgagggctgagccagcagggtccgtgcccaggagggatgcatgggtggccacagcccagcctgcactgatcttgtctgtccccttctttggaaggaaggagccccaaaccagggtgcaagacagtgggtgggggtgccttgagcatgacctcaagtgatttccagcccctgccagtgctgacttctctggggaagggctgggacttccttctgggctcaagtcacgacccttggatggaatttcctgggagcttttctgttttttctggagttttcagttttttcctaaccagacagggacttggtacagaatctcatattctaattatgcctaggagcagcctctccccaccactcacagtgtttagcatgtgacaggaatcgattaaggcatgagtgattaaattaaagccaggcattgacttggatggtgtaatattctgacatctgtttggtgtcaaaggcacggggcaggcgcgttaattgaactgcttgcacctggcatttgaattgagccagagcggggctaaagtcagtttgccttcaccctgtaaatggagggtttctccggagcgtggatggtgggaggtatttcagggtgtatgcataacccccaccctgacaatggcccatctcttctccagcgtggccaggtttgagtgccagtcctgggtgtccagtggccccatagccttgcgttttagtaaaatgctgcccccattaccacctggtctgtgcacttcggtcactggaatttgccatcttccagtcccgaatgtggcaagccatggagccttaagctcttctccctccacatcctggaacagacccgccagtttcttccaggcattgcctcagtttgcccctctgtttccagtcacactctcaccagcgataaaatgattttagaccttatcatctcaccctcggatccttatggaaacaataatgagttgttccctgtttcaattccaaaattcatatccaatccgttttgcatgccattgccaaattcctcccagagcaaccccgtcacctgccctggccctctccaagtgtggtcctgccatgggcatcgcctgctaagccaagctggcctcgagctgcctgcccgggtccccacaccttggctcacctccctgcccagtcccgcctcctgccagcctgccctgtggctccttcatagatgccgtgctctttctgccccttgctcacccatggcagccttgcccctctctccctgccccaccccctatttaaattgacctgaccttcctcagtgtccatcttccccgaagctttccccagccttggcactcaaggtccagaggctacgcgtttcctctcacctgtggcagcgccgtgctccccagtgcctcacagtttccttcttgcccccgcttcctgtgtaggactcatctgcccacaggttgcacgtcctgtgagggcaaggactgtgtcttatgtgactttccttctccagtcacagagctgggcacatagatagctcaaaaccctctttattaacacagttggatgttgagaaatcaaacaggccaatgtcaaatgagctctccttatttaaatcaagtcagttctccacctcctagcactcagttccagtactctatatacatggaaataataaaaaacacatttcctttgaaacattctataatcgttcctttgccctacttcagaccaacttaacgcactccccattggtccaaatgagttttgctatacgaagatgctgataataatagcagcagtggattattctgctaaaaccattgcctcgttaatcctcagtcccgaggtggggattattatcctcattttgcagagaagcaaactgagactcagagatttcacagctggggagggagccagctcatccctctgtccaggcccaagctctctcccgcttgccttcctgcctctgcaacctcagagcatcccccatctggttctactgcctgtgctagtcgtgcaggagccaaaagacacgtctttagtgctaaggactggagaagccatgccctccagcctctgtgaatgggtcatatgtaacatgagcctggagaaattatttgaaaccaaaggcaagcctctaaaccaggctgctgcttcatggcgccggtgacggcagaaccaaatttagtgctgtgggcaggtccacacttatcaaatagagaagctcatttttcttccggctcacatcaagcatgaaaaatgttcacacataccccccacacacacatgctttccggaggggtccatgtggctagaggctggaagatgtggatgagaggagcctggcaggtaagcccagggaagatgacattcagcttcccagacagcatctacagggagaaatttaattaaaagtggggcggtttccctgagcaaggcagacaaagtcagccctctactgttaagaaaaagggtcacagtgagaggggaggtgaggagactgagtctgtattttctagtctgttgggctacactacctgatcccccttcctcaaaaatccactttactttccccatgtctacaccaatgtggttcacactctgggaccaggaaaagggggagtgatggggaacagagaagggaggagctcacacagctgaggctggggttatgcatatcgaattacttagaatttgcaacctcacagggtactttcatggcgttgaaatacacttcccacagccaccctccctctaactaaaagcaagagtcatttctcagttctggtcttgcctcccacgttctcctccacatttaagaaaatccaccagctacaaagtgaagataccatatgtgatatcccaccctagtttctgttttatcagggtttggagcaggtggagcaggcagagggatcatttcagcctataaattgtattaagggtgagtactgagtcattcttcaagaaaagttttagaagcatccaaaactgaagggtggagccacctggagacagtatcatcagtcctggccccgagcatggcctgcataggcccccatggatcccagcgggagctgcagagtgcgggcaccttggcacacagccctgagtgcaaaattaggagctgggcagagggcatctctctgtcgccattgggcagcccagggcacactggtcatagccttagaccacgaacaccctgtgcccgggggacagatgcaaccagtgtgccctgggctgcccaatggcaacagagagatcgacacctggaccccatgtcacggggactccactactaaggctcctaagactgccaccttccagtgggataagccctgcctcctactgggcccacaatgtgcagagaacacttgggactacctggctttctggatacacaaatattgatccaatctggactaattagaaggtcagtcccaataacaaatcgaagtcagctgggcgtgatggctcactcctataatcccagcactttgggaggctgaggtgggcagatcatttgaagccagaagttcaagaccagcctgggcaacatagcaaaaccctgtctctactaaaaatacaaataattaggctgggtgtggtggctcatgcctgtaatcccaacagtttgggaggctgaggcaggtggtcacctgaggtcaggagtttgagaccagcctggccaacagggtgaaaccccgtgtctactaaaaacataaaaattagccaagcatgatggcatgtgcctataatcctggctactagggaggctgagacaggagagaatcgcttgaatccaggaggtggttgcagtgagctgagatggtgccactgcactccagcctggttgacagagcaagactctgtctcaaaaaaaaaaaaaaaaaaaaaaaaagccatgcctggtggagcactacgtgtaatctcagctatttgggaggctgaggcacgagaatcacttgaacctgggaggcagtggttgcagtgagctgagatcgcgccactgcactccagcctgggcgacagagtgagtgagactccatttcaaaaaaataataaatctgagtcactttaatattgttatttggatgtcaacctctaggtgtttgagacaggagagtgatatgggggcactggaaacacacaggcacggggtgtcctcacacttgggtagcccacacgatgtgatttcagggtgctgggaggtccccccactccccaaattactaacaagtggatagtactttacagtttatatgatctcatttgattcttaacatgagcctgtgagtgaaaaattccttcccctcttctacagattaggacgttgagattcagggaggttcagagggattcagggaagtcaagtggcacctggagtcccgtggctaatttgaggccggtaggggattcgaacccaggatttgtgcttcttatgcctgggcttctgctccctggggcatggtcttccccctagctttcccattcactgctttagcctaggggtcctaccctttattaaactgccagtgcctcactgcttttctcccccaaagacaaaaaaaaagtgtttttgcttttgttttgtttttcatgggcagagacctggaatttcagcttgagaatttgtgccatatgataaataaatcaacagatggctttttccttaaaaaaaaaaaaaaaaaaaactaagatgtatttgcagtgaggcataatttgtaccaaaaagtgctcaccacactgtagtcatgggggcaggaggcagccgcgggtgaagggagaaatcttggagtccaggcagcccccttctgggctgaactggggagctgggggtgctgccagccctgccaggttctcctaggaggcggcagctcatatggctgtgggaggaggcagagggagcctcatatgcacccacatttccagggatctagaagacagaaggaggaaaaccaccatcatgttaaagcagacagttaggtaacacatcctgtaatacaagttattttttccacatctaaaggctaaaaatagttgttagaatttaaagataattggtaaatgagtttctatccttctagtttcacatcaaatggaatcatgctgccttcacatcactagtgcccgttatttgtgtttaatttccacaatgttgtctaattccactctttgggcttccccagggatccagcctccctcactcgcccatcgcagggagatgctttattcatctttgtgtcttctgtgccgggcatagcgcatggcacagaataagcactcagtaattgattcacgagtgaataaatggatgagtgggtgagttcaatattgactacaaaaaccctaaggccacactggtgagtggctgcgcctgtagtcccagctgctggggaatctgaggcaggaggatctcttgagcccaggagtttgaaactagcctgggcgatatagcgagaacctgtctcaaatgacaaaaacagggccaggtgcagtggctcacgcctggaatcccagcactttaggaggccaagatgggaggatcacttgaggccaggagtccgagaccagcctgggcaacatagggagaccctgtctctacaaaaaattttttaaaaattagctgggcatggcggtgtgcgcttgtagtcccagctactcaggaggctgaggcaggaggatcacttgagcccaggaaattgaggctgcagcgagccatgatggcaccactgcactgcagcctgggcgtcagaacgagacctgctctcaaaaaaacaaacaaacaacaaaaaaaaaggctttcttaaagagacttgagaacagaaaggggaacagatacataacttatatatttatttgttcatctttccaccttcctggagggtggaggggaacaggtctgtatttggagttttgaatgctaaaagtgggaatacatgtactgtttgccatgatctgttcaaaagttaagccaaatgccttagattctcctgaaaactggaatgccactgtaaactataagccccacttcaaagataaaagatcttgatgaacagggctgggtctgtggactgggcctctccccaccacacaaggaagggtggtgccagttgaaggaaaatcacttaaatccttgctgtctcctaataaggtgtggtcccaggtagggctgtcagaattagcaaattaaaacacagggcatctgtgaaaattagaatttcagataacaacaaataattggcataggctgcataatgtccctcaaagatatcaggtcctaatctccagaacctgtaaatgtgatcttatttggaaaaggggtctttgtagatgtggttaaattaaggattttgagatggggggattatcctgtattatctaggtaggtcctaaatgcagtcacactcatccttgtaagaggaaggaagagagagatggaaaacacagaagagaagacaatgtggtgatggaggcagagattggagtgaggtggccacaagccaaggactgctggcagctaccagcagccagaaaagtccaggaaccaattctctcttggagctccagagggagtgtggccctgctgacaccttagcttcaacctagtgatcctgattttggactttggccttcagaagtgtgagggaatgaatatctgttgttttaagccaccaagtttatggtcatttcctacagcagccacaggaatcaaaaacagtaagtatgtcccatgcaatgtttgtgacacacaccaaaaatattacttgttgttcacctgaaattcaaatttaactgggtctcctgtattttatttggccaacctagttcccaggcccaaagaaagaggcttttgaaatttgcaagaaagctggttggagctgtcagaaagtggactttgtaaacacagtaccaccgaaccaatttgaactgtactacctctagacaaaagagagggcagtcagacagttgttcgtgatttcttctttcaacagtcatttgagcacttactacaaaacagaagctatgtgtaagggtggaggcgttagctgttaatcaggacctccaggctaagtttctgtattagtccgttttcacgctgctgataaagacatacccgagactggggaatttacaaaagaaagaggtttaattggacttacagttccaagtggctggggaagcctcacaatcatggcagaaggcaaggaggagcaagccacatcttacatggatggcagcagacagacagggagagagagcttgtgcaggggaactcctctttttaaaaccatcagatctcgttagacttattcactatcaagagaacagcacagaaaagacctgcccccatgattcagttacttcccaccagatccctcccacaacatgtgggaattcaagatgagatttgttaccatatcagttaccaacccttccagataaatcacgtgaaatatcgccattaacagagtgagctcaggtggttcttcagtgcatttctgatacctgaaccttccctgggaatttcacagaccatcaggctctccaccctttgatagcaggatagcagggcccaggttctgcaggaggagatgttaccacaggcctgaaagggagggaggggcagatgctacaggaagatgctggctctggattcgctggaggagctttcaagggaagtagatacacactgtctccatcatttcatgtccatcacactctaaaatgctttggacaagaagcaaatgttaaagacaaatgtggcccattttcctgtacaaagagggctgctcccatgccaggctattggcactggtgggcatgaggcttctctgctgccctggccggggggttctctcactcaccattggctctctgacacctggagagaccaccacccttgggctttcatgatgctcacagaatccacactgttggagctttaaggagcctggatcaactggaacaggcagggagtactaggacagcccagcattgccccaaaatatccaggcctgataaaagagaaaaacaggtagctcacaggaaaaggataaaaaaaggaggagggatttaacatgaaaaggtgcttgatctccctcataataaaaagactgctgattccatccaggcaagtgacagaaaaaaaaaatttaatttaaaaagactgctgataaaaccacagcgagacactgctgctcagggatctgagggtgtgggcagccaggctgccacgcatcatgggtcggagaggaagaccacacccctggagcagagggcggctgatctgtcagatgccctttgacagcacctcagcttccaagaattaaccctttctatgtgagcagaggcatccatggggggacacactggtgaatcatctgttatgtagaagtctggaaaacatcaggatggaactggtgaaataagtgtggcctctgacggaatggagcggtccgtctgcactgctgcgggtgcccctcagatcctgtgggtcagtgagaaaagcagtgaggaacaaggcaggtactgtgtactgtcctctgcgtgcaaggaaggccagcgcatgcaacagagtccacacagacatagcctaactctggaaggaagaatgagaatgcagtttcagtggtggcctctggtggggagaaactgggtgaagggagatgtcatttccatttctctactattaattttgtattaccatgcttaaatgttactttttacctttttttttttttttgagacagggtctctctctgttgcccaggcaggagtgcagtggtacaatcatggttcactgcagcctgaacctcccaggctcaagcaatcctcccacctcagcctcctgagtagctgggactataggcacgcataccaccgtgcccagctattttttttaatcaagatggagtttttctatgttgcccaggctggtctcaagctcctggactcaagcaatcctcctgcctcagcctcccaaagggctgagattaaaacgtgagtcaccctgcccagccaattgctttttaaaaaagattaaatgcatgtatacgctcaggcatcagcacacttggaaaggatgaaaatatccggaagaagggttcttttaaaaggctcctcaagtgatgctggcaggcatgacgaatgtccctggtcacaaaagctctgatctggcctaaccctgtcatgttagagactggagtgcgtgtgtgtgcgcgcaaagtgtggggggatgggggtgagtgtgtgtggtgtgtaagcatgagtgtgtatgtgtgtggtgtgggggtgtgtgctgtgtgagcgtgtgtgagtctgtgtgtgtagtgtgtgtgtgaagtatgtggtgtgtatgtgtgacgtgaggtgtgtgtggtgtgtgagttgtgtatggtgtgtgcatgagcatgtgtgtgggcatgtgatgtgtgtgtggtgtgtaagcatgtgtgagtgtgtatgtttgagcatgtgtggtgtgttgtgatatgtgtgtggtgtgtgagcatgtgtgtgtgatgtgtctgtgtgtggtgtgtgtgagcatgtgtgttgtgtgtgtggtgcatgtgtgtggcgtgtgagcgtgtgtgtgcattgtgtctgtgagcatgtgtgagtgtgtgtgtgttcagcatatataaggcatgtaactgaacacagcactttagagggctctcctggagtcagagggggtgggtaggaggagaagggaggtgggctagtgtgctgaagtatctactccttgtcatagtctgtgacaacccagactagcccatgagccaccctgttccctgcatttccaatgagacctcggtggacatgttccctgaggtgaggctgactgatgtcatttgacgatcttgatgccaaatccttttatatcaaaaacaaccagaacactctcttttctcttagtgctttcacccagatgaccacatttcatcctcccagccactctgggccaggtggcactgctggtttgaaagggaggtctcccctggagtaacttccgtgggcggattcacaccctgcccacagtcctgtcccagtcagcccaccatggtggtctccggttcctccagaattcccgcttttcagctcatccccacattcccggagggactgagagcgcagccccagggccctgctctttgggggccgtctctacacccagagaagcagcaaggcattcctaggtttctctttcagatgcagaacttcagtgttcagagatgttcccactggtcctgagagggctcagttcagctttaatgactgcgctgttgcgtgtgctctgcagagggcgggtggcccagcgtggctgactgcagttttcctgacgtggagcccgagcctgccccgctgtttattaattaaggatcactctgcttgcagaaccctgaactccccagaactgtgaggtgggagaaccccgagaggccacctggccccacttcccacctgctgcccaaaccccctctctgccttcctgacagtcaccccaactcccagtgatccccatcaaccatctgacaaggggactgagagggaagagaaaggaggggcccaaagaggaaggtaaaactgtcgggaacagcccccaaatgtgtgacagccttcagtggagttgcccactttcccttttctcctccctgcaggacctcccttctccccagtcctccccaacttctgaggttacattgagaaaagtctgcagagaggtgccagcatcacaaggtgttaaggaccacgagtttggcattttaacagatgccagagccacttgagaaatgtggtaactaagcccagagaggtacagttaacctccccagagtcacacagcaggttcatggcaaagctggactagcacaggtgtccttcccctgcagatccccttctgtgccccacatcacctccctccagtgtctgggccacctggagatgggccctcagactcacccggccagaggtgccatctcatgggagaggtctggccaggaagcatcgatatttgagatcccaagaaatgaagacttggcctgtcagatgacagacttcggtcatgggaacacgtgatctgttttacacatgcgtcccctcagcagcagctttccagaacattcccactttcttctgtagtgagaagaactctttccctgcagcctcctgcccaactcctccttcagtgtctttgcttcagtgtctttgataaaccattctgctttgcagagtgcgagctctgccttgcagggttcgcatctgcctgtgctgagtaaccaacgctaaggtcgagtggtcggtcacctctcataagagctagggttgtctcatgctgatgactaggacttgccctcaaggagaaaaataaatcaaaacaaaagcaaaaacagcaaacatgcatctcttaaagaaggctctgagtccaggtaaatttccttccactgaagcagccaggctgaattcgaattatctttgcccctgcttaaaaactaatgcaaattttcctagagaatatccactaattcctggagggggcatgggcattcctgatgcccatgagaggaccatttgctcttccctcagtatgctaaataacagaagcgacatttgttgctggaaagtatcagtgaagttaataaggtttttcttgcccagggtgagggaacagttcccaatgacaaatgctgtatgggaaggggctgtagaactgccagcccctttggtccatccgtaaagtgaactctgtggatcctggaggattccagcgtctttttttttttttctttttttttaagacagagccttgctgtcacccaggctggagtgcagtggcacgatctcagttcactgcaacctccgcctcccgggttcaagcgattctcatgtctcggcctcccgagcagcaagactacaggtgcgcaccaccatgcccgactaatttttgtattattagtagagacgggggtttcactctgttggccaggctggtctcaaactcctgacctcaggtgatccacccgcctcagcctcccaaagtgctgggattacaggcatgagccaccatgcccagccagcatctttcatttttctgtctgctttggccctttcctctctcactgtcttccttttccatttccaaagtcagtccatctcactattagcacaaaaactgctagagcgcttgtcattggtcatctctccctgcacctggctggtctgttcttggccactgaagcgtttcccccagctgttgctttaatcattttattgttattatgccttacttaagaaatggatatgagatgcatttacctgtctcttcctgccactctgcagagccagtaagatgtggtggaaagggcccaggctttggaggagggctggctggggttggatcttggctgccccctactagctgtgtgaccttgggtaagtagctggacctctctgagcctggttcggaatcatagcacctctctttcagggctgctgtaaggaatagcagtggtgtgtataaagcagagcgcacagccagcaactggcccctagccacactgctgagcacctactgtgataagctgccattgtggtgtgtgaagcaaaggggaaacatgcctgctgtagtgagcttcctgtagggcaggttgtagaaccagaggtgggttccaaggttacaaagggactcttagtgtattagtctgttctcacattactataaagacctacctgagactggatcatttataaagaaaagaggtttaattggctcacattggctgggtgcggtggctcacgcctgtaatcccagcattttgggaggccaaggccggcggatcacttgaggtcaggaatttgagaccagcctggccaacatggtgaaaccctgtctcttctaaaataaaatacaaaaattagctggccatggtggtgtgcgcctggaatcccagctactcaggaggctgaggtggaagaattgcttgagcccgggaggtggaggttgcagtgagccaagatcgccccactgcactctagcctgggcagcagactgagactctgtctcaataaaaaaaaaaaaaaagaaaagaaaaagaattgcaagaaataaattattgtttatgagctatatggtctgtggtaccttgttgtgggactgggagtcttggcgtctccctgaccctgcctgttgctgcagcaccgctcagccctgcctgctccctacctgcctcccctcggcctctcctgcctccaccgggcccctggtgcctcctctagagacagtcctcctgggaccgattgtgttctcacttacacgaggcatccaggactacagataaccagaggaaggggcgccccccccgcctgccctcctccctggcatcctcacgctgcagaggtcagagcctcatcccagccccttacctgcccctactctgtggagaaccgtggtcagttcgccaggccggatccacgaacggccttgtggaagatggtgagctcacacccagagctggctccgatgaccctgtctcctttacatgtttctaccttcccctccctaccttcccccactgctgggcgcagagtggaggcagatgaggtttaaagctcagaagggcttaaacgggttggggcgcagtggctcatgcctgtaatcccggcactttgggaggccaaggcagaggatcacttgagcccaggagttcgagaccaacctgagcaacatagtgagaccgcgtctctacaaaaaataaaataaataaaattagctttgcagggtggcatgcacctgcagtccctgctactcagaaggctgaggtgggaggatcgcttgtgcccaggagtttgaggctgcagtgagctatgctggcaccacagcactccagcctgagtaacagaatgagatcctgtctcaaaacaaacaaacaaacaaacaaaagaaggcttaaagggggctccaggtgggcttggcagcacaaagctatgaagttctatcttagacacaagttctgttactgggcctttgcaggctggcctgggtacctggctgccatagacagggaaccttccagatgagctgcaggcgtggagcacaggagccagggtgctcttcctgggctctgtccacaggcagaacgtacacagtctttgtacacgtccggcggctctggtgcctatttttgtttgtgtttttcttttgtttggggggatggatttggtttcccccgagccctctgtcctcctgtcacctggctggtgctcggcaatgttgaccagctgcctggctggagttggcagtggctaaggctgtgacagctaacatgttcctgagtcctctcatttcttcaccataatgccctgttgagtttgcagatactgtctctgtttttatctcccggggaaactgaggctcagagtggctaggccaccttcccatggtccctcagctcatgagggccacacagggcattgcggtggccttctcctcagccttgaccctccggccccagcattgctgcctcaaggggtctcctctgctgagccgtgcaccttctgcctggcagctccaactctgtggctgtgttcagtggctcagcactgccccttgaccctccctggccttctgcggatgccagactggagcactctgacaaggtctggggtggttgtatgggtcctgtgacctctatacacctcccagtgcctgggaatcctgcagatacaccctccttagccgtccctaaccatagaggacatttctgaggtccccgagagagtggggcacccctgcaggatccaactgctgggcccaggaaggatagcagcagcatgaggggttccattagccacaaactcacggcatggaaccttcacccacctcgcccctcatctgctgtttagcacctggcacgccgtgtatacttactgattattacattttaatggcaaattatagtggcaaacgtatgcatctttgcacaattgttgtacagcatgatgaacaagtcattaatagtaaagaataaatgtgaaagtgagaaaaatctgactgccaaagtttttactccttccttccctccccagacttttaaatgaaagtttagggataatcccttagttgtcctgctagtaggacttgcaattaaaagaattgggccaagaacacttctacgcttctccttttaggtttgggtgtaaattcggggtatttctcactgatgaaagcctggtgcagggcagaccgtgggaagctttcatttccggaatggaccatcaacatcccttggagaagaattctcttctccagacccagacctggtgtcctggcacccattgggcaagtgggtcctagaagacaaacctggtcagagcctggaggctgcttagcattccccacgcacattagcagctcggagagctcaggaagccgcagcccctccttgcctcaccagcctggatcaggacagcatcccctggaagacacacagggcctggcctctgattacccagcctggagggaaagctcaatcgagcatcatgtcacccggtgcccccatgcagggtggcactggtgagacccccaagccaatgataccacctcacaggagtgcaggcccattgtggccagatcatcttgacttttcaagataaatcagaaatcgtatttccatgagatatccctatttgcaagtgatggtgactaaattagaagtttttgaatattgtaacatgttcgtaggctgtttgtctggtttaaactctatctggaggaattcaagctagacttcaggaataacttcttgaggcaaggattttgagaccttagggaaagaaggacgtcttgggggtattctgactgttgtcctcctggaagggaagaacagagaactagaagactgcccttagcgaagttcaaagcacctaagcccgggaccctcagcaagtgttcttgagtcacagattctccctgaggcgcctctttctggctccatagaatggctgattctgtaactcggtgagtttgctttttttttttcctccatcacccaggctggagtgcagtgaagctggagtgccgtggagcgatcactgcaacctctgtctcccaggttcaagcaattctccttcctcagcctcccaagtagctgggattacaagcatgcagcaccacacctggctaatttttgtgtttttaatagagacggcccgaagtgctaggattacaggcatgagccaccgcggccagccataactctgtgactcttgttacaaaggccttatattttgctctttgagggtggttttggtttgatgcctgttggttgccatcttttaactagggatgttttatcaaaatgcccagccaaagtgtccaaacaaattataccttaaagtttgaaaatgtctggcacttctaattcaatgcctgttgtgccaggcactgggctgctgaggaactgagtcccgtccctgcaggctagctagagaacacacacacacacacacacacacacacacacagagtggtcttacaagtcagttttatattctacctatatgcaataaaggtattattatgttgaggtgccttgatataaaaatttttcttaaaggagaggatgcctaaaacaggcattacctgaaacctcctctctccagcattggttgtcttctgtcatgactcagggttttcactgagaatgggatggaaatgtggtctaaagatagggccaatgttgggactggatcccctctgggaagtcagaccaggctagggcaggtccttgaagccatcaggaaaagcctctggagccagaaacaaaacaaaaaaaaaatggtgttaactaaactcagtctcaaatcctgaataggactcaagtcaagcaaaataattaaaggagttagcaaagggcaagtcagagagaccgagcaacaccaatgtcttccgggagccctgtggcgagtgacagagcctggactctggagtagaactcatcttgtgtcttcttctgccactcgttagctgggtgaccttgagccaagccccttaacctcttggaccctatgttcttatctctaagtaggggctggtaatatcttcccctttgaggaatgccctctaaggggtgttgtgaagattcggtaaggtggcaggggtaggactcctggccagaaacaggcacataataaatgctaagtctctccttctctccacctgctggatgctgtagatactaaggatttcgatgtgaatgagacaaaacccctgccttccaggagcctttgagaatcagagaactagacccatttccagaacaaggggatgcagggtctggataaagttttggggatcaatagagcagagggctcccagaggatcccatagggttgactcctaactcaagggcatgagacaacccccaggaagggcaccctggaaggggtccggctgtccctgatttacttgtgggcactgggggaatgcccggagccatccagccctcagggctctgtgtgattctgggttcctcccataaaagataatcagattctttcacgttaatgtctttctccacctcattgcacatcatgcagctattcattgactcagcaagtatcagctttgcatgcgaccttggcctacccactttagcttttagtaatagctcccttcttgaataatacaaccagtggggaaacagaacctaactcttacctctgggaggcttatttgctttgagaacatatgtcctgcagttttgttcatatggcagtgaagtttcgtgcacacactctagagccaggcagcctgggttcaaagcgcagctctgccaggtcctaactgcatgaatttgggcaagtcgctcaacctctccatgcctgagtttcctcatctgtaagattggagcaatggtaatacctgctttttagggttgagaagagaattaaatgaattaagatgggtaaagtgcttagagtggagctttgcaagtagtaagtgctatgtaagtgttcgatttaaaatgaaagacccttaaatacattctttgttcatttcacaagcccttcatttcacaaccttacatttcacaaccaagctctgtctcccctggaatccagccataactctgctcacaagtgtgagacaggccccagcagagctgcacgaagaggagagaaggcagccccccagactcccaaccccctgtccaagatggcaaaaccagaacacagcctctgtaccaccccagcaggtattcagaatctgcaatctccaaagcccacttcaattgtaaatgtagagccacgtgcgctttaagtcacctgtcactctggaggctcttttgctcagttcctcaccattagcagggatgacagggagtgcaggagtgcggtcgactcccagatattggagagcgctgggctagctgcccattctcccggcctccactcctctttgctgtccagccatcacttgctctttgaaggcaaacaaaacagaaaacagtgccaaaagtatgggaagaaagccagcttctcccctggggtgcctgtgatgccatgcccaccctccctgaccacgcagcccctgtggaccctcagggccccaagcccccatttccatcacatgcgtacacccatgtgtgtccatagccgcccatctcagtcaataaggctgctcctgcccacttggaatagtggtgacaaccaggagtggcttatgggaactatcccaatggcctgacagcatgtccgctgcaaaccgctgaggtaggacactgccctcatgtctagctgatcagcaagaggcgcagttgctttcttaggtaacattgctgctgtgtcctggccattgctggggggtggcacttaatctacaccagatttttccctcctgtatcttccaagctgcttggatcttggtgctgaattaggttggactttgtcttgtggggaagggaggactatagaccctcaacgtaagcaatggtcagactattctaagaaaactcgccgaattaaagcatgaggtaaatttagttctgacttctgtccaccccactgccactgtccccttttatcccatgatcccttgcttttcttttcctcctctctccctatctcttgtgtttgacgcatgataggaattcagaaatatatgtttgtggatttgtttattcacgtagcaaaccatttcttgagtgcctaccatgggccaggtagaatgggcggccccgggctgcagtggtttcttcagcccctctccagggtttacactgtgcaagacggtttgtgatgggtcctcccatcgaggaccacactcttctttctctgtgccccttggtcctcagtctctgaccccacttcaaaggcagcattcactcagggaagctcccatacaatgctagtcagagtaaaagtttggacaaattgccaggaagcagcttgtcagtatgcataaacagcctttaaaatattactactctttgacccagaatttcacttctaggaatctgtcctaaggaagtagtcacatgcaaaagatttatgtaccaagatgttcatcaaagtgttgttttataacaggaagtctcagaagctggataaatatccaacctctggaaatggttagatagaatagtatgtagccattagaaaattatgtctatggggtttaaaatgtcatgggaaaacacttctgacataaaagagcatgagaactgtatatttagcataatcttaactatgttttagaatgcacaggaaaaaaatgtacaaacatattcatagtgatgtctctggtggtaggattatgatcagtaagtacttctgtctcttcatattttcctgtatttgataatacatgcatatgttgtttttaaaataagaaaaattttaagtttaaaattggagctgaaaagtgtttttaggtcaggcgaggtggctcacacctgtaatagcaccactttgggaggctgaggcagtcagatcacttgagcccaggagttcgagaccagcctggccaacatggtgaaaccccatctctactaaaaataaaaaaattagccatgtgtggtggcacacatctgtaatcccagctacttgggaggctgaggcatgagaattgcttgaacccaggaggtggaggttgcagtgagccaagatcgtgccactgcactctagtctgggcaacagagtaagactctatgtcaaagaaaaaaaaaaaagaaaagcctttttaaacagtagcagacataactatataatccttactaagctgtcggtcaaatttttatttatatatttattttattcatttattatttttagacagggtctcactctgttgcccaggctggagtacagtggcgtgatcatggctctcttcaaacttgacctcccgggctcaagtgatcctcccatcttagcctcccaagtagatgggaccacaggtgcataccaccacacctggctaattttttttattttttatttttagagatggtgtttactatgttgcccaggctagtctcaaactcctgggctcaagctatcctcccacctcggcctcccgaagtgctggggttaccagcatgagccactgtacccagccctcaaatttttaaaaatctataagagacattattggacaattagagaaattcacatatggacttataatagtatcagagtgtgtggtgtgatggttctggagggaatggactttttctttggagacaggcttttctatgcccacccttttatcttgctaacttatcatcatccaggttccagcagaaacattacttcccccaggaaatttcttaagggtgcagtatcatgatgtctgcagcaaattctcaaatagctcaggaaaaaagtacgtgtgtggtatgagtgtgtgtatgtatgtgtgtatatatatacacatatatacacatatatatacatatatgtgtatatatatacatatatgtgtatatatatacacacacatacacatatatatacacacacacatacatacatgtatttttatataattatatatgcagagagtgcaaatgttgccaagttaaagattggtgagtctaggtgaagggaatatggtatttattgtattatttgtgcaacttttcttaagtttgaaaattttcaaaacaaaaaattggaggaagaaggcatgccagtctaccccaagccctccattggaatgctgaaaatctaaacaatgtgatttggcaatttcatttcttttctgttgtgggccagtagtccttagatgttggggaagggggtagtcgctgaggtgtggttgacttaggatggaagaagcagaagtcaagactcccagggtcaaagtggtttgctctgctgacccaagtgtgggaggcccagagtcagcgtttcaggtgtgctaattcagcatggttctattcacggccaaagtccaccctgggcacctctctggcagcaatcttgggtgactctactaaggccaggcctccatgaccctatgtctggatcccatatctccacctctcccactgtctcaggaacggtgcttagctttttcttttccctctcctgtcttctttgccagcatgtagaaagtttaaataattcccctctttacaacaaaacaaaacatacccccttcagtcaaccaccctagctctcttctccttttcccagccagatttttttaaaagcatcctaggccaggcgcggtgactcacgcctgtaattccagcactttgggaggccaaggtgggtggatcacaaggtcaggagatcgagaccatcctggctaacatggtgaaaccccatctctactaaaaatacaaaaaagtagccgggagtggtggcaggtgcctgtagtcccagctactcgggaggctgaggcaggagaatggcgtgaacctggtaggcggaggttgcagtgagccgagatggcgccactgcactccagcctgggtgacagagtgagactccgtctcaggaaaaaaaaaaaaaaaaaaaaaaaaagcatcctcagcactttggcaactccatctcctcccaacatgtccctgttactggaatccagccaggactcagccccgatctttctactctaaccagttgtctcagttaacaaggacaggtttatgctgcagtgacaaacaagatcccaaattcttgtggcttcacacatctggcaccacctcatcttccagccttaggagtcatcttttagttccttgaaaactctttacagttttctgttggggccttgtcatatactattcccctggaatgttctttcctatcccctccctttcaccttgctaacttgtgcccatccttcaggtctcagcagaaacatcacttccttggggaagttttctccaacacccacactacacaggtgtcccatctacactcctatgactttgtggtacttgtctcacttcattttccactgccttccccacaaggcacctgcacaagggcaaggaccgtaccactgtacctatgtcactcattgctgtggtcacctgcactctggctgcctaccttaactacacattagaatcacctgaggagcttttaaagccacaatgcaagactccaccctaggccaattggatccaaatccctggggtagggccagacatcagtggagttatatatacatatatatattttgtttgtttgtttgtttgttttttgagacagagttttgctctgtcacccaggctggagtgcagtggcgcgatcttggctcactgcaagctccgcctctcgggttcacaccattctcctgcctcagcctcctgagtggctggaactacaagtgctcgccaccacgcccagctaatttttttgtgtttttagtagagatggggtttcaccgtgttagccaggatggtctcgatctcctgacctcatgatctgcctgcctcatcagcctcccagagtgctgggattacaggcatgagccactgcacccggccatcagtggatatatttttaaagcactgcagagaattctgttgcatcagcttgagaaccactgatctgccttgtgcttcacatttaaaacttttttttaatgaataaataaaccccaaaaaattaatctccctaagcctccctagaagataggatggtaaggatattttcctaggtaaaaatatgttaatttcatatttcatgaaatttcatgtttcatttcaatcaagctctgtcatacaccttacatggggcaagcccagtgcctgggcagggtgtaattatactcattacacaggcaaggaaaagtcacattaggtgatggagcacaaataggcagttaatggtttcagggctagttaggatatgtttgtctttcaattgcaagtaatagaagcccaaagaaattggttatttatataatataattgattggttcccaaatttgaaaaattcaggaatagacccagcttaggtacagctggatccagtcactcaaacaatgtcacaaagaaccctttgacaggaatgtatcctgtgttgactctactttgctctgagtagtctttccccaggtgatgataaaaatggtcatcatcgccaggcttgtgtcctgtttagtaggaatatacaagaagagctcagtaaatgctggccccaccactaagcaaaaacaaaacttttgttgttgttattgttgttttaaataacagcttagacctttcttctttccttgttattctctttcatctgtaatccagttttctacttctgaagtatagaatgttctgatgatttattcttcattacccacaacttgcacatgtttatttaaaaatgccaggattgcctggccgttgtgtgctgttaacctttgtttgctgttagtggatccctgaagttcaggctcccaggggagcagataatgggtatccagttcctgcaatatccaccctctggcaagccaagttccttcctgggtaaggttttgcctacctgcattcctagggaagtttctgggcctgaccaccaagccagctctgagaaggggtgcataagccccaccatgctttggctctgtccctatagaatattttatgttgttactgaaaactaaaggaagatgggtgcggtggctcatgcctgtaatcccagcactttgggaggccaagacagattgatcactcgatgccaggagttcaagaccagcctggccaacatggtgaaaccttgtctctacaaaaacaaaacaaaacaaaaattagccgggtatggtggcatgcacctgtggtaccagctactcaagaggctgaggcacaagaatctcttgaacctgggaggtagaggttgcagtgagccgagatcgcactactgcattccagcctgggtgacagagcaagattctgtctccaaaaaaaaaaaaaaaaagaaaaggaaagctaaaggagagagactaaaatgatatcaggttcctggagaacaaacagacatgattttgcttcatggcaggacagccggaagaagtgggattatatcctcacattacaaataagaaaactgagactcagaatggttaagtcacttgtcccaggccacacagccagtaaattacagaaacagaatttgaacccaaatcttccagctccaaagcttgtgttcttttcactacctcctgcttaattttttaatttctaagattagacccttcatctatccatgacacctgcctgtcatcccctgaaaaaaggtgaacgccgttcagaaatttttctagcctgagctcactcccagttcacttatttttgctttgtcatggctgcccagtccccacttgtagaccaggaataggtcatggctgcggggactacacgctgtcgctgctgcaagggccggcctctgtttccggggctgagtgggggccagacctgccaggagcaccatcttctgtgggtcctgcctggatgtcacatcccggccccaagaagtcactgcaaaccttcgtattattgagcttcacatcctagaatttgctgtcactgtggctgctgcatgaagttgtcctgagagaaacgggcattgtcattaacagggaaattgatggtctgggggaaaagtcatcctcattctcttgcagatctatgggtgattgagactggctgatgttgaaggggtttctcagccatcgtgtgccatgttatggaacagtggtgtagccagccatttgacacccagcgctgacctttgtttaacaacctcacctatatatgacaaaatgattgtcagaaataatcgtgtaatgaaatgactgtaataatggccagaaaagaaacgcagatagtaaaatgtttctcttgttgaactctgtacatataattgcaccaggatttttttcaaataaaaagtaaatattatactacaaaaaagggaaaaagcacaagcatttattaaatagctttctatatctttctgagttttgatcctttgattgcagactgatgtaatattttatgtaaatcattgcttggttactaagtgaactttaagaaaagtgagacgtctgcagaagttgcccataatttagcagctactgtattgtaccattgatgtacggctttattttcttgattaattatttaaacaatataattcacaattttaaaataataaatttccacttaaaatggtatttaaactcagcaaaatatatcatctatgagtaaaatttgtatttaccaagcaaaaatattacagtttgtggttcacatgctgtctcactgttttaaattttaaatacaaaaactccaagtaggctgggtgtggtggctcacacctgtaatcccagtactttgggaggctgaggcaggcatatcgcttgagttcaggagttcaagatttgcctgggcaacatagtgagatcctgtctctactgaaaacaattagctgggtgtggtggcacatgcctgcggtcccagctactcaggaggctgagataggaggatcacttgaaccctgggggacagaggttgcagtgaggcaagattgcaccactgcactccagcctgggtgacagattgagaccctgtctcaaaaaaagaaaaaaaaaaaagaaacacaaaaactccaggtggtcgcacagaatgacaggactgaagtaacttagctccaatttctgtcttcataatcactgtcctaccattgtctgtgcttagaatctacttgcttaatgcaggaacatgtgttctcacagagatggaaaatgcaaatggcgccagaagcaagctggaaattctgaaccattaagaatttactctctgccaggcacggtggctcacgcctgtaatcccaggactttgggaggctgaggcaggcagatcatctgaggtcaggagttcaagaccagcctggccaacatggtgaaacttcatctctacaaaaatacaaaaattagccaggcatgatggtgggtgcctgtaatcccagctactcgggaggctgaggcaggagaatcgcttgcacctgagaggtggaggttgcagtgagccgagatctatctgcaccattgcacttcagcctgggagacagagtaagactccatctcaaaaaaaaaaaaaaaaaaaaagaacttactctcaaaataaatacgtgtggctgactccacatatggtagggccaactgtataactagaagttctccaaataacttctgtggagaaaaaaaagtttattaaaggttaacttttttaaagtgctaactagaaccttactaacactgagatcgcaccaattgtttataacttagacagggccgggtgcagtggctcatgcctataatcccaacactttgggaggccgaggcaggtggatcacttgatgtcaggagttcgagaccagcctaaccaacatgatgaaaccccatctctactaaaaatacaaaaattagccaggcacggtggtacacgcctgtaatcccagctactggggagggtgaggcaggagaatctcttgaacccaggaggcggagattgcagtgggccaagatcgcaccattgcactctagccccagcaacaagagtgaaactctgtttcaaacaaacaaacaaaaaaaaaaacctcttggaccaggaaaatattttttaagggaggagtattttatcactggcattgtttaggattgcaggcacatgatgctaatgaaaagcagactaactattagttggttttattactgtttttgaactctctctctcccttttttttttttttgagacagagtctctctctctgtcacccaggctggaatgcagtgactgcagtctcagctcactacatcctctgcctcctcagttcaagtgattctcgtgcctcagcctcccgagtagctgggattacagggcaccacaccaggctaagtttttgtatttttagtagaggcagggtttcaccatgttgcccaggctggtctcaaactcctggcctcaagcgatctgcccatcttgacctcccaaagtgttgggattacaggcgtgagccaccgtgcctagccctgtttttgaactctctagagacagtccagccccttattacttgtcctgaggcagctgctcccttcacctggccccccgcattgtgttccggacccttgtcctggtggtgctaaagaatatctctgtcgatcctttggggactggggaaactgaggcccagtgccacgcgatgccatttgttcagggaagattaggtcatctgctaggtccccagtcacttgaccttcttcccagacaggaagaagctgctctgggtctctcagtgctccacgtgtctttgcacattgaaatgttttctgatttttttttttttttttttgctgttacatttacttttaaaaaataacaagcaataaaatgttacatttgagaaggttgaaatgagaattgatttgagttaaattctagcagatttttcttagaagaatgatatcatcatctccagctacctgcaattgatctactctgaattaagaaagagacttccatttgttgtttatattttgcactcttgatgtgtttctttaaattatggtcatgggccaggtgtaggagctcacacctgtaatcccagcaccttgggactctgaggagggaggatcactggaggccaggagttcaagacctcgtctgtacagtaaattttaaaaattagccaggcatggtagcattcacctgtagtcttagctacttgggaggctgagatgggaggattgcttgagccagaactttgaggctacagtgagttattttcacgccactgccctctagcctggctgacagagcaagacctgcctcaaaaaaataagtaaaaaataaattaaatttcaatcattagcagtcattaggatatttaaatacagtatgttgaatcaaagttacgcatgtgtgtatttttttttccagagagttgtttatcatgtgggttttaatttaactttaaaaaaatgttggctggacagttgcccaaatggtatcatcagccatttggttgagaacgtatgtcctgcgggctcctctgtcactggagttttgctagctgacagccactggctagttagagactgcagtcagcacagatgcaggcgtggacttgcgcacgtaaccatgtcaatgcaaagccatcacttcttaaaaattctgaaccctgctgtctgagatggtggtgcagcggatagaactctgctctaagaggcagtagctaattccatgtcttctttgcccttgactagctgagtgactttgcacatggggcttgcctctctgttgccttgtctgcaaagtggaatcatcttttccttgctagacagaaggtggaccctggacctatggcctttttgagtttcccccccgcttcttagaaggacctctgatcctactgagtttaatacccacgggttaataattgggaaaagcaaaggaagcgcttctgtttaggtaattatatgcatgtttttgtctttttctggctggaaagatatccaagccactgggaaggtccgtggctacccagggtagccctctctggggagggctgctatatccaagagcccctcatgagaatttgaaaatcgaccatggtagggcctgctgacttttgacagctaatggtgtgctgagaattgtccctccaaagatgcctttccattccctcgggagagtctgggcagcccctactgggggctgggatgctggctcttccctcagcctccaccccaactgctctcttccctcctcccctccccagccccctaatttctctcacaaggctttgttctgcagcaacctttcctaatgcagtcctggcctcttcgcagcttcattacataaccttccgtggactcctggtccaaggatcaccccagaaagccagtcagaggtaggcacgcagctggggtccatttacttaccttccccaccccctcggaactcagaggtggtgcaggaatttggactccaagaattaacagctccaccaccatcaccagagccaaaactcaggatgcatgtgcttcatctgctgcttatttccagctgagagccagtggtgccatggttccttagggagccggtcccctgatgccggctcctggccccaaatctctctgatccgggctcttccagaatgtcttgtctccaccatcgcctttgaccaatggtgtccctttgcctggtaatgtcccctttgcctgatgatggccctgtcactcctctctttagcacagaggaggctgtttcatcccttcaagcctgccctcccttcaagtcttagctcaagttcaccttctccgcagagccttctccaatcttcttgactacgtctcctctcagctccagcaacctctgtctctggcactgattccttacttagctaagagaatcacagacacttggggctcaggacaatctgctttctctcttcttacccatggccttggactgtgtgtacctctttgtctccactcccaaacccaacccccagagggcagagagcatgttgtctgtccctttgctcagcatgaagccatgcgtgtggtagatcggcagagttccataacttgtgttgaccgaggggtcactttgctctgaaattacccctgtgtccttcagtatttgcacagatagcttcctggccagaccgaatatatccaagggcatggcccacctctgctcctgtttccaggtccctggtgggggttagttcatgccttcctcataatctgcccactggcctggtcctcaaggtcttcccaactgctcagccagagttgagaaaatgggtcgctccatcctgtttgtgtcgttctctccttcctggcccactctcctgcccacaggtatccaggggctgcctgtagcattagaggacatacatgcacatgcgtgggcatgggacactcacgtagcctccaagcacagcatcaataatgcattctgtgctttatagcatggaaagctgctctaaactttattacacagtggacatgtctgaagcagctcccaaatccacccctgagtgtgttggaattggcaagcctatcacttgggagtctagtttttttgttcgttaataatagatgcttcctgtggccccagcttggcaattttgatttaaagtgatcttaactgaagagactaatggacgggtctgaatttgtgccttttaagcacaaagtattgctcttaattaactggattctatcctttgagcaggcagaggccttcccccaagggcgtcattaacgatccacatctggacatcttccaaagccttcttctgtttcaggccaaccgcaggtgtgttcctgaacacccaggaggctatgagagccacatatgcctcccaaatacacacagtgtgcatgcccagggacatagagcagtgtgcaaagtcccattccatctctctccacctgggagaggatggctcttctgtctgattcatggctcaaagtggtaaaggagctccccactccccgtcccacgcctactcagagtctgcaaatatgtatgcgatatgagagctcgtcagttagctgtcttcagtgtggcgcacatttgaggagtctgactcccctccagcacaggccaatgtgcactgctctcctatctttgtacccccactgttgcactgtgcagaggttggagccatagaagtaccagagctgtgaaaggagaggccccctctcacctctgccctggtctccatccccactttctctaggaagctagtaggtgctgacaggggagagaagggaggggaggggtccagaaacagtggctcatgcctgcaatcctagcactttgggaggctgaggcaggaggatcatttgaggtcaggagtttgagaccagcctgggcaatgtagcaagaccctatctctacaaaaagaaaaaatgtaattagctgggtgtggtggtgggcacctgtagtcctagctacttgggaggatgaggtgggaggattgcttgagcccaagagtttgaggttacagtaagctgtgattgcaccactgcactccagcctgggcaacagagctgagaccctatctcaaaaaaagaaaaaaaaaaagaaaggagagagagagaaagaaaagaaaagaaaaaaaaaaaagaagggaagggaaagcccagaagagtgtggggagaggaggcggccgtcattctggggccctcagtgtgcacaaccagataacacatgctctgtgggcttttgtaccattttgcttgagcataaagaaaggaaggctgcccctaaatagaaagcactctggaggcaaacaaatctgactccaatcctggccctgccactttcccagctgaggacttagacaagcaccctagcctcttggacattctcagagccatctgctgcaagtgggtgctgccatacccaccttactgggcaggcttgggggaccaagggtggtaaatggctcagtctttcatgatgcggccacacagcaggtgcgccatccaggtccatttctttccttcctttcccccaaatcaagttgtcattaaagtactagtccacattaatgaaatcaactgtattaattttctatttgctgctataataaatcatcagaaatttagtggcttaaaccaacacaaatgtattaccttacagttctggaggccagaagccctccataggtgtcactgggctgaaatcaaggttttggcaaggttgcggtcctttctggagggtccaggggagaatccattttcttcctttttccagcttctaaaggtttcatgcattccttggctcatgatcttctatagctatagtcagaaaaattttccatcaatcatcttcaaagccagcaatggcaggatgagtcctcacatcaccttgctctgacaccagttctctgcctccctcttccacatgtcaggaccctcatgattactttgggctcactctgataatctgggatgatctctctattttagagtcagctgactgggaaccttaattccatctacaaccccaattcctctttgccatgtacagtgacatattcacaggttctggggattaggacgagcctgtctctgaaaggctactttacatgaaaattcatttttttaattaagatttttttttcctcttgagacaaggtctcactctatggttcaggctggagtgcagtggtatgatcacagctcactgcagcctcgacgtctctgggctcaggtgatcctcccacctcagcttccctagtagctggaactacaggggtgagcccccatgcccagctaatttttttttttttttttttttgagacagagtctcactcagtcacccaggctggtgtgcagtggtgcaatctcagctcacagcaacctccgcctcctgggttcaagtgattcttgtgcctcagcctcccaaggagctgggactacaggtgtgcaccaccacgcccgactaatttttgtatttttagtaaagatggggtttcaccatgttggccaggctggtctcaaactcctgatctcaagtgatccaccaacctcagcctctcaaagtgctgggattacaggtgtaagccaacatgcccggccccagctaatttttaaatattttttttgtagagatggggttttaccattttgtctaggctggtcttgaactcctgggctcaagcaaacctcccaccttggtctcccaaagtgctgggattacagcatgagccactgcactcggccttaagagaagatttaataattaatactttacaacaagatctggaagaggtgggatgagtaactaaatgaggatacaagtaacccgggtcatatttgctaatacccttggtcacattgaacttgatatcttatcagattttcctaatcagctcctttagcagcagtgttgcagcatcttatctcattttgttttttgtttttttgcctagcacatgcctgtaaatcactggattgaggtgtttagatgtttgttgtcctttggatgcttcttataaatccatatttcatggctccctggaaagtgctatgcaaatgataagctgcaaggatggaaaggaaattgcagtgctcctgaattgtaaatgggcttttacgaggaggtttctaattactcgctctttctcttgaactgaggagttgaagtgtaggtggcagatccataacagataatcatgtgtgtgatgtgacttcagcctgagcgtcgaggaccaagtcacagagcaggaacagccactctccagtgtccttggggctacgtctgaggagaacctgggatttcatatatgacctgcactggctggggggctctcttgacgtaacgtgttccctctgagcatgttacagattctgacattcttatgttccttctgtggagagacatgtacttagtgacctaactcactttagcatatttttgctcatcgtttgtgtagcttaaaggaatcagataattaccccctccccactactttcggaagcacaaatgcaatgccctagaattgtactggggactcaaaaagaaaagagagtagtaaaatctattaaaggggacaaagacagcctatatactacaagctttctatttttatggcagagaatgccattttctaagtaaacagagaactgcatttgacctgcaatatcaaatgcatggatttgatgctttggaaagcaactgttttctgcgttaatctgggtgtcttccgtgaaatgtcctcctgcctttggcttaaacactagctttgtctacagccattccatcctgaacctgcccaatcttgtctgaatcctggtttcaccactgacaagctgtgtgtccttgggcaagttacttcacctgtctgtgcttcagagtcctcatctgtgagttggggaatctggacagaatctaccccatagggcgtagtgaggatgtgttgaattatcccaagtggctacacagagtaagcactcaaatgatgtcatcgttgtcatgattgctgttaccagagcctagagttcattctgatactcgagtctgtggcccatccagcccaggtaaggaatagttggaggagttgggcatgttcagcttgaagaggagacgacaggggatatgggatagttgaatctgtgaagggccccctgggatgaagaactggcatgttctgtgtggctccagggcactgagcaggacccatttgccaaagtctcagggacacagtttctagctatagacagaaaaattttctgtcactcagaggatgaaaatagaatgagcccccttaagaggtaatgagctccctgtcattggaaggattccagaagagctaggtaaccactttaggtgctatcaaggggcttttttctttaaagtcctttccaaaagcttctgagattgcataaacaataggaagccatcttggtgctttaacacaaactctccccagtgatgagggttgagccaaagccagattggcaagcagagaggagacttgtgtacaaggagttcctcgagtcaattgctttttccttgttctagccagccagagggctcctgttggaaaacaggagaccggagaggctgaggcctgaccaaaccagcttctgcaggccagctgggaggccacaactcctacctacgggaaaactgaagggcatctctatttttagattagcaaaagaaaataaatttaagtttgagtctcctttgcaacttttaaaagacatctttattgagatgatcattcacattctataaaattcccccactttgagttacaattcagtggttttagtcttccttgatgattttgatggtcttttcttaaggctcttggaagacccagaagcctctcagacacaggtgggtgtggagggcgtagcacagaggcagacttctcatttcctgggtctcccctttaatgactctcagagacccctccttccccctgcccctggcttctaccccaggggtgtagagttttgccattttccaagcagaacttcatttcctcttctgtgtctacactctttgtgcttctttcttgccagctttttctcctttgcccgcccttccttccttccttccctccctccctccttccctccttccctctttccctccttccccccttccacccttccccccttccccccttccctccttccttccttccctccttccttccttccttcctgccttccttccttcctgccttccttccttcctgccttccttccttccttccttccttccttccttccttcctggtatgtgactaatttctgtttcaggacataaatgttgtccaggctgttctttggtctttctgttggataatggacatttggcattgagagaggctgctttttctgaaatcatgttcttggggcccagaacctaggtgtgtgcttctgactttgttttcttcctgatccaaattctgatatgtccatttaaattgatctagacccacagggcactgtgggacagatcctcagtggaacatgactctgtaacgagagcattttgttttgtcaaaatgagaacatattattgcctttcatctgattgtaaacataatacatgtttataaaacagtataatgagacaaaaatgtagacactaataagggaaaatctccctaattgtatttctcttcacagagaaagcccctgttgggcatatatactctagtttgtttatttgtttgactacacatatatgtattcttttcttatgtataaaaattctgaacatgcacatttctgcaactactgttttcacttgatgatgcatggacctctctagagtgtacgtttcttcttccttacaaagcagttggcttcgcccagggtacaccaggacacggttttggctctgtccccagggtgtcacgggaccaggggatgatctcacagggtctgccatctgccctgcctggccggaggctgcatcgagagggccaaggggcaccacgtgtcgtgggtactgtcaaacaagagccttcagagccttccacagtctttcttttgcttcccagcattgcttccccgctggtggactctgaatctagaactagctccaggcgcctctccaaattcagacgggagctggggcactattataatgcaaatctaggcaaagccctcccaataccaggatccagaatggggtggggccctttgccctgaaaagctgtttagtttgaaaatacaaacaggagacagaaaagtttggctaaattaatggataaagttttaacgatggtaaccatagtagggttcatcgacagccagcgatggttctgaacacttgacatgtattaactcacctaatccccacattttacagacaatgcaaaggaggctctgggaggttgagtgacttgccccaaagtcgcacagctcctaagtgaaggattcggagtggactccaggcagcctggtctgactccctgcactgcgctgtgcttatctctggccccaatgccgccatgcagaagtgtctgggggcactttgtctctgtcagacagaattcggagatgtgtatgcttgccctggtatggcacttctctttttttgagacagaatctcactctgtcaccctggctggagtgcagtggcatgatctcagctcactgcaacctccgcctcccaggttcaagcaattcttgtgcctcagcctcccaagtagctgggattatagatgtgcaccatcgtgcctagctaaatttttgtacttttagtaaagatgttgttttgctgtgttggccaagctgatctcgaacttttggcctcaagtgatctgcctacctcagcctcccaaagtgctgggattacaggcatgagccaccatgcctggcagtgtggcacttcttacgtgtgttcagcggacactgtttatcttctgtccctccaagacggtgctgagctcaggtcgttcattactggcagacaactgctgatttccaacagaattgccatcctcttctcccctgcgactttcagagtgtgacctcagactcaaaaattagaagtgaaaacatcttaaaaactatcaccttttcttcctaatcctcctctcccctccctgtcttccttgttgtccccatctaatgaactatcatggcaaaaagagcccatttctggtcattttctgtggcctttcaaactcccacctaccccactgctcctgggtgcattacccgaaagctgagacttcagtgcagaaagtgccaggccctctgtccccccagatcgccttccttgtcttccctgtgcttgcctgtcacattgtgtgggttccagcgctggaaggaatgaggaacagattctctggttctccttttgaagtttaccttcgctccaccacttctgagaccttcccggaagttgccccttgtttctctcctctccagggctgccccagagctgcctctcacctcttcctgctgtcaccccaccaccatcagggcagaagttgggacaaagcctctcctactggctcctgcttttctcccttaggtccagcctcctcttctccatcttcaggagtctccttctccactcacacgtcatgacttcagcacctcgcatcagtccagaatatgactgcttgttcaagtgccacctttctcatgcatttttttctagtgacaatcacagccaccctgtggggcaggagtgtcatcatccccatgtttcaaatgaagaattgcagttcagagagggcaagtgactggcccagcctcaacagctagccagtggaccccaccagggcttctgactccagtccgggttccctttccacccaaatccatggagggagctgagccgagaacaggtgtccttcaggaagacgtgaagccaaagcctccacctccaaactcaggggcccagggagtccaggcacccatccactcacaaggctggatatggtgcattccaggagaggggttgggggcgagtggcctctctgtgtacccgtggggatagatgcgcaagtggcatcgccacatcgtgagtcctggcttcatgggtgagctccaggtccaacgagaagccaagcagggggcccttcaagctcagctttgggcccgggtcggggtacagggtagagcgggcctccccagcccctgccatgaggccaaggcagtgcatcgttcgcagcgtacattcagaaaccaaagcctaggagctggttatcattccggtttacagctgatggaagagcaggtgcttccgagaacccacagtgctctttggccagtgacccaagggtgcctctgagaggcctcgcagcacccggaggtgctgctgaggcaacgccctgactgtaagaaggaccattcatcctcagagagtggccgtgatgctgctgcgacagtcccaccatccctcccgactctcactcccaacagacttcccactgtaaagctgaactctccagcaaatcacctctcgccagactctctcctcactctctctgggtccactagaggttcctcagcctctctttgccttggttttcccagctgtaaaatggagcaaagagggcctatgtacccacaaaggtgtggttggagcgactcctcctacattagggcctcgagtggggcttcatgattggttggtggaggtctccaaacccacccagtgccaccgaaggctgagactgcagatgcaatgccacaggtgtccttcctcagcctgggcagctgaacatcatgtgtaaaacggggataataagataataacagccccttgcacctatgtggctgtgaggattaaacaagataaatgtgtaacagtgcctggctatagaaatatttactcttgttattaagggaagaatatgtgtggctaaaaagggatcgaagatgtaaaagccaatccctccccctctagcatatttaagggtaatgttgagttggtttgtggaccatttgctgcctgttagagctggaaggtagggaccccctctcaacagcgatgctacaaattatacccattggaggtcaaccaaaagacaaagcttattggctggacatggtggctcacacctgtaatcctagcactttgggaggccaaggcaggcggatcacttgagatcaggagttcgagaccagcctggccaacatggtgaaaccccatccctactaaaaatacaaaaattagctgggcgtggtggtgcacacctgtaatcccagctactcaggaggctgaggcaggagaatcactagaacccaggaggtgaaggttgcagtgagccgagatcgcaccactgtactcaaaccgaggcaacagagggagacgcaatctcaaaaaaaagaaaaaaagacaaagcttgttaataccagcatattgttaagggaataaagtaggctgcagaacaactggtgtaatatggtgccatgtagggaaaattacatgtgtgcataggagaggggtctgcaaggttgtgccctaagatgttagagtggttcctttgcttttctcttttataattttgtatttgacttttaaataaggaccataaatcacttttataaaatacattctctccagcccctactactcctttaaagaataagagtggtttgcccaagaaagacagttttttttgctctggttttcttgattctgacatcagaggaaactccttctcatccacttggggctctgggttcaggggattcatttcaggcagattaaagtggtgaccaggggcattcgtggacacagggagggacaggagcaccatcagtttgtctcacacaaccactgtcatcctcactgaaggctgttgcctgatcaaaaacagtattgggccaggcacggtggctcacacctgtaataccaccactttgggaggctgaggtgagtggatcacttgaggtcaggagttcgagatcaacctggccaacatggtgaaaccttgtctctactaaaagttcaaaaattagccaggcgtggtgggtgcctgtagtcccagctacttgggaggctgaggcaggagaattgcttgaacccgagaggtagaggttgcagtgagccgagatggcaccaccacactccagcctgggcgaccgagggggactctgtcttaaaaaaaaaaaaaaaaaaaaaaaatatatatatatatatatgtcaaaaatggggtagtttttagatctatagtagttctaaaaacaaaggccatccaagcatgacagatttacaagcactattggctattccagtagttacaatggaggagagaagcttttagttaaaacaaacaaacaacacaacaaacccagaaaccttaggtcaaaaccaaaattgtcctctcagacacaatctgggaattttctcatgacagtgggcattagccaactgacatcagcagcaaccatccgtgtgcacacagtggcaccacctcctcccaaaaagcagccttcatctatgccctcatacaatcgttgattattctctttggattgaggcccggaattatttaagtttcttcttgccagcatgagtctttcctttctgtatgctccttatcttctctctttaatttggcagttctgcttgaaatctgggtctttcattagtagtagttcaatttggttccagaacattctgtggtgtgatgcaatgtgaccagagctcacacttcagagctcttcaagggccagtcttactgagcacctcccagtggctgcctgtgtgctgggcgccacttgtggtgggcaggagagaggaggggacacaaaaggagacacagctccttcttagaagctcaaagttggggaccagctgccacagaagagtatgtttagcatctgagacaccaagatccagcgtcacaagggtgtttattaagcctcctcatctctttctttttctttttttttttttttttcctcaggcagtcttactctgtcacccaggctggagtgcagtggcatgatctcggctcactgcatgcaaccaccacctcccgggtttaagcaattctcctgcctcagcctccccagtagctgggattacaggtgcccaccaccacacccagctaatttttgtgtttttagtagagacagggtttcaccatgttggtcaggctggtctcgaactcctgacctcagatgattcacccacctcggcctcccagtgtgctgggattacaggtgtgagccaccgcgcctggccttgctgttgattcatctatagtatgtttgacttgatgacctccagttaccttagacagaggttctcatctaagctccaactttccatttcctttgtcctcgtctttccccttaacccctccacatttctctcaaaatcaccccacttctaaaaaatactgtttatttttcttttaaatttcaaattatctatactcattgaaataaatcaaaatagcatggaataagcgaaaaaaatggatcccacccttccccactcccattccctagggctaaccatagttaaccatttaatgactaggtttttttgttgttgttattttttatttatttattttgagacagagtcttactctgtcacccaggctggagtgcagtggtgtgatctcggctcactgcaacctctgcctcccaggttcaagcattctcctgcctctgcctcctgagtagctgggattacaggtgcctgccaccacacctggctaatttttgtacttttggtagagacagggtttctcaatgttagccaggctggtctcgaactcctggcctcaagtgatctgcccaccttggccttccaaaatactgggattaaggtatgagccaccgcacccagccctcctgggctcttttcctttagttgcactcgctccccgctcctggagtagagggatttccgagagactgtgggctccagccttcacctaggcccaggactaggatgcctgccctaacatttatctttataccttaaagcaaaacagctggaccataagcattcaagaacaaactgtgaataaggagaaagttctcccaggaaacaagagctttagttatgttgggccagcccttatattccttagctgttaccagtcactgcttgatttaatctcggctatcacttggcctgacaggtctgctgctggtgccaggatgtctgggttttgaagcctggctccattacatacttcctgtgtgaccttgggcaacttactcaacctgtctgttcctcagtttccccagctgtattatgtcagcataatagtttgttgtgtgaattaaatgaggtaataactggaaatgcttcaaacatggttcctatcatgagaaatcctgctttccgcctaaatgtgctggaaaattcctggtggtgcagaacaggagaccagagcaaaggaaagacagggtgcagaagccaaaaattaccttggagaacaaagcgcatgttaaggttatttttggattctaggtttatctctgcttggtcttcagttacctacaagagatccatttaggggatttttgtttgtttttaacgatagctttattgagatataattcatatgccataaaagtcactcttttaaaatgtttccggtatattcacaaggctgtgcagccttccctgtccttgattccagtctgagtttttaactgaagggataaggaggaccacgctttccccagaccagaaccgcgggccagggggcgattccgctgagtcaccgcgggcgcctggtgcgcggcggcggagcccgggaccttccttggctgccccctagcgagggccgcagcgcagcctgagacacccgccggggccgctccacggccgtcggatttagactggaagctcggtccaggtccccagcttgatgcgcccgcggtgtaggagaccagcccgactcgagcttcccctgagcccctggactcttgactccagcagggcctgggtaatgaacgtcagctcccctttcccaaaggggttgctctgttgggaaggcacccgtttgatacagtagcatagagatgggttttagcatcaaaatatcagaattcaagccttgctctctgcttactagctgtgtgaccctaaaaaggtttctgaacgtctctgagcttcagtttcctcatcattccttctcacggggtggttgtgagcattacagagatcctctctgtgaagcccctgtgagtggctcatcctgagggctgaaataaacatgttattaataatccaaaactggcaagggatgttgactggtccccctcccttgcccaaggagctttctagaacctgagttatcattaccaaactgtactgccttgagtaagaaagttagaaggaatgggaaggatggtggcaggtggaggaaggcggattggtcatcacctccttgcagcaagaaacagccccagatcgtgggaaacctacagacctgctagacagactaggagcaaaagctggggctttaagaatccccagggaggttctcctgagagagtagccagttggattttgtaagcagagatttgtttggggaggaggtgacaacgtagggagcagaggggcaaagctgtcgggaatcctgccttgagggcagggatgtgtgttggggggagttgggtcactggggctcggtggccttgggcaagtttctacctctcaggtcctttacccacctagggtcgccatcctgcccacctcacaggttacagtgagcctggatgcactgtcatgggcaggtgcccaggaaaatggcagacatgttccaaacagcacgcagcattccccagtgatgcccagggtcaccttggaggtgggcgagatgcctggggtttctcgtccaccccacaacacctcaggggacagccaaagctgtcccttcaggtaagctgcacagaagatgtgaactctgctgcaaagactctattctttgggagcaaaagggacccagggtctcacctgcacatccctgtccctgagggcctaggggttcttggaggccccagccttggcaaaatgaggaagaaggtgaaggttgtctgggcccctgccaggctccttcctcggccacgcactccccttcctgcacacacacccttctccctccaccccatctccattgttgtcagaaaagtcacaataaaaaggtccatattgtctagttcccatacttttaatttttaaaattttatttatttatttatttatgtattttttgagacagagtcttaacccaggctggagttcagtggcatgatctaggctcactgcaacctctccctcctgggttcaagtgattctcatgcctcagcctcccgagtagctgagattacagatatgtgccactatgcccagctaatttttgtatttttagtagagacggggtttcaccatgttggccaggctggtctcgaactcctggcctcaagtgatctgcctgcctgagcctccggaagtgctgggatttcaggtgtgagccaccgcactcggctccacacttttcacttattaaaagactgtggtgtccatcaatggatgaatgaataaaccaatgtggactatccctcccattacccaaggaatgaagcacggagccgtgccaagatctggattcacagtgaaagaagccagtcaccaaaagccacgtgctgtgtgacttcccttatacgaaatatccagaagagatacatccatggtgacagaaagtagatgagcagctggggactggcgaaggggagaagggggagcagctgtctatgaggtccagcctttcttctgggtttggtgagaatgttttggaactagatagaggtgatagttgtacaacattgtgaatgtactaaatgccactgaatcattcattttaaatcgttctttacgttgcatgaattttaagtcaatcaaaaacagttgtttgaaaagagaaaagcctatgggtagcggcagcagtgattggatttatgattcgattccatggctcatccctcccctgcctcaccccctcgccctccgacgtcttcttcttttactctgaactgttatctttgttctcatctctctctctctctctcaaccctgcagacacttttccctttctttgtctgcccccaccctccagatttccgtgtctccagtgtctccctacgaggcatgaattgagactgggagggtgtgattctgaagaaggcaccaacagtgactcagctagccccttcccccaccccgccccccgggcctcaatttagctaaaaaaccacagggacggactcaggaggcaatacctttccaagggtccctaaaaaatgtcccattttagtgtccaggtttcactcaactttagtgcctcccctaaaatgtgttccttacctcccaccccactgcatctaagtcactgcctgagaaaacaggattgaggaaaggagaaaggaagagagagagagaggaggagagagagagagagggaggaaggctgatggatttagaaaagaagaaaacaagtggtctgaggaaaacagccttggtgtgtttattttcctgtctgtgtatcgcttctcggccttttggctaagatcaagtgtattttcctgtctgtgtgtctcgcttagattacagggatctgtgggtgatgacacgtctggtccaggctgcgtagtcacctcaagggcatgcttattgatgtgtttttcaattcactatctttgcatgggagtcccaggccaagaggcacagctgcgccatttgtctgttggtttagatatcctttatccagttcttccagagaaatcatcctgcccttctggaggaggtgggcagcaggggtcagagatgggagggaaaggaaggagccaggtccttggctaggatgccagggtcccctgcctctcacctggcctgggctggaggcctcctgctgtcctgtcactgatcactaccccgccccagcctcctgagttagaagacacaggctaaagtagagtatttcttcattgaaaaacccatacaaaataaaggttcataaaaaataaaaatttagactgggtgctgtggctcacacctgtgatcccagcactttgggaggccaaggcaggtggatcgcttgagccctggggttcatgaccagcctgggcaacatagtgaaaccccatctctacaaaaaatacaaaaaattagccaggcatggtggtgcatacctgtggtcccagcttctcagcctatggacccacatagaatacaatgtcagcataagaagggagccctggggtcaccaaatggtttgggcggcaaagaacctgaaggttgagagaagtggcttggttacccagctgttggatgtgagacctggccactgcttcttccataccctagacctgcaccctgacatctcaagtaaaaagttgggggatgttttatggtccaggatgaaggaagggcagtgaggggcagcggagcatcactttgcatttctgtctgcctcttactggctgtgtgacctggggcaggtaacttcccagactcctgggaatcataacacctatgatgatgatgatgatgatgatgatgatgatgatgacacctacctcaaggattgccctgaagggtcacagagatgcctgcaaggcacctgcatggagcaagcgccccttctctggcaggtgctgggtgagcactacctgctgccaggccctggggctatggcactgcgtgaccctgcaagtcctacctggcgaagctgtcgttcttgtgctcagtcagtgttggttgtaagactgagaagagtcacttcattttgctctccagggacatctttctgggtcctattttctgcctatgtcaagtagcgcctcaaggatgctcctgaaaatgggcttgtctttcttaacatggcaggtaggtcccaaagcattagcatggggcagctgacctagcccagccaatgcagtgcagtgactcttgcaaccgagtctaatcagaaggtccatgaacctacgagcatttcctgtcccaggatcagggtggaggctgagcctccctgcttagagattcttcccatgcattccacttttttccccaaaagaaaatattgacccttgagaggcacacagtttatttattttgcatagtaaatagtagcctgtattttaaggatgagttgatttctgcatcagcccctgtaggtcatcagccttctattggtgcatctgactctctctagccctgcagggatggtggagggggaggggaaggagggatctttattggaaaccaggacagtgagactcattgccctgtcatctgctctgtggtgctgaatgaggcagcccaacagagaaataccctgagcgagcatccccagcctccaaaacagtggcgcattgccctgagtcctgggaatgacctttgattctcctgctcctgacttggaacccatggaaacctctagaagcagctgaggaaaacccaacatgaaaagcagaactccacactgagaatataggaggtgatcggaacatacaatgattcttgctaagaccgattcacagtttttcttttttttcgatcgaagaaatactggagaagcctaaagaaggagtctaaaaactctggcacgtgggccaaaactgtccttgagctaagaatgattttcacatttttaagtggttgaaaaatgaaataaaataagatgatgttttgtgacacatgaaagctatgggaaattcaaattctaatatctataaatagtgttttatcagaacacagtcatgctcatttatttatgctcgatggctgctttcccgctacaattacgttgagcagttacaacagagaccacgtggcccacaaagccttacaatatttactatctggccctttccagaaaaaaatgtgccgactcttgaccttaacctcagcaatttgggaggccgaggcaggcggatcgcttgagctctggagttcatgaccagcctgggcaacatagtaagactccatctctacaaaaaatacaaaacattagccaggcatggtggtgcacacctgtggtcctagccactcgggagactgaggtgggaggatcgcctgagcccaggaagtcgaggctgcagtgagctgtgatggcaccactgcacctcagcctgggcgacagagcaagaccttgtctccaaataaataaataatgcaaagtaaaataaataaaaccatataaaaaggaatcaatttaaaattataatgaaagctggccgggcatggtggctcacgcctgtaatcccagcactttgggaggctgaggtgggtggatcacgaggccaggagatcgagaccatcttggctaacacggtgaaaccccgtctctactaaaaatacaaaaaaaaaattagccgggcacagtggcgggcgcctgtagtcccagctactcgggaggctgaggcaggagaatgtcttgaacccgggaggtggagcttgcagtgagccgagatcgtgccacttgcagtccagcctgggcgaaagagcgagactccgtctcaaaaacaaaaacaaaaacaaaaacaaaaaaaaattataatgaaagccaaggggcatagtagaacaaattttctagagctcattaagtcaaatgagtcaccagttagtaaaacgcagtcacggggaagagagggcaggattctttgaagcagcggctctcctaaaaacaacccacccttgtccagctgccttccctcctgagggtgttccctttgactgtgtgacccccatcccctatttcccaaccgtccaagcccacctctagcataatacgagcttttaatccctctccctgaccccaacccgattttgaagcccagtctagtattttctcaaatacacttcttggctccattccttcctttccatcacctctgccttttcactgcatgcttggaccactgcagtcagctccctatgaacagttgctctctacccatccaatcggccccgcctgctgctgccaaattcaccgagggcacctctgtggtgctgcctgtggacaaagtccaagccagccacctcacccacctacaggtgagtggggagcagccagcgtgtccagtggtttaccccatcgccacagacttggtgatgtgtcgatgtgcagagaaggggtgttggcagccacaacacaagcaaccccgccccatgtgagatctaagatgggcgtgctgggagccacctctgagaatccaacagaaggcagaggggagaacggctcacacggcacaaacactccttccttttttttttttctttttcctttttgaaaggagtctcactctattgcccaggcaggagtgcagtggtgcaatctcagctcactgcaacctccgcctcctaggttcaagcgattctccagcctcagcttcccaagtagctgggattacaggtacactccaccatgcccggctaatttttgtgtttttagtagagacggggtttccctatgttggccaggctggtcttgagctcctgacctcaggtgatctgcctgccttggcctcccaaagtgctgggattacaggtgtgagccatggggcctagcctccttccatttaaatgtatgcctaatttgcccattgagaacggctgagacgcattttaagtggccagggtctacttagagttagtgctcatgaccaggcccaggtcaagcctggctggccagatggtgcctttgacctgctctgtctctgtgcaaaggaatgagctgaaggatgggggtgcagtgtgtgggcagtgggctggggctggcaggactcagtgactaagggaagagaactttcctcactaccagcctgtcttttcagggcaccgcggggggctttgggacttggtgatgaacacagcacagagagctgtccagcatgcgggtccctggcttctcacacttcccaggctccttcagaggctctctccaaagggagctgctctctctagaacccatgaatttggaatataggcaaccactgcattggggaccactgacctcaaacatagagaccagagcaaatggggctcatcacgtgaaactcatctggaactctagcaggttcttttatatatatatatatatatatatattttttattattatactttaagttctagggtacatgtgcacaacatgcaggtttgttacatatgtatacatgtgccatgttggtgtgctgcacccattaattcatcatttacattaggtatatctcctaatgctatccctccccactccccccaccccacaacaggccccagtgtgtgatgttccccttcctgtgtccaagtgttctcattgttcaattcccacctacgagtgagaacatgctgtgtttggtttttttgtccttgcgatagtttgctgagaatgatggtttccagcttcatccatgtccctacaaaggacatgaactcatcattttttatggctgcatagtattccatggtgtatatgtgccacattttcttaatccagtctatcattgttggacatttgggttggttccaagtctttgctattgtgaatagtgccgcaataaacatacgtgtgcatgtgtctttataacagcatgatttatattcctttggttatatacccagtaatgagatggctgggtcaaatggtatttctagttctagatccctgaggaatcgccacactgtcttccacaatggttgaactagtttacagtcctaccaacagtgtaaaagtgttcctatttctccacatcctctccagcagctgttgtttcctgactttttaatgatcgccattctaactggtgtgagatgttatctcatggtggttttgatttgcatttctctgatggccagtgatgatgagcattttttcacatgtctgttggcgaactctagcagcttcttttcacaagttcatggagagaggtttcccactgagggaatcacatctgtctgatcaaaagaggcttgggaaatggctctcctgttcattccctgaaaacctctgatggaaccactgccactgtggcagccccagcactggcaccccagccatgattggtgccccagccacatctctgctgtgagccccagagccctggttaattaatcatccacgtgttgatggggagaggcccattcacaaaagcgacataaagcccagggagacgtggccgtggcaagaagggtgtgggactacattccgcccccaactgagagattcagaaaccagaaaaaaatggaaaaacatactgtgctcttgggtgggaaaactaaatatcatgaagggagcaatttttatagttttggcctataatacaattccagccgaaatcccagtggaactttgagaatttgcaggaaaaaaaaaaatgtctaaagtacatctggaagacaaacttacaagaaggtcaaataattttgaaaaagaaaatgatatctaagcccacctagagaataagacttgagatccaaagctaaatcaggaggctctagcaaaattgacagataagcaggacagagtgcatggtgcattcacctggggaagagggcagattggtctacaaataggcctgggtccactgactttagctgttatatttggggagaaacttttcaacctcactccatcttaaacctaaaaatattccagatgaattaataaatataaaaaattagaccactaaaaatgtagaagaaaatggatgatctttctataccatagagcaatggaataaatcacaaaggaaaacagatttgactatataaaacttaaaccctgcccatcaaaaaccatcagaaaccaaaataaaaggcaaccaactggagaagatagttgccacaaatatgatcaagggttaatgttattcataaattaagagcccacacaagtcattagaataagcactgagacctgaacagacaagcaaaaagaatgagagtgggtcggcgcggcggctcatgcctgtaatcccagcactttggaaggctgaagcaggcggatcacttgatcccaggagttccaacaccagcctgagcaacatggtgaaaccctgcctctacaaaagtcataaatattagccgggtgtgatggcacacgcctgtagtcccagctactcaggaggctgaggtgggtggatcacttgagcccgggaggtagagtctgcagtgagccaagatcacaccgctgcactccagctggagcaacagagtgagaccctgacttaaaagaaaaaaaaaaaaaaagaggagaaaaatgctgatctcactagtaattaaaacatcaggccaggcgcagtggctcacacctttaatcccagcactctgggaggctgaggcaggcagatcacttgagatcaggagttctagaccagcttggccaacatggtgaaatcccgtctctacaaaaaatacaaaaattcgccaagcgtggtggcacatgcctgtgatcccagctactcgggaggctgagacaggagaattgcttgaacacgggaggcagaggttgcagtaagctgagatcgtaccattccagtccagcctgggctacagagcgagactctgtcccagaaaaaattaaaacatcacatatttaaacaactctaggatatcatttaaaaaaacattaatagactgttttttagagcacttttaggttcacagtgaaactgagtggaaggtacagagacttcccgtatgttccctgccctccacgtacagcctcccccactgccaacgtcctgcaccagagtggtacacttgttacaaccaatgaatcctcattaacatatcattatcacccaagttcatagtttacattagtaaaacatcatctttcatctataagcacaaaaattttttggcatttatttaggtgtatgattaactcagtgttgacaagactcacacttcatacccacttgcactgcatctgagaagcaattggtgtctacagccgctacaccctcaacaagcccgatcttgtttgaaaagcaattggtgatgcttctcaaaattctatggacaaagtcagccgggcatggtggctcatgcctgtaatccctaaactttgggaggccgaggcaggcagatcacctgaggtctggtgaaaccctgtctctactaaaaatgcaaaaattacccaggcatggtggctggggcctgtaatcccagctactcgggaggctgaggcaggagaatcgcttgaagcaaggaggcggaggtttcagtgagccaagattgcaccactgcactccagcctgggtgacaagagtgaaactccatctaaaaaaaaaaaattatggacaaagtttttcaaaaagatatttaatgcaactttatttgtaatattggaacatctgaggccatttcagtgctaactattaggggatggttaggaaaatatggtacatatgtggaaaggaacatttggtagttagtgcccctgatgtttacaaaggcttttagtgaccaacaaatgctcatgctataatcttatgtgaaaaaagcaagtagcataattgcaactatatttttaatgcatagaataaaaggctagaaggaaatatcacagatccttgacatacattcccaaacctttgtaaatccgcggattcatgaaaacagacacatttgcacaagtgcctgatcttttctgttatacattcattagaagtcaagccctggtgccacaaagtatctgccttttcaaatgtgatcagaatgttctcttttgcttcaaggccatttttcacgaagcagtggcatttttgcctcttcatcagagtcaccgtgtgccctggaggactgagaacagcagagccgttttaggatgggacagggcagccaggaggattgggctcactccctactgagtgcctcactcccgtacagcccccatagaggaagaggggttcaaatttattcctcagccagatggcatgtgccgcctgtcctggaatttcacatcacttatgatggaccaaaattccaaaagctgaatccatgattgtcaaagtctggtatggcaggatgtcaacagtaatcgtttctgggcagagggatgattttctcttcccatcttgctttgtataaatacattttctataataaggttgtattacttttctcatcaagaaatagcaaagtactgttttactcaaaatatgaatagagccaggcatggtggcagcttatgcctgtaatcccaacactttgagaggcggatatgggaggatcactttagcccaggagtttgagaccagcctgggcaacatagtgagacccccgtccccactcccccaaagaaaacccacaaagcatttatcctggattattcacaggggccaaaaaaaaaaaaaaattcaggcctcctatagccatgagctacgaatatgaaaatatgcaaatgtgtaagaaaagccagcacatccgatttttacttttactttcacacctctgtccaccatgttccaagagaagaaacttggtcattgaaaggaatagatcaaatccaaagaacaaaaccactgtgctcattaaacttcttagtgttcacaaagctttagctgcaggttgaatggggcaacccgaattggctggctcacctgggctgcagggagcagagatcgcgacactgcactccagcctgggcaacaaagcgagactctatctcaaaaaaaaaaaagttcataaattcaaagttatgaattatttttaaaataataataatttacaataaagatgaggacaaagtgtgagtaaatggtggtttctatccagctctgttgagctgaagtggcatctccctgctggggcttttggggaagaagggtgtgtgttgctcttcagatcccaagcctcatgcccctactgggccctgtggggtgcttctcagcccaccaggagagccaccgttggaacacacacgtgggggacctggtgggtgccggtgtggtgaatgggggccacagcctgactccaggaagccagcaaactcggagctggaggagtcaggacacccccgatgagtcaagagttggttttgctgccagttgacatctgattgaaccatctcttcacttctccgtgcctcactttccttaccagacaggctctgctgatgctgtccctctcctgttcagtcgtgccctcaccgttaaagagaaagagcaaactgctgggcagcagcattgatttttttaatgaagtggaaagagagctgggaataacaagtcgggcccacctcacctgcctcacctggtgggtttatttgttttgtttttttttttttgttttgagacagagtttcaccctgtcacccaggctggagtgcagtggtgtaatctcagctcactgcaacctccacctgccaggttcaattgattctcctgcctcagcctccccagtagctgggattacaggcacctgccacatgcctggctaattattgtatttttagtagagatggggttttaccatgttggccaggctggtctcgatctcctgacctcaggtgatccacccacctcggcctcccaaagtgctgagatcacaggcgtgagccaccatgcctggccgtcacctggtggtgttgaatatgaactgctgcggtgttggtaaattaagcaagcagatagatgtaaataacgcttgggcaggaatatggagcacgggatgaggatgggcggccaactgttagagagggtagcagggaggctgagatctgcctgccatgaactgggaggagaggctcctctctctcttcacccccactctgccccccaacactcctcagaacttatcctctcctcttctttccccaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaaggttagtggacagccatgcacagcaggcccagatcactgcaagccaaggggtggcgggaacagtttgcatccagaattgcaaagaaattttaaatacattattgtcttagactgtcagtaaagtaaagcctcattaatttgagtgggccaagataactcaagcagtgagataatggccagacacggtggctcacgcctgtaatcccagcactttggaaggcccaggcaggaggatcccttgaggccaggaatttgagaccggcctgggcaacatagcaagaccccgtctctaaaataatttaaaaattagccaggtgttgtggtgcatgtctatagtcctagctactcaggatgctgaggcagaaggatcacttgagcccaggagttcaaggttgcagtaagctgtgattataaaactgcactccagcctgagcaacagagcaagaccctgtcaaaaaaaaaagaaaagaaaaaagaaagaaagaaatttaccttgagttacccacatgagtgaatgtagggacagagattttagggccttaacaatctctcaaatacagggtactttttgaggcattagccacacctgttagcttataaatcagtggtattgattagcatgtaaaatatgtgactttaaacattgctttttatctcttacttagatcaggcctgagtggcctctctttagcaagagttggttagccctgggattcttactgtagccacattaataaacaacatcgacttctaaacattctataataccatcttttggccaaattgacttcgcctcttcctctctctttccaaatgaaatgtgtttcatttcactgtcagaccacatggttggggaccccacagagcacacagccctccctctgccttcccatgctggcccttcacccactgctggagtgccaggttggtccaagggttggaccaagttgtctgaggttgtctcaaggttggtcgaggctgtctccgcgctgggttgtgctacaaggagcccttctttccatgggtgtggctggcagtgagtgctcacagcaacagcccacagtgcagcccgagggcaggatggactcagtccctgcctccatacccatttctaaggaggcaaaatggcaaacactctacttttctcttttaatgctaaaaataagaaaacaccttgcagcccagggtatgggtagtgcatggaagccgtggagttgtgaggtgggaagtgacctctgctggatatgtctattcaggaagattgctggagtgggtggggtctctgggaggtcccctgagtgtgggaagctgggaccaccagctttctcgcacagggagtggccatcccagcttggagaggttccaggactggttgggaggcacgtttcagatttctatctgttgaatcagcgaagatattggattatgaggaatttgggaattaggaaagtgggtgcaggtgggttgggggtaggtgaaggaagacatgggcgtattgggggagcaggggctgctcagaggtgttccagaagctctgggtgaggaggtgagagggaccggggaatgcagctcggcccagcctccctgcctgaggtcagccatcacgtggtgatggcaagatggaaatgtgctttctgactgctccagccagtgctgccagattcagctccccagggagggcacctgagaggctccaagccaggagatctgttttctcctttgttttgtttttttttgttttgttttgttttattatactttaagttctagggtacatgtgcacaacgtgcaggtttgttacatatgtatacatgtgccatgttggtgtgctgcacccatcaacttgtcatttacattaggtatatctcctaatgctatccctcccccctccccccaccccctgttttctcctttgaatccttcttagaggccgggtgcggtggctcacgcctgtaatcccagcactttgggaggctgcggcaggaggattgcttgagcccaggagttccagaccagcctgggcaacatagtgagacctcgtctctacagataataattttaaaaattatccgggcatagtggcatgcacctatagtcccagctactcaagaggcagaggcaggaggatcacttgagcccaggaggcggaggttgccgtgagccaagatcccaccactgcactccagcctgggcgacagagacccccatgtcaaataataataataataaataaatccttctcagtcccttcctcactgtgtccccctccactgaatttttccacctcctctcccacttcccccactcccgctttccctctccttctctccccactccatctttttctttctctgctgtttctcgtccctccctcctctccatcccacaacactgcctaccctgtccctgccccaccctggtgctcaggatgtgtgaagtgaggggtggtagcccccaagacctcaaccccgaaggttagcctgttgaaaccactttctcccagctgcccccctggcagttggtgctgctgggggaaactgggattgggggccagattttgcctcttttcctgacaaagagagatgaagagttctctcaccaggtgcctgggactggggtgtgggtgtcccagcctatcccagcgcatctgttctgcatcatgattaatagtgctgctttcagccgggcgcggtggctcacacctgtaatcccagcactttgggaggctaaggtgggcagatcacaaggtcaggagttcgagaccagcctggccaacatggtgaaacctcgtctctactaaaaatacaaaaattaaccaggtgtggtggtgggtgcctgtagtcccagctacttgggaggctgaggcaggagaatcacttgaatctgggaagcagaggttgcagtgagccaagatcgtgccactgcactccagcctgggtgacagagcgagactccgtcctaaaaaaaaaggagttttgctctgtcgcccaggctggagtgtagtggcgccatctcggctcaccgcaacctgcgcctcccgggtgcaagcgattctcctgcctcagcctcccaagtagctaggattacaggcgcctaccaccacgcccggccagttcttgtatttttagaagagacggggtttcaccctgttggccaggctcgtctgggactcctgacctcaggtaatccgcccacctcagcctcccaaagtgctgggattgcaggcatgagccaccgtgcccagtcaactccttctcaaaaaaaaaaaaatagtgctgctttctctttcaagtgtcctgatttgggtgatagtaaatgccactctacttataagggatctacctcagaatgctaattgggacatttttgtagcactctactgttggcagcaggtgatgctcacaacagcccgtgagggtggatgacgtccgcttcacagatgacaaaggagcctcatgctcagaccgtgggctgccagagcaggtccatggctgcagccccacatggaccatatttcccccttgtcactctttccaccaagctcccttggaacttcagttattaagctctcttgggtggaatccaagttagaatcacaacatgtgcctcatatggattgtgccagtgaaaaatgacattctatttagaggcagggcagcctggcttagagtcagtttaaaatatgtattatgctgcaacaaatgtaccatgatcctgtaagatgttcacaacaagggaactggatgtggggtatactgtctgtactaacttcacaagttttctgtaaatctaaaactgttccaaaataacaagttcgtttaaaattaactccaggagaccaggtacggtagctaatgcctataatcccagcacttcggaaggctgaggcaggtggattgcttgagcccaggagtttgagacaagcctgggcaacatggtgaaatcctgtctctaaaaaaaatcacaaaaattagccaggtgtggtggcgcattcctgtagtcccagctacttgcggggctgaggtgggagaatcatctgagcccaggagtttgaggctgcagtgagctgtgattgtaccactgcactccaacctgggcaacagagcaagaccctgtctcaaaaaacaaaaatgaaataaagtccaggaaagaagtaggttttaccactcttattttctgaagagaaaactaaatttaatgtgtaaagtgaggacaagttcaccaagttagtgtttgagttgcctaaaatatgtttgctaaaactattcaaagctttcacataaaacatgatcagaagttctatgccaaaacatatgtgtgtgtatatatatatgcactatatatactgtatataaaaatgcaaaatctaaattgccaaccttttagaaattgctctgaaaggaaagcatttcaagataatttgcttacccaaagaatatactttccaagaaagcaagtaatacttaaggtgttcataatcctcatcaaattaattcttgctactgaaagcttacaaggagctgttttgatgtcgggtgtgacaggtttgacttggcagaaggtgtcactttactaacaacattttaaataagtgacagaagacaagaaactacacgttaaatgccagaacaaagagtgtctaagtggatgctaagagttgaaatatggctggatacctgcccaagagagctgaaaagtagatgaaagttggttacctataaactagtgcaccctaatgaattaaaaggtgttgatgagttaacttgttatgccttccagataagacatgcaaatggggcttcttcctccttcactacttccaagggatttaacaaggagaccaatgcaaatgataaggactgtagggctcaagctggggacagattggggaaagggggaccatcatgcccatatagatgtccctgtgccctggcagtcaaggctgctgaaaaataacaaaacccagaagtctgcgtgatgctgcctctccatttgtccaaagccttcttgcggcagtttgcaggcttttgcaaaagctccaggaccaaggagctatgttcatgctggaagcttgttcaggattagctgttctttgtgggatgggtgcagccagggccaggtgtccagggacagtgttttaacaaagggcatgaggtgtctgatctcacagtggaactccacttgcctttttttcatcttctcattctgcttcatgcacagaaccagccccatcctgaaactgactctaaattactcccgccccaggtggagtgcctttctcggagttcaacagagccttcctgtcgcccaagggacaactccactgaatgcccaagccacacccaaaacctaacaagtaaaaaccaaattctgtgctcccccatcctgggccattcctggtttctctactgctgttggtgataccaccatcagcttgtccatcatgaccctggccagttcctcccacaaccctccacagcacccagggacctcacctccattccatccgacacagatctcctcaccacaaaccttggttttgcaacagcagccatgagacctttacaccctccgcccttcatcctgtcccccactgaggccccagagccattccttaaagcagcgcgccacaaactataacccacaagccaattctggtacccagcctgttttgcacagccagtgaactgacaatgatcttttcatacagccagaaaaacaaaacaaaacaaaaaacaacaaaaaaaaaccccaccattctgagcatgtgacttccatgttcaagatgtctcatgttcagaaaggcccctggaaaaggaggaaggggagctgggcacaaagggagaccctctcagctgagctcctcccatccagacattttcctggacttcctatccaatgacttcccttagcttcttatcagccacccctgtctgcccaggaggctggaagatgtggccttttaactgggcacagctctgtcctctatcatatcagggctctgttcccaaggagggtagagagaatggacaccaggtggaccctcagcagtctgtgccacagagggagtgtttgcaatttccagactaaaagtccccatgtgcttgacggggtatgtgactacaacgtgatgcttgacttttcctcatatgaccagagccactttgtccatctggtacaatgtcagctatctgctaggggccctccaggattcccagtcaattccatatctgcatcaccaccattggcactaaataaaataaaatactcaagttcctgctggtgagcatgagcagtgctacactgggcccttcaaccaaggtgacatgataatgactgaaaataatcactgccacttattggggacgtctcatctgccaggcatggtacaaagtgctttaaataagcattcaacaatttcatgctgacagaagccctgtgagccagtggagctactactatgcccattatacaggggagaaaactgaggcagagagaggttaggtaattcgctcagcctcacacaaccaataggtggtggagccaggatttgggccccatctgcctgactctctagaggctctatcttccagtcttccagagttgagtctaagccatgaataggacaattagacagcagaggaaacccattcagccaccatgtgcatgaagagtaaggaatttctgtcatacagaggggagtgaattcactgagctgagagctgaggaaccattgatctgatggctgagacaccactgggaagactggagaggcttttctgggcatgcagtgccaggcacaggaggagctgagggaagatgactaagaggtactggcaaagaattcagaaattctgatggaagctttacatgttaccatcacatccatccatctatccacccatccatccacccatatcttcctccctccacccaatcatgcatacatccagtcatctatacaccacccacccacccatccatccatccatccatcccttcatccatcccatcatccatccaattatacatacatccaatcatatatctgtacataatccattcttccctcggttcatccatccatccattcatccatccatccacccatcccttccttcatccttcctatcatccatccaatcatatatctgtacataatccattcttccctcggttcatccatccatccattcatccatccatccacccatcccttccttcatccttcctatcatccatccaatcatacatatatccaatcatacatctgcacatcaccagctcatccatctatccatttatccatccatccttccttccatccatcattcatccatcatacatacatctaaccatacatctctacatcattcattcttccatcgattcatccaattatccatcattccttcctccatccatcccattatccatttgatcatacatatatcatctatacatcatccattcatccatccatccatccatccacccatatcttcatccaatcaatcatacatacatcgaatcatctacacatcacccatccatccatccatccattcatctatccacccatccatccatccatccatccattcatctatccacccatccatccatccatccatccatccatccatgtaaccatccagtcatatatccaattacacatccatccagttatacattcatacatgcatctaatcattcaattatacatacacacatccatataattctacatccaattatacctccatccaattacacattcatacacccacctaataaattattaattcatatatccatccatataattatacatcaattatacatccatctaatcattcagtaattcacccaccatccagtcatctatccaataatacattcatccaatcatccatccatccatccacccattcatccatccatccgtccgtccacccatcatggtatgagccatgatttaccacgatggtcccctgtggacagcccaggtggggcagaactgaagggaagcccagggctgcccccataaacatttgcctcctttacatggatgagaactagatccacatgtataaatcctcatgatttgaaggtgcttttaccaacattcactcatgggattctcccaggagctctaggaggaggcaggtagagttgaggtcatctcacgcattttacagatgaggaaacggaggccctgagaggcaggtccaaggccacctgaccagaaagaagtggaactgggacttgaacccagccatcttgccccttggtcccatgctctctagcctgtaactcctgcttcctggtggggcatctccaggaggaccctatcggctggccatgggcctgccctggagtcttttgctctgtgtggccatccttcctccctcaggagagtgtgtgctcccagagcacaggctgtatcttctgagcattttgtcccttcccagtacctagcactcagctctgtatacattgggctctcaagaattctcaaccttccagagtgtaaggccttgacctgctcagccctggatactgcatgatgcattgataagcccataaaataaccagggcagattgactcccagtggccaaagtgccacagggaagggacaattcagcccttctaggaggaggaggaggtagttttctcatttctattaaggcaacaaaagctgccttactaaggacattcttggtggagggcgtgactgtcaaccactgtgatcatttgggcctctcttgcccaggcttcccattctgaaaggacagttttattgtaggtacacatggctgccatttcaaatgtaactcacagcttgtccatcagtccttggaggtctttctatgaaaggagcttggtggcgtccaaacaccacccaatgtccacttagaagtaagcaccgtgtctgccctgagctgactccttttccaaggaaggggttggatcgctgagtgtttttccaggtgtctacttgttgttaattaatagcaatgacaaagcagaaggttcatgcgtagctcggctttctggtatttgctgcccgttgaccaatggaagataaacctttgcctcaggtggcaccactagctggttaagaggcactttgtcctttcacccaggagcaaacgcacatcacctgtgtcctcatctgatggccctggtgtggggcacagtcgtgttggcagggagggaggtggggttggtcccctttgtgggtttgttgcgaggccgtgttccagctgtttccacagggagcgattttcagctccacaggacactgctccccagttcctcctgagaacaaaagggggcgctggggagaggccaccgttctgagggctcactgtatgtgttccagaatctcccctgcagacccccactgaggacggatctgaggaaccgggctctgaaacctctgatgctaagagcactccaacagcggaaggtgggccccccttcagacgccccctccatgcctccagcctgtgcttagccgtgctttgagcctccctcctggctgcatctgctgctccccctggctgagagatgtgctcactccttcggtgctttgcaggacagcgtggtgggagctgagccttgcgtcgatgccttgcttgctggtgctgagtgtgggcaccttcatcccgtgtgtgctctggaggcagccacccttggacagtcccgcgcacagctccacaaagccccgctccatacgattgtcctcccacacccccttcaaaagccccctcctctctctttcttcaggggccagtaggtcccagagcagccatttggctgagggaaggggcaggtcagtggacatctgatcttggtttagtatccttcattttgggggctctgggtgtggcctgggcctctggactttggccacggtgtttgttccagcccttctcctaacctgtcctttccagacactcggcatctaggttattagcacctcgcatactttctgacatgctcctcagtcctgattttgaccatcttctcttgcttcccatctgtgtcagtcaagactgcatttggctgtaagaaacagaaaccccaactaactgtggcatttacatgaagaggtttacttttctcacataatcagatgtctagacttggccagcacctcaagggtcattgatgctctcctgtctttattttctgtcatctttagtggttggattgttgcctcatggttacaaagtggctgctgcacttccaggcatcacatctgcctttgaagcaggaacaagttgcaaagtaaagtggccaaaagggccctgaaactaaatgtgtccccttaggaaagcaggagttttcttgcaagtggcaatcttctgcttatgtctcattggccagagctgggtcttacggccaccccttgctgcgagcaaggctgggacattgagcattttgccgtccaacctctttagcagaataaaccaagggggaagaacgttaatagtggcttttgagtcactagttggcagtatctgcccctctatctttccatcctccccatggagtttcaaggttcctttctcagtacttcttcaggctctgcacgttcatttggatcttgtgtcttggggtgaaaaactggcccaagtgtctccccaagcatccacctttggattaatttggaaaatggctgtcaagtgcccgcctcttgcttggtataatgctacagctttagaggacgcagcaggcatgggccttgccgctgaggttcttagcctcatgagaatatccagatcagattctcttggctccttcttagagccagtgatgcaagacacttcctgctcatcttgtcgggacggttttacaagttgcctgccatcctgagaaagtctacaaaacgatgccagacctcatgccagcttcccaagccttgactctcagtgctccctcaacaggattctggaagaatctcccaaacaagtcgcaatcccctctggaccctgtgcaggcatgagactcaagagcattggctcccacccctggtggagggaacactgctggggctgggatcttgcctggttgctccgcctgcacccaagacaaccataattaaaatgtccttcattgaacttggaaagccttcaaagctgacaactccttatgtgtacccggaaaggcctgggagtgtgccagggcattgctcgggagggacgctgatttggaagcatttacctgatgagagactgacagcagctcctggtagccgagctttccctcctgcctctgctgtgaaggtggacccatccaacagtcaaatgcctgactctggacaggagcggacctatttattgccatgcaagggactctgcacttttgaattgtgggtcatgggcttggatttaggggttagagctgggagaagtcttggaagtcacctagagatgacactgccattttgcagatgaggaaaccgtccaatcaaaatggaccaaggacttgcccaaagcctcacagcaaaaccataggcccccgcactaaccccagagtccctgtgctgtcttaagaatcaaatagttgtaagcaatcatctggttttcagtatttcttcttttaaaatgcctggggccatgcccagcagtctgtttcactgcagcgtttacacagggctgccgggctttcctggtggatgagctgggcggttcatgagccagaaccactcagcagcatgtcagtgtgcttcctggggagctggtagcaggggctccgggccctacttcagggctgctttctggcatatggctgatcccctcctcactcctcctccctgcattgctcctgcgcaagaagcaaaggtgaggggctgggtatggctcgtcctggcccctctaaggtggatctcggtggtttctagatgtgacagcacccttagtggatgagggagctcccggcaagcaggctgccgcgcagccccacacggagatcccagaaggaaccacaggtgagggtaagccccagagacccccaggcagtcaaggccctgctgggtgccccagctgacctgtgacagaagtgagggagctttgcgtgtttatcctcctgtggggcaggaacatgggtggattctggctcctgggaatcttgggttgtgagtagctcgatgccttggtgctcagttacctccctggctgcctgccagcctctcagagcatttagggccttctggacttctagatgctcctcatcttgcctcagtcagcgcgtcagttccagagacttctctgcagggttttctggggcaggtggtggcagacccgtgccttcttgacacctgaggtcagtccaccctcctgctcagactgcccagcacagggtcacctcccaaggggtggaccccaagatcacctgagcgcacagagggtgcagatgactggaccacaccttttggtgatcttaatgaggtggtcccagaggagctcagacatgcaatctagcatccagttctgggactctgtctccttttcaaacgtattcatgtagaacaggcatgacgagaatgccttgtcaacatgggtgatggggaatcaatcagacagggcgccgggctcaaggctgcagtcacccaagagtggctcagcccaccaggccctaggaaacgcctgcacagcctggagctcctggagtcatttccttcatgtcttcttcactgcacttacgtaaagatgccagccattggtttggtgatttggagggtgcccagttgcccaacaagaaatgcagaagaggcctagccaggatttcaccagcagtggagagtagagaagatgtggccagaaaagagtttcctttccctcctaaagatggtactccctgcagctactggggaagcctgcagcattctctagggctctgtgtgttgagagcagccccaccctggccccttctgagtgcatttctgctttgtgacttgatccgtgaagtcccctgagatgggcagaggggatgtcctcgaagctggggcagagcctcatccttgaacgtgaaggacgtttgaagactgtggcatgatcacaggatgagatcacagggaacttgagtttctctcctcctctcccttcacagttatttcactgagggaaatccctcccctgcccagaatgaaaactctagccaactcttgacttttccatcactccaaagtagttgaaagtacattagtctccacagtggcaaaacagtgtgcaaaagctaaataattagaacagccagtcccatgtgacagtcaaagcttctaactccattcaaagttgcagccattcccctcgagggctggcagggaggggaggggtaagagaaacaggaaggttcttactgagttggtcctggtgtgagctgcgtcacactccctgcagaggtttcaaggagactctctctctctctgtctccatggggaccttatttgaattcttctactcttaccccagcctgccatctccagctatcctcccctgaagagcccttctgctgcgctggattctggtggccatgtcatctcctcggccccgtgggagtctgaagatctggctgcagcctcacctctgaggtcctgctagttgccacctcttaaacatgatctgaggctcccatgcactctgacctgtgcccacatggggcccacgggaaacacgctggcaagcaaactgtgggtgtgcagacggttctcagggctgcagcacctgtcctttgctctgcccccaaagcaaggccagcccatcttccatcctctagtgttccttggtggggccctgaccacagtccaccaggtccctaaccagaggggacacacaccaggtgtcctcaatgtattgccttgaaacagttgtgctgggactgtgatggggggtggccatgtagccacccccaccacccccaagccactctctccaaggaaatcctcctaaagatccctttacatcctccatgtggtggggaggttctagagttgggtgcatgtgtcttcagctactgacaatgcagaccttagttggcacctcgctctggcctatcctgtttgctgttcttggcgctccagtgaaactccccatgggccatccagttggggtgcagtgtggccacccccttgcaggttcctgccttgctggagagcacagggccctcctggctcttgtaaaacactccccatggtacagagaggccagcagtgatgtgaggcccaacctccctccatggtgttcccaagcagctccctttctggggtcaaggggtggcaaagacagtgcagcgtccaatttctgactcaagccgggcctggctatcgcagctctgcactgtgtgtgacagcaaggcaactcacccagtgccgtggcagtgaccgtgtccgaggaagcctcctcacaccctctgtctcaaggactctggcatttagctggacttgctgtagctctgagcctttctgccattgccatcaccttgtcagaaactcaggccgaatctgcactcagagttgtgcccaggcagttgagccaacacttgctcagcgatattgtcacatgacaaggcactgtcaccactgggcgtcgtgggtagcgcagtgtcggctggatggacccggagggtgtctgtgtcatgctagtgctagtgatgggagccccgtgagcccattgcccgccctcccatgccctcagcagctgcctggggacagccaatggcctgggtgtttctgaggctaccacatggcttccaggaaactcgagaacctttctctcccttgcctacactcttcacacaggcctgtgctggccagcggtggggatccggcattcctatcttaggtgcagaaagtgactgactcattgcaggcctgggagataagactgatggcccagccagcaagatgtatggatttctcagaggcagtggcctctgtcattgtcctcaggaaatgctggtgattctggtggcctgaggtcaatgcatgtcaacgtggccaacttgccttataaactttttttctggacaattgcgtgcactgtcctgtaacagtgtcctgttgtttatgatgcagaaataggtgtttttaaagcctattgattttggtactattaatgtggtcaggaactttctcagtctttcttgtttggggtgagctgtggcttcctaaacaggaacccaagacacccccaaaagctgctcaccagcactgccagcctccctcttaccaagtagcacccgttcaggacattctgcgaaaggcatttgcccagaagttgggaggaaggaaatgtaacattttggggcacctaccatatgccaggcaccaggctaaacgtgttcacacaaattctcttactaaccctcaccatccttctacaagacaaactagtatcttcatcttggggttcaagatgaggaaatggaggctcagagaggttgaatgaatgccggtgcctggatatgaaccccatctgcctgactccgcaacccaggcaaagtctttccttgaacttcccagcagccactgcttagacacagcctccacaaccatggctcagcagcaaattgcttctctgacctcactcagcctgtgtgtccttgttgagtgaggcattcaggaccctggtcccaaagtggagaaagtctttcctactaggtcatagctacacctgcatgtgggtgctgtgccttttgtttagtgaacttttatcaccagcatcctcagcaatgacatttgcagagaagccagagctgaggcaccttggtattcttgggatgtgactttcctgaatgtttaagggaaaatgcccgaaggtacagagagcttggtttctagtaaacaataactgtcttgcttttaccccccttcatttgctgacacatacaccagctgaagaagcaggcattggagacacccccagcctggaagacgaagctgctggtcacgtgacccaaggtcagtgaactggaattgcctgccatgacttgggggttggggggagggacatggggtgggctctgccctgaaaagatcatttggacctgagctctaattcacaagtccaggagattttagggagttggttcttatcaaaggttggctactcagatatagaaagagccctagtggtttttttctaataccatttctgggtaattcctaaggcatttagtgttctgaaagatgctagccttgtccagcctgggagttgagaatgaatgtctaacagaaactctaggccgggcgtggtggctcacgcctctaatcccagcactatgggagacccaggtgggcagatcacctgaggtcaggagtttgagaccagcctggccaacatgtgaaatcctgtctcactacaaataaaaaaattagccgggtgtggtggtaggtgcctataatcccagctactcaggaggctgaggcaggacaatcgctcgaacccaggaggtggacgttgcagtgagccgagatcgcatcattgcactccagcctgggcaacaaaagcaaaactccgtctcaaaaaaaaaaaagaaactcaaatatgtgtgacaggcgattctcactgcaggctgccctgtggctgatccaggagcaaggccttaaccatgtcatccccaagcgattgcttgtaaactttcttctgtgcagccttcaacccttattatgattttcttctcaggaaccaaactgctgtattcaagaaaggcagctttgtgtaatcatttatcataaatatcttaagaaaaatcctagagattcctaattttaggaaatgggagacctatggtactgatataatgtgggctgggcttgttttctgtcatttgctagataaatgaacttgagagcctactgtaaaatgtggaagcttctagattgcagaagggctggaaagacactgttcttttctcccgagtgatgggatctgtccagtatttagagctgcctctgaggccatctgattctaggagactctgcctcgttgaggatattttgaggcctaactacacattcctgcccccagagaggtcacagcctatagcaggctgatgtttctcatgtcacatggcacagaaaggcacattttcgttctcaggctaacaaagagcttcaaaaactattagaagggacagtggctataagagaagaacctcagtcaatgtgtgaaattaactaggaacctggctcctgtttcttttaggtcatgtttttcagcttaggtaaaactagaggctttgataaagcatgacctctagaaatcattgcttttcataaatggaagtgggtttgagttttttctactgattgttagtgcaggtgatgtctacatgcccccagaacatattccatgcaacaaaaaaagcccaggtcaccgtctttgctgggaacttgacttttgtgctcactgaattttaagctttctgacagcagcctggaatcatggagggataaagtacctattagtaagatggaaaaaggtgtttcaggttggagctgcagtctgttgagagtaagctatgggaaggcctgtatacgaggggtggacttttcttctgtaagtgtccagagaccaggcctcctgaagagggcatgggggcttaacttacctggactactgtgtttacaatactcatttatcttgaactcctcctaacccctgagaattgctacatttagtatttgctgagtacttcctagcatcctagggaatcaatagaacattctcccaaccaggctgggtgcggtggctcatgtctgtaatcccagcactttgggaggccaaggtaggcagatcccttgaggccaggagtgcaagactagcctggctgacatggtgaaaccccgtctttactaaaaatacaaaagttagccaggcatggtggtacacacctgtaatcccagctacatgggaggagtaggaggcaggagaattgcttgaacctgggaggtggaggttgctgtgagccgagatcatgccactgcactccagcctgggcgacagagtgagtgagactctgtttaaaaaaaaaaaaaaaaaagaacattctcctaacctggcttcttcctccaggggtgtaattaatcatgtcagtttcctcattgatacacacacacacacactacaatcctgtatccattacttttcaaggtacatttactatttacgtttggggtccttgtctcttttttaatagtgtttcttaaagtcttgtattatatcagagtacagtaacatcccagtcaagagcactctagtaagctctaggaggaaagcgacttccggaaggcagtggagacctgtcctgttggggcagcataggggcagcccctgcctctggtcagttctggcgctcaggctcagggttgcctctgggctgttcttcccagagactgacaaagggctcccataaggcacctgcagagcctgtgagaagctgaagtcaatgttttcctgacaccagttgatctgtgcaggatccattgatttaaccacctgctgtgtggcatgcactgtggtcgatgccaggaacaggaattggaggggcccatgagcatggccagtatcacaggctggaggtgctgctgcgctctgaccgggcctcttggggatgagcccatgtcaaccaccttgcctccgatggggtcgggcccacaggttacctttgtgtgtccatgaccacaccttcctccccgacctcatccaaatctctttcttttccaagcccctgaatccttcagggctgcaggttttgtttaaagcagagctggtgagttgcataggttgttgcgttgggactagatggggtgttcaaagagttgggagttaaaaaacataaagggtatttattaggagaaccaaggagtgtaattctcctgttcttaatatgcggccaggttaatgaatgtcacgtgaatgaaccagaaaaaaatgaagtgtgcccttgatcagctgggttggtgtgcagcaagctgtgtgaccaggggacagcagtggtcctgagggccgtcactgtctgccgtgcagagcccttcctcccacgggggcctacctcacctgtgccaagggcttgtctgtggtcagtgacctggatagatctgaatggggcttctttttcgaggagtcttatggcaggtctctcagtaaagactccattcttgatgatcacacattttggattttccaaatctgtcagagaatgggcttgaggcggggtttgtgggcactagtttcactggtttcatttaccaaaaaggggagcagaagtcaagtatggtggctcatccctgtaatcccagaggcaagagaattgcttgagcccaggagttcgagaccagcctgagcaacataaggagaccccgtctccacaaaaatgaaaaataacattttagtcagacgtggtggcatgcatctgtggtcccagctgcttgggagggtgagatgggagggttgtttgagccctggagttaaagttgcaatgagctgtgattgcaccactgcactctagcctgggtgacagaacgagaccctgtctcaaaaaaaaaaaaaaagaaagaaaaaaaggaaaaaaaaaactcatgcctgtaatcccagcactttggggaccggggtgggcagatcacgaggtcaggagatcaagactatcctagccaacatggtgaaaccccgtttctactaaaaatacaaaaattagccaggtgtggtggcacgtgcctgtaatcccagttactcgggaggctgaggcaggagaatcgcttgaaccagggagtcagaggttgcagtgagctgagatcgtgccactgtactccagcctgggcgacagagtgagactctgtctcaaaccaaaaaaaaggggtggggggcgggggcaggagaacagtgagaggtagggagaggaaaggggattctcgctacacccaaaccagataccatctagaggctagaatctttgggaggctcaaattccctagaaagcaggagaagcttctgtagccctcccgctttcccagtagattaagcccagggcggctccagatgtgtgacatgctctgtgcccaaccagagcccatcataggcagaggaataacacccacaccagaagggccctcggaggtcaccacgtccaagaaccctctttacagatgaggaaactgaggcccagagaggggagagccacctagcgagctggtggcggctagaccaggagagctgtcattccaagcaagcaaaggcaacgagacgagcccagagctgtgctcccatctctttgttagggggcctgggatgccctctcagtgtcattttgtccaggatgatgctccctctcttaagcgattaatgcgcccttgctaaccttttgctatcgctgcctcttcaaaccagaggagttgagagttccgggccggcagaggaaggcgcctgaaaggcccctggccaatgagattagcgcccacgtccagcctggaccctgcggagaggcctctggggtctctgggccgtgcctcggggagaaagagccagaagctcccgtcccgctgaccgcgagccttcctcagcaccgtcccgtttgcccagcgcctcctccaacaggaggccctcaggagccctccctggagtggggacaaaaaggcggggactgggccgagaagggtccggcctttccgaagcccgccaccactgcgtatctccacacagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaaggtctgtgtcttgagcttcttcgctccttccctggggacctcccaggcctcccaggctgcgggcactgccactgagcttccaggcctcccgactcctgctgcttctgacgttcctaggacgccactaaatcgacacctgggtgcagctgctccactccctcggcctcctcccgtgctcaggctgtggccgcacgcgcccctcacgcttgcccgccactctgcatgtcaccagcacccccgctccgtgctccccaccttgtttgactctctggccacttgatttgtccacaacggcccatcagcccacaggaggtttggtgggtgccttccaccgacaggatgacgggtgccctcatggtgtctagaactctccaaccctcccatgtaggcataagcagccccactttgcagatgaggaaacggaggctcagagaagtacagtaacttgccgaaggccaatgagtagtaagtgacagagccaggtttgggatccaggtaggttgtctctgaaagacacgcctgtcctgcatcccacaacgcctcccaggaggtgctggagtgtggacgcctaacacagagatgtgcagggcacacacagcaggtgacacacacagcatccagaggtggcccagagctcatgctgtgcctttggcccagtgccctgcccccacccactctgccttgtggcaggaagacaaggagcagacacaagatctccctggtccacatgccaccacctccctctgcagaggacaaggggatcctcatgctggcattggagggggttgagcagggcccaccttgagccctcaggagcacgaccacagcagccctgcagggagggattggtgggaggagagtcccaagtatcagggagaggagagttggtgtcccacaggagacctcagagccacaaggcgagcttgttcataaatttgggacccttagcatttcacagttatttgcagagcccagaaatggatgttactgaagctcacagttgcaagcatctgttaaatttttattagattttacttttagggaaaactttgaaatgctataaagaagcctgtgtttaaaagttaagacagaggctgggggcgatggctcacgcctgtaatctcagcactttgggaggccaaggcaggtggatcatttgaggttaggagttcgagaccagcctggccaacatggtgagaccctgtctctactaaaattacaaaaaattagctgggcgtggtggcgggcacctgtagtcccagctactggggaggctgaagcaggataagtgcttgaacccaggaggcggaggttacagtgagccaagatcacaccactgtaccctaagcctgggcgacagagtgagactctgtctcaaaaaataaaataaaataaagttaagagagaaaaaaatatatcctatatcctttgttaaattccaaaacagtaggggacaaataactgacttgacaggttactacaatatttcctgaaatgatgttttcttgaatactggcctactagaggttcataggtgtgtttggattaaaaaagagttccatggcccagtgactgggggaaaaaaataaaagactaaagtaagttaaacaggcttttctgctgcaggacttgtcagagcctttaatgtactaatggccattgtgaccctctgagaaggtcacagagtgggtttcccaaacttacttgattctacctgctaacatttcctggaggaagtttgggaaatgccgatttagcagattcttttgttgtgccgtggatggtgctggttgatgtgggcaaaacaaagaacacgtgagtcagatccgcctggggctcttactaaagtgcaggttcccaggtgccactttaggcttacagacccagttgtggggtaagcctgggagtcttttagcaggtgattctgccacatagtatagttggaaaacctctgggcatactcattgctggtccctctagaaatccaggtgacaatagccaatgagaagctccaagagacccagttgtccatggggtagagggaatgtgatattgaaaccaaagaagaaaatctatgatcagttttcagcagtgactgtcaagagaaggagaagggtgagttagcgctgatgctggctgacaggtcagcgggttggtttcaccaaggagtgtgatgaaggctgatgttgtctgtgggaatgtatgatggtaactggtttgtagctaatttggggaagcagtgagaattcgtgccctttgaagaccagtaagtggcaagaaacccaccaggcctggctcagggctgggctgggcttggctcgtctcagagcagctggggctggtggccaaagccaccattagtgaggggcaggccctgggggtacaaccagcaactaggggacaaagacaaccctgccagcctctcctattctggaggcgtgtgaccagaaatggagatgggttggtcagcataagatggccaggaaggtggaaatcaggactgctggcaatctagccacatgggcaggggagccgggtggttccaggcagtttccaaggccaagagggtgagcaggcacctcacagggaatcagggccaagcctggctgcagtgtggagacaatgcacccacccccatccttggatcttgcaggaggctgggtcctcactgagctaccaacatccatggccctgaggcttttaaaacacccatccatggagtggggctggtcccagtggggtgaggctgaccctggcagaaacagggcaggagcctgtgggttagggagactgcaccttccttagatagcctccatgccatcatgtccccgtgacagtttctgctgcgtcccctctgcatggtcccaccctcggccagcctgctgccccctcttgccaggttgcgctaatcagtgaccccagtgtgctgtgttgatactaacaatgcgaggcctagcagattcaagggaaaagagaaccaactgggtttccaccagacccaactaaacaaacatggacctatcccagagaaatccagcttcaccacagctggctttctgtgaacagtgaaaatggagtgtgacaagcattcttattttatattttatcagctcgcatggtcagtaaaagcaaagacgggactggaagcgatgacaaaaaagccaaggtaagctgacgatgccacggagctctgcagctggtcaagtttacagagaagctgtgctttatgtctgattcattctcatatataatgtggggagtatttgtcactaaagtacagctgtcatttaaagtgctttgtattttggggcaggcttttaaaaagtccagcatttattagttttgatacttaccccagggaagagcagttggcaggttcatgaagtcatgctcctaattccagctttcttagtgtactttcagtgagaccctgacagtaaatgaaggtgtgtttgaaaaccaaacccaggacagtaaatgaaggtgtgtttgaaaaccagccctaggacagtaaatgaagccatcttctcactgcataaactgcacccagatctttgcccatccttctcagtatttcacttcacccattgtttactgtctcaatgactggggaaatgtctggggaaatgctcccgtaattgcacagtggcgtttttcctggaaaatcccaccatggctctagataagacctatttttcttaaaggtatctaaaatttccagcataaattctgtctgaaacacctgaattttaatcagtactggagcccggagggcatctccagttgccacatagctctgagcattcagtggtgtgttgagggctgctcccggaagtgcctgcagagtcagggctccccagcctcatctagtgaggcagtggaagggcctgtggggatttggagagctggcctgggtctctgaagtgatagtgacagctgcttgtcaatcacggtgcacatttagtgccgggggcagggggcagggaataccagcctcatgcatgcatgcattcatttgttccttccttcattcattcattcagtacacatgggtacaacatccctgccctggagttgcccagagtctagggaggggaaagatctattaccctgggcctcggccagctggggagtgctgctggtggagaggggccgtgtgcagcgagggaaggaggagtcgtcaatacccccaccccagctttgctttcttgtcatcagccccagggccccagcctgtgtccctcctctcccattgctacttcatctcctgggtcctccttaccaagcctgaccacacagagggccttggccgcttccatggggaattggaaagcaataagatagcatcccctagaagcccagtgaagtctgggacaggacccttctctgagctctgacttgctcttggaaacacttcgaggcttagcctccccactttgtttcccaagagtgtgacctgttcccctccaaacacccccttctcctccagggccatgcccacccgtcaaaatcccccacgggcaggacgaactgtgggtgtcagtcaccatctatcctgcatcctggttccagggccccccccagccccgcctccatagggacaggcgtgcagacacccgtccctggctgcttcctcttgtggaatgggttcaaaagtaagcagtgttgtttacactgacaaactgaaaaaaaaagaaaaagagataacattggaggcttggcacagtggctcatgcctgtaatcccagcactttgggaggctaaggtgggaggatgtccccagcccaagagttctagaccagcctgggcaacatagcaagaccccatctcaaaaaaaaaatttaattggccaggcagaggtgggaggatcacttgaacccaaagggtggaggctgcagtgagccgtgatggcaccactgcactccagccagggcaacagagggagaccctgtctctaaaacaaacaaacaaacaaacaaacaaaagagttaacattggccagattaggattcaccagatagtgttaatattagtttgatttgagactttaatcagaaagcacatgtgtggtgggggtgggtgtaacctaagtcaggtagaatctttccaacttggggggggcacactcctgattgtagccatatgagtctgtcagtgtggtggaagagaccatgggttaatgggcaggtaaaaaagcaccttgcctggaattgagtagaaagtaaggcccttcagaccccgtgacacacttggggacattttcttgagtaacatcctaagattcatgtaccttgatgatctccatcaacttactcatgtgaagcacctttaaaccagtcgtctccaaattcaggggcacagtaacatccaacaggctggagaaagaacgtactagaacttccattcctttttcatgtcctcttctaaaagctttgtcagggccaggcgcggtggctcacgcctgtaatcccagcactttgggaggccgagacgggtggatcacgaggtcaggagatcgagaccatcctggctaacacagtgaaaccccatctctactaaaaatacaaaaaaacgagccgggcgtggtggtgggcgcctgtagtcccagctactcgggaggctgaggcaggagaatggcgtgaacccaggaggcagagcttgcagtgagccgagattgcaccactgcagtccagcctgggcgacagagcgagactccgtctcaaaaaagaaaaagaaaaagaaaaagaactgtgattggggaggacggtcactttcctgttcttactgatcagaagggatattaagggtacctgattcaaacagcctggagatcactgctttcaaccattacctgccttatttatttttagttactgtccttttttcagtttgtttccctcctccatgtgctgacttttattttgattttatttatgtttatgtttaagacatccacacgttcctctgctaaaaccttgaaaaataggccttgccttagccccaaacaccccactcctggtagctcagaccctctgatccaaccctccagccctgctgtgtgcccagagccaccttcctctcctaaacacgtctcttctgtcacttcccgaactggcagttctggagcaaaggagatgaaactcaaggtaaggaaaccacctttgaaaagaaccaggctgctctgctgtggtttgcaaatgtggggtttgtttatttgttttttagcctcaaagacctttcttcaaatgagttctggcatagaagcaccgtgtaaaatagttagaattctgggcaaaggggaaaagagagctgggggccatccctctcagcaccccacaggctctcatagcagcagctcctaagacacctggtgggaccttggtttcgaaatcgctactctaaggctgggcacggtggctcacacctgtaatcccagctctttaggaggccgaggagggtggatcacctgagatcaggagttcgagaccagcctggctaacatggcaaaaccctgtctctactaaaaatacaaaaattagccgggcgtggtgttatgcgtggtggtaatcgcagctactcgggaggctgaggcacaaggattgcttgaaccccagaggcagaggttgtagttagctccagcttgggcgacagagcaagaccctgtcgcaaaaattgtttaaaaaacaaacccaaaattgctactctcattgggttcctttgcccattcctgattttggcaagagaaatgcttccagattgccctgatctgggtaggacagcatcacgccatagcaacactgccccgtgagctcactgccccctcaactagcttgtggtccttggttaatgtcagtttcttttttgagtttgtgttatgtctaagggtcatctgctgggtaacggaacccagggactgccctagtccctagactgtgccatgcccgactctgccagctttgtcagtgatgctggtgctcgcctcctcgggtgctcgcctggtctgagcacacccaaggagttcttgaggccttagggttgtttgcgagagaatgaaagaacacgacctagctctctttagcatccttggtcaggttcaacactgcccccaggggcctctggtggagccaaccaccatcagccaaataaatccataattagagtcagaaaatggatgtctgcatatgtgtagtgcactaatgtcctgccgatgattgacatggagtggagagtgacctgatcattgctgtgagctctgctggccttggcacaactcatgctgataactaatgcacacagttcctctgggaggaaatgtcctcagggaacttggagtttgggtggggatgtgggtttgtgtgcccagcaagcccttgtggttgtagcagacactagtggcatctaggaggcaaagggtcaccccagtcttagccacgttttgagtcaaggtggcggagtggggctggtgttgactcttggtggcagtaacttttcccaatggtgaaaaacccctctatcatgtttcatttacagggggctgatggtaaaacgaagatcgccacaccgcggggagcagcccctccaggccagaagggccaggccaacgccaccaggattccagcaaaaaccccgcccgctccaaagacaccacccagctctggtaagaagaacgttctcttgaatcttagaggaagctgaagctctcagaggtacagccttcattttaggaggccttaggccactgagaatgaataacccctggcagctggtcagcagcttgcagtttactaagcactggagtcttcattgccttctcagtccttttgatttctgaggcaaatgttgaatccctacctttttttttttttttcttttgagacagagtttcgcttttgttatccaggccggagtgcagtggtgtgatctcagctcactgcatcctccacctcccaggttcaagcgattctcctacctcagcctccctagtagctgggattacaggcacctgccactatgcccggctaattttttgtatttttagtagagacagggtttcaccatgttggccaggctggtctcgaacgcctgacctcaggtgatccacctgcctcggcctcccaaagtgctgggattacaggcatgagccaccactcccagcctgaatcctcactttttatcaatgaagaaattgaggctgattctgcagcatgataaaaaaaaatacagaaaaaggaaaaaaaagaaagaaatcgagcctctgagagtttgcttgactgagtctaaccagctcattttaaacccgaggaaaatgcagtcacatgactactaagtggcagctctcggagcctctctggccccaagtccagggttccatagaggcagccccagcatggcatgttttcagtccccaaatgagactctggagacaaatgtctctggagacagagcagcagcctggataagtcacaatgggtgacgtcactcagggctcaacccctgggcagcttaacttgctagggacgttaggagtctgctgcaaaacctgagggtcttagctgagcagtcacaggctgggcccgttgccctgggctcctgtgagtaaaacccagtcaattttgagtacccagtaaggcatccattgagttattttgcagccaggagtgctattaagaacagtcgcggctgggcgtggtggctcatgcctgtaatcccagcactttgggaggccaaggtgggcggatcacctgaggtcaggagttcgagaccagcttggccaacatggcaaaaccccgtctctaataaaaatacaaaataattagctgggcgtggtggcgggcgcctgtaatcccagcttctcaggagggtgaggaaggagaatcacttgaacccaggaggcagaggttgcagtgagctgagatcgcaccattgcactccagcctggatgacaaaagtgagattccttctcaaaaaaaaaaaaaaaaaaacagtcgtcctctttggggattagggacagcctgcctgcctgcccgagcacttctctcttccattgccccagtgaagtattccaggcccctgggtttagactctgcaccatgtaggggtgtctgacctgcacttgctccttggtggcacgggcagcctatggcacttgctgcgggctgtgaccaaagcctggcctggatcttggatcttggtgactctgcttctccctggcctgagggagctgcccagagcctgcccaccacctgctgcgtgtctttgcggtggcatttctcgcacacatgccgtgcggtggcacccccaaggatggccattcactaaggcccattgtttttgtcttttcgcttcgtgttttctggcctggtgtttttctcatatacatgtgatccagggataattcccagaattttgacaggattttaagtagcgtttggatcctgctgtttttttttcacttaacatcgggccagttgactcacactctgttttttgttgttgtttttttgagacggagtctcactgtgtcacccaggctgaagtgcagtggcacaatcttggcatactgcaacctctgcttcccaaattcaagcagttttcctgcctcagcctcctgagtagctgggactacaggcacaggccaccacgccctgctaatttttgtatttttagtaaagacagggtttcaccattttggccagcctagtctcgaactcctgacctcaagtgatccgcccacctcggcctcccaaagtgctgggattacaggggactcacactttgtaacaacctgaaacaacgtgatgcatttccctttgggtcttacctgctcttcggtggctgcctgcaggtggagagaccctcccccttgggcccctcgaccttgtttcagaatggggcccctgctgggccagctgtgggtgcctgccacgtgaaggactcattaaggccctgtttaagcctgatgataataaggctttcgtggatttttctctttaagcgactaagcaagtccagagaagaccaccccctgcagggcccagatctgagagaggtactcgggagcctacttcgctgggagcagcctccctttgcgtgtgtggccattcactggcttgtgtttctagagccgggaggacccttttctgcaatgcagggttcacacagggttcgcagcctgaagatggagcagtccgaattctcttccctgtgcagtttgcgcagctgtgtttgtctgatgggctttctaatcctgtgtgctctccttgacttcagggacaatggcattacaggcatgagccaccatgcctggctgtctccctatgtttcagatgaagacataggcttaaggaggtcaggtgacttgcccacgaccactctgtaaataagaggcatgaaaagtatttggagccaccaccaccaagcccactggtcaccctgggtctctgaagtcagggaggcaggaggatgggaggtctgaggaggcagagaggctgagcctggaggccctggaggccgaggccccatctgttgtttccttatgtggaaaataagaggcttcatttgtcctattgccacagagcgtactacttcaggaacatccaagacatggaaatccgcagggcacggtggctcacgtctataatcccggcactttgggaggttgaggtgggagaatcgcttgaggccagaagttcaagaccagcctgagcaacatagtcagaccccgtctctataaaaaacattatttttaaaaaagacatggaagtcaaattctaaaaactggtgctggctgggtgcggtggctcatgcctataatcccagcactttgggaggccgaggcgggtggatcacctgaggtcaggagttcaagaccagcctggccaacatggtaaaacctctactaaagaaatctttactgaaaatacaaaaatccagtctctactaaaataagtctctactaaaaatacaaaaattagccaggcgtggtgctgcacacctgtaatatcagctactcgggaggctgaggcaggagactcgcttgatcccatgcagcggaggttgcagtgagccgagatcacgccattgcactccagcctgggcatcagaataagactccgtctcaaaaaaaaaaccacaaaaaaacaaaacaacaacaaaagaaaactagtgcttattcgtcactggccaagctgcccattggctacatgggtgcttcaaagagctgcccttctccaggtctggccagcaggtatgtgttacagcaaatgcctggggcagcggcaggggcattgctgcgggaagcttctggacttgcaggaaagctaagttctcagactgcaggggagctaagcacacctcggcacagggtgaggcctgcggttctcagacttcagtctttgtggagcttgagaaaaatgaggctttgcaggtcccacccctagagattctgctctatccactcttgaaggggatcgagaaatttgcattttgcaactcccactttcctccttgaaagctccggagattctgacgcagggttccgtgggccacactttggaaaatacagacccatgagatagaataccagactgttgaagtgtaacgggggcctgggaagtgcagtaacagaagcaagtttgagggtaaaggacacccagaggagggagggacagcatctgcatggagaggagaagagaccccccagcagcttccagggtgttggaagggtgcgctagtaactgctatgcatggcaggtggggaactgtacgtcagggcacagcagcatgaagcggtatggctcgtgtggacagctagggacaggcaggcgtggagcaggcatcctgttctgaaggccaaatcccacagaggagccagggtgctggcaggagccctgaactagccgaacagctgaacagctgaacattcaccctgtggggaaagggtcagaagcgtccaggcttgagggcacagctgggtctcgtcactgcatcacccttatttaggataaaggccctgaagaattgtattagaggttggcaaagcatatctaccacctcctggagccacgctggccgcagggattataattatttccattttcaaattaaggcctctgagctcagagaggggaagttacttgtctgaggccacacagcttgttggagcccatctcttgacccaaagactgtggagccgagttggccacctctctgggagcgggtattggatggtggttgatggttttccattgctttcctgggaaaggggtgtctctgtccctaagcaaaaaggcagggaggaagagatgcttccccagggcagccgtctgctgtagctgcgcttccaacctggcttccacctgcctaacccagtggtgagcctgggaatggacccacgggacaggcagcccccagggccttttctgaccccacccactcgagtcctggcttcactcccttccttccttcccaggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgagagtggctggctgcgcgtggaggtgtggggggctgcgcctggaggggtagggctgtgcctggaagggtagggctgcgcctggaggtgcgcggttgagcgtggagtcgtgggactgtgcatggaggtgtggggctccccgcacctgagcacccccgcataacaccccagtcccctctggaccctcttcaaggaagttcagttctttattgggctctccactacactgtgagtgccctcctcaggcgagagaacgttctggctcttctcttgccccttcagcccctgttaatcggacagagatggcagggctgtgtctccacggccggaggctctcatagtcagggcacccacagcggttccccacctgccttctgggcagaatacactgccacccataggtcagcatctccactcgtgggccatctgcttaggttgggttcctctggattctggggagattgggggttctgttttgatcagctgattcttctgggagcaagtgggtgctcgcgagctctccagcttcctaaaggtggagaagcacagacttcgggggcctggcctggatccctttccccattcctgtccctgtgcccctcgtctgggtgcgttagggctgacatacaaagcaccacagtgaaagaacagcagtatgcctcctcactagccaggtgtgggcgggtgggtttcttccaaggcctctctgtggccgtgggtagccacctctgtcctgcaccgctgcagtcttccctctgtgtgtgctcctggtagctctgcgcatgctcatcttcttataagaacaccatggcagctgggcgtagtggctcacgcctataatcccagcactttgggaggctgaggcaggcagatcacgaggtcaggagttcgagaccaacctgaccaacagggtgaaacctcgtctctactaaaaatacaaaaatacctgggcgtggtggtggtgcgcgcctataatcccagctactcaggaggctgaggcaggagaatcgcttgaacccaggaggcagaggttgcagtgagccgagatagtgccactgcactccagtttgagcaacagagcgagactctgtctcaaaacaaaataaaacaaaccaaaaaaacccaccatggcttagggcccagcctgatgacctcatttttcacttagtcacctctctaaaggccctgtctccaaatagagtcacattctaaggtacgggggtgttggggaggggggttagggcttcaacatgtgaatttgcggggaccacaattcagcccaggaccccgctcccgccacccagcactggggagctggggaagggtgaagaggaggctgggggtgagaaggaccacagctcactctgaggctgcagatgtgctgggccttctgggcactgggcctcggggagctagggggctttctggaaccctgggcctgcgtgtcagcttgcctcccccacgcaggcgctctccacaccattgaagttcttatcacttgggtctgagcctggggcatttggacggagggtggccaccagtgcacatgggcaccttgcctcaaaccctgccacctccccccacccaggatcccccctgcccccgaacaagcttgtgagtgcagtgtcacatcccatcgggatggaaatggacggtcgggttaaaagggacgcatgtgtagaccctgcctctgtgcatcaggcctcttttgagagtccctgcgtgccaggcggtgcacagaggtggagaagactcggctgtgccccagagcacctcctctcatcgaggaaaggacagacagtggctcccctgtggctgtggggacaagggcagagctccctggaacacaggagggagggaaggaagagaacatctcagaatctccctcctgatggcaaacgatccgggttaaattaaggtccggccttttcctgctcaggcatgtggagcttgtagtggaagaggctctctggaccctcatccaccacagtggcctggttagagaccttggggaaataactcacaggtgacccagggcctctgtcctgtaccgcagctgagggaaactgtcctgcgcttccactggggacaatgcgctccctcgtctccagactttccagtcctcattcggttctcgaaagtcgcctccagaagccccatcttgggaccaccgtgactttcattctccagggtgcctggccttggtgctgcccaagaccccagaggggccctcactggcctttcctgccttttctcccattgcccacccatgcacccccatcctgctccagcacccagactgccatccaggatctcctcaagtcacataacaagcagcacccacaaggtgctcccttccccctagcctgaatctgctgctccccgtctggggttccccgcccatgcacctctgggggcccctgggttctgccataccctgccctgtgtcccatggtggggaatgtccttctctccttatctcttcccttcccttaaatccaagttcagttgccatctcctccaggaagtcttcctggattcccctctctcttcttaaagcccctgtaaactctgaccacactgagcatgtgtctgctgctccctagtctgggccatgagtgagggtggaggccaagtctcatgcatttttgcagcccccacaagactgtgcaggtggccggccctcattgaatgcggggttaatttaactcagcctctgtgtgagtggatgattcaggttgccagagacagaaccctcagcttagcatgggaagtagcttccctgttgaccctgagttcatctgaggttggcttggaaggtgtgggcaccatttggcccagttcttacagctctgaagagagcagcaggaatggggctgagcagggaagacaactttccattgaaggcccctttcagggccagaactgtccctcccaccctgcagctgccctgcctctgcccatgaggggtgagagtcaggcgacctcatgccaagtgtagaaaggggcagacgggagccccaggttatgacgtcaccatgctgggtggaggcagcacgtccaaatctactaaagggttaaaggagaaagggtgacttgacttttcttgagatattttgggggacgaagtgtggaaaagtggcagaggacacagtcacagcctcccttaaatgccaggaaagcctagaaaaattgtctgaaactaaacctcagccataacaaagaccaacacatgaatctccaggaaaaaagaaaaagaaaaatgtcatacagggtccatgcacaagagcctttaaaatgacccgctgaagggtgtcaggcctcctcctcctggactggcctgaaggctccacgagcttttgctgagacctttgggtccctgtggcctcatgtagtacccagtatgcagtaagtgctcaataaatgtttggctacaaaagaggcaaagctggcggagtctgaagaatccctcaaccgtgccggaacagatgctaacaccaaagggaaaagagcaggagccaagtcacgtttgggaacctgcagaggctgaaaactgccgcagattgctgcaaatcattgggggaaaaacggaaaacgtctgttttcccctttgtgcttttctctgttttcttctttgtgcttttctctgttttcaggatttgctacagtgaacatagattgctttggggccccaaatggaattattttgaaaggaaaatgcagataatcaggtggccgcactggagcaccagctgggtaggggtagagattgcaggcaaggaggaggagctgggtggggtgccaggcaggaagagcccgtaggccccgccgatcttgtgggagtcgtgggtggcagtgttccctccagactgtaaaagggagcacctggcgggaagagggaattcttttaaacatcattccagtgcccgagcctcctggacctgttgtcatcttgaggtgggcctcccctgggtgactctagtgtgcagcctggctgagactcagtggccctgggttcttactgctgacacctaccctcaacctcaaccactgcggcctcctgtgcaccctgatccagtggctcattttccactttcagtcccagctctatccctatttgcagtttccaagtgcctggtcctcagtcagctcagacccagccaggccagcccctggttcccacatcccctttgccaagctcatccccgccctgtttggcctgcgggagtgggagtgtgtccagacacagagacaaaggaccagcttttaaaacattttgttggggccaggtgtggtggctcacacctaatcccaacacctggggaggccaaggcagaaggatcacttgagtccaggagttcaagaccagcctgggcaacatagggagaccctgtctctacaattttttttttaattagctgggcctgttggcactctcctgtagttccagctactctagaggctgaggtgggaggactgcttgagcctgggaggtcagggctgcaatgagccatgttcacaccactgaacgccagcctgggcgagaccctgtatcaaaaaagtaaagtaaaatgaatcctgtacgttatattaaggtgccccaaattgtacttagaaggatttcatagttttaaatacttttgttatttaaaaaattaaatgactgcagcatataaattaggttcttaatggaggggaaaaagagtacaagaaaagaaataagaatctagaaacaaagataagagcagaaataaaccagaaaacacaaccttgcactcctaacttaaaaaaaaaaatgaagaaaacacaaccagtaaaacaacatataacagcattaagagctggctcctggctgggcgcggtggcgcatgcctgtaatcccaacactttgggaggccgatgctggaggatcacttgagaccaggagttcaaggttgcagtgagctatgatcataccactacaccctagcctgggcaacacagtgagactgagactctattaaaaaaaaaatgctggttccttccttatttcattcctttattcattcattcagacaacatttatggggcacttctgagcaccaggctctgtgctaagagcttttgcccccagggtccaggccaggggacaggggcaggtgagcagagaaacagggccagtcacagcagcaggaggaatgtaggatggagagcttggccaggcaaggacatgcagggggagcagcctgcacaagtcagcaagccagagaagacaggcagacccttgtttgggacctgttcagtggcctttgaaaggacagcccccacccggagtgctgggtgcaggagctgaaggaggatagtggaacactgcaacgtggagctcttcagagcaaaagcaaaataaacaactggaggcagctggggcagcagagggtgtgtgttcagcactaaggggtgtgaagcttgagcgctaggagagttcacactggcagaagagaggttggggcagctgcaagcctctggacatcgcccgacaggacagagggtggtggacggtggccctgaagagaggctcagttcagctggcagtggccgtgggagtgctgaagcaggcaggctgtcggcatctgctggggacggttaagcaggggtgagggcccagcctcagcagcccttcttggggggtcgctgggaaacatagaggagaactgaagaagcagggagtcccagggtccatgcagggcgagagagaagttgctcatgtggggcccaggctgcaggatcaggagaactggggaccctgtgactgccagcggggagaagggggtgtgcaggatcatgcccagggaagggcccaggggcccaagcatgggggggcctggttggctctgagaagatggagctaaagtcactttctcggaggatgtccaggccaatagttgggatgtgaagacgtgaagcagcacagagcctggaagcccaggatggacagaaacctacctgagcagtggggctttgaaagccttggggcggggggtgcaatattcaagatggccacaagatggcaatagaatgctgtaactttcttggttctgggccgcagcctgggtggctgcttccttccctgtgtgtattgatttgtttctcttttttgagacagagtcttgctgggttgcccaggctggagtgcagtggtgcgatcatagctcactgcagccttgaagtcctgagctcaagagatccttccacctcagcctcctgagtagttgggaccacaggcttgcaccacagtgcccaactaatttcttatattttttgtagagatggggtttcactgtgtcgcccaggatggtcttgaactcctgggctcaagtgatcctcctgcctcagcctcgcaaattgctgggattacaggtgtgagccaccatgcccgaccttctctttttaagggcgtgtgtgtgtgtgtgtgtgtgtgggcgcactctcgtcttcaccttcccccagccttgctctgtctctacccagtcacctctgcccatctctccgatctgtttctctctccttttacccctctttcctccctcctcatacaccactgaccattatagagaactgagtattctaaaaatacattttatttatttattttgagacagagtctcactctgtcacccaggctggagtgcagtggtgcaatctcggctcactgcaacctccgcctcccaggttgaagcaactctcctgcctcagcctccctagtagctgggattacaagcacacaccaccatgcctagcaaatttttatatttttagtagaggaggagtgtcaccatgtttgccaagctggtctcaaactcctggcctcaggtgatctgcctaccttggtctcccaaagtgctgggattacaggtgtgagccaccacgcctgcccttaaaaatacattatatttaatagcaaagccccagttgtcactttaaaaagcatctatgtagaacatttatgtggaataaatacagtgaatttgtacgtggaatcgtttgcctctcctcaatcagggccagggatgcaggtgagcttgggctgagatgtcagaccccacagtaagtggggggcagagccaggctgggaccctcctctaggacagctctgtaactctgagaccctccaggcatcttttcctgtacctcagtgcttctgaaaaatctgtgtgaatcaaatcattttaaaggagcttgggttcatcactgtttaaaggacagtgtaaataattctgaaggtgactctaccctgttatttgatctcttctttggccagctgacttaacaggacatagacaggttttcctgtgtcagttcctaagctgatcaccttggacttgaagaggaggcttgtgtgggcatccagtgcccaccccgggttaaactcccagcagagtattgcactgggcttgctgagcctggtgaggcaaagcacagcacagcgagcaccaggcagtgctggagacaggccaagtctgggccagcctgggagccaactgtgaggcacggacggggctgtggggctgtggggctgcaggcttggggccagggagggagggctgggctctttggaacagccttgagagaactgaacccaaacaaaaccagatcaaggtctagtgagagcttagggctgctttgggtgctccaggaaattgattaaaccaagtggacacacacccccagccccacctcaccacagcctctccttcagggtcaaactctgaccacagacatttctcccctgactaggagttccctggatcaaaattgggagcttgcaacacatcgttctctcccttgatggtttttgtcagtgtctatccagagctgaagtgtaatatatatgttactgtagctgagaaattaaatttcaggattctgatttcataatgacaaccattcctcttttctctcccttctgtaaatctaagattctataaacggtgttgacttaatgtgacaattggcagtagttcaggtctgctttgtaaatacccttgtgtctattgtaaaatctcacaaaggcttgttgccttttttgtggggttagaacaagaaaaagccacatggaaaaaaaatttcttttttgtttttttgtttgcttgtttttttgagacagagtttcactctgtcgcccaggctggagtgcagtggtgcgatctccgcccactgcaagctccacctcccgggttcatgctattctcctgtctcagcctcccaagtagctgggactgcaggtgcccgccaccacacctggctaatttttttgtatttttagtagagacggggtttcaccgtgttagccaggatggtctcaatctcctgacctcgtcatctgcctgcctcggcctcccaaagtgctgagattacaggcgtgagccaccgtgcccggccagaaaaaaacatttctaagtatgtggcagatactgaattattgcttaatgtcctttgattcatttgtttaatttctttaatggattagtacagaaaacaaagttctcttccttgaaaaactggtaagttttctttgtcagataaggagagttaaataacccatgacatttccctttttgcctcggcttccaggaagctcaaagttaaatgtaatgatcactcttgtaattatcagtgttgatgcccttcccttcttctaatgttactctttacattttcctgctttattattgtgtgtgttttctaattctaagctgttcccactcctttctgaaagcaggcaaatcttctaagccttatccactgaaaagttatgaataaaaaatgatcgtcaagcctacaggtgctgaggctactccagaggctgaggccagaggaccacttgagcccaggaatttgagacctgggctgggcagcatagcaagactctatctccattaaaactatttttttttatttaaaaaataatccgcaaagaaggagtttatgtgggattccttaaaatcggagggtggcatgaattgattcaaagacttgtgcagagggcgacagtgactccttgagaagcagtgtgagaaagcctgtcccacctccttccgcagctccagcctgggctgaggcactgtcacagtgtctccttgctggcaggagagaatttcaacattcaccaaaaagtagtattgtttttattaggtttatgaggctgtagccttgaggacagcccaggacaactttgttgtcacatagatagcctgtggctacaaactctgagatctagattcttctgcggctgcttctgacctgagaaagttgcggaacctcagcgagcctcacatggcctccttgtccttaacgtggggacggtgggcaagaaaggtgatgtggcactagagatttatccatctctaaaggaggagtggattgtacattgaaacaccagagaaggaattacaaaggaagaatttgagtatctaaaaatgtaggtcaggcgctcctgtgttgattgcagggctattcacaatagccaagatttggaagcaacccaagtgtccatcaacagacaaatggataaagaaaatgtggtgcatatacacaatggaatactattcagccatgaaaaagaatgagaatctgtcatttgaaacaacatggatggaactggaggacattatgttaagtgaaataagccagacagaaggacagacttcacatgttctcacacatttgtgggagctaaaaattaaactcatggagatagagagtagaaggatggttaccagaggctgaggagggtggaggggagcagggagaaagtagggatggttaatgggtacaaaaacgtagttagcatgcatagatctagtattggatagcacagcagggtgacgacagccaacagtaatttatagtacatttaaaaacaactaaaagagtgtaactggactggctaacatggtgaaaccccgtctctactaaaaatacaaaaattagctgggcacggtggctcacgcctgtaatcccagcactttgggaggccgaggcgggccgatcacgaggtcaggagatcgagaccatcctagctaacatggtgaaaccccgtctctactacaaatacaaaaaaaagaaaaaattagccgggcatggtggtgggcgcctgtagtcccagctactcgggaggctgaggcaggagaatggcgtgaacccgggaggcggagcttgcagtgagccgagatcgcgccactgcactccagcctgggcgacaaggcaagattctatctcaaaaaaataaaaataaaataaaataaaataataaaataaaataaaataaaataaaataaaataaataaaataaaatgtataattggaatgtttataacacaagaaatgataaatgcttgaggtgatagataccccattcaccgtgatgtgattattgcacaatgtatgtctgtatctaaatatctcatgtaccccacaagtatatacacctactatgtacccatataaatttaaaattaaaaaattataaaacaaaaataaataagtaaattaaaatgtaggctggacaccgtggttcacgcctgtaatcccagtgctttgtgaggctgaggtgagagaatcacttgagcccaggagtttgagaccggcctgggtgacatagcgagaccccatcatcacaaagaatttttaaaaattagctgggcgtggtagcacataccggtagttccagctacttgggagaccgaggcaggaggattgcttgagcccaggagtttaaggctgcagtgagctacgatggcgccactgcattccagcctgggtgacagagtgagagcttgtctctattttaaaaataataaaaagaataaataaaaataaattaaaatgtaaatatgtgcatgttagaaaaaatacacccatcagcaaaaagggggtaaaggagcgatttcagtcataattggagagatgcagaataagccagcaatgcagtttcttttattttggtcaaaaaaaataagcaaaacaatgttgtaaacacccagtgctggcagcaatgtggtgaggctggctctctcaccagggctcacagggaaaactcatgcaacccttttagaaagccatgtggagagttgtaccgagaggttttagaatatttataactttgacccagaaattctattctaggactctgtgttatgaaaataacccatcatatggaaaaagctcctttcagaaagaggttcatgggaggctgtttgtattttttttttctttgcatcaaatccagctcctgcaggactgtttgtattattgaagtacaaagtggaatcaatacaaatgttggatagcaggggaacaatattcacaaaatggaatgggacatagtattaaacatagtgcttctgatgaccgtagaccatagacaatgcttaggatatgatatcacttcttttgttgttttttgtattttgagacgaagtctcattctgtcacccaggctggagttcagtggcgccatctcagctcactgcaacctccatctcccgggttcaagctattctccttcctcaacctcccgagtagctgggttgcgcaccaccatgcctggctaacttttgtatttttagtacagacggggtttcaccacgttggccaggctgctcttgaactcctgacgtcaggtgatccaccagccttgacctcccaaagtgctaggattacaggagccactgtacccagcctaggatatgatatcacttcttagagcaagatacaaaattgcatgtgcacaataattctaccaagtataggtatacaggggtagttatatataaatgagacttcaaggaaatacaacaaaatgcaatcgtgattgtgttagggtggtaagaaaacggtttttgctttgatgagctctgttttttaaaatcgttatattttctaataaaaatacatagtcttttgaaggaacataaaagattatgaagaaatgagttagatattgattcctattgaagattcagacaagtaaaattaaggggaaaaaaaacgggatgaaccagaagtcaggctggagttccaaccccagatccgacagcccaggctgatggggcctccagggcagtggtttccacccagcattctcaaaagagccactgaggtctcagtgccattttcaagatttcggaagcggcctgggcacggctggtccttcactgggatcaccacttggcaattatttacacctgagacgaatgaaaaccagagtgctgagattacaggcatggtggcttacgcttgtaatcggctttgggaagccgaggtgggctgattgcttgagcccaggagtttcaaactatcctggacaacatagcatgacctcgtctctacaaaaaatacaaaaaatttgccaggtgtggtggcatgtgcctgtggtcccagctacttgggaggctgaagtaggagaatcccctgagccctgggaagtcgaggctgcactgagccgtgatggtgtcactgcactccagcctgggtgacaaagtgagaccctatctcacaaagaaaaaaaacaaaacaaaaaacccaaagcacactgtttccactgtttccagagttcctgagaggaaaggtcaccgggtgaggaagacgttctcactgatctggcagagaaaatgtccagtttttccaactccctaaaccatggttttctatttcatagttcttaggcaaattggtaaaaatcatttctcatcaaaacgctgatattttcacacctccctggtgtctgcagaaagaaccttccagaaatgcagtcgtgggagacccatccaggccacccctgcttatggaagagctgagaaaaagccccacgggagcatttgctcagcttccgttacgcacctagtggcattgtgggtgggagagggctggtgggtggatggaaggagaaggcacagcccccccttgcagggacagagccctcgtacagaagggacaccccacatttgtcttccccacaaagcggcctgtgtcctgcctacggggtcagggcttctcaaacctggctgtgtgtcagaatcaccaggggaacttttcaaaactagagagactgaagccagactcctagattctaattctaggtcagggctaggggctgagattgtaaaaatccacaggtgattctgatgcccggcaggcttgagaacagccgcagggagttctctgggaatgtgccggtgggtctagccaggtgtgagtggagatgccggggaacttcctattactcactcgtcagtgtggccgaacacatttttcacttgacctcaggctggtgaacgctcccctctggggttcaggcctcacgatgccatccttttgtgaagtgaggacctgcaatcccagcttcgtaaagcccgctggaaatcactcacacttctgggatgccttcagagcagccctctatcccttcagctcccctgggatgtgactcgacctcccgtcactccccagactgcctctgccaagtccgaaagtggaggcatccttgcgagcaagtaggcgggtccagggtggcgcatgtcactcatcgaaagtggaggcgtccttgcgagcaagcaggcgggtccagggtggcgtgtcactcatccttttttctggctaccaaaggtgcagataattaataagaagctggatcttagcaacgtccagtccaagtgtggctcaaaggataatatcaaacacgtcccgggaggcggcagtgtgagtaccttcacacgtcccatgcgccgtgctgtggcttgaattattaggaagtggtgtgagtgcgtacacttgcgagacactgcatagaataaatccttcttgggctctcaggatctggctgcgacctctgggtgaatgtagcccggctccccacattcccccacacggtccactgttcccagaagccccttcctcatattctaggagggggtgtcccagcatttctgggtcccccagcctgcgcaggctgtgtggacagaatagggcagatgacggaccctctctccggaccctgcctgggaagctgagaatacccatcaaagtctccttccactcatgcccagccctgtccccaggagccccatagcccattggaagttgggctgaaggtggtggcacctgagactgggctgccgcctcctcccccgacacctgggcaggttgacgttgagtggctccactgtggacaggtgacccgtttgttctgatgagcggacaccaaggtcttactgtcctgctcagctgctgctcctacacgttcaaggcaggagccgattcctaagcctccagcttatgcttagcctgcgccaccctctggcagagactccagatgcaaagagccaaaccaaagtgcgacaggtccctctgcccagcgttgaggtgtggcagagaaatgctgcttttggcccttttagatttggctgcctcttgccaggagtggtggctcgtgcctgtaattccagcactttgggagactaaggcgggaggttcgcttgagcccaggagttcaagaccagcctgggcaacaatgagacccctgtgtctacaaaaagaattaaaattagccaggtgtggtggcacgcacctgtagtcccagctacttgggaggctgaggtgggaggattgcctgagtccgggaggcggaagttgcaaggagccatgatcgcgccactgcacttcaacctaggcaacagagtgagactttgtctcaaaaaacaatcatataataattttaaaataaatagatttggcttcctctaaatgtccccggggactccgtgcatcttctgtggagtgtctccgtgagattcgggactcagatcctcaagtgcaactgacccacccgataagctgaggcttcatcatcccctggccggtctatgtcgactgggcacccgaggctcctctcccaccagctctcttggtcagctgaaagcaaactgttaacaccctggggagctggacgtatgagacccttggggtgggaggcgttgatttttgagagcaatcacctggccctggctggcagtaccgggacactgctgtggctccggggtgggctgtctccagaaaatgcctggcctgaggcagccacccgcatccagcccagagggtttattcttgcaatgtgctgctgcttcctgccctgagcacctggatcccggcttctgccctgaggccccttgagtcccacaggtagcaagcgcttgccctgcggctgctgcatggggctaactaacgcttcctcaccagtgtctgctaagtgtctcctctgtctcccacgccctgctctcctgtccccccagtttgtctgctgtgaggggacagaagaggtgtgtgccgcccccacccctgcccgggcccttgttcctgggattgctgttttcagctgtttgagctttgatcctggttctctggcttcctcaaagtgagctcggccagaggaggaaggccatgtgctttctggttgaagtcaagtctggtgccctggtggaggctgtgctgctgaggcggagctggggagagagtgcacacgggctgcgtggccaacccctctgggtagctgatgcccaaagacgctgcagtgcccaggacatctgggacctccctggggcccgcccgtgtgtcccgcgctgtgttcatctgcgggctagcctgtgacccgcgctgtgctcgtctgcgggctagcctgtgtcccgcgctctgcttgtctgcggtctagcctgtgacctggcagagagccaccagatgtcccgggctgagcactgccctctgagcaccttcacaggaagcccttctcctggtgagaagagatgccagcccctggcatctgggggcactggatccctggcctgagccctagcctctccccagcctgggggccccttcccagcaggctggccctgctccttctctacctgggacccttctgcctcctggctggaccctggaagctctgcagggcctgctgtccccctccctgccctccaggtatcctgaccaccggccctggctcccactgccatccactcctctcctttctggccgttccctggtccctgtcccagcccccctccccctctcacgagttacctcacccaggccagagggaagagggaaggaggccctggtcataccagcacgtcctcccacctccctcggccctggtccaccccctcagtgctggcctcagagcacagctctctccaagccaggccgcgcgccatccatcctccctgtcccccaacgtccttgccacagatcatgtccgccctgacacacatgggtctcagccatctctgccccagttaactccccatccataaagagcacatgccagccgacaccaaaataattcgggatggttccagtttagacctaagtggaaggagaaaccaccacctgccctgcaccttgttttttggtgaccttgataaaccatcttcagccatgaagccagctgtctcccaggaagctccagggcggtgcttcctcgggagctgactgataggtgggaggtggctgcccccttgcaccctcaggtgaccccacacaaggccactgctggaggccctggggactccaggaatgtcaatcagtgacctgccccccaggccccacacagccatggctgcatagaggcctgcctccaagggacctgtctgtctgccactgtggagtccctacagcgtgccccccacaggggagctggttctttgactgagatcagctggcagctcagggtcatcattcccagagggagcggtgccctggaggccacaggcctcctcatgtgtgtctgcgtccgctcgagcttactgagacactaaatctgttggtttctgctgtgccacctacccaccctgttggtgttgctttgttcctattgctaaagacaggaatgtccaggacactgagtgtgcaggtgcctgctggttctcacgtccgagctgctgaactccgctgggtcctgcttactgatggtctttgctctagtgctttccagggtccgtggaagcttttcctggaataaagcccacgcatcgaccctcacagcgcctcccctctttgaggcccagcagataccccactcctgcctttccagcaagatttttcagatgctgtgcatactcatcatattgatcacttttttcttcatgcctgattgtgatctgtcaatttcatgtcaggaaagggagtgacatttttacacttaagcgtttgctgagcaaatgtctgggtcttgcacaatgacaatgggtccctgtttttcccagaggctcttttgttctgcagggattgaagacactccagtcccacagtccccagctcccctggggcagggttggcagaatttcgacaacacatttttccaccctgactaggatgtgctcctcatggcagctgggaaccactgtccaataagggcctgggcttacacagctgcttctcattgagttacacccttaataaaataatcccattttatcctttttgtctctctgtcttcctctctctctgcctttcctcttctctctcctcctctctcatctccaggtgcaaatagtctacaaaccagttgacctgagcaaggtgacctccaagtgtggctcattaggcaacatccatcataaaccaggtagccctgtggaaggtgagggttgggacgggagggtgcagggggtggaggagtcctggtgaggctggaactgctccagacttcagaaggggctggaaaggatattttaggtagacctacatcaaggaaagtgttgagtgtgaaacttgcgggagcccaggaggcgtggtggctccagctcgctcctgcccaggccatgctgcccaagacaaggtgaggcgggagtgaagtgaaataaggcaggcacagaaagaaagcacatattctcggccgggcgctgtggctcacgcctgtaattccagcactttgggaggccaaggtgggtggatcatgaggtcaggagattgagaccatcctggctaacacagtgaaaccccgtctctactaaaaatacaaaaaattagccgggcgtggtggtgggcgcctgtagtcccagctactccggaggctgaggcaggaaaatggcgtgaacccggaaggcggagcttgcagtgagcggagtgagcagagatcgcgccactgcactccagcctgggcgacagagcgagactccgtctcaaaaaaaaaaagcacatgttctcgcttctttgtgggatccaggagatagagaatagaaggatggttaccagaggctgggaagggtagtgaggggatggtggggggatggtcaatgggtacaaaaaaaatagaataagacctagtatttgatagtgcaacagggtgactatagtcaataataatttaattgtacatttaaaaataactaaaagatagccgggtgcagtggcttacgtctgtaatcccagtactttgggaggctgaggtgggcgtttgagaccagcctggccaacatggtgaaaccccatctctactaaaaatacaaaaattagccaggcatggtggcgggcgcctgtaatcccagctactcgggaggctgaggcaggagaatcacttgaacctgggaggcagaggttgcagtgagccgagatcttgccactgcactccagcctgggtgacagtgaaactccgtctcaaaaataaaaataaaaatacagctgggcacggtggctcacgcctgtaatcccagcactttgggaggccgaggcgagcggatcacaaggtcaggagatatagaccatcctggctaacacggtgaaacccggtctctactaaaaatacaaaaaattagccaggcgtggtggcaggtgcctatagtcccagctactcacaaggctgaggcaggagaatggcatgaacctgggaggcggagcttgcagtgagccgagattgtgccactgcactccagcctgggcgagagagtgagactccgtctcaaaacaaaaacaaaaacaaaaacaaaaacaaacacacaacaaaaacctaaaagaatataaatggattgtttgtaacacaaaggacaaatgtttgaggggatggataccccattttccatgatgtgattattatacattgtgtgtctgtatcaaaacatctcatgagccccataaatatatacacctaactatgtacccacaaaaattaaaaaaatatattttttaaggtgaagagggaggcgagatgctggccttaacccctaacccgttgttctccctgcaagctgtccacagggcctctcagactcgaggttcagctatatggatgcatgagcttggtccccagccaacatgggagacacttcaccatcggcagcagctacagcacaggaaccctgggtcactgccatgtcccctctgtgactttgtttaaacagaaaatgatgctctgggccggctgtggtggcccacacctataatcccagcaccttgggaggcgggggtgggcagattgcctgaggtcaggagttggagatcagcctggccgacatggcgaaaccccatgtctactaaaaatacaaaaactagccaggcatggtggcacatgcctgtaatcccagctacttgggaggctgaagcaggagaatcacttgaacccaggaggcagaggctgagtgagccaagatcgtgccaatgcactccagcttgggtgagggagtgagactccgtctcaaaaaaaaaaaaaaagaaagaaaaagaaaagaaagtgatcctactggaaccatgcttactcccctccccacctcacactgtgtagaaattagtgctgtcggccaggcgcggtggctcatgcctgtaatcgcagcactttgggaggccaaggcaggcggatcacgaggtcaggagatcaagaccatcctggctaacacagtgaaaccctgtctctactaaaaatacaaaaaattagccgggcatggtggcaggcacctgtagtcccaactacttgggaggctgaggcaggagaatggcatgaacctgggaggcggagcttgcagtgagccaagatcgcgccactgcataccagcctaggtgacagagtgagactcagcaaaaaaagaaagaaagaaagaaagaaatcagtgctgtctatacttctttctgcagtgatggaaatattctgtatctgtgctgtccagtatagtagccactagctacatgtggcacttgaaacatggctggtacagttgaggaagagtggctgccatatcggacgacacagctatagattctgtcaccccaccccgagagtccagagcggggacttctgccttaggccctattcagggctgatttttacttgaacccttactgtgggaagagaaggccatgagaagttcagtctagaatgtgactccttattttctggctcccttggacactttgtgggatttagtctccctgtggaaagtattccacaagtggtgccactaccccagctgtgagagcagctgggagctgcttttgtcatctttccctggaaagtcctgtgggctgtctcttcctcatgccttgtcccatgcttgggcatggtgtcaagcgtcaggagggagaaagggtccttatttatttatttagagagggacccttcttctgttcccaggctggagtgcagtggtgcgatctcggctcactgcaacctccgcctcctgggttcaagtgattctcctgcctcagcctcctgagtagctgagattacaggcacatgccaacatgcccggctaattttttttttttttttttttttttttttttttttttttttgagatggagttgtactctcattgcccaggctggaatgtaatggcacaatctcggctcactgcaacctccacctcctggattcaagcaattctcctgtctcagcttcccaagtagctgggattacaggtgcccgccaccatgctcaactaatttttgtattttttttttagtagagacgaggtttcaccatgttggtcagactggtctcaaactcctgacctcaggtgatccacctgcctcggcctcccaaagtgctaggattacaggcatgagccaccacgcccggcctgaaagggttcttatttagtgtgcattttgacattcaatttaattccaaggtcttgtggggtcatggtttacaggatgttgatatagaaaagacttcacttaatgggccgggcgcagtggctcatgcctgtaatcccagcactttgggaggccgaggcaggcagatcaggaggtcaggagattgagaccatcctggctaacacagtgaaaccccatctctactgaaaatacaaaaaattagctgggcgtggtggcaggcacctgtagtcccagccactcggttggctgaggcaggagaatggcatgaacccgggaggcggagcttgcagtgagcagagaccatgccactgcactccagcctgggcgacagagcaagactctgtctcaagaaaaaaaaaaaaaaacagactttacttactggaagccaaccaatgtatatttagagtaatttttcctgggctgagctgtcatttacttttgcagtatctcaagaagaagagtttacagtgtaaatatttgatgcacactttgattatatagatgaagcaaactattttcaagagctttgcaaggacttacttgtatccaaacaccattctaaaaggagtcttacctacttctaaaggctggtctctacttggaaccacttgcttggccctggttcaagtcctgctgcaaacctggaagtcctgtcattgtcttcttccctccagagcagtggcacccaatctaatttttgctgtgccccagcagcccctggcactttgccctgtagactgcagacctcatgtaatgtatgttaagtccacagaaccacagaagatgatggcaagatgctcttgtgtgtgttgtgttctaggaggtggccaggtggaagtaaaatctgagaagcttgacttcaaggacagagtccagtcgaagattgggtccctggacaatatcacccacgtccctggcggaggaaataaaaaggtaaagggggtagggtgggttggatgctgcccttgggtatatgggcattaatcaagttgagtggacaaaggctggtccagttcccagaggaggaaaacagaggcttctgtgttgactggctggatgtgggccctcagcagcatccagtgggtctccactgcctgtctcaatcacctggagctttagcacgtttcacacctgggccccaacctggagaggctgaccaatgggtctcaggggcagctcggttgctggagtttttgtttttatttatttttatgtatttaaggcagggtctctgtattagtccattctcacactgctaataaagacatacccaagactgggtaatttataaaggaaagaggtttaatggactcacagttccacatggctggggaggcctcaaaatcatggcggaaggcaaaggagaagcaaaggcatttcttacatggcgacaggcaagagagcgtgtgcaggggaactcccatttataaaaccatcagacctcatgagatttattcactatcatgagaacagcatgggaaagacccgcccccatgattcagttacctcccactgggtccctcccatgacacatggaattatgggagctacaattcaagatgagatttgggtggggacacagccaaaccatatcagtctccctctgtcatccaggctggagtgcactggcatgatctcggctcactgcagcctctacctccctgggtcaggtgatcttcccacctcagcctcccaggtagctggaactacaggtacctgccactatgcctggctaaatattttgtatttcctgtggagacgaggttttgccacgttgcccaggctggtcttgaactcctgaggtcaagcaatatgcccacctcggcctcccaaggtgctgggattacaggtgtgagccacagtgctcggcctaagtcactgcagtttttaaagctcccaggtgattcttcagtgcagtcaaaagtgagaactggctgggtgcggtggctcatgcctgtaatcccagcaccttgggaggcgaaggtgggcagatggcttgaggtcaggagttcaagaccagcctggccaacatggtaaaaccccatctctactaaaaatacaaaagttagctgggtgtggtggtgcgtgcctgtaatcccagctacttgggaggctgaggcatgagaattgcttgaacccaggggacagaggttgtagtgagccgagatcgtgccactgcactccagcctgggcaacagagtgagattccatctcacaaaaaaaaaaaaaagcgagaaccactgtcctaggccctgatgtttgcaggcaactaaaaaaggaagtggacatccccagtcagctgtggcgcaccaagaacaagtcatgggaacataacctaattttctaaatgggttactaggcacttagagcaaaacaatgatgccgaaatcctgatttcagcaaagcctctgcctgcctgtcttggaagtatccacatgaggctgctggggccttggtgtccccagcagtttctagtctctaggtcttgctgtgggtgtctgtgcagtgagggtgtgtgtggcgctgggtgagctctgtctaggcctggcacaggatgcggtctggtagctgctgcttctcttctgcagaagcgcagccaagcaccctctggggtttcaggcccacacccagcctgaagttctgggagtggctcactttccaaccttcagggtctcccagcagctgactggggagtggtggagggaaaagggattgtattagtccgttttcacgccgctgatgaagacatacccgatactgggcagtctaaaagatagaggtctgatggactcacagttccacgtgactggggaggcctgacaatcatggtggaaggtgaaaggcttgtctcacacggtggcagacaagagaaaagagcttgtgcaggggaactcccctttataaaaccatcagatctcgggagacttattcactatcatgagaacagcacgggaaagaccctcctctatgattcaattacctcccaccaggtccctcccacaacatgtaggaattgtgggaactacaattcaagatgacatttgggtggggacacagccaaaccatatcagggcgtcccagaaagggtatagggtctgagacccaagtcagcatgagaaagtatgcttctcatggtggcccagttgggtggaagtggcagccgggccgtctttccaccaggccactcaagtagcagctgagagacccctgccctggccagtccccgccctcccctcttgccactgcctctggttctgaacagatgggcaccctcatcttgtatttgtgattaatgtctaacaatgtagttttgtgagaagggtttgctgatacagccttgctgcagatgctgcgaactgtggcctggggcagaccttacctccagacacgccctgaggcaggggagggcactggcccgtagctggccgagagctctcgggttgcgcgacagggatacttttcagcggctgggtcgctatccaaagtgagaaaacgaggagggaccaggaggctgtccgcctcaagagatgtgggggccaggtccagttatctggggaagcagtaagcttctctgctgtttctaaccccaggcctcccctggtctaaggcagggcctcccagcctcggggcactttaaagatatctgggcctggccccatccccacagtctgactgagtgggtctggatagggcctgagcattggtgatttcctgggtgaaaggaggcccctcacagtctctggaagcttctctgtgttaggaaaagctctgggcttgactctgctttgaaagtcaagatccgcaaatcctctcagcctcagtttctccttcagcaagatgaaatggaaatgctgtacctacgtcccggggtggttgtgagacccaaaaaagacaatgttctggaaggttcctggtgcgttgcagtcctctaagaacctgagttagagccacgctgagtctcagcttcttggctccttctgtttcaaactcgtccatgtgatagctcaggaagggtaggcagggccctgccccctactcagaaaacaccatcctggtcctggggatccccgcagcattagtcccctgttttcccagtgtattgagaaaaattgctaacaagcagtggggcacaccaccagcctcctgggttcctttcagtttggggatttttggacattcccaggaatgtcttaaaaaacacttcaaaaaacattaacataaatatttttatcaaagcctgtattaaatggtctttcaagaaaatacagtaacaggtcaggcatggtggctcatgcctgtaaccccagcactttgggaggccaaggcaggcagatcacctgaaatcaggagttcaagaccaacctggccaacacagccaaatcccatctctacaaaaaatacaaaaattagctgggtgtggtggcacacacctgtagtcccagctacttgggaggccgaggcaggagaattgcttgatcccggaggcggaggttgcagtgagccgagatcgtgccactgcactccagcgtgggtgacaaggtgaatctttgtctcaaaaaaaaaaaaaaaaaaaagataaaatacagtatacagtaatagagaacaatccttttttcaaagtagtgaccccaaatgaacaaaatatgcatctagcttaaatgcgaacctggttttctctacgcccattcaagcccctgcaataggggcccttcaccccgcatccatggactcctaaaattatatggaaaatggctgtgtgtgagtgtggatggacatgtgcacacatatttttggctttaccagatgctcaaagagcctaggacccaaaaagggctgagaatgaccgtgtcggccacttcagggtcatcaggaattgctgtgcactgctcacttctccagtgaacactttctgcttctgtgtttcctggtatcctttgggactcctggctaggtcatgtgtttctctactttcaaaagggcttcagccaggcacgatggcatgagcctgtagtcccagttgctctggaggttaaggtgggaagattgcttgagcccaggaatttgaggccagcctgggcaagtagataggtagatgattgatagatagatagatagataaatagatggatagataagtcgctagacagtcatccatccacccatccacacataaaaaggcctttgtcatgtcatgttttgtggcccacctgccagtgttgcccacagttgctgcccctccaaactcatcagtcactggcaaacaggaggaatgtgtggctcatgtctgggcatcagtggctgtgggagacatccttgatcttctccagcttctccttccacattttcctttgcaatctggcaatatctattaaaataaaatgtgcatgccttttgacctaagagcttcacttctaggacccacttacacgtgtgtgacatgatgttcatacgggtttatttatctgaggttgttcatacacaccattgcctgtaatcactaaaggcgggagcagcctacacatccatccacagaggagtagatgccttttggtacatccgtggcgacggaatactaagcagcctgtgtatctatacactcacacgtgtttgtttatgtgtggaatatctctggagggtacacaagaaacttaaaatgatcactgtctctggggagggtacctgggtgcctgggaggcaggtcagggaaggagtgggcacaggtattaccaattggaagacaataaaaacaacagctcctggccaggcgcagtggctcacgcctgtaatggcagcactctgagaggctgaggcgggcagattgcttgcgtccaggagttcaagaccagcctgggcaacatagcaaaaccccgtttctattaaaaatacaaaaaattagccaggtgtggtggcatgcacctgtaatcccagctactcgggaggctgaggtgggagaatcacctgagcctgggaggtcaaggctgcagtgaggtgagattgtgccaccgcactctagcctgggcgatagagcaagaccctgtctcaaaaacaaacaaaaaacagtccctggcactctgggccaggcctggcagggcagttggcagggctggtctttctctggcacttcatctcaccctccctcccttcctcttcttgcagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcacccctgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtgggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcggcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaataaggtctccattcatggattccaaggacaagaaagtcatatagaatgtctattttttaagttctttcccacgcacccttagataatttagctcagaacaggaaatgatagtattaataaaagctggacatcaggattaacagctctctctggggccctgaaggtgagagttctcagacttgctcatttgcagttgcttctttgtgatgctggcaaaccatcctagtcccattcaaagggcaatacaaagccttgtggctgacctcacgatgcagcactcagtttgcaagaccggcaccagtgtatgcaaacctgagaaggttggggatgaggatatgggatctttcatccctggaaatttagtccagaggcctggggctggagcagaacaccaagccaatcagcttaatgaatggcttagattcctgctaggtttgcagagctgccttctttcctttggtaccttattatagattgaggagtatttctgctaaaccaagatagggataaccagatagcatcttcatagcaatgccacaaaggaaaacaaaaacaaaacagtaatccatcatattattccttagtaactatgccaaggtcatgatactgaatccttagattgtttcaaaatactacttttctttgctcttcctgatgtgtttgccaccgcaggcagatgtttaagtaaaacagattttaactgcagctacaaaagcagcaacaggccagcaaaagagaagtgctatctcagagagcatggctttcagagccacaagagacagcctcactggctgtttcagcttgactgccatgcaaagaagagagcagagggagaaccagccccacccacttattcatcttgtacaaaaaaaaagcacctaccagcctaggctacatagtgagacactatctccacaaaaaacccacgaaaactagctgggtatggtggcacatgcctacagtcccagctactggtaaggctgtggtgggaggatctcttgaggccaggaaggagatccaggctgcagtgagccaagattgcaccactgcactccagtctggacaatcgagcaagatcccatctcaaacaataaaaaaaaaaagcgtgtaacctcctcagaagaaagatgttataatctcaggcagcaggcaagaaccaatccaggctctaagc

SEQ ID NO: 3 - protein, 833 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQEELRVPGRQRKAPERPLANEISAHVQPGPCGEASGVSGPCLGEKEPEAPVPLTASLPQHRPVCPAPPPTGGPQEPSLEWGQKGGDWAEKGPAFPKPATTAYLHTEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 4 - DNA, 18 bp, synthetic construct
  misc_feature 1..18: note = Synthetic oligonucleotide
  source 1..18: mol_type = other DNA
ccgttttcttaccaccct